(19) United States
(12) Patent Application Publication
(10) Pub. No.: US 2026/0224529 A1
(43) Pub. Date: Aug. 6, 2026

(54) SMALL MOLECULAR COMPOUND HAVING SUBSTITUTED PHENYLSPIRO[INDOLINE-3,3'-PYRROLIDINE] STRUCTURE

(71) Applicants: SHANGHAI INSTITUTE OF MATERIA MEDICA, CHINESE ACADEMY OF SCIENCES, Shanghai (CN); ZHONGSHAN INST. FOR DRUG DISCOVERY, SHANGHAI INST. OF MATERIA MEDICA, CHINESE ACADEMY OF SCIENCES, Zhongshan, Guangdong (CN)

(72) Inventors: Yujun ZHAO, Shanghai (CN); Jia LI, Shanghai (CN); Yubo ZHOU, Shanghai (CN); Jing CHENG, Shanghai (CN); Ziqin YAN, Shanghai (CN); Xilin LYU, Shanghai (CN); Shiyan ZHANG, Shanghai (CN); Feilong ZHOU, Shanghai (CN); Chen LIU, Shanghai (CN); Zhitong LIN, Shanghai (CN); Kailong JIANG, Zhongshan, Guangdong (CN); Dehua XU, Zhongshan, Guangdong (CN); Xiaobei HU, Shanghai (CN)

(21) Appl. No.: 19/120,890

(22) PCT Filed: Oct. 2, 2023
(86) PCT No.: PCT/CN2023/123063

§ 371 Date: Apr. 14, 2025

(30) Foreign Application Priority Data
Oct. 13, 2022 (CN) 202211255892.0
Aug. 10, 2023 (CN) 202311004867.X

(51) Int. Cl. A61K 31/407; A61K 31/4439; A61K 31/454; A61P 35/00; C07B 59/00; C07D 487/10

(52) U.S. Cl. CPC A61K 31/407; A61K 31/4439; A61K 31/454; A61P 35/00; C07B 59/002; C07D 487/10

(57) ABSTRACT

A small molecular compound has a substituted phenylspiro[indoline-3,3′-pyrrolidine]structure. The compound is of formula I. The compound can inhibit the protein-protein interaction between MDM2-p53 and MDMX-p53, is used as a small molecular inhibitor for the protein-protein interaction between MDM2-p53 and MDMX-p53, and is used in the preparation of a drug for preventing and/or treating diseases related to MDM2 and MDMX, especially tumors.

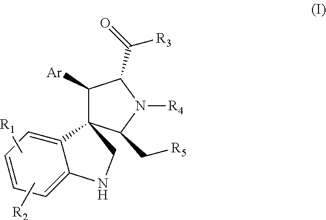

### TECHNICAL FIELD

The present invention relates to the field of pharmaceutical synthesis, specifically to a class of compounds with substituted phenylspiro[indoline-3,3′-pyrrolidine]structure, a stereoisomer, enantiomer or pharmaceutically acceptable salt thereof, and a preparation method and use thereof.

### BACKGROUND

Tumor suppressor p53 exerts anti-proliferative effects in response to various stresses, including cell growth arrest, DNA repair, and apoptosis. Mice lacking p53 develop normally but are susceptible to various tumors. The TP53 gene encoding the p53 protein is mutated or deleted in nearly 50% of human cancers, rendering cells unable to function as p53 tumor suppressors. Although p53 remains in wild-type state in the remaining 50% of human cancers, its function is inhibited by various inhibitory factors. Research has shown that two proteins are critical for p53 regulation—MDM2 and MDMX (also known as MDM4). The embryonic lethality caused by knockout of MDM2 and MDMX genes in mice can be rescued by simultaneous knockout of the TP53 gene, elucidating the role of MDM2 and MDMX as major endogenous negative regulators of p53. In vitro studies have shown that MDM2 and MDMX inhibit p53 protein's gene transcription function by interacting with p53 protein's transcriptional activation domain (TAD) through their amino-terminal p53 binding domains. Additionally, MDM2 can promote ubiquitination of MDM2 itself, MDMX and p53 protein and proteasomal degradation. Conversely, p53 specifically binds to the MDM2 P2 promoter and activates its transcription, forming an autoregulatory feedback loop—the MDM2-p53 feedback loop. MDM2 can also promote p53 protein transport out of the nucleus, preventing p53 from accessing its target DNA, thereby reducing its transcriptional capacity. As for MDMX, although it cannot function as an E3 ubiquitin ligase like MDM2, it can interact with MDM2's carboxy-terminal RING domain through its own carboxy-terminal RING domain to form stable heterodimers, promoting MDM2-mediated p53 ubiquitination.

Research has shown that compared to normal cells, cancer cells have abnormally elevated levels of MDM2 and MDMX oncogenic factors, inhibited p53-mediated gene transcription function, and reduced p53 levels. These characteristics are closely related to the overgrowth of tumor cells.

Compounds reported in the literature such as RG7112 (NCT00559533, NCT00623870, NCT01677780, NCT01164033, NCT01605526, NCT01143740, and NCT01635296), RG7388 (Ding et al., J Med Chem 2013, 56(14), 5979-83), MI-77301 (NCT01636479 and NCT01985191), AMG 232 (NCT01723020 and NCT02016729) and the like can selectively block MDM2/p53 interaction. Compounds reported in the literature such as SJ-172550 (Reed et al., J Biol Chem 2010, 285 (14), 10786-96; Bista et al., PLoS One 2012, 7(6), e37518), CTX-1 (Karan et al., Mol Cancer Ther 2016, 15 (4), 574-582), K-178 (Uesato et al., Bioorg Med Chem 2016, 24 (8), 1919-26) and the like can selectively block MDMX/p53 interaction. Compounds reported in the literature such as WK298 (Popowicz et al., Cell Cycle 2010, 9 (6), 1104-11), ATSP-7041 (Chang et al., Proc Natl Acad Sci USA 2013, 110 (36), E3445-54), RO-5963 (Graves et al., Proc Natl Acad Sci USA 2012, 109 (29), 11788-93), ALRN-6924 (Carvajal et al., Sci Transl Med 2018, 10 (436)) and the like can simultaneously inhibit MDM2/p53 and MDMX/p53 interactions. Small molecule inhibitors that block MDM2/p53 and MDMX/p53 interactions have potential for treating related diseases.

### SUMMARY OF THE INVENTION

The purpose of the present invention is to provide a small molecule inhibitor that blocks MDM2/p53 and/or MDMX/p53 interactions.

The first aspect in the present invention provides a compound of formula (I), an enantiomer, diastereomer, racemate, or pharmaceutically acceptable salt thereof,

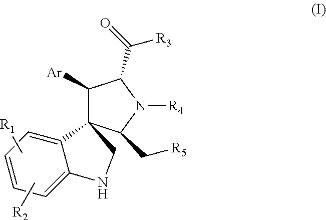

wherein, Ar is a substituted or unsubstituted phenyl, wherein said substituted refers to one or more hydrogen atoms on the phenyl being substituted by groups selected from the group consisting of halogen, deuterium, cyano, hydroxyl, amino, nitro, substituted or unsubstituted C₂-C₄ alkynyl, substituted or unsubstituted C₁-C₆ alkyl, substituted or unsubstituted C₃-C₈ cycloalkyl, substituted or unsubstituted C₁-C₄ alkoxy, substituted or unsubstituted C₂-C₁₀ acyl, substituted or unsubstituted C₁-C₄ alkylcarbonyl;
R₁ and R₂ are each independently hydrogen, deuterium, halogen, cyano, substituted or unsubstituted C₂-C₄ alkynyl, substituted or unsubstituted C₁-C₄ alkyl, substituted or unsubstituted C₃-C₈ cycloalkyl, substituted or unsubstituted C₁-C₄ alkoxy;
R₃ is

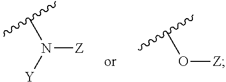

Y and Z are each independently hydrogen, —(CH₂)ₘ-substituted or unsubstituted 6-10 membered aryl, —(CH₂)ₘ-substituted or unsubstituted 5-12 membered heteroaryl, substituted or unsubstituted C₁-C₆ alkyl, —(CH₂)ₘ-substituted or unsubstituted C₃-C₈ cycloalkyl, —(CH₂)ₘ-substituted or unsubstituted 5-13 membered heterocyclyl; R₄ is substituted or unsubstituted C₁-C₈ alkyl, —(CH₂)ₘ-substituted or unsubstituted C₃-C₈ cycloalkyl, —(CH₂)ₘ-substituted or unsubstituted 4-13 membered heterocyclyl, —(CH₂)ₘ-substituted or unsubstituted 6-10 membered aryl, —(CH₂)ₘ-substituted or unsubstituted 5-12 membered heteroaryl, C₂-C₅ alkynyl, C₂-C₅ alkenyl;
m is, at each occurrence, independently 0, 1, 2, 3, or 4;
R₅ is substituted or unsubstituted C₁-C₈ alkyl, —(CH₂)ₘ-substituted or unsubstituted C₃-C₈ cycloalkyl, —(CH₂)ₘ-substituted or unsubstituted 4-12 membered heterocyclyl;
unless otherwise defined, each substituted as mentioned above independently refers to one or more hydrogen atoms on the group being substituted with groups selected from the group consisting of halogen, deuterium, cyano, hydroxyl, amino, nitro, C₁-C₄ alkylsulfonyl, C₁-C₄ alkylsulfonamidocarbonyl (C₁-C₄ alkyl-SO₂NHCO—), carboxyl, —CONH₂, C₁-C₄ alkyl, C₃-C₆ cycloalkyl, C₁-C₄ alkoxy, C₂-C₄ alkenyl, C₂-C₄ alkynyl, C₁-C₄ alkylethynyl, mono(C₁-C₄ alkyl)amino, di(C₁-C₄ alkyl)amino, C₁-C₄ alkylcarbonylamino, C₁-C₄ alkoxycarbonylamino, C₁-C₄ alkoxycarbonyl, C₁-C₄ alkyl-SO₂—, C₁-C₄ alkyl-S(O₂)—C₁-C₄ alkylene-, carboxyl-substituted C₁-C₄ alkyl, hydroxyl-substituted C₁-C₄ alkyl, C₁-C₄ alkyl-S—, C₂-C₁₀ acyl, C₁-C₄ alkylcarbonyl, C₁-C₄ alkylaminocarbonyl, 5-12 membered heteroaryl, 5-12 membered heteroarylcarbonyl, C₁-C₄ alkyl-5-12 membered heteroarylcarbonyl and C₁-C₄ alkyl-CO—O—C₁-C₄ alkylene-O—CO—.

In another preferred embodiment, Ar is a substituted or unsubstituted phenyl, wherein the substituted refers to 1, 2 or 3 hydrogen atoms on the phenyl being substituted with groups selected from the group consisting of halogen, C₁-C₄ alkyl, C₃-C₆ cycloalkyl and C₁-C₄ alkoxy. In another preferred embodiment, Ar is a substituted or unsubstituted phenyl, wherein the substituted refers to 1, 2 or 3 hydrogen atoms on the phenyl being substituted with groups selected from the group consisting of fluorine, chlorine, methyl, ethyl, n-propyl, isopropyl, methoxy and ethoxy.

In another preferred embodiment, R₁ and R₂ are each independently hydrogen, deuterium, halogen, cyano, C₁-C₄ alkyl. In another preferred embodiment, R₁ and R₂ are each independently hydrogen, deuterium, halogen. In another preferred embodiment, R₁ and R₂ are each independently hydrogen, deuterium, chlorine, fluorine.

In another preferred embodiment, R₃ is

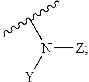

Y is hydrogen, C₁-C₄ alkyl; Z is —(CH₂)ₘ-substituted or unsubstituted phenyl, —(CH₂)ₘ-substituted or unsubstituted 5-7 membered heteroaryl, —(CH₂)ₘ-substituted or unsubstituted C₆-C₈ cycloalkyl, —(CH₂)ₘ-substituted or unsubstituted 5-8 membered heterocyclyl. In another preferred embodiment, Z is —(CH₂)ₘ-substituted or unsubstituted phenyl, —(CH₂)ₘ-substituted or unsubstituted 6-membered heteroaryl, —(CH₂)ₘ-substituted or unsubstituted C₅-C₈ cycloalkyl, —(CH₂)ₘ-substituted or unsubstituted 6-8 membered heterocyclyl. In another preferred embodiment, the substituted refers to 1, 2 or 3 hydrogen atoms on the group being substituted with groups selected from the group consisting of hydroxyl, carboxyl, C₁-C₄ alkoxy, halogen, amino, deuterium, C₁-C₄ alkyl, —CONH₂, C₁-C₄ alkylamino-CO—, cyano, carboxyl-substituted C₁-C₄ alkyl, C₁-C₄ alkyl-CO—O—C₁-C₄ alkylene-O—CO—. In another preferred embodiment, m is 0, 1, 2 or 3.

In another preferred embodiment, R₄ is hydrogen, substituted or unsubstituted C₁-C₆ alkyl, —(CH₂)ₘ-substituted or unsubstituted C₃-C₆ cycloalkyl, —(CH₂)ₘ-substituted or unsubstituted 4-6 membered heterocyclyl, —(CH₂)ₘ-substituted or unsubstituted phenyl, —(CH₂)ₘ-substituted or unsubstituted 5-7 membered heteroaryl, C₃-C₆ alkynyl, C₃-C₆ alkenyl. In another preferred embodiment, m is 0, 1, 2 or 3. In another preferred embodiment, the substituted refers to 1, 2 or 3 hydrogen atoms on the group being substituted with groups selected from the group consisting of halogen, deuterium, cyano, hydroxyl, C₁-C₄ alkoxycarbonyl, C₁-C₄ alkyl, C₂-C₄ alkynyl and C₂-C₄ alkenyl.

In another preferred embodiment, R₅ is substituted or unsubstituted C₁-C₆ alkyl, substituted or unsubstituted C₃-C₆ cycloalkyl, substituted or unsubstituted 4-6 membered heterocycloalkyl. In another preferred embodiment, the substituted refers to 1, 2 or 3 hydrogen atoms on the group being substituted with groups selected from the group consisting of C₁-C₄ alkyl, C₁-C₄ alkyl-CO—and C₁-C₄ alkyl-COO—.

In another preferred embodiment, R₅ is C₁-C₆ alkyl, C₁-C₄ alkyl-substituted C₃-C₆ cycloalkyl, C₁-C₄ alkyl and/or C₁-C₄ alkyl-CO-substituted 4-6 membered heterocycloalkyl. In another preferred embodiment, R₅ is tert-butyl,

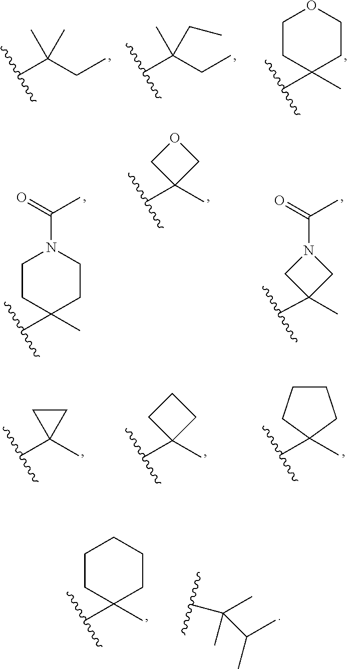

In another preferred embodiment, the compound has a structure as shown in formula II, III, IV, V, VI, VII or VIII:

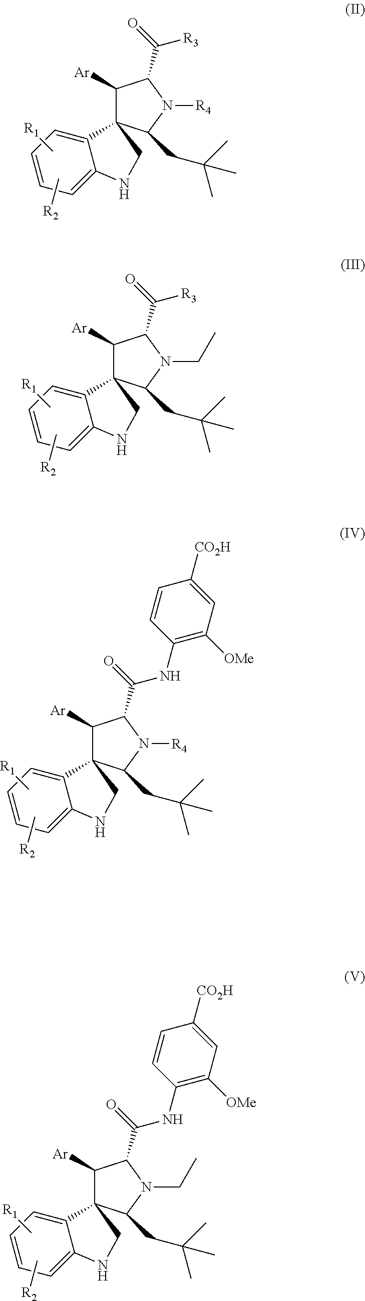

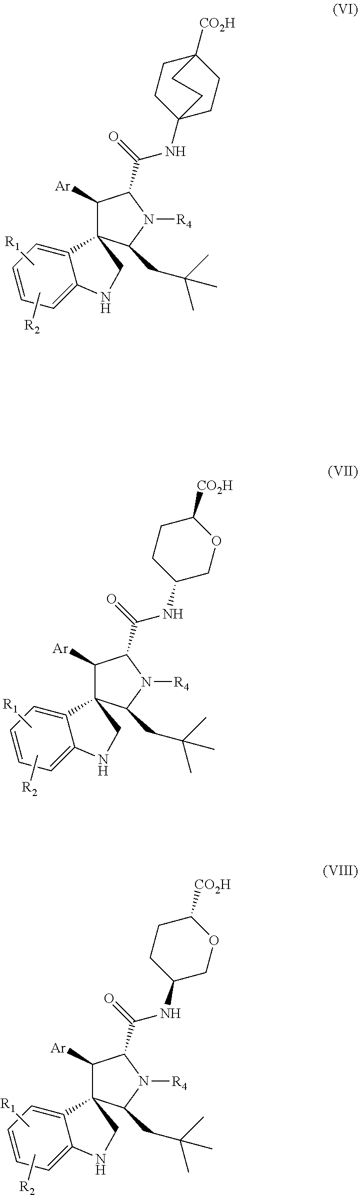

wherein the definitions of the groups are as described above.

In another preferred embodiment, each substituent is the corresponding group in the specific compounds.

In another preferred embodiment, the heterocyclyl and heteroaryl each independently contain 1, 2, 3 or 4 heteroatoms selected from N and O.

In another preferred embodiment, the compound is selected from any one of the compounds of sequence numbers 1-97.

The compounds provided by the present invention can be used as small molecule inhibitors for inhibiting MDM2-p53, MDMX-p53 protein-protein interactions.

The second aspect in the present invention provides a pharmaceutical composition comprising the compound as described in the first aspect, the enantiomer, the diastereomer, the racemate or the pharmaceutically acceptable salt thereof, and a pharmaceutically acceptable carrier.

In another preferred embodiment, the pharmaceutical composition optionally further comprises a pharmaceutically acceptable excipient selected from the group consisting of binder, filler, diluent, disintegrant, suspension, suspending agent, sustained (controlled) release agent, lyophilization protectant, coating agent, enteric material, lubricant, glidant, anti-adherent, sweetener, flavoring agent, plasticizer, light-shielding agent, solubilizer, humectant, solvent, osmotic pressure regulator, colorant, pigment, surfactant, emulsifier, water-soluble matrix, lipid-soluble matrix, oleaginous matrix, pore-forming agent, gelling agent, preservative, buffering agent, chelating agent, antioxidant, or combinations thereof.

The third aspect in the present invention provides a use of the compound of the first aspect, the enantiomer, the diastereomer, the racemate or the pharmaceutically acceptable salt thereof for preparing a small molecule inhibitor for blocking MDM2/p53 and/or MDMX/p53 interactions; or for preparing a medicament for treating a disease related to the activity or expression level of MDM2 or MDMX protein.

In another preferred example, the disease related to the activity or expression level of MDM2 or MDMX protein is selected from the group consisting of glioma, liposarcoma, skin melanoma, squamous cell carcinoma, retinoblastoma, breast cancer, esophageal cancer, lung cancer, ovarian cancer, gastric cancer, bladder cancer, liver cancer, soft tissue sarcoma, chronic lymphocytic leukemia, acute myeloid leukemia, lymphoma, osteosarcoma and colon cancer.

Compared with the prior art, the main advantages of the present invention include.
(1) providing a class of structurally novel small molecule compounds having substituted phenyl spiro[indoline-3,3′-pyrrolidine]structure and similar structures, wherein the preparation method has advantages of mild reaction conditions, abundant and readily available raw materials, simple operation and post-processing, and good enantioselectivity.
(2) providing a small molecule inhibitor that simultaneously inhibits MDM2-p53 and MDMX-p53 protein-protein interactions, which has strong inhibitory ability against the proliferation of p53 wild-type and MDM2 overexpressing cells, and has good solubility, high bioavailability, and excellent metabolic properties, making it a class of potential anti-tumor drugs.

It should be understood that within the scope of the present invention, the above-mentioned technical features of the present invention and the technical features specifically described below (such as in the examples) can be combined with each other to form new or preferred technical solutions. The features disclosed in the specification may be replaced by any alternative features that provide the same, equivalent, or similar purpose. Due to space limitations, they will not be enumerated one by one.

### BRIEF DESCRIPTION OF THE DRAWINGS

FIG. 1 shows the absolute stereochemical configuration of JM085-CF2.

FIG. 2 shows the absolute stereochemical configuration of JN110.

FIG. 3 shows the compound concentration-peak area ratio standard curve.

FIG. 4 shows the results of mechanism of action study of 18(JN122) in HCT116 cells.

FIG. 5 shows the results of mechanism of action study of 18(JN122) in various solid tumor cell lines.

FIG. 6 shows the results of mechanism of action study of 18(JN122) in MOLM-13 cells.

FIG. 7 shows the results of pharmacodynamic study of 18(JN122) in MOLM-13 mouse xenograft tumor model.

### DETAILED DESCRIPTION OF THE INVENTION

After long-term and in-depth research, the inventors of the present invention have unexpectedly developed small molecule compounds having substituted phenyl spiro[indoline-3,3′-pyrrolidine]structure and similar structures, which can inhibit MDM2-p53 and MDMX-p53 protein-protein interactions, regulate p53-mediated gene expression in tumor cells, and therefore can be used for the prevention and treatment of diseases related to MDM2-p53 and MDMX-p53 interactions, such as cancer. Based on the above findings, the inventors have completed the present invention.

### Terms

In the present invention, the halogen is F, Cl, Br or I.

In the present invention, unless specifically indicated, the terms used have the general meanings known to those skilled in the art.

In the present invention, the term “C₁-C₆” refers to having 1, 2, 3, 4, 5 or 6 carbon atoms, “C₁-C₄” refers to having 1, 2, 3 or 4 carbon atoms, and so on. “4-12 membered” refers to having 4, 5, 6, 7, 8, 9, 10, 11 or 12 ring atoms, and so on.

In the present invention, the term “alkyl” refers to a saturated linear or branched hydrocarbon moiety, for example, the term “C₁-C₈ alkyl” refers to a straight or branched chain alkyl having 1 to 8 carbon atoms, non-limitingly including methyl, ethyl, n-propyl, isopropyl, n-butyl, isobutyl, sec-butyl, tert-butyl, pentyl and hexyl, etc.; preferably ethyl, n-propyl, isopropyl, n-butyl, isobutyl, sec-butyl and tert-butyl.

In the present invention, the term “alkoxy” refers to an —O-(alkyl) group. For example, the term “C₁-C₆ alkoxy” refers to a straight or branched chain alkoxy having 1 to 6 carbon atoms, non-limitingly including methoxy, ethoxy, n-propoxy, isopropoxy and butoxy, etc.

In the present invention, the term “alkenyl” refers to a straight or branched chain hydrocarbon moiety containing at least one double bond, for example, the term “C₂-C₆ alkenyl” refers to a straight or branched chain alkenyl having 2 to 6 carbon atoms and containing one double bond, non-limitingly including ethenyl, propenyl, butenyl, isobutenyl, pentenyl and hexenyl, etc.

In the present invention, the term “alkynyl” refers to a straight or branched chain alkynyl containing one triple bond, non-limitingly including ethynyl, propynyl, butynyl, isobutynyl, pentynyl (2-methyl-3-butynyl, 2-pentynyl, 3-pentynyl) and hexynyl, etc.

In the present invention, the term “cycloalkyl” refers to a saturated monocyclic, bridged cyclic or spirocyclic cyclic hydrocarbon moiety, for example, the term “C₃-C₈ cycloalkyl” refers to a monocyclic, bridged cyclic or spirocyclic cyclic alkyl having 3 to 8 carbon atoms in the ring, non-limitingly including cyclopropyl, cyclobutyl, cyclopentyl, cyclohexyl, cycloheptyl, cyclooctyl and cyclodecyl, etc. The term “bridged cyclic group” includes, not limited to bicyclo[1.1.1]pentanyl, bicyclo[2.1.1]hexanyl, bicyclo[2.2.1]heptanyl, bicyclo[2.2.2]octanyl, bicyclo[3.2.2]octanyl, etc.

In the present invention, the term “aryl” refers to a hydrocarbon moiety containing one or more aromatic rings. For example, the term “C₆-C₁₀ aryl” refers to an aromatic ring group having 6 to 10 carbon atoms in the ring without heteroatoms, such as phenyl, naphthyl, etc.

In the present invention, the term “heterocyclyl” refers to a saturated or unsaturated, non-aromatic cyclic group containing at least one (such as 1, 2, 3 or 4) ring heteroatom (such as N, O or S), for example, tetrahydropyridinyl, pyrrolinyl, dihydropyridinyl, dihydrofuranyl, dihydrothiophenyl, morpholinyl.

In the present invention, the term “heteroaryl” refers to an aromatic cyclic group containing at least one (such as 1, 2, 3 or 4) ring heteroatom (such as N, O or S), for example, furanyl, pyrrolyl, thiophenyl, oxazolyl, imidazolyl, thiazolyl, pyridinyl, quinolinyl, isoquinolinyl, indolyl, pyrimidinyl, pyranyl.

### Compounds

The compound of the present invention has a structure as shown in general formula I:

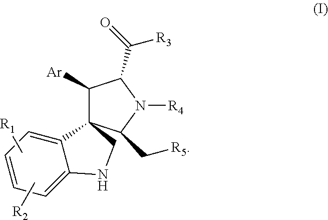

In another preferred example, the compound has a structure as shown in formula 1-1:

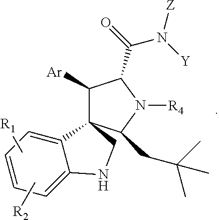

In another preferred example, Ar is a substituted or unsubstituted phenyl, wherein the substituted refers to 1, 2, 3, or 4 hydrogen atoms on the phenyl being substituted by groups selected from the group consisting of fluorine, chlorine, bromine, methoxy, methyl, ethyl, n-propyl, isopropyl, n-butyl, isobutyl, halogenated C₁-C₄ alkyl (such as trifluoromethyl); R₁ and R₂ are each independently hydrogen, deuterium, fluorine, chlorine, or bromine;
R₃ is OH or

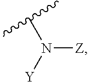

Y is H; Z is H, substituted or unsubstituted C₁-C₄ alkyl, —(CH₂)ₘ-substituted or unsubstituted phenyl, —(CH₂)ₘ-substituted or unsubstituted 5-7 membered heteroaryl, —(CH₂)ₘ-substituted or unsubstituted C₃-C₈ cycloalkyl, or —(CH₂)ₘ-substituted or unsubstituted 5-7 membered heterocyclyl; or Y, Z together with N form a substituted or unsubstituted 5-7 membered heterocyclyl; wherein the substituted refers to being substituted by 1, 2, 3, or 4 groups selected from: fluorine, chlorine, bromine, amino, hydroxyl, carboxyl, C₁-C₄ alkoxy, —CONH₂, C₁-C₄ alkyl-CONH—, C₁-C₄ alkyl-NHCO—, C₁-C₄ alkyl, C₁-C₄ alkyl-S(O₂)—C₁-C₄ alkylene-, mono(C₁-C₄ alkyl)amino, di(C₁-C₄ alkyl)amino, C₁-C₄ alkylSO₂NHCO—, carboxyl-substituted C₁-C₄ alkyl, hydroxyl-substituted C₁-C₄ alkyl; each m is independently 0, 1, or 2;
R₄ is substituted or unsubstituted C₁-C₄ alkyl, —(CH₂)ₘ-substituted or unsubstituted phenyl, —(CH₂)ₘ-substituted or unsubstituted C₃-C₆ cycloalkyl; each m is independently 0, 1, or 2; wherein the substituted refers to being substituted by 1, 2, 4, or 3 groups selected from: fluorine, chlorine, bromine, cyano, amino, hydroxyl, nitro, carboxyl, C₁-C₄ alkoxy, —CONH₂, C₁-C₄ alkyl-CONH—, C₁-C₄ alkyl-NHCO—, C₁-C₄ alkyl, C₁-C₄ alkyl-S(O₂)—C₁-C₄ alkylene-, C₁-C₄ alkylSO₂—, 5-7 membered heteroaryl, carboxyl-substituted C₁-C₄ alkyl, hydroxyl-substituted C₁-C₄ alkyl, C₁-C₄ alkylSO₂NHCO—;
R₅ is substituted or unsubstituted C₁-C₆ alkyl, substituted or unsubstituted C₃-C₆ cycloalkyl, substituted or unsubstituted 5-7 membered heterocyclyl, wherein the substituted refers to being substituted by one or more groups selected from: C₁-C₄ alkyl, C₁-C₄ alkylcarbonyl.

In another preferred example, the heteroaryl in the present invention is selected from: tetrazolyl, isoxazolyl, oxazolyl, pyridinyl, imidazolyl, pyrazolyl.

In another preferred example, the heteroaryl in the present invention is selected from:

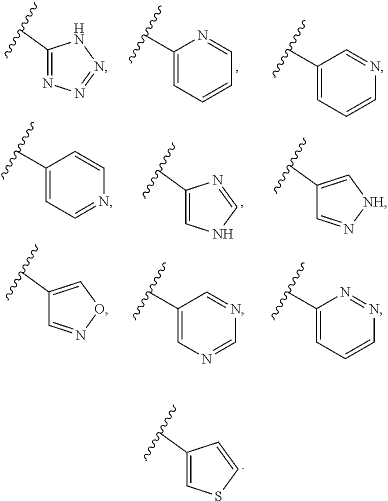

In another preferred example, the heterocyclyl in the present invention is selected from:

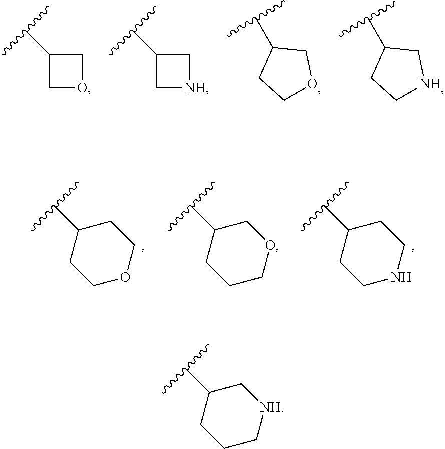

### Preparation Method

The compounds of the present invention can be prepared by the following reaction routes.

### Route One: Route for Preparing Compound (I)

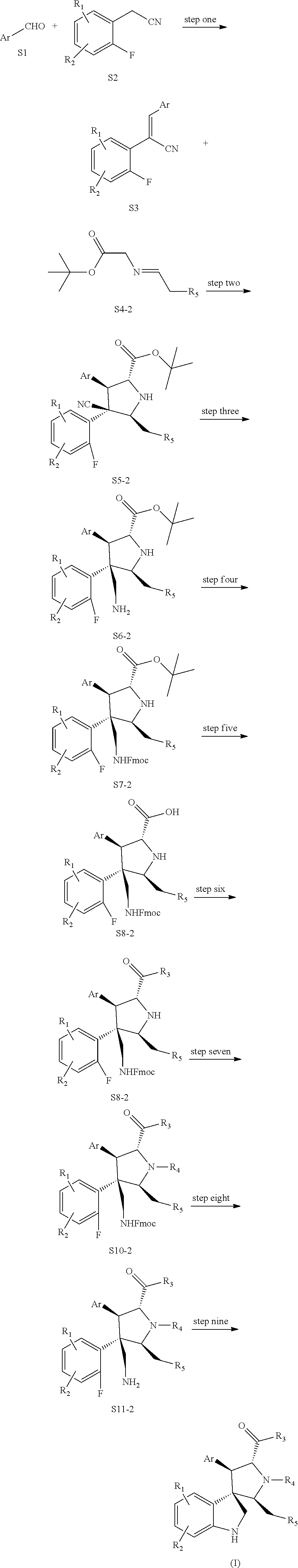

Step one: Aldehyde S1 and substituted 2-fluorophenylacetonitrile S2 are mixed in an appropriate solvent (e.g., methanol, ethanol, tetrahydrofuran, 2-methyltetrahydrofuran, dichloromethane, or N,N-dimethylformamide, etc.). An appropriate base (sodium methoxide, sodium ethoxide, etc.) is added, and the reaction is carried out at room temperature or under appropriately elevated temperature condition (e.g., 40-60° C.) to obtain intermediate S3. Step two: S3 and corresponding starting material S4-2 are mixed in an appropriate solvent (e.g., tetrahydrofuran, 2-methyltetrahydrofuran, dichloromethane, or acetonitrile, etc.), silver fluoride and a base (triethylamine, N,N-diisopropylethylamine, and DBU, etc.) are added, and the reaction is carried out at room temperature to obtain intermediate S5-2. Step three. S5-2 is subjected to hydrogenation reduction to obtain intermediate S6-2 (e.g., Pd/C hydrogen hydrogenation reduction, Raney nickel hydrogen hydrogenation reduction, and Raney nickel hydrazine hydrate hydrogenation reduction, etc.). Step four: S6-2 and FmocCl are mixed in an appropriate solvent (e.g., tetrahydrofuran, 2-methyltetrahydrofuran, dichloromethane, or acetonitrile, etc.), a base (triethylamine, N,N-diisopropylethylamine, and DBU, etc.) is added, and the reaction is carried out at room temperature to obtain intermediate S7-2. Step five: S7-2 is dissolved in dichloromethane, trifluoroacetic acid is added, and the reaction is carried out at room temperature to obtain intermediate S8-2. Step six: S8-2 is dissolved in an appropriate solvent (e.g., tetrahydrofuran, 2-methyltetrahydrofuran, dichloromethane, N,N-dimethylformamide, or acetonitrile, etc.), a base (triethylamine, N,N-diisopropylethylamine, and DBU, etc.) and a coupling reagent (diphenylphosphinic chloride, CDI, PyBOP, HATU, and EDCI, etc.) are added, an amine is added after half an hour of reaction, and the reaction is carried out at room temperature to obtain intermediate S9-2. Step seven: S9-2 and R₄CHO are dissolved in a suitable solvent (e.g., methanol, tetrahydrofuran, ethanol, 1,2-dichloroethane, and N,N-dimethylformamide, etc.), a reducing agent (sodium borohydride, sodium borohydride acetate, sodium cyanoborohydride, etc.) is added, and intermediate S10-2 is obtained through reductive amination. Step eight. S10-2 is dissolved in an appropriate solvent (e.g., N,N-dimethylformamide, etc.), piperidine is added, and the reaction is carried out at room temperature to obtain intermediate S11-2. Step nine: S11-2 is dissolved in an appropriate solvent (e.g., acetonitrile, dimethyl sulfoxide, N,N-dimethylformamide, toluene, and benzene, etc.), a base (e.g., potassium carbonate, cesium carbonate, and sodium carbonate, etc.) is added, and the reaction is carried out under elevated temperature condition (e.g., 80-120° C.) to obtain the final product I.

### Route Two: Route for Preparing Compound (II)

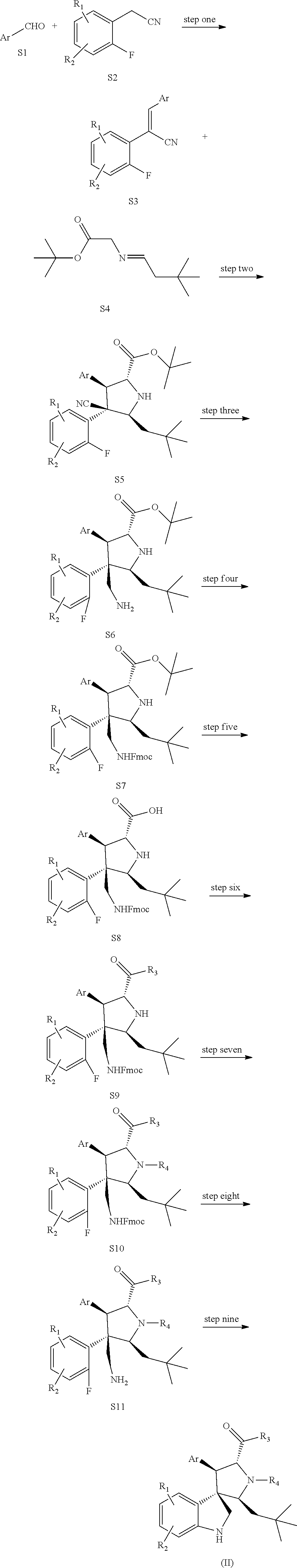

Step one: Aldehyde S1 and substituted 2-fluorophenylacetonitrile S2 are mixed in an appropriate solvent (e.g., methanol, ethanol, tetrahydrofuran, 2-methyltetrahydrofuran, dichloromethane, or N,N-dimethylformamide, etc.). An appropriate base (sodium methoxide, sodium ethoxide, etc.) is added, and the reaction is carried out at room temperature or under appropriately elevated temperature condition (e.g., 40-60° C.) to obtain intermediate S3. Step two: S3 and corresponding starting material S4 are mixed in an appropriate solvent (e.g., tetrahydrofuran, 2-methyltetrahydrofuran, dichloromethane, or acetonitrile, etc.), silver fluoride and a base (triethylamine, N,N-diisopropylethylamine and DBU, etc.) are added, and the reaction is carried out at room temperature to obtain intermediate S5. Step three: S5 is subjected to hydrogenation reduction to obtain intermediate S6 (e.g., Pd/C hydrogen hydrogenation reduction, Raney nickel hydrogen hydrogenation reduction, and Raney nickel hydrazine hydrate hydrogenation reduction, etc.). Step four: S6 and FmocCl are mixed in an appropriate solvent (e.g., tetrahydrofuran, 2-methyltetrahydrofuran, dichloromethane, or acetonitrile, etc.), a base (triethylamine, N,N-diisopropylethylamine and DBU, etc.) is added, and the reaction is carried out at room temperature to obtain intermediate S7. Step five: S7 is dissolved in dichloromethane, trifluoroacetic acid is added, and the reaction is carried out at room temperature to obtain intermediate S8. Step six: S8 is dissolved in an appropriate solvent (e.g., tetrahydrofuran, 2-methyltetrahydrofuran, dichloromethane, N,N-dimethylformamide or acetonitrile, etc.), a base (triethylamine, N,N-diisopropylethylamine and DBU, etc.) and a coupling reagent (diphenylphosphinic chloride, CDI, PyBOP, HATU and EDCI, etc.) are added, an amine is added after half an hour of reaction, and the reaction is carried out at room temperature to obtain intermediate S9. Step seven: S9 and R₄CHO are dissolved in a suitable solvent (e.g., methanol, tetrahydrofuran, ethanol, 1,2-dichloroethane and N,N-dimethylformamide, etc.), a reducing agent (sodium borohydride, sodium borohydride acetate, sodium cyanoborohydride, etc.) is added, and intermediate S10 is obtained through reductive amination. Step eight: S10 is dissolved in an appropriate solvent (e.g., N,N-dimethylformamide, etc.), piperidine is added, and the reaction is carried out at room temperature to obtain intermediate S11. Step nine: S11 is dissolved in an appropriate solvent (e.g., acetonitrile, dimethyl sulfoxide, N,N-dimethylformamide, toluene and benzene, etc.), a base (e.g., potassium carbonate, cesium carbonate and sodium carbonate, etc.) is added, and the reaction is carried out under elevated temperature condition (e.g., 80-120° C.) to obtain the final product II.

### Route Three: Route for Preparing Compound (II)

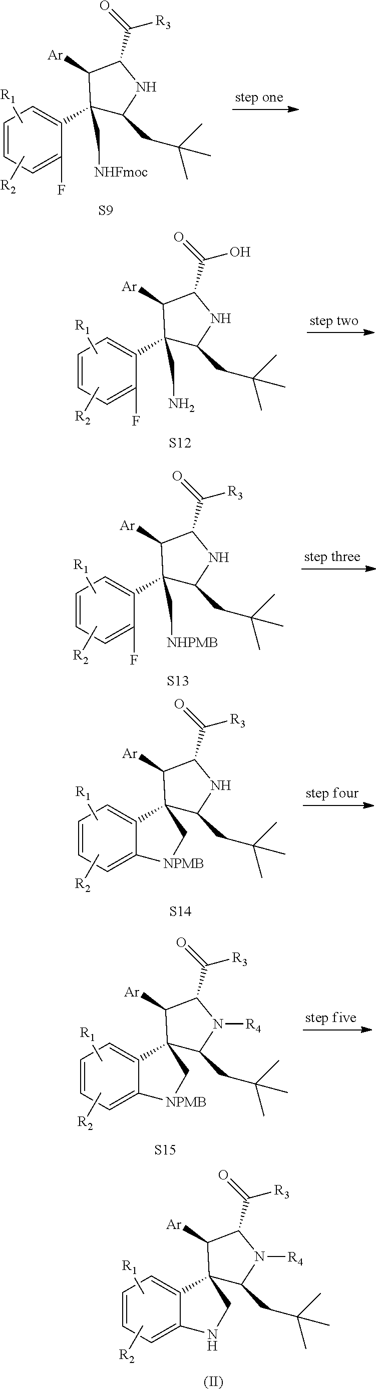

Step one: S9 is dissolved in an appropriate solvent (e.g., N,N-dimethylformamide, etc.), piperidine is added, and the reaction is carried out at room temperature to obtain intermediate S12. Step two: S12 and p-methoxybenzaldehyde are dissolved in a suitable solvent (e.g., methanol, tetrahydrofuran, ethanol, 1,2-dichloroethane and N,N-dimethylformamide, etc.), a reducing agent (sodium borohydride, sodium borohydride acetate, sodium cyanoborohydride, etc.) is added, and intermediate S13 is obtained through reductive amination. Step three: S13 is dissolved in an appropriate solvent (e.g., acetonitrile, dimethyl sulfoxide, N,N-dimethylformamide, toluene and benzene, etc.), a base (e.g., potassium carbonate, cesium carbonate and sodium carbonate, etc.) is added, and the reaction is carried out under elevated temperature condition (e.g., 80-120° C.) to obtain intermediate S14. Step four: S14 and R₄CHO are dissolved in a suitable solvent (e.g., methanol, tetrahydrofuran, ethanol, 1,2-dichloroethane and N,N-dimethylformamide, etc.), a reducing agent (sodium borohydride, sodium borohydride acetate, sodium cyanoborohydride, etc.) is added, and intermediate S15 is obtained through reductive amination. Step five: S15 is dissolved in trifluoroacetic acid, and the reaction is carried out under elevated temperature condition to obtain the final product II.

I and I-1 can be synthesized using the above three synthetic strategies.

### Route Four: Preparation of Optically Active Compounds (II) by Chiral Catalytic Synthesis

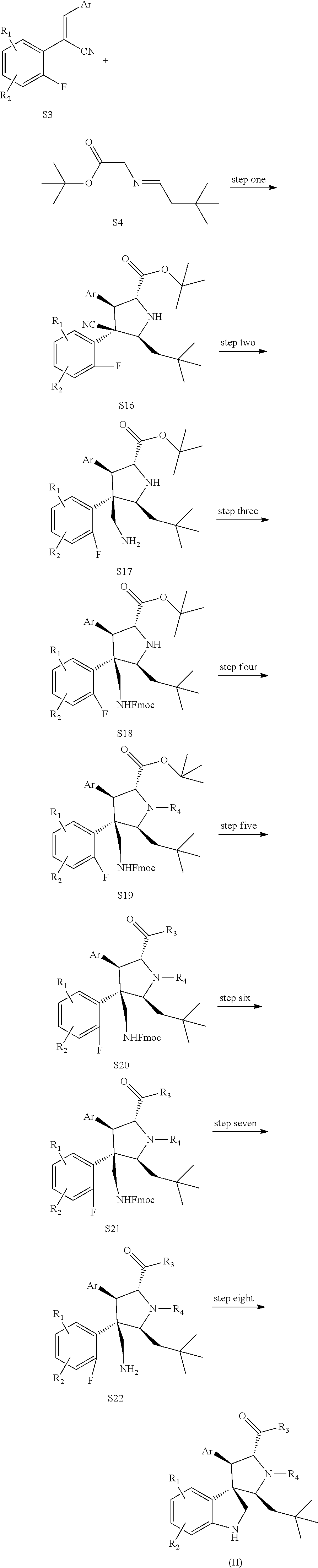

Step one: S3 and corresponding raw material S4 are mixed in an appropriate solvent (e.g., tetrahydrofuran, 2-methyltetrahydrofuran, dichloromethane, or acetonitrile, etc.), deoxygenated, and purged with nitrogen, followed by addition of cuprous acetate and R—(+)-1,1′-binaphthyl-2,2′-bis(diphenylphosphine) (BINAP), dropwise addition of a base (triethylamine, N,N-diisopropylethylamine, and DBU, etc.), reacting at room temperature to obtain intermediate S16, wherein S16 is a single enantiomer or an enantiomeric mixture with ee>50%. Similarly, the bisphosphine ligand can also be R—(+)-1,1′-binaphthyl-2,2′-bis(di-4-methylphenyl)phosphine, R—(+)-1,1′-binaphthyl-2,2′-bis(di-3,5-dimethylphenyl)phosphine, R—(+)-1,1′-binaphthyl-2,2′-bis(di-4-iodophenyl)phosphine, R—(+)-1,1′-binaphthyl-2,2′-bis(di-4-methoxyphenyl)phosphine, (R)-(+)-2,2′-bis(diphenylphosphino)-5,5′,6,6′,7,7′,8,8′-octahydro-1,1′-binaphthyl, etc. Step two: S16 is subjected to hydrogenation reduction to obtain intermediate S17 (e.g., Pd/C hydrogen hydrogenation reduction, Raney nickel hydrogen hydrogenation reduction, and Raney nickel hydrazine hydrate hydrogenation reduction, etc.). Step three: S17 and FmocCl are mixed in an appropriate solvent (e.g., tetrahydrofuran, 2-methyltetrahydrofuran, dichloromethane, or acetonitrile, etc.), a base (triethylamine, N,N-diisopropylethylamine, and DBU, etc.) is added, and reacted at room temperature to obtain intermediate S18. Step four: S18 and R₄CHO are dissolved in a suitable solvent (e.g., methanol, tetrahydrofuran, ethanol, 1,2-dichloroethane, and N,N-dimethylformamide, etc.), a reducing agent (sodium borohydride, sodium borohydride acetate, sodium cyanoborohydride, etc.) is added, and intermediate S19 is obtained through reductive amination. Step five: S19 is dissolved in dichloromethane, trifluoroacetic acid is added and reacted at room temperature to obtain intermediate S20. Step six: S20 is dissolved in an appropriate solvent (e.g., tetrahydrofuran, 2-methyltetrahydrofuran, dichloromethane, N,N-dimethylformamide, or acetonitrile, etc.), a base (1-methylimidazole, triethylamine, N,N-diisopropylethylamine, and DBU, etc.) and a coupling reagent (ethylsulfonyl chloride, diphenylphosphinic chloride, CDI, PyBOP, HATU, and EDCI, etc.) are added at zero degrees Celsius, an amine is added after half an hour of reaction and reacted at room temperature to obtain intermediate S21. Step seven: S21 is dissolved in an appropriate solvent (e.g., N,N-dimethylformamide, etc.), piperidine is added and reacted at room temperature to obtain intermediate S22. Step eight: S22 is dissolved in an appropriate solvent (e.g., acetonitrile, dimethyl sulfoxide, N,N-dimethylformamide, toluene, and benzene, etc.), a base (e.g., potassium carbonate, cesium carbonate, and sodium carbonate, etc.) is added and reacted under elevated temperature condition (e.g., 80-120° C.) to obtain the final product II, wherein the final product II is a single enantiomer or an enantiomeric mixture with ee>50%.

### Route Five: Preparation of Optically Active Compounds (I) by Chiral Catalytic Synthesis

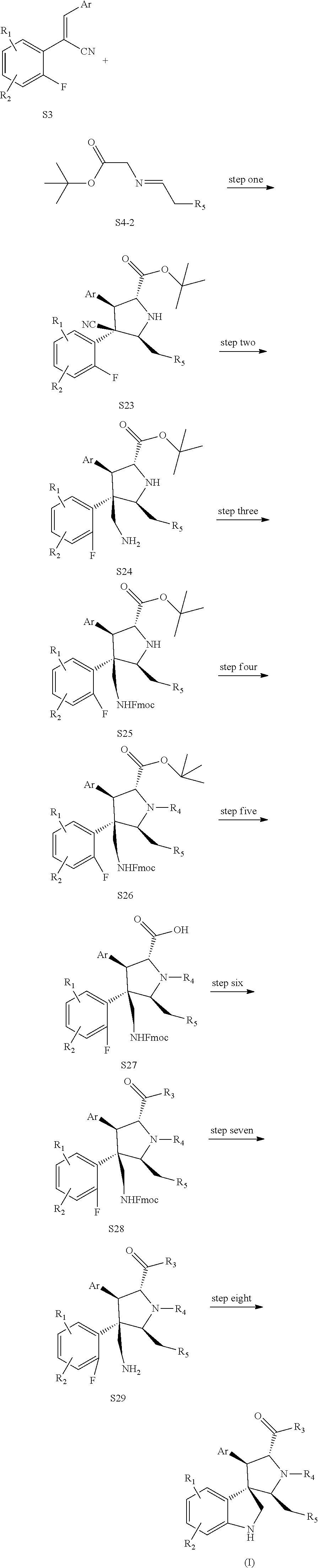

S23 to S29 are optically active enantiomeric mixtures with ee>20%

Step one: S3 and the corresponding raw material S4-2 are mixed in an appropriate solvent (e.g., tetrahydrofuran, 2-methyltetrahydrofuran, dichloromethane, or acetonitrile, etc.), deoxygenated, and purged with nitrogen, followed by addition of cuprous acetate and R—(+)-1,1′-binaphthyl-2,2′-bis(diphenylphosphine) (BINAP), dropwise addition of a base (triethylamine, N,N-diisopropylethylamine, DBU, etc.), reacting at room temperature to obtain intermediate S23, wherein S23 is a single enantiomer or a mixture of enantiomeric mixture with ee>20%. Similarly, the bisphosphine ligand can also be R—(+)-1,1′-binaphthyl-2,2′-bis(di-4-methylphenyl)phosphine, R—(+)-1,1′-binaphthyl-2,2′-bis(di-3,5-dimethylphenyl)phosphine, R—(+)-1,1′-binaphthyl-2,2′-bis(di-4-iodophenyl)phosphine, R—(+)-1,1′-binaphthyl-2,2′-bis(di-4-methoxyphenyl)phosphine, (R)-(+)-2,2′-bis(diphenylphosphino)-5,5′,6,6′,7,7′,8,8′-octahydro-1,1′-binaphthyl, etc. Step two: S23 is subjected to hydrogenation reduction to obtain intermediate S24 (e.g., Pd/C hydrogen hydrogenation reduction, Raney nickel hydrogen hydrogenation reduction, and Raney nickel hydrazine hydrate hydrogenation reduction, etc.). Step three: S24 and FmocCl are mixed in an appropriate solvent (e.g., tetrahydrofuran, 2-methyltetrahydrofuran, dichloromethane, or acetonitrile, etc.), a base (triethylamine, N,N-diisopropylethylamine, and DBU, etc.) is added and reacted at room temperature to obtain intermediate S25. Step four: S25 and R₄CHO are dissolved in a suitable solvent (e.g., methanol, tetrahydrofuran, ethanol, 1,2-dichloroethane, and N,N-dimethylformamide, etc.), a reducing agent (sodium borohydride, sodium borohydride acetate, sodium cyanoborohydride, etc.) is added, and intermediate S26 is obtained through reductive amination. Step five: S26 is dissolved in dichloromethane, trifluoroacetic acid is added, and the reaction is carried out at room temperature to obtain intermediate S27. Step six: S27 is dissolved in an appropriate solvent (e.g., tetrahydrofuran, 2-methyltetrahydrofuran, dichloromethane, N,N-dimethylformamide, or acetonitrile, etc.), a base (1-methylimidazole, triethylamine, N,N-diisopropylethylamine, and DBU, etc.) and a coupling reagent (ethanesulfonyl chloride, diphenylphosphinic chloride, CDI, PyBOP, HATU, and EDCI, etc.) are added at zero degrees Celsius, an amine is added after half an hour of reaction, and the reaction is carried out at room temperature to obtain intermediate S28. Step seven: S28 is dissolved in an appropriate solvent (e.g., N,N-dimethylformamide, etc.), piperidine is added, and the reaction is carried out at room temperature to obtain intermediate S29. Step eight: S29 is dissolved in an appropriate solvent (e.g., acetonitrile, dimethyl sulfoxide, N,N-dimethylformamide, toluene, and benzene, etc.), a base (e.g., potassium carbonate, cesium carbonate, and sodium carbonate, etc.) is added, and the reaction is carried out under elevated temperature condition (e.g., 80-120° C.) to obtain the final product I, wherein the final product I is a single enantiomer or a mixture of enantiomeric mixtures with ee>20%.

### Example 1: Synthesis of Compounds

### Synthesis of Intermediate 1: Methyl 4-((2′,3S,4′,5′-R)-6-chloro-4-((2,3-difluorophenyl)-1-(4-methoxybenzyl)-2-neopentylspiro[indoline-3,3′-pyrrolidine]-5′-carboxamido)-3-methoxybenzoate (JM158)

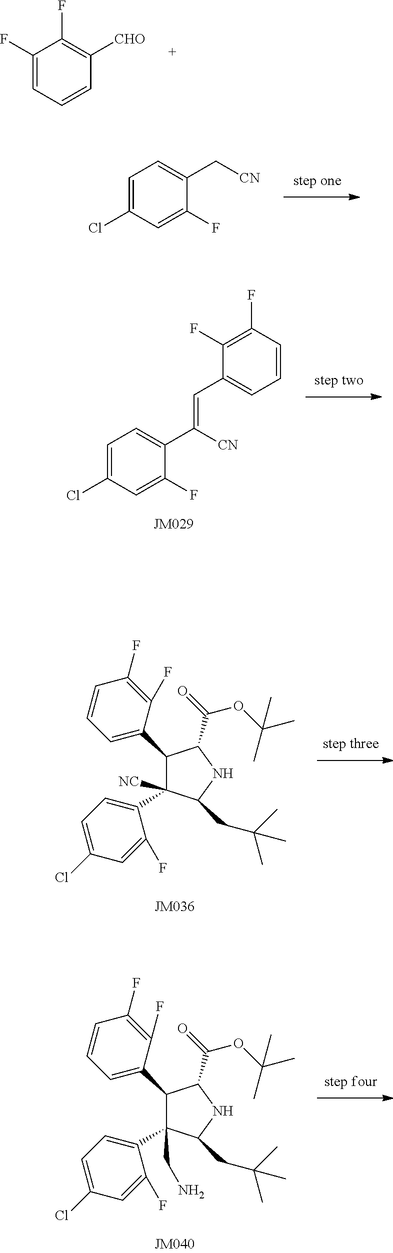 [SMILES: O=Cc1cccc(F)c1F ; N#CCc1ccc(Cl)cc1F ; N#C/C(=C\c1cccc(F)c1F)c1ccc(Cl)cc1F ; CC(C)(C)C[C@@H]1N[C@@H](C(=O)OC(C)(C)C)[C@H](c2cccc(F)c2F)[C@@]1(C#N)c1ccc(Cl)cc1F ; CC(C)(C)C[C@@H]1N[C@@H](C(=O)OC(C)(C)C)[C@H](c2cccc(F)c2F)[C@@]1(CN)c1ccc(Cl)cc1F]

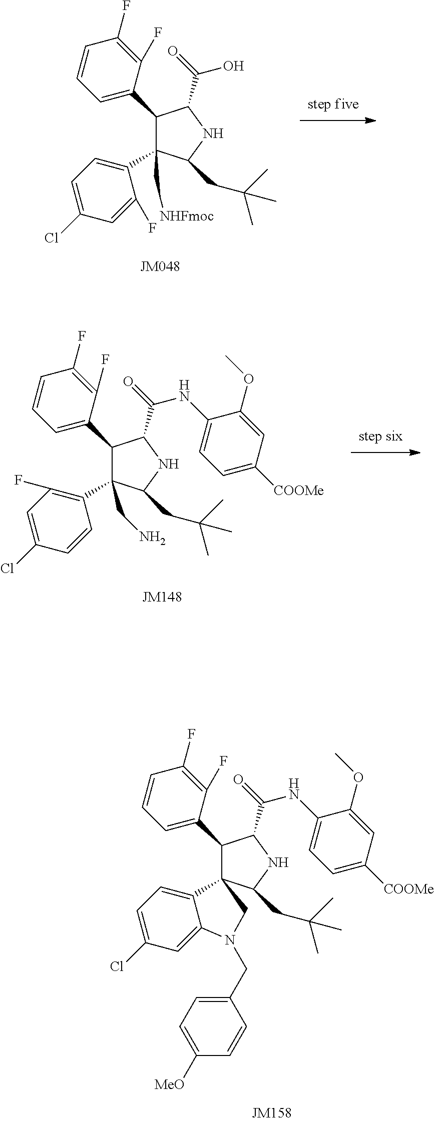

### Step one: Synthesis of (Z)-2-(4-chloro-2-fluorophenyl)-3-(2,3-difluorophenyl)acrylonitrile (JM029)

2,3-difluorobenzaldehyde (2.9 g, 20 mmol) and 4-chloro-2-fluorophenylacetonitrile (3.4 g, 20 mmol) were added into a 250 mL round-bottom flask, 150 mL of methanol was added to dissolve the reactants, and 4.8 mL of 5N sodium methoxide in methanol was added dropwise. The reaction system was stirred overnight at 50° C. After the reaction was complete, the mixture was cooled to room temperature and filtered to obtain the crude product (5.8 g, 99% yield). ¹H NMR (500 MHz, Chloroform-d) δ 8.00 (dd, J=8.0, 6.2 Hz, 1H), 7.78 (s, 1H), 7.56 (t, J=8.4 Hz, 1H), 7.35-7.16 (m, 4H).

### Step two: Synthesis of tert-butyl (2R,3R,4R,5S)-4-(4-chloro-2-fluorophenyl)-3-(2,3-difluorophenyl)-4-cyano-5-neopentylpyrrolidine-2-carboxylate (JM036)

JM029 (5.7 g, 20 mmol) and AgF (2.54 g, 20 mmol) were weighed into a 100 mL round-bottom flask, 30 mL of anhydrous dichloromethane was added to dissolve, triethylamine (4.4 mL) and tert-butyl (E)-2-(((3,3-dimethylbutylidene)amino)acetate (4.4 g, 20 mmol) were added dropwise, and the reaction was carried out at room temperature for 24 h. After the reaction was complete, saturated ammonium chloride was added to the reaction mixture, extracted with dichloromethane (30 mL×3 times), the combined organic phases were washed twice with saturated sodium chloride and dried over anhydrous sodium sulfate. The organic phase was dried by rotary evaporation and the residue is purified by column chromatography to obtain the target product (6.8 g, 67% yield). ¹H NMR (500 MHz, Chloroform-d) δ 7.45 (dd, J=7.9, 6.0 Hz, 1H), 7.35 (t, J=8.5 Hz, 1H), 7.19-7.02 (m, 4H), 4.68 (dd, J=7.5, 1.9 Hz, 1H), 4.16 (d, J=7.5 Hz, 1H), 4.08 (d, J=8.9 Hz, 1H), 1.62 (ddd, J=14.2, 9.1, 1.7 Hz, 1H), 1.38 (s, 9H), 1.28 (dd, J=14.3, 0.9 Hz, 1H), 0.89 (s, 9H).

### Step Three: Synthesis of tert-butyl (2R,3R,4S,5S)-4-(aminomethyl)-4-(4-chloro-2-fluorophenyl)-3-(2,3-difluorophenyl)-5-neopentylpyrrolidine-2-carboxylate (JM040)

JM036 (1.6 g, 8.5 mmol) was added to a 100 mL round-bottom flask, dissolved in tetrahydrofuran/EtOH (30 mL/10 mL), heated to 55° C., followed by addition of Raney nickel (4 g) and hydrazine hydrate (10 mL). The reaction was carried out for 2 h. Then the mixture was filtered, the solvent was evaporated, and the target product was purified by normal phase column chromatography (1.6 g, with a yield of 24%). ¹H NMR (500 MHz, Chloroform-d) δ 7.49 (t, J=6.7 Hz, 1H), 7.09 (t, J=8.6 Hz, 1H), 7.06-6.94 (m, 4H), 4.20-4.13 (m, 3H), 3.29 (dd, J=13.1, 1.5 Hz, 1H), 3.19 (d, J=13.2 Hz, 1H), 1.50-1.44 (m, 2H), 1.25 (s, 9H), 0.94 (s, 9H).

### Step Four: Synthesis of (2R,3R,4S,5S)-4-((((((9H-fluoren-9-yl)methoxy)carbonyl)amino)methyl)-4-(4-chloro-2-fluorophenyl)-3-(2,3-difluorophenyl)-5-neopentylpyrrolidine-2-carboxylic acid (JM048)

JM040 (1.4 g, 2.7 mmol) was added to a 50 mL single-neck flask, dissolved in dry tetrahydrofuran, followed by addition of diisopropylethylamine (1.4 g, 10.8 mmol) and FmocCl (1.1 g, 4.1 mmol), and the reaction was carried out overnight at room temperature. The reaction mixture was concentrated under reduced pressure by rotary evaporation, dissolved in 5 mL dichloromethane, followed by addition of 4 mL trifluoroacetic acid, and left overnight at room temperature. The reaction solution was evaporated, saturated sodium bicarbonate solution was added, the mixture was extracted three times with dichloromethane, the organic phases were combined, evaporated, and purified by normal phase column chromatography to obtain the target product (1.31 g, with a yield of 73%).

### Step five: Synthesis of methyl 4-((2R,3R,4S,5S)-4-(aminomethyl)-4-(4-chloro-2-fluorophenyl)-3-(2,3-difluorophenyl)-5-neopentylpyrrolidine-2-carboxamido)-3-methoxybenzoate (JM148)

JM048 (515 mg, 0.8 mmol) was added to a 50 mL single-neck flask, dissolved in dry tetrahydrofuran, followed by addition of diisopropylethylamine (504 mg, 3.9 mmol), stirred for 5 minutes, then diphenylphosphinic chloride (555 mg, 2.3 mmol) was added, stirred for half an hour, followed by addition of methyl 4-amino-3-methoxybenzoate (562 mg, 3.1 mmol), and the reaction was carried out overnight at room temperature. Saturated sodium bicarbonate solution was added to the reaction mixture, extracted with dichloromethane, the organic phase was evaporated and purified by normal phase column chromatography. The crude product was dissolved in DMF (2 mL), piperidine (0.4 mL) was added, and the reaction was carried out at room temperature for 15 minutes, washed three times with 1N HCl solution, washed three times with saturated sodium chloride solution, the organic phase was evaporated and purified by normal phase column chromatography to obtain the target product (285 mg, with a yield of 73%). ¹H NMR (400 MHz, Methanol-d₄) δ 8.26 (d, J=8.5 Hz, 1H), 7.61-7.53 (m, 2H), 7.47-7.36 (m, 1H), 7.34 (d, J=8.5 Hz, 1H), 7.32-7.05 (m, 4H), 4.65 (d, J=10.3 Hz, 1H), 4.46 (d, J=10.9 Hz, 1H), 3.99 (d, J=12.4 Hz, 1H), 3.92 (s, 3H), 3.86 (s, 3H), 3.78 (dd, J=14.3, 3.2 Hz, 1H), 3.56 (d, J=14.5 Hz, 1H), 1.73 (d, J=13.7 Hz, 1H), 1.69-1.57 (m, 1H), 1.23 (s, 9H).

### Step six: Synthesis of methyl 4-((2′S,3S,4′S,5′R)-6-chloro-4′-(2,3-difluorophenyl)-4-fluoro-1-(4-methoxybenzyl)-2′-neopentylspiro[indoline-3,3′-pyrrolidine]-5′-carboxamido)-3-methoxybenzoate (JM158)

A 50 mL single-neck flask was dried, JM148 (285 mg, 0.46 mmol) and 4-methoxybenzaldehyde (252 mg, 1.85 mmol) were added, the reactants were dissolved in 5 mL methanol, followed by addition of sodium cyanoborohydride (117 mg, 1.85 mmol) and 0.1 mL acetic acid, and the reaction was carried out overnight at room temperature. The reaction mixture was evaporated, saturated sodium bicarbonate was added, extracted with ethyl acetate, washed with saturated brine, dried over anhydrous sodium sulfate, and concentrated by rotary evaporation. The crude product obtained from the reaction was added to a dried 50 mL single-neck flask, dissolved in 5 mL DMF, followed by addition of potassium carbonate (197 mg, 1.42 mmol), and stirred overnight at 110° C. After the reaction was completed, the mixture was cooled to room temperature and water was added. The mixture was extracted with ethyl acetate, washed with saturated brine, dried over anhydrous sodium sulfate and the organic phase was evaporated and purified by normal phase column chromatography to obtain the target product (186 mg, with a yield of 72%). ¹H NMR (400 MHz, Chloroform-d) δ 10.50 (s, 1H), 8.48 (d, J=8.4 Hz, 1H), 7.65 (d, J=8.5 Hz, 1H), 7.55 (s, 1H), 7.05 (d, J=8.1 Hz, 2H), 7.03-6.96 (m, 1H), 6.98-6.90 (m, 2H), 6.84-6.73 (m, 3H), 6.65 (d, J=7.9 Hz, 1H), 6.34 (s, 1H), 4.39 (d, J=9.1 Hz, 1H), 4.07 (d, J=14.7 Hz, 1H), 4.01-3.94 (m, 2H), 3.94-3.85 (m, 6H), 3.81 (s, 3H), 3.27 (d, J=10.2 Hz, 1H), 3.23 (d, J=9.5 Hz, 1H), 3.09 (d, J=10.1 Hz, 1H), 1.46 (d, J=14.4 Hz, 1H), 1.20-1.08 (m, 1H), 0.96 (s, 9H).

### Synthesis of Intermediate 2. Methyl 4-((2′S,3S,4′S,5′R)-6-chloro-4′-(3-chloro-2-fluorophenyl)-1-(4-methoxybenzyl)-2′-neopentylspiro[indoline-3,3′-pyrrolidine]-5′-carboxamido)-3-methoxybenzoate (YM155)

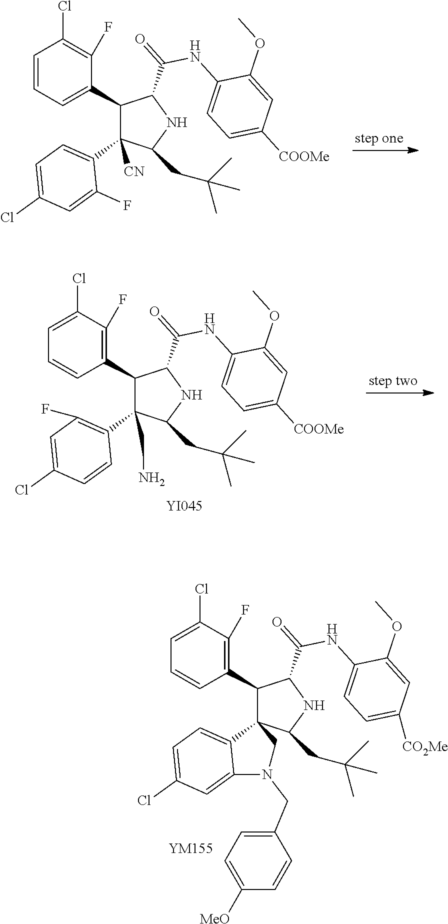

### Step one: Synthesis of methyl 4-((2R,3S,4S,5S)-4-(aminomethyl)-3-(3-chloro-2-fluorophenyl)-4-(4-chloro-2-fluorophenyl)-5-neopentylpyrrolidine-2-carboxamido)-3-methoxybenzoate (YI045)

Methyl 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoate (2.5 g, 3.96 mmol) was dissolved in tetrahydrofuran/ethanol (10/10 mL) and Raney nickel (2.5 g) was added. The mixture was heated to 55° C., and hydrazine hydrate (10 mL) was added. The reaction was continued until no gas was produced. After filtration, the filtrate was concentrated under reduced pressure and purified by normal phase column chromatography to obtain the target compound (637 mg, yield: 25%). ¹H NMR (500 MHz, Methanol-d₄) δ 8.24 (d, J=8.2 Hz, 1H), 7.59-7.50 (m, 2H), 7.50-7.43 (m, 1H), 7.40 (t, J=8.6 Hz, 1H), 7.36-7.26 (m, 2H), 7.26-7.13 (m, 2H), 4.59 (d, J=10.2 Hz, 1H), 4.41 (d, J=11.1 Hz, 1H), 3.97 (d, J=10.3 Hz, 1H), 3.91 (s, 3H), 3.85 (s, 3H), 3.78 (dd, J=14.4, 3.2 Hz, 1H), 3.52 (d, J=14.4 Hz, 1H), 1.72 (d, J=13.6 Hz, 1H), 1.60 (dd, J=13.8, 11.3 Hz, 1H), 1.23 (s, 9H). ¹C NMR (126 MHz, Methanol-d₄) δ 172.68, 168.02, 163.96 (d, J_{C-F}=249.5 Hz), 161.84 (TFA, q, J_{C-F}=36.5 Hz), 158.22 (d, J_{C-F}=248.2 Hz), 149.58, 136.71 (d, J_{C-F}=11.3 Hz), 132.46, 131.36, 130.89 (d, J_{C-F}=5.0 Hz), 130.48, 127.23 (d, J_{C-F}=1.3 Hz), 126.76, 126.01 (d, J_{C-F}=10.1 Hz), 125.95, 123.89, 123.12 (d, J_{C-F}=11.3 Hz), 122.73 (d, J_{C-F}=20.2 Hz), 119.34, 118.88 (d, JC-F=29.0 Hz), 117.50 (TFA, q, J_{C-F}=289.8 Hz), 111.83, 63.89, 63.55, 58.24 (d, J_{C-F}=3.8 Hz), 56.34, 52.62, 43.20, 38.24 (d, J_{C-F}=7.6 Hz), 32.14, 30.68. ESI-MS calculated for C₃₂H₃₆³⁵Cl₂F₂N₃O₄ [M+H]⁺=634.2, found: 634.1.

### Step two: Synthesis of methyl 4-((2′S,3S,4′S,5′R)-6-chloro-4′-(3-chloro-2-fluorophenyl)-1-(4-methoxybenzyl)-2′-neopentylspiro[indoline-3,3′-pyrrolidine]-5′-carboxamido)-3-methoxybenzoate (YM155)

YI045 (322 mg, 0.51 mmol), 4-methoxybenzaldehyde (136 mg, 1 mmol), sodium cyanoborohydride (126 mg, 2 mmol), acetic acid (0.1 mL) and potassium carbonate (207 mg, 1.5 mmol) were reacted according to the procedure described in step 6 of intermediate 1 to obtain the target product (363 mg, yield 93%). ¹H NMR (500 MHz, Chloroform-d) δ 10.47 (s, 1H), 8.46 (d, J=8.6 Hz, 1H), 7.62 (dd, J=8.5, 1.9 Hz, 1H), 7.52 (d, J=1.9 Hz, 1H), 7.30-7.20 (m, 1H), 7.03 (d, J=8.0 Hz, 1H), 6.95-6.87 (m, 3H), 6.86 (d, J=8.6 Hz, 1H), 6.79-6.73 (m, 2H), 6.62 (dd, J=7.9, 1.8 Hz, 1H), 6.31 (d, J=1.8 Hz, 1H), 4.34 (d, J=9.1 Hz, 1H), 4.03 (d, J=14.9 Hz, 1H), 3.96-3.90 (m, 2H), 3.87 (s, 3H), 3.86 (s, 3H), 3.78 (s, 3H), 3.23 (d, J=10.1 Hz, 1H), 3.18 (d, J=9.4 Hz, 1H), 3.02 (dd, J=10.0, 1.3 Hz, 1H), 1.42 (dd, J=14.2, 1.3 Hz, 1H), 1.15-1.06 (m, 1H), 0.93 (s, 9H).

### Synthesis of Intermediate 3: Synthesis of methyl 4-((2S,3R,4R,5R)-3-(3-chloro-2-fluorophenyl)-4-(4-chloro-2-fluorophenyl)-4-(((4-methoxybenzyl)amino)methyl)-5-neopentylpyrrolidine-2-carboxamido)-3-methoxybenzoate (YN20-CF1)

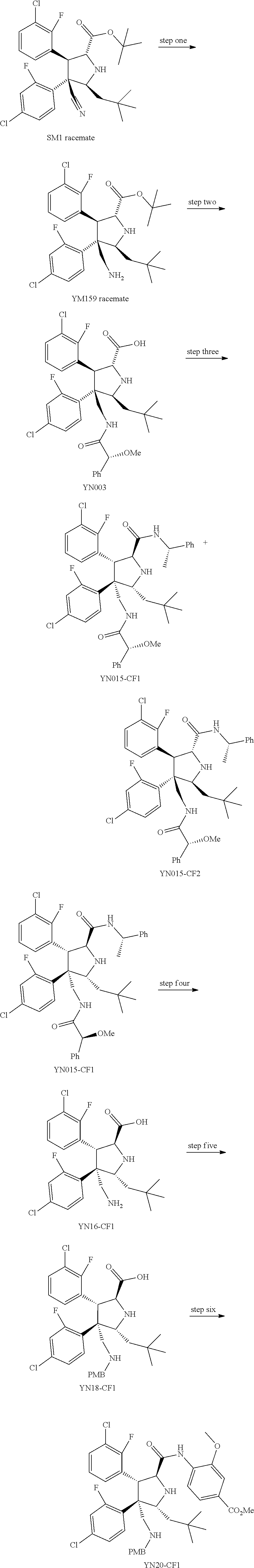

Step one: Synthesis of enantiomer tert-butyl (2R,3S,4S,5S)-4-(aminomethyl)-3-(3-chloro-2-fluorophenyl)-4-(4-chloro-2-fluorophenyl)-5-neopentylpyrrolidine-2-carboxylate and enantiomer tert-butyl (2S,3R,4R,5R)-4-(aminomethyl)-3-(3-chloro-2-fluorophenyl)-4-(4-chloro-2-fluorophenyl)-5-neopentylpyrrolidine-2-carboxylate (YM159)

SM1 (5.45 g, 1.04 mmol) was added to a 100 mL round-bottom flask, followed by addition of Raney Nickel (2 g). The mixture was dissolved in tetrahydrofuran/ethanol, heated to 55° C., and hydrazine hydrate (10 mL) was added. The reaction was continued until no more bubbling was observed. The mixture was filtered, and the filtrate was dried by rotary evaporation. The target compound (1.9 g) was obtained by normal-phase column chromatography with a yield of 36%. ¹H NMR (500 MHz, Methanol-d₄) δ 7.54-7.42 (m, 2H), 7.34 (dd, J=8.7, 2.2 Hz, 1H), 7.31-7.22 (m, 3H), 4.72 (d, J=10.3 Hz, 1H), 4.67 (d, J=9.9 Hz, 1H), 4.30 (d, J=9.9 Hz, 1H), 3.66-3.60 (m, 1H), 3.60-3.52 (m, 1H), 1.77 (dd, J=14.4, 10.4 Hz, 1H), 1.67-1.54 (m, 1H), 1.31 (s, 9H), 1.08 (s, 9H). ¹³C NMR (126 MHz, Methanol-d₄) δ 170.86, 163.01 (TFA, q, J_{C-F}=35.3 Hz), 162.47 (d, J_{C-F}=250.7 Hz), 157.99 (d, J_{C-F}=248.2 Hz), 137.10 (d, J_{C-F}=11.3 Hz), 131.82, 130.70 (d, J_{C-F}=5.0 Hz), 129.59, 127.05 (d, J_{C-F}=3.8 Hz), 126.50 (d, J_{C-F}=3.8 Hz), 124.95 (d, J_{C-F}=13.9 Hz), 123.09 (d, J_{C-F}=10.1 Hz), 122.71 (d, J_{C-F}=18.9 Hz), 119.01 (d, J_{C-F}=30.2 Hz), 118.00 (TFA, q, J_{C-F}=292.3 Hz), 84.70, 63.16 (d, J_{C-F}=3.8 Hz), 61.59, 55.58 (d, J_{C-F}=5.0 Hz), 51.79, 42.42, 39.29 (d, J_{C-F}=6.3 Hz), 31.83, 30.01, 27.88. LRMS (ESI) calculated for C₂₇H₃₅³⁵Cl₂F₂N₂O₂ [M+H]⁺=527.2, found: 527.2.

### Step two: Synthesis of epimer (2R,3S,4S,5S)-3-(3-chloro-2-fluorophenyl)-4-(4-chloro-2-fluorophenyl)-4-(((R)-2-methoxy-2-phenylacetamido)methyl)-5-neopentylpyrrolidine-2-carboxylic acid and epimer (2S,3R,4R,5R)-3-(3-chloro-2-fluorophenyl)-4-(4-chloro-2-fluorophenyl)-4-(((R)-2-methoxy-2-phenylacetamido)methyl)-5-neopentylpyrrolidine-2-carboxylic Acid (YN003)

YM159 (210 mg, 0.4 mmol) was dissolved in dichloromethane, followed by addition of (R)-(−)-alpha-methoxyphenylacetyl chloride (110 mg, 0.6 mmol) and triethylamine (162 mg, 1.6 mmol). The mixture was stirred at room temperature overnight. After completion of the reaction, water was added, and the mixture was extracted with dichloromethane. The organic phase was concentrated by rotary evaporation and purified by normal-phase column chromatography to obtain the crude product. The crude product was dissolved in dichloromethane (5 mL), followed by addition of trifluoroacetic acid (2 mL), and the mixture was stirred at room temperature overnight. After completion of the reaction, water was added, and the mixture was extracted with dichloromethane. The organic phase was concentrated by rotary evaporation and purified by normal-phase column chromatography to obtain the target compound (226 mg) with a yield of 91%.

### Step Three: Synthesis of (2S,3R,4R,5R)-3-(3-chloro-2-fluorophenyl)-4-(4-chloro-2-fluorophenyl)-4-(((R)-2-methoxy-2-phenylacetamido)methyl)-5-neopentyl-N—((S)-1-phenylethyl)pyrrolidine-2-carboxamide (YN015-CF1) and (2R,3S,4S,5S)-3-(3-chloro-2-fluorophenyl)-4-(4-chloro-2-fluorophenyl)-4-(((R)-2-methoxy-2-phenylacetamido)methyl)-5-neopentyl-N—((S)-1-phenylethyl)pyrrolidine-2-carboxamide (YN015-CF2)

YN003 (226 mg, 0.36 mmol) was added to a flask and dissolved in tetrahydrofuran, followed by addition of diisopropylethylamine (232 mg, 1.8 mmol) and diphenylphosphinic chloride (255 mg, 1.08 mmol). After stirring at room temperature for 30 min, (S)-1-phenylethylamine (176 mg, 1.46 mmol) was added, and the mixture was stirred at room temperature overnight. Water was added, and the mixture was extracted with dichloromethane. The organic phase was concentrated by rotary evaporation and purified by normal-phase column chromatography to obtain a mixture of the target compounds (200 mg) with a yield of 77%. The above operation was repeated to accumulate a total of 400 mg of the mixture. The mixture was purified by reverse-phase HPLC to separate YN015-CF1 (172 mg), ¹H NMR (500 MHz, Methanol-d₄) δ 7.73 (t, J=7.0 Hz, 1H), 7.67-7.54 (m, 1H), 7.38 (t, J=8.0 Hz, 1H), 7.31-7.19 (m, 5H), 7.17-7.08 (m, 3H), 7.04-6.97 (m, 2H), 6.94 (t, J=8.8 Hz, 1H), 6.89-6.82 (m, 2H), 6.75 (dd, J=8.5, 2.6 Hz, 1H), 5.22-4.96 (m, 2H), 4.88 (dd, J=10.4, 5.7 Hz, 1H), 4.52 (d, J=9.4 Hz, 1H), 4.45 (s, 1H), 4.18 (d, J=15.3 Hz, 1H), 3.74 (dd, J=15.2, 2.5 Hz, 1H), 3.06 (s, 3H), 1.82 (dd, J=15.3, 9.5 Hz, 1H), 1.46-1.35 (m, 4H), 0.82 (s, 9H). YN015-CF2 (172 mg), ¹H NMR (500 MHz, Methanol-d₄) δ 7.65 (t, J=7.2 Hz, 1H), 7.56 (t, J=7.5 Hz, 1H), 7.40-7.18 (m, 10H), 7.17-6.97 (m, 4H), 5.03 (d, J=10.0 Hz, 1H), 4.97 (q, J=6.8 Hz, 1H), 4.85-4.62 (m, 2H), 4.52 (s, 1H), 4.17-3.95 (m, 1H), 3.85 (d, J=15.3 Hz, 1H), 3.10 (s, 3H), 1.97 (dd, J=15.2, 9.9 Hz, 1H), 1.60 (d, J=15.0 Hz, 1H), 1.23 (d, J=7.0 Hz, 3H), 0.95 (s, 9H). The above operation was repeated to accumulate a total of YN05-CF1 (383 mg) and YN05-CF2 (383 mg).

### Step Four: Synthesis of (2S,3R,4R,5R)-4-(aminomethyl)-3-(3-chloro-2-fluorophenyl)-4-(4-chloro-2-fluorophenyl)-5-neopentylpyrrolidine-2-carboxylic acid (YN16-CF1)

YN015-CF1 (383 mg, 0.5 mmol) was dissolved in 10 mL of concentrated hydrochloric acid, ethanol was added, and the mixture was refluxed for 18 h. After the reaction was completed, the trifluoroacetate salt of YN16-CF1 was obtained by HPLC purification, yielding 187 mg (75%). ¹H NMR (500 MHz, Methanol-d₄) δ 7.55-7.48 (m, 1H), 7.47-7.41 (m, 1H), 7.40-7.36 (m, 1H), 7.36-7.20 (m, 3H), 5.07 (d, J=11.1 Hz, 1H), 4.97 (d, J=10.9 Hz, 1H), 4.29 (d, J=11.1 Hz, 1H), 3.82 (d, J=14.2 Hz, 1H), 3.72-3.63 (m, 1H), 2.09-1.94 (m, 1H), 1.84-1.70 (m, 1H), 1.13 (s, 9H).

### Step five: Synthesis of (2S,3R,4R,5R)-3-(3-chloro-2-fluorophenyl)-4-(4-chloro-2-fluorophenyl)-4-(((4-methoxybenzyl)amino)methyl)-5-neopentylpyrrolidine-2-carboxylic Acid (YN18-CF1)

YN16-CF1 (187 mg, 0.4 mmol) was dissolved in methanol, and p-methoxybenzaldehyde (109 mg, 0.8 mmol), sodium cyanoborohydride (101 mg, 1.6 mmol), and 1 mL of acetic acid were added. The mixture was stirred at room temperature overnight. After the reaction was completed, the mixture was purified by HPLC to obtain the trifluoroacetate salt of the target compound (133 mg, yield: 47%).

### Step six: Synthesis of methyl 4-((2S,3R,4R,5R)-3-(3-chloro-2-fluorophenyl)-4-(4-chloro-2-fluorophenyl)-4-(((4-methoxybenzyl)amino)methyl)-5-neopentylpyrrolidine-2-carboxamido)-3-methoxybenzoate (YN20-CF1)

YN18-CF1 (133 mg, 0.22 mmol) was added into a flask, dissolved in tetrahydrofuran, and diisopropylethylamine (144 mg, 1.12 mmol) and diphenylphosphinic chloride (156 mg, 0.66 mmol) were added. After stirring at room temperature for 30 min, methyl 3-methoxy-4-aminobenzoate (163 mg, 0.9 mmol) was added, and the reaction was continued at room temperature overnight. Water was added, and the mixture was extracted with dichloromethane. The organic phase was dried by rotary evaporation and purified by normal phase column chromatography to obtain the crude product YN20-CF1, which was used directly in the next step.

### Synthesis of Intermediate 4: Synthesis of methyl 4-((2R,3S,4S,5S)-3-(3-chloro-2-fluorophenyl)-4-(4-chloro-2-fluorophenyl)-4-(((4-methoxybenzyl)amino)methyl)-5-neopentylpyrrolidine-2-carboxamido)-3-methoxybenzoate (YN22-CF2)

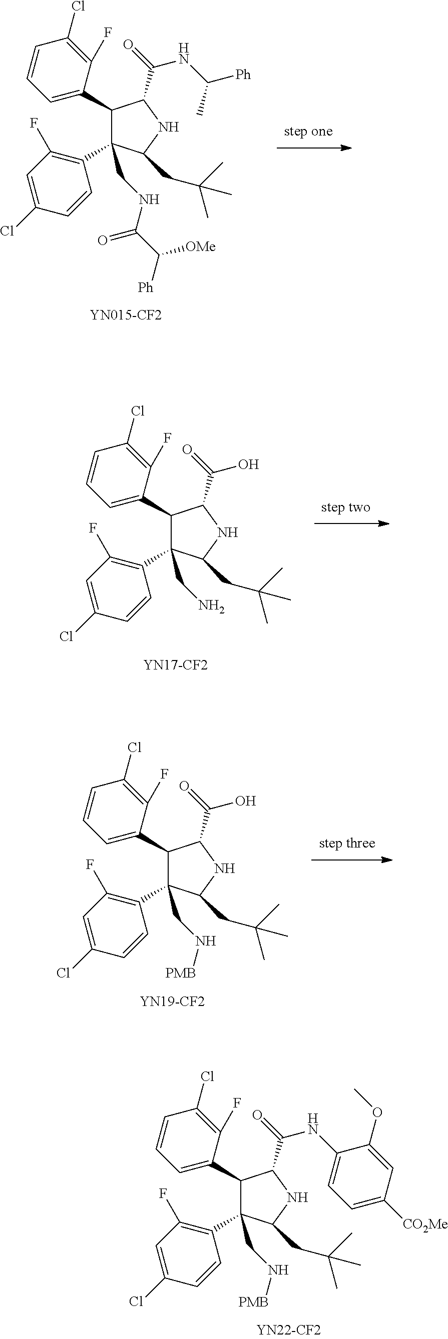

Step one. Synthesis of (2R,3S,4S,5S)-4-(aminomethyl)-3-(3-chloro-2-fluorophenyl)-4-(4-chloro-2-fluorophenyl)-5-neopentylpyrrolidine-2-carboxylic acid (YN17-CF2)

Following the synthesis method of YN16-CF1 in Step Four of the synthesis of Intermediate 3, using YN015-CF2 as the starting material, the trifluoroacetate salt of YN17-CF2 was obtained (203 mg, yield 81%). 1H NMR (500 MHz, Methanol-d₄) δ 7.55-7.48 (m, 1H), 7.47-7.41 (m, 1H), 7.40-7.36 (m, 1H), 7.36-7.20 (m, 3H), 5.01 (d, J=11.1 Hz, 1H), 4.95 (d, J=10.9 Hz, 1H), 4.28 (d, J=11.1 Hz, 1H), 3.81 (d, J=14.2 Hz, 1H), 3.71-3.61 (m, 1H), 2.09-1.94 (m, 1H), 1.84-1.70 (m, 1H), 1.12 (s, 9H).

### Step two: Synthesis of (2R,3S,4S,5S)-3-(3-chloro-2-fluorophenyl)-4-(4-chloro-2-fluorophenyl)-4-(((4-methoxybenzyl)amino)methyl)-5-neopentylpyrrolidine-2-carboxylic Acid (YN19-CF2)

Following the synthesis method of YN18-CF1 in Step Five of the synthesis of Intermediate 3, using YN17-CF2 as the starting material, the trifluoroacetate salt of YN19-CF2 was obtained (181 mg, yield 60%).

### Step Three: Synthesis of methyl 4-((2R,3S,4S,5S)-3-(3-chloro-2-fluorophenyl)-4-(4-chloro-2-fluorophenyl)-4-(((4-methoxybenzyl)amino)methyl)-5-neopentylpyrrolidine-2-carboxamido)-3-methoxybenzoate (YN22-CF2)

Following the synthesis method of YN20-CF1 in Step Six of the synthesis of Intermediate 3, using YN19-CF2 as the starting material, the crude product of YN22-CF2 was obtained.

### Final Product 1: 4-((2′S,3S,4′S,5′R)-6-chloro-4′-(3-chloro-2-fluorophenyl)-1′-methyl-2′-neopentylspiro[indoline-3,3′-pyrrolidine]-5′-carboxamido)-3-methoxybenzoic Acid (YM157)

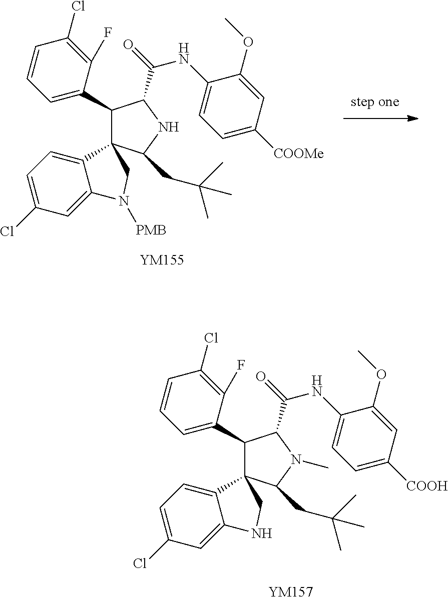

YM155 (40 mg, 0.054 mmol), formaldehyde (41 mg, 0.5 mmol), sodium borohydride acetate (106 mg, 0.5 mmol) and 0.1 mL of acetic acid were added to a 50 mL round-bottom flask, dissolved in 1,2-dichloroethane, and stirred overnight at room temperature. Water was added, the mixture was extracted with ethyl acetate, and the organic phase was evaporated by rotary evaporation. 1 ml of trifluoroacetic acid was added, and the mixture was stirred at 50° C. for 1 h. Saturated sodium bicarbonate solution was added, the mixture was extracted with ethyl acetate, and the organic phase was evaporated by rotary evaporation. Lithium hydroxide monohydrate (8 mg, 0.2 mmol) was added, the mixture was dissolved in tetrahydrofuran/H₂O (v/v=2 mL/mL), and stirred overnight at room temperature. HPLC purification afforded the target compound (12.4 mg, 38% yield). ¹H NMR (500 MHz, Methanol-d₄) δ 8.26 (d, J=8.4 Hz, 1H), 7.65 (dd, J=8.4, 1.8 Hz, 1H), 7.61 (d, J=1.7 Hz, 1H), 7.51-7.43 (m, 1H), 7.43-7.30 (m, 2H), 7.19 (t, J=8.0 Hz, 1H), 6.73 (dd, J=8.1, 1.9 Hz, 1H), 6.49 (d, J=1.8 Hz, 1H), 5.19-5.05 (m, 1H), 4.68-4.48 (m, 1H), 4.30-4.04 (m, 1H), 3.87 (s, 3H), 3.75 (d, J=10.9 Hz, 1H), 3.57 (d, J=11.0 Hz, 1H), 3.06 (s, 3H), 2.01-1.71 (m, 2H), 0.82 (s, 9H). ESI-MS calculated for C₃₂H₅³⁵Cl₂FN₃O₄ [M+H]⁺=614.2, found: 614.2.

### Final Product 2: 4-((2′S,3S,4′S,5′R)-6-chloro-4′-(3-chloro-2-fluorophenyl)-1′-ethyl-2′-neopentylspiro[indoline-3,3′-pyrrolidine]-5′-carboxamido)-3-methoxybenzoic Acid (YN11)

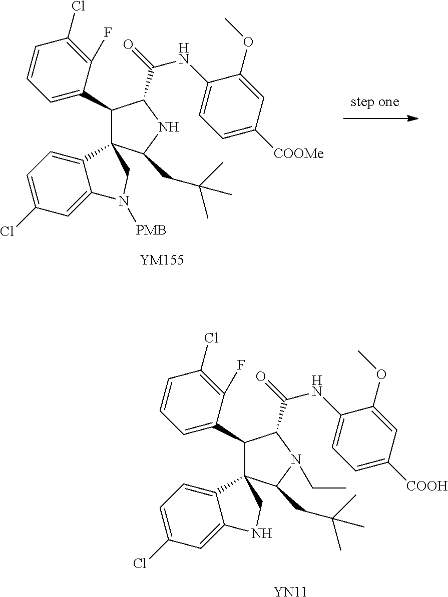

YM155 (50 mg, 0.068 mmol), acetaldehyde (30 mg, 0.68 mmol), sodium borohydride acetate (144 mg, 0.68 mmol), acetic acid (0.1 mL), trifluoroacetic acid (1 mL) and lithium hydroxide monohydrate (14 mg, 0.34 mmol) were reacted according to the procedure described in final product 1 to obtain the target product (20.2 mg, 47% yield). ¹H NMR (500 MHz, Methanol-d₄) δ 8.20 (d, J=8.4 Hz, 1H), 7.65 (d, J=8.9 Hz, 1H), 7.61 (s, 1H), 7.45 (t, J=7.5 Hz, 1H), 7.37 (s, 1H), 7.32 (d, J=8.1 Hz, 1H), 7.19 (t, J=7.9 Hz, 1H), 6.72 (dd, J=8.1, 1.9 Hz, 1H), 6.50 (d, J=1.8 Hz, 1H), 4.57-4.31 (m, 1H), 4.26-4.04 (m, 1H), 3.86 (s, 3H), 3.76 (d, J=11.1 Hz, 1H), 3.61 (dd, J=11.1, 6.9 Hz, 1H), 3.51 (d, J=10.7 Hz, 1H), 3.31-3.16 (m, 2H), 2.01-1.61 (m, 2H), 1.40 (t, J=7.0 Hz, 3H), 0.83 (s, 9H). ESI-MS calculated for C₃₃H₃₇³⁵Cl₂FN₃O₄ [M+H]⁺=628.2, found: 628.7.

### Final Product 3: 4-((2′S,3S,4′S,5′R)-6-chloro-4′-(3-chloro-2-fluorophenyl)-1′-propyl-2′-neopentylspiro[indoline-3,3′-pyrrolidine]-5′-carboxamido)-3-methoxybenzoic Acid (YN51)

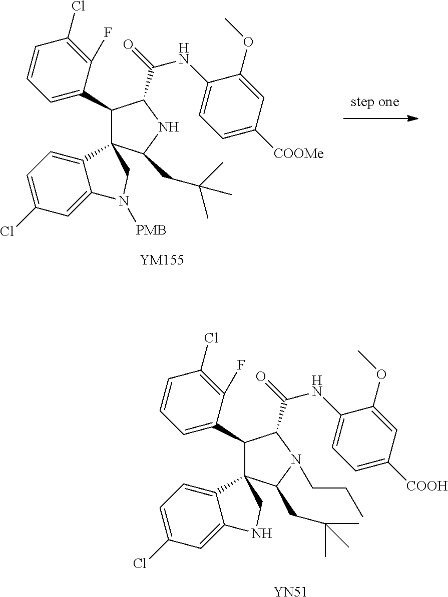

YM155 (50 mg, 0.068 mmol), propionaldehyde (39 mg, 0.68 mmol), sodium borohydride acetate (144 mg, 0.68 mmol), acetic acid (0.1 mL), trifluoroacetic acid (1 mL) and lithium hydroxide monohydrate (14 mg, 0.34 mmol) were reacted according to the procedure described in final product 1 to obtain the target product (5 mg, 11% yield). ¹H NMR (400 MHz, Methanol-d₄) δ 8.27 (d, J=8.4 Hz, 1H), 7.65 (d, J=8.4 Hz, 1H), 7.63 (s, 1H), 7.39 (t, J=7.4 Hz, 1H), 7.32 (t, J=7.5 Hz, 1H), 7.23 (d, J=7.8 Hz, 1H), 7.15 (t, J=7.9 Hz, 1H), 6.69 (dd, J=8.0, 1.9 Hz, 1H), 6.46 (d, J=1.9 Hz, 1H), 4.90-4.43 (m, 1H), 4.42-4.18 (m, 1H), 3.90 (s, 3H), 3.89-3.85 (m, 1H), 3.67 (d, J=10.8 Hz, 1H), 3.56-3.36 (m, 2H), 3.11-2.79 (m, 1H), 2.02-1.89 (m, 1H), 1.86-1.46 (m, 3H), 1.03 (t, J=7.3 Hz, 3H), 0.92 (s, 9H). ESI-MS calculated for C₃₄H₃₉³⁵Cl₂FN₃O₄ [M+H]⁺=642.2, found: 642.3.

### Final Product 4: 4-((2′S,3S,4′S,5′R)-6-chloro-4′-(3-chloro-2-fluorophenyl)-1′-butyl-2′-neopentylspiro[indoline-3,3′-pyrrolidine]-5′-carboxamido)-3-methoxybenzoic Acid (YN52)

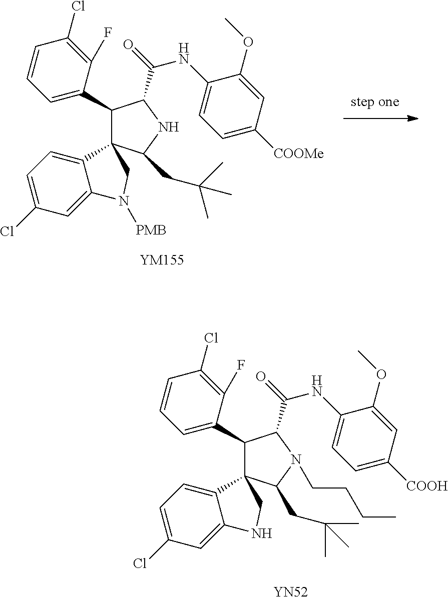

YM155 (50 mg, 0.068 mmol), butyraldehyde (49 mg, 0.68 mmol), sodium borohydride acetate (144 mg, 0.68 mmol), acetic acid (0.1 mL), trifluoroacetic acid (1 mL) and lithium hydroxide monohydrate (14 mg, 0.34 mmol) were reacted according to the procedure described in final product 1 to obtain the target product (9.3 mg, yield 21%). ¹H NMR (500 MHz, Methanol-d₄) δ 8.24 (d, J=8.4 Hz, 1H), 7.66 (dd, J=8.4, 1.8 Hz, 1H), 7.63 (d, J=1.7 Hz, 1H), 7.41 (t, J=7.4 Hz, 1H), 7.34 (t, J=5.7 Hz, 1H), 7.27 (d, J=8.1 Hz, 1H), 7.17 (t, J=7.9 Hz, 1H), 6.70 (dd, J=8.1, 1.9 Hz, 1H), 6.47 (d, J=1.9 Hz, 1H), 4.89-4.57 (m, 1H), 4.44-4.18 (m, 1H), 4.08-3.95 (m, 1H), 3.89 (s, 3H), 3.71 (d, J=10.4 Hz, 1H), 3.56-3.40 (m, 2H), 3.15-2.83 (m, 1H), 2.01-1.87 (m, 1H), 1.82-1.71 (m, 1H), 1.71-1.54 (m, 2H), 1.52-1.41 (m, 2H), 0.95 (t, J=7.3 Hz, 3H), 0.89 (s, 9H). ESI-MS calculated for C₃₅H₄₁³⁵Cl₂FN₃O₄ [M+H]⁺=656.2, found: 656.3.

### Final Product 5: 4-((2′S,3S,4′S,5′R)-6-chloro-4′-(2,3-difluorophenyl)-1′-methyl-2′-neopentylspiro[indoline-3,3′-pyrrolidine]-5′-carboxamido)-3-methoxybenzoic Acid (JN05-2)

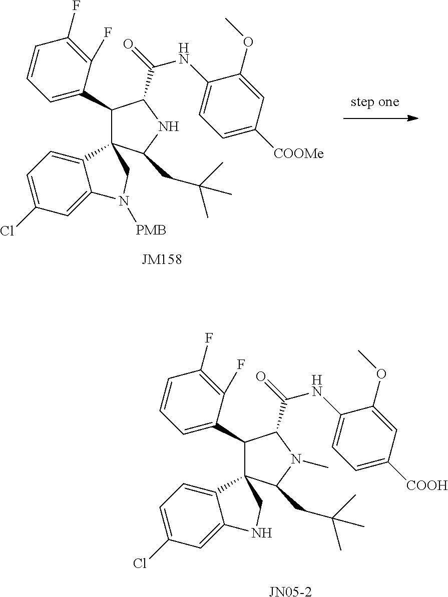

JM158 (80 mg, 0.11 mmol), formaldehyde (90 mg, 1.1 mmol), sodium borohydride acetate (236 mg, 1.1 mmol), acetic acid (0.1 mL), trifluoroacetic acid (1 mL) and lithium hydroxide monohydrate (14 mg, 0.34 mmol) were reacted according to the procedure described in final product 1 to obtain the target product (17.2 mg, yield 26%). ¹H NMR (400 MHz, Methanol-d₄) δ 8.23 (d, J=8.4 Hz, 1H), 7.64 (d, J=8.7 Hz, 1H), 7.60 (s, 1H), 7.39 (d, J=8.1 Hz, 1H), 7.30-7.10 (m, 3H), 6.72 (d, J=8.1 Hz, 1H), 6.49 (s, 1H), 5.15 (d, J=10.2 Hz, 1H), 4.63 (d, J=10.0 Hz, 1H), 4.23-4.06 (m, 1H), 3.87 (s, 3H), 3.76 (d, J=11.0 Hz, 1H), 3.61 (d, J=11.0 Hz, 1H), 3.07 (s, 3H), 2.06-1.77 (m, 2H), 0.80 (s, 9H). ESI-MS calculated for C₃₂H₃₅³⁵ClF₂N₃O₄ [M+H]⁺=598.2, found: 598.2.

### Final Product 6: 4-((2′S,3S,4′S,5′R)-6-chloro-4′-(2,3-difluorophenyl)-1′-ethyl-2′-neopentylspiro[indoline-3,3′-pyrrolidine]-5′-carboxamido)-3-methoxybenzoic Acid (JM151)

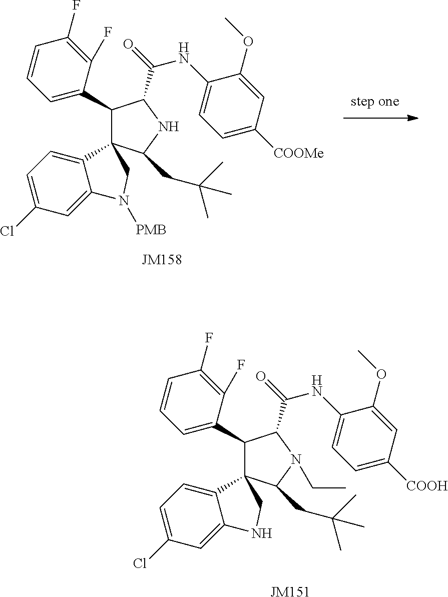

JM158 (60 mg, 0.08 mmol), acetaldehyde (36 mg, 0.8 mmol), sodium borohydride acetate (160 mg, 0.8 mmol), acetic acid (0.1 mL), trifluoroacetic acid (1 mL) and lithium hydroxide monohydrate (13 mg, 0.3 mmol) were reacted according to the procedure described in final product 1 to obtain the target product (8.6 mg, yield 18%). ¹H NMR (400 MHz, Methanol-d₄) δ 8.20 (d, J=8.4 Hz, 1H), 7.66 (d, J=8.6 Hz, 1H), 7.61 (s, 1H), 7.31 (d, J=8.2 Hz, 1H), 7.29-7.09 (m, 3H), 6.72 (d, J=8.1 Hz, 1H), 6.49 (s, 1H), 5.21-4.90 (m, 1H), 4.59-4.31 (m, 1H), 4.23-4.03 (m, 1H), 3.86 (s, 3H), 3.75 (d, J=11.0 Hz, 1H), 3.67-3.58 (m, 1H), 3.55 (d, J=11.4 Hz, 1H), 3.33-3.05 (m, 1H), 2.02-1.63 (m, 2H), 1.40 (t, J=7.0 Hz, 3H), 0.85 (s, 9H). ESI-MS calculated for C₃₃H₃₇³⁵ClF₂N₃O₄ [M+H]⁺=612.2, found: 612.2.

### Final Product 7: 4-((2′S,3S,4′S,5′R)-6-chloro-4′-(2,3-difluorophenyl)-1′-propyl-2′-neopentylspiro[indoline-3,3′-pyrrolidine]-5′-carboxamido)-3-methoxybenzoic Acid (JM156)

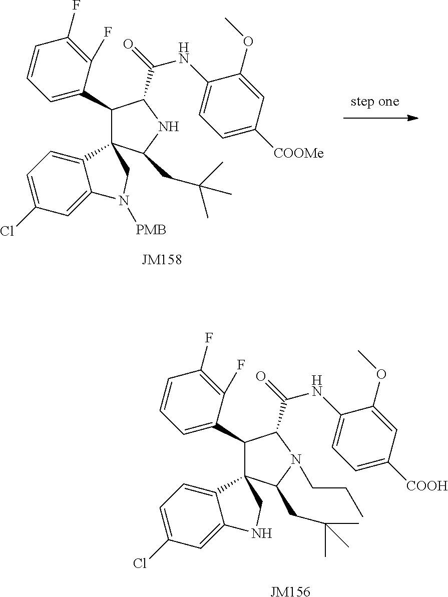

JM158 (58 mg, 0.08 mmol), propionaldehyde (47 mg, 0.8 mmol), sodium borohydride acetate (170 mg, 0.8 mmol), acetic acid (0.1 mL), trifluoroacetic acid (1 mL) and lithium hydroxide monohydrate (9 mg, 0.2 mmol) were reacted according to the procedure described in final product 1 to obtain the target product (4.1 mg, yield 8%). ¹H NMR (400 MHz, Methanol-d₄) δ 8.24 (d, J=8.4 Hz, 1H), 7.66 (d, J=8.8 Hz, 1H), 7.62 (s, 1H), 7.28 (d, J=8.2 Hz, 1H), 7.24-7.05 (m, 3H), 6.71 (d, J=8.0 Hz, 1H), 6.49 (s, 1H), 4.90-4.53 (m, 1H), 4.48-4.20 (m, 1H), 4.16-3.97 (m, 1H), 3.88 (s, 3H), 3.72 (d, J=11.0 Hz, 1H), 3.63-3.50 (m, 1H), 3.50-3.38 (m, 1H), 3.12-2.89 (m, 1H), 2.02-1.90 (m, 1H), 1.89-1.47 (m, 3H), 1.03 (t, J=7.4 Hz, 3H), 0.90 (s, 9H). ESI-MS calculated for C₃₄H₃₉³⁵ClF₂N₃O₄ [M+H]⁺=626.2, found: 626.3.

### Final Product 8: 4-((2′S,3S,4′S,5′R)-6-chloro-4′-(2,3-difluorophenyl)-1′-butyl-2′-neopentylspiro[indoline-3,3′-pyrrolidine]-5′-carboxamido)-3-methoxybenzoic Acid (JN06)

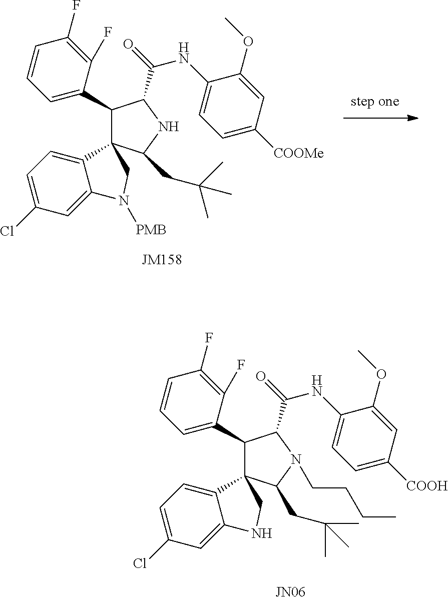

JM158 (70 mg, 0.1 mmol), butyraldehyde (73 mg, 1.0 mmol), sodium borohydride acetate (212 mg, 1.0 mmol), acetic acid (0.1 mL), trifluoroacetic acid (1 mL) and lithium hydroxide monohydrate (21 mg, 0.5 mmol) were reacted according to the procedure described in final product 1 to obtain the target product (10.3 mg, yield 16%). ¹H NMR (400 MHz, Methanol-d₄) δ 8.22 (d, J=8.3 Hz, 1H), 7.65 (d, J=8.9 Hz, 1H), 7.62 (s, 1H), 7.29 (d, J=8.2 Hz, 1H), 7.24-7.11 (m, 3H), 6.71 (d, J=8.1 Hz, 1H), 6.48 (s, 1H), 4.93-4.66 (m, 1H), 4.54-4.24 (m, 1H), 4.13-3.96 (m, 1H), 3.88 (s, 3H), 3.73 (d, J=11.0 Hz, 1H), 3.61-3.42 (m, 2H), 3.20-2.91 (m, 1H), 1.97 (d, J=16.3 Hz, 1H), 1.88-1.73 (m, 2H), 1.72-1.55 (m, 2H), 1.54-1.37 (m, 2H), 0.94 (t, J=7.6 Hz, 3H), 0.87 (s, 9H). ESI-MS calculated for C₃₅H₄₁³⁵ClF₂N₃O₄ [M+H]⁺=640.2, found: 640.2.

### Final Product 9: 4-((2′S,3S,4′S,5′R)-6-chloro-4′-(2-fluorophenyl)-1′-ethyl-2′-neopentylspiro[indoline-3,3′-pyrrolidine]-5′-carboxamido)-3-methoxybenzoic Acid (JN22)

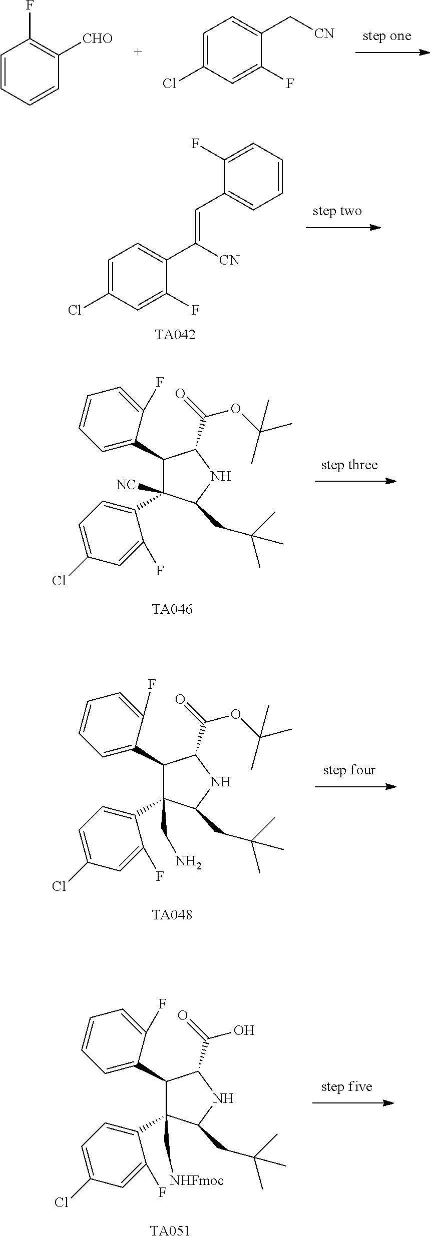

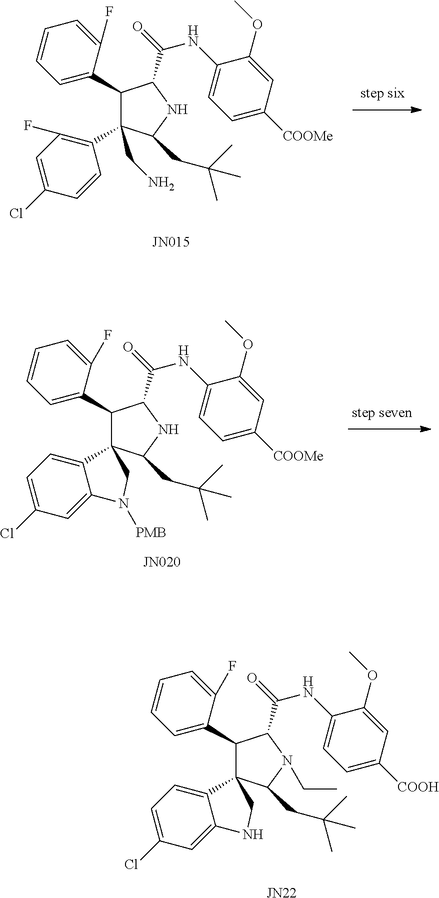

### Step one: Synthesis of (E)-2-(4-chloro-2-fluorophenyl)-3-(2-fluorophenyl)acrylonitrile (TA042)

2-Fluorobenzaldehyde (2.5 g, 20 mmol), 4-chloro-2-fluorophenylacetonitrile (3.4 g, 20 mmol) and 5N sodium methoxide in methanol (4.8 mL) were reacted according to the procedure described in step one of intermediate 1 to obtain the target product (5.3 g, yield 96%). ¹H NMR (500 MHz, Chloroform-d) δ 8.27 (td, J=7.7, 1.6 Hz, 1H), 7.82 (s, 1H), 7.56 (t, J=8.3 Hz, 1H), 7.51-7.42 (m, 1H), 7.29 (t, J=7.6 Hz, 1H), 7.25 (dd, J=8.4, 2.0 Hz, 1H), 7.22 (dd, J=10.8, 2.1 Hz, 1H), 7.19-7.13 (m, 1H).

### Step two: Synthesis of tert-butyl (2R,3R,4R,5S)-4-(4-chloro-2-fluorophenyl)-3-(2-fluorophenyl)-4-cyano-5-neopentylpyrrolidine-2-carboxylate (TA046)

TA042 (5.3 g, 19.3 mmol), AgF (2.54 g, 20 mmol), triethylamine (4.4 mL) and tert-butyl (E)-2-(((3,3-dimethylbutylidene)amino)acetate (4.4 g, 20 mmol) were reacted according to the procedure described in step two of intermediate 1 to obtain the target product (3.16 g, yield 33.5%). ¹H NMR (400 MHz, Chloroform-d) δ 7.69 (t, J=7.5 Hz, 1H), 7.34 (t, J=8.5 Hz, 1H), 7.29-7.12 (m, 3H), 7.08 (d, J=8.6 Hz, 1H), 6.88 (t, J=9.0 Hz, 1H), 4.68 (d, J=7.6 Hz, 1H), 4.22 (d, J=7.6 Hz, 1H), 4.12 (d, J=8.9 Hz, 1H), 1.70-1.58 (m, 1H), 1.37 (s, 9H), 1.30 (d, J=14.4 Hz, 1H), 0.91 (s, 9H).

### Step Three: Synthesis of tert-butyl (2R,3R,4S,5S)-4-(aminomethyl)-4-(4-chloro-2-fluorophenyl)-3-(2-fluorophenyl)-5-neopentylpyrrolidine-2-carboxylate (TA048)

TA046 (3.16 g, 6.47 mmol), Raney nickel (8.0 g) and hydrazine hydrate (10 mL) were reacted according to the procedure described in step three of intermediate 1 to obtain the target product (0.67 g, yield 21%). ¹H NMR (400 MHz, Methanol-d₄) δ 7.48 (t, J=7.7 Hz, 1H), 7.34-7.29 (m, 1H), 7.27-7.13 (m, 4H), 7.01 (t, J=9.6 Hz, 1H), 4.37-4.19 (m, 3H), 3.38 (d, J=14.1 Hz, 1H), 3.24 (d, J=14.1 Hz, 1H), 1.57-1.41 (m, 3H), 1.33 (s, 9H), 1.00 (s, 9H).

### Step Four: Synthesis of (2R,3R,4S,5S)-4-((((((9H-fluoren-9-yl)methoxy)carbonyl)amino)methyl)-4-(4-chloro-2-fluorophenyl)-3-(2-fluorophenyl)-5-neopentylpyrrolidine-2-carboxylic Acid (TA051)

TA048 (0.67 g, 1.4 mmol), FmocCl (0.52 g, 2.0 mmol), diisopropylethylamine (0.7 g, 5.4 mmol) and trifluoroacetic acid (4 mL) were reacted according to the procedure described in step four of intermediate 1 to obtain the target product (0.36 g, yield 40.6%).

### Step five: Synthesis of methyl 4-((2R,3R,4S,5S)-4-(aminomethyl)-4-(4-chloro-2-fluorophenyl)-3-(2-fluorophenyl)-5-neopentylpyrrolidine-2-carboxamido)-3-methoxybenzoate (JN015)

TA051 (239 mg, 0.4 mmol), methyl 4-amino-3-methoxybenzoate (268 mg, 1.5 mmol), diphenylphosphinic chloride (264 mg, 1.1 mmol), diisopropylethylamine (239 mg, 1.9 mmol) and piperidine 0.4 mL) were reacted according to the procedure described in step five of intermediate 1 to obtain the target product (56 mg, yield 25%). ¹H NMR (500 MHz, Methanol-d₄): δ 8.27 (d, J=8.4 Hz, 1H), 7.62-7.56 (m, 2H), 7.43-7.34 (m, 2H), 7.34-7.27 (m, 2H), 7.23 (t, J=7.5 Hz, 1H), 7.17 (dd, J=13.1, 2.2 Hz, 1H), 7.07 (dd, J=10.9, 8.2 Hz, 1H), 4.63 (d, J=10.4 Hz, 1H), 4.42 (d, J=11.2 Hz, 1H), 3.96-3.89 (m, 4H), 3.88 (s, 3H), 3.74 (dd, J=14.4, 3.2 Hz, 1H), 3.56 (d, J=13.7 Hz, 1H), 1.71 (d, J=13.7 Hz, 1H), 1.59 (dd, J=13.8, 11.3 Hz, 1H), 1.23 (s, 9H). ESI-MS calculated for C₃₂H₃₇ClF₂N₃O₄ [M+H]⁺=600.2, found: 600.2.

### Step six: Synthesis of methyl 4-((2′S,3S,4′S,5′R)-6-chloro-4′-(2,3-difluorophenyl)-4-fluoro-1-(4-methoxybenzyl)-2′-neopentylspiro[indoline-3,3′-pyrrolidine]-5′-carboxamido)-3-methoxybenzoate (JN020)

JN015 (56 mg, 0.1 mmol), 4-methoxybenzaldehyde (64 mg, 0.5 mmol), sodium cyanoborohydride (32 mg, 0.5 mmol), acetic acid (0.1 mL) and potassium carbonate (42 mg, 0.3 mmol) were reacted according to the procedure described in step six of intermediate 1 to obtain the target product (53 mg, yield 76%). ¹H NMR (500 MHz, Chloroform-d) δ 8.51 (d, J=8.5 Hz, 1H), 7.65 (dd, J=8.5, 1.7 Hz, 1H), 7.55 (d, J=1.7 Hz, 1H), 7.21 (dd, J=9.1, 3.7 Hz, 1H), 7.11-7.06 (m, 2H), 7.04 (t, J=7.4 Hz, 1H), 6.96 (dd, J=11.3, 8.2 Hz, 1H), 6.92 (d, J=8.4 Hz, 2H), 6.78 (d, J=8.5 Hz, 2H), 6.64 (dd, J=7.9, 1.8 Hz, 1H), 6.29 (d, J=1.7 Hz, 1H), 4.40 (d, J=9.2 Hz, 1H), 4.10 (d, J=15.1 Hz, 1H), 4.01 (d, J=9.1 Hz, 1H), 3.90 (s, 3H), 3.89 (s, 3H), 3.89-3.85 (m, 1H), 3.81 (s, 3H), 3.29 (d, J=10.0 Hz, 1H), 3.22 (d, J=9.6 Hz, 1H), 3.13 (d, J=9.9 Hz, 1H), 1.47 (d, J=14.2 Hz, 1H), 1.14 (dd, J=14.4, 9.7 Hz, 1H), 0.97 (s, 9H).

### Step seven: Synthesis of 4-((2′S,3S,4′R,5′R)-6-chloro-4′-(2,3-difluorophenyl)-2′-neopentylspiro[dihydroindole-3,3′-pyrrolidine]-5′-carboxamido)-3-methoxybenzoic Acid (JN22)

JN020 (53 mg, 0.08 mmol), acetaldehyde (34 mg, 0.8 mmol), sodium borohydride acetate (162 mg, 0.8 mmol), acetic acid (0.1 mL), trifluoroacetic acid (1 mL) and lithium hydroxide monohydrate (15 mg, 0.4 mmol) were reacted according to the procedure described in final product 1 to obtain the target product (10.3 mg, yield 21%). ¹H NMR (400 MHz, Methanol-d₄) δ 8.18 (d, J=8.3 Hz, 1H), 7.65 (d, J=8.5 Hz, 1H), 7.60 (s, 1H), 7.51-7.27 (m, 3H), 7.22 (t, J=7.7 Hz, 1H), 7.10 (t, J=9.7 Hz, 1H), 6.71 (d, J=8.0 Hz, 1H), 6.48 (s, 1H), 5.22-4.91 (m, 1H), 4.56-4.34 (m, 1H), 4.28-4.03 (m, 1H), 3.83 (s, 3H), 3.77 (d, J=11.1 Hz, 1H), 3.69-3.49 (m, 2H), 3.40-3.33 (m, 1H), 2.02-1.67 (m, 2H), 1.42 (t, J=7.2 Hz, 3H), 0.81 (s, 9H). ESI-MS calculated for C₃₃H₃₈³⁵ClFN₃O₄ [M+H]⁺=594.3, found: 594.3.

### Final Product 10: 4-((2′S,3S,4′S,5′R)-6-chloro-4′-(3-fluorophenyl)-1′-ethyl-2′-neopentylspiro[indoline-3,3′-pyrrolidine]-5′-carboxamido)-3-methoxybenzoic Acid (JM159)

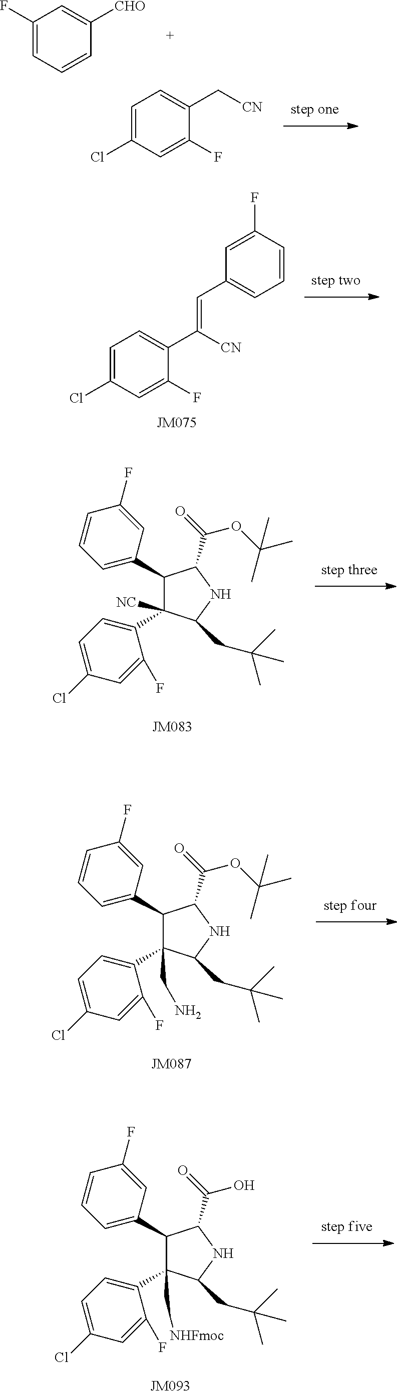

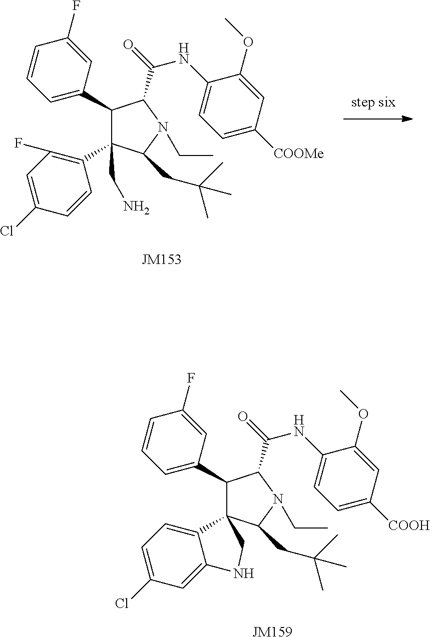 [SMILES: CCN1[C@@H](CC(C)(C)C)[C@](CN)(c2ccc(Cl)cc2F)[C@@H](c2cccc(F)c2)[C@@H]1C(=O)Nc1ccc(C(=O)OC)cc1OC ; "JM153" ; CCN1[C@@H](CC(C)(C)C)[C@@]2(CNc3cc(Cl)ccc32)[C@@H](c2cccc(F)c2)[C@@H]1C(=O)Nc1ccc(C(=O)O)cc1OC ; "JM159"]

### Step one: Synthesis of (Z)-2-(4-chloro-2-fluorophenyl)-3-(3-fluorophenyl)acrylonitrile (JM075)

3-Fluorobenzaldehyde (2.5 g, 20 mmol), 4-chloro-2-fluorophenylacetonitrile (3.4 g, 20 mmol), and 5N sodium methoxide in methanol (4.8 mL) were reacted according to the procedure described in step one of intermediate 1 to obtain the target product (5.3 g, yield 96%). ¹H NMR (400 MHz, Chloroform-d) δ 7.66 (d, J=7.9 Hz, 1H), 7.62 (dt, J=9.9, 2.1 Hz, 1H), 7.59-7.53 (m, 2H), 7.47 (td, J=8.1, 5.8 Hz, 1H), 7.31-7.22 (m, 2H), 7.19 (td, J=8.3, 2.2 Hz, 1H).

### Step two: Synthesis of tert-butyl (2R,3R,4R,5S)-4-(4-chloro-2-fluorophenyl)-3-(3-fluorophenyl)-4-cyano-5-neopentylpyrrolidine-2-carboxylate (JM083)

JM075 (5.3 g, 19 mmol), AgF (2.4 g, 19 mmol), triethylamine (3.2 mL) and (E)-tert-butyl 2-(((3,3-dimethylbutylidene)amino)acetate (4.9 g, 23 mmol) were reacted according to the procedure described in step two of intermediate 1 to obtain the target product (2.4 g, yield 26%). ¹H NMR (500 MHz, Chloroform-d) δ 7.34 (t, J=8.5 Hz, 1H), 7.25-7.18 (m, 1H), 7.18 (dd, J=12.4, 2.1 Hz, 1H), 7.11 (dd, J=8.5, 2.2 Hz, 1H), 6.94 (dd, J=7.8, 2.6 Hz, 1H), 6.91 (dt, J=10.0, 1.5 Hz, 1H), 4.24 (d, J=7.8 Hz, 1H), 4.16 (d, J=7.7 Hz, 1H), 4.05 (d, J=9.0 Hz, 1H), 1.60 (ddd, J=14.3, 9.2, 0.7 Hz, 1H), 1.37 (s, 9H), 1.28 (dd, J=14.1, 1.2 Hz, 1H), 0.88 (s, 9H).

### Step Three: Synthesis of tert-butyl (2R,3R,4S,5S)-4-(aminomethyl)-4-(4-chloro-2-fluorophenyl)-3-(3-fluorophenyl)-5-neopentylpyrrolidine-2-carboxylate (JM087)

JM083 (2.2 g, 4.5 mmol), Raney nickel (2.7 g) and hydrazine hydrate (10 mL) were reacted according to the procedure described in step three of intermediate 1 to obtain the target product (594 mg, yield 27%). ¹H NMR (500 MHz, Chloroform-d) δ 7.16 (td, J=7.9, 6.1 Hz, 1H), 7.10-6.99 (m, 2H), 6.93-6.82 (m, 3H), 4.27 (d, J=8.7 Hz, 1H), 4.11 (d, J=9.1 Hz, 1H), 3.91 (dd, J=8.6, 2.1 Hz, 1H), 3.25 (d, J=13.3 Hz, 1H), 3.04 (d, J=13.3 Hz, 1H), 1.48-1.38 (m, 2H), 1.28 (s, 9H), 0.88 (s, 9H).

### Step Four: Synthesis of (2R,3R,4S,5S)-4-((((((9H-fluoren-9-yl)methoxy)carbonyl)amino)methyl)-4-(4-chloro-2-fluorophenyl)-3-(3-fluorophenyl)-5-neopentylpyrrolidine-2-carboxylic Acid (JM093)

JM087 (494 mg, 1.0 mmol), FmocCl (389 mg, 1.5 mmol), diisopropylethylamine (516 mg, 4.0 mmol) and trifluoroacetic acid (5 mL) were reacted according to the procedure described in step four of intermediate 1 to obtain the target product (396 mg, yield 60%).

### Step five: Methyl 4-((2R,3R,4S,5S)-4-(aminomethyl)-4-(4-chloro-2-fluorophenyl)-1-ethyl-3-(3-fluorophenyl)-5-neopentylpyrrolidine-2-carboxamido)-3-methoxybenzoate (JM153)

JM093 (131 mg, 0.2 mmol) was added to a 50 mL single-neck flask, dissolved in dry tetrahydrofuran, diisopropylethylamine (129 mg, 1.0 mmol) was added, stirred for 5 minutes, diphenylphosphinic chloride (143 mg, 0.6 mmol) was added, stirred for half an hour, then methyl 4-amino-3-methoxybenzoate (147 mg, 4.0 mmol) was added, reacted overnight at room temperature. Saturated sodium bicarbonate solution was added to the reaction mixture, extracted with dichloromethane, the organic phase was dried by rotary evaporation and purified by normal phase column chromatography to obtain the crude product (116 mg). The crude product (116 mg), acetaldehyde (47 mg, 1.1 mmol), sodium borohydride acetate (234 mg, 1.1 mmol) and 0.1 mL acetic acid were added into a 50 mL round-bottom flask, dissolved in 1,2-dichloroethane, reacted overnight at room temperature, and then saturated sodium bicarbonate solution was added, extracted with ethyl acetate. The organic phase was evaporated by rotary evaporation, the obtained crude product was dissolved in DMF (2 mL), piperidine (0.4 mL) was added, reacted at room temperature for 15 minutes, water was added, extracted with ethyl acetate, the organic phase was evaporated by rotary evaporation and purified by normal phase column chromatography to obtain the target product (32 mg, yield 38%). ¹H NMR (400 MHz, Methanol-d₄) δ 8.26 (dd, J=8.5, 2.2 Hz, 1H), 7.68-7.56 (m, 2H), 7.51 (t, J=7.6 Hz, 1H), 7.39-7.30 (m, 2H), 7.27 (d, J=13.6 Hz, 1H), 7.06 (t, J=7.6 Hz, 1H), 6.96 (d, J=10.4 Hz, 1H), 6.90 (d, J=7.8 Hz, 1H), 4.42 (d, J=8.8 Hz, 1H), 4.36 (d, J=9.2 Hz, 1H), 4.09 (d, J=9.2 Hz, 1H), 3.93 (s, 3H), 3.88 (s, 3H), 3.58-3.37 (m, 2H), 3.29-3.20 (m, 1H), 3.09-2.93 (m, 1H), 2.00 (dd, J=15.4, 9.0 Hz, 1H), 1.45 (d, J=15.0 Hz, 1H), 1.24 (t, J=6.1 Hz, 3H), 1.02 (s, 9H).

### Step six: Synthesis of 4-((2′S,3S,4′S,5′R)-6-chloro-4′-(3-fluorophenyl)-1′-ethyl-2′-neopentylspiro[indoline-3,3′-pyrrolidine]-5′-carboxamido)-3-methoxybenzoic acid (JM159)

JM153 (32 mg, 0.05 mmol) was added into a flask, dissolved in 2 mL DMF, and potassium carbonate (29 mg, 0.2 mmol) was added. The mixture was stirred overnight at 110° C. After the reaction was complete, the mixture was cooled to room temperature, water was added, and the mixture was extracted with ethyl acetate. After the organic phase was evaporated by rotary evaporation the crude product was dissolved in 10 mL of water/tetrahydrofuran/MeOH (V/V/V=1/1/1) mixed solvent, and lithium hydroxide monohydrate (21 mg, 0.5 mmol) was added and stirred overnight at room temperature. The reaction solution was evaporated by rotary evaporation, and the target product was purified by HPLC to yield 13.4 mg (45%). ¹H NMR (400 MHz, Methanol-d₄) δ 8.18 (d, J=8.3 Hz, 1H), 7.63 (d, J=8.5 Hz, 1H), 7.58 (s, 1H), 7.42-7.23 (m, 2H), 7.06 (d, J=9.4 Hz, 1H), 7.05-6.95 (m, 2H), 6.73 (d, J=8.0 Hz, 1H), 6.45 (s, 1H), 5.25-4.91 (m, 1H), 4.32-4.01 (m, 2H), 3.82 (s, 3H), 3.77-3.60 (m, 2H), 3.58-3.48 (m, 1H), 3.32-3.13 (m, 1H), 2.01-1.90 (m, 1H), 1.89-1.59 (m, 1H), 1.41 (t, J=7.1 Hz, 3H), 0.86 (s, 9H). ESI-MS calculated for C₃₃H₃₈³⁵ClFN₃O₄ [M+H]⁺=594.2, found: 594.5.

### Final Product 11: 4-((2′S,3S,4′S,5′R)-6-chloro-4′-(2,3-dichlorophenyl)-1′-ethyl-2′-neopentylspiro[indoline-3,3′-pyrrolidine]-5′-carboxamido)-3-methoxybenzoic Acid (JN17)

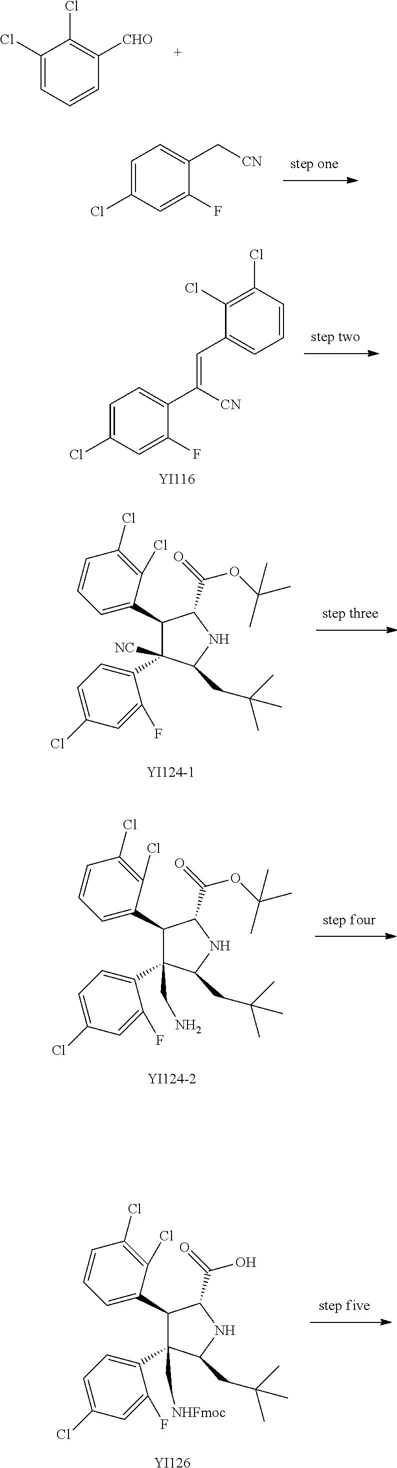

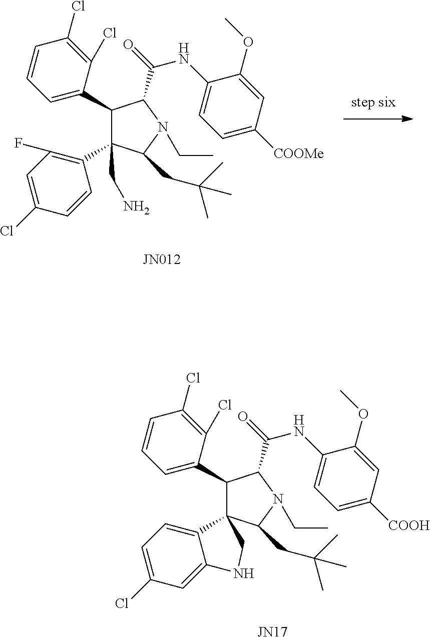 [SMILES: CCN1[C@@H](CC(C)(C)C)[C@](CN)(c2ccc(Cl)cc2F)[C@@H](c2cccc(Cl)c2Cl)[C@@H]1C(=O)Nc1ccc(C(=O)OC)cc1OC ; "JN012" ; CCN1[C@@H](CC(C)(C)C)[C@@]2(CNc3cc(Cl)ccc32)[C@@H](c2cccc(Cl)c2Cl)[C@@H]1C(=O)Nc1ccc(C(=O)O)cc1OC ; "JN17"]

### Step one: Synthesis of (Z)-2-(4-chloro-2-fluorophenyl)-3-(2,3-dichlorophenyl)acrylonitrile (YI116)

2,3-Dichlorobenzaldehyde (3.5 g, 20 mmol), 4-chloro-2-fluorophenylacetonitrile (3.4 g, 20 mmol), and 4.8 mL of 5N sodium methoxide in methanol were reacted according to the procedure described in step one of intermediate 1 to yield the target product (6.23 g, 95%). ¹H NMR (500 MHz, Chloroform-d) δ 7.98-7.89 (m, 2H), 7.62-7.54 (m, 2H), 7.39-7.32 (m, 1H), 7.28 (dd, J=2.1, 0.9 Hz, 1H), 7.23 (d, J=2.1 Hz, 1H).

### Step two: Synthesis of (2R,3R,4R,5S)-4-(4-chloro-2-fluorophenyl)-3-(2,3-dichlorophenyl)-4-cyano-5-neopentylpyrrolidine-2-carboxylic Acid Tert-Butyl Ester (YI124-1)

YI116 (6.23 g, 19 mmol), AgF (2.41 g, 19 mmol), triethylamine (4.24 mL) and tert-butyl (E)-2-(((3,3-dimethylbutylidene)amino)acetate (4 g, 19 mmol) were reacted according to the procedure described in step two of intermediate 1 to yield the target product (4.57 g, 45%). ¹H NMR (500 MHz, Chloroform-d) δ 7.72 (dd, J=7.9, 1.6 Hz, 1H), 7.39 (dd, J=8.0, 1.6 Hz, 1H), 7.38-7.29 (m, 2H), 7.14 (dd, J=12.3, 2.1 Hz, 1H), 7.12-7.08 (m, 1H), 5.02 (d, J=6.4 Hz, 1H), 4.09 (d, J=8.9 Hz, 1H), 4.03 (d, J=6.3 Hz, 1H), 1.74-1.61 (m, 1H), 1.42 (s, 9H), 1.29 (dd, J=14.3, 1.0 Hz, 1H), 0.91 (s, 9H).

### Step Three: Synthesis of tert-butyl (2R,3R,4S,5S)-4-(aminomethyl)-4-(4-chloro-2-fluorophenyl)-3-(2,3-dichlorophenyl)-5-neopentylpyrrolidine-2-carboxylate (YI124-2)

YI124-1 (4.57 g, 8.5 mmol), Raney nickel (4.5 g), and hydrazine hydrate (10 mL) were reacted according to the procedure described in step three of intermediate 1 to yield the target product (1.04 g, 23%). ¹H NMR (500 MHz, Chloroform-d) δ 7.87 (dd, J=7.9, 1.6 Hz, 1H), 7.34 (dd, J=7.9, 1.4 Hz, 1H), 7.22 (t, J=7.9 Hz, 1H), 7.11 (t, J=8.6 Hz, 1H), 7.05 (dd, J=8.7, 2.2 Hz, 1H), 6.97 (dd, J=12.9, 2.2 Hz, 1H), 4.44 (d, J=8.9 Hz, 1H), 4.20 (dd, J=9.1, 1.8 Hz, 1H), 4.01 (d, J=8.9 Hz, 1H), 3.36-3.29 (m, 2H), 1.60-1.47 (m, 2H), 1.26 (s, 9H), 1.00 (s, 9H).

### Step Four. Synthesis of (2R,3R,4S,5S)-4-((((((9H-fluoren-9-yl)methoxy)carbonyl)amino)methyl)-4-(4-chloro-2-fluorophenyl)-3-(2,3-dichlorophenyl)-5-neopentylpyrrolidine-2-carboxylic acid (YI126)

YI124-2 (1.04 g, 1.91 mmol), FmocCl (740 mg, 2.87 mmol), diisopropylethylamine (986 mg, 7.62 mmol), and trifluoroacetic acid (4 mL) were reacted according to the procedure described in step four of intermediate 1 to yield the target product (1.08 g, 80%).

### Step five: methyl 4-(2R,3R,4S,5S)-4-(aminomethyl)-4-(4-chloro-2-fluorophenyl)-1-ethyl-3-(2,3-dichlorophenyl)-5-neopentylpyrrolidine-2-carboxamido)-3-methoxybenzoate (JN012)

YI126 (200 mg, 0.29 mmol), diisopropylethylamine (188 mg, 1.45 mmol), diphenylphosphinic chloride (207 mg, 0.87 mmol), methyl 4-amino-3-methoxybenzoate (209 mg, 1.15 mmol), acetaldehyde (39 mg, 0.9 mmol), sodium borohydride acetate (191 mg, 0.9 mmol), acetic acid (0.1 mL) and piperidine (0.3 mL) were reacted according to the procedure described in step five of the final product 10 to obtain the target product (10 mg, yield 8%). ¹H NMR (400 MHz, Methanol-d₄) δ 8.27 (d, J=8.3 Hz, 1H), 7.70-7.63 (m, 2H), 7.63 (s, 1H), 7.52 (d, J=8.0 Hz, 1H), 7.46-7.39 (m, 2H), 7.29 (d, J=8.7 Hz, 1H), 7.18 (d, J=13.5 Hz, 1H), 4.62 (d, J=10.7 Hz, 1H), 4.57 (d, J=8.1 Hz, 1H), 4.14 (d, J=9.2 Hz, 1H), 3.98 (s, 3H), 3.89 (s, 3H), 3.65-3.55 (m, 2H), 3.41 (d, J=14.5 Hz, 1H), 3.19-3.06 (m, 1H), 2.16-2.04 (m, 1H), 1.52 (d, J=14.8 Hz, 1H), 1.25 (t, J=6.0 Hz, 3H).

### Step six: Synthesis of 4-((2′S,3S,4′S,5′R)-6-chloro-4′-(2,3-dichlorophenyl)-1′-ethyl-2′-neopentylspiro[indoline-3,3′-pyrrolidine]-5′-carboxamido)-3-methoxybenzoic Acid (JN17)

JN012 (10 mg, 0.02 mmol), potassium carbonate (12 mg, 0.08 mmol) and lithium hydroxide monohydrate (8 mg, 0.2 mmol) were reacted according to the procedure described in step six of the final product 10 to obtain the target product (3.4 mg, yield 27%). ¹H NMR (500 MHz, Methanol-d₄) δ 8.37-8.21 (m, 1H), 7.67 (dd, J=8.4, 1.7 Hz, 1H), 7.63 (s, 1H), 7.61-7.53 (m, 1H), 7.49 (d, J=7.8 Hz, 1H), 7.40 (t, J=7.7 Hz, 1H), 7.33-7.18 (m, 1H), 6.68 (dd, J=8.1, 1.9 Hz, 1H), 6.45 (d, J=1.8 Hz, 1H), 4.82-4.62 (m, 1H), 4.6-4.2 (m, 1H), 4.20-3.93 (m, 1H), 3.89 (s, 3H), 3.71-3.60 (m, 1H), 3.60-3.46 (m, 2H), 3.14-2.85 (m, 1H), 2.02-1.48 (m, 2H), 1.34 (t, J=6.3 Hz, 3H), 0.93 (s, 9H). ESI-MS calculated for C₃₃H₃₇³⁵Cl₃N₃O₄ [M+H]⁺=644.2, found: 644.7.

### Final Product 12: 4-((2′S,3S,4′S,5′R)-6-chloro-4′-(4-chlorophenyl)-1′-ethyl-2′-neopentylspiro[indoline-3,3′-pyrrolidine]-5′-carboxamido)-3-methoxybenzoic Acid (JN01)

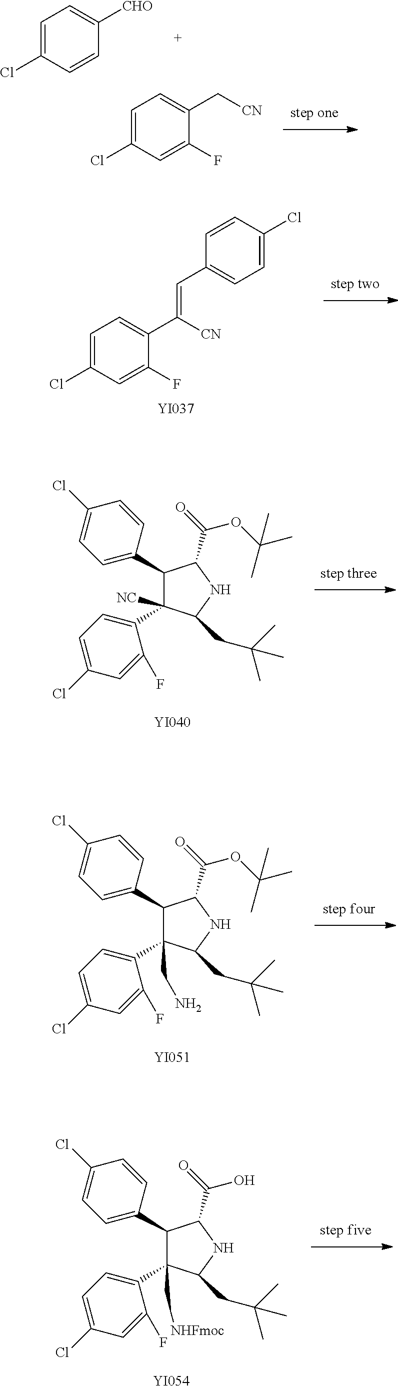

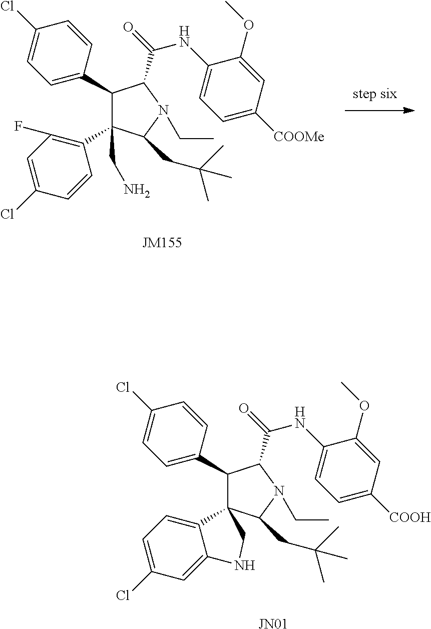 [SMILES: CCN1[C@@H](CC(C)(C)C)[C@](CN)(c2ccc(Cl)cc2F)[C@@H](c2ccc(Cl)cc2)[C@@H]1C(=O)Nc1ccc(C(=O)OC)cc1OC ; "JM155" ; CCN1[C@@H](CC(C)(C)C)[C@@]2(CNc3cc(Cl)ccc32)[C@@H](c2ccc(Cl)cc2)[C@@H]1C(=O)Nc1ccc(C(=O)O)cc1OC ; "JN01"]

### Step one: Synthesis of (Z)-2-(4-chloro-2-fluorophenyl)-3-(4-chlorophenyl)acrylonitrile (YI037)

4-Chlorobenzaldehyde (1.4 g, 10 mmol), 4-chloro-2-fluorophenylacetonitrile (1.7 g, 10 mmol) and 5N sodium methoxide in methanol (3 mL) were reacted according to the procedure described in step one of intermediate 1 to obtain the target product (2.78 g, yield 95%). ¹H NMR (500 MHz, Chloroform-d) δ 7.85-7.78 (m, 2H), 7.57-7.50 (m, 2H), 7.48-7.42 (m, 2H), 7.25-7.18 (in, 2H).

### Step two: Synthesis of tert-butyl (2R,3R,4R,5S)-4-(4-chloro-2-fluorophenyl)-3-(4-chiorophenyl)-4-cyano-5-neopentylpyrrolidine-2-carboxylate (YI040)

YI037 (2.7 g, 9.1 mmol), AgF (1.16 g, 9.1 mmol), triethylamine (2.1 mL) and tert-butyl (E)-2-(((3,3-dimethylbutylidene)amino)acetate (1.94 g, 9.1 mmol) were reacted according to the procedure described in step two of intermediate 1 to obtain the target product (1.57 g, yield 34%). ¹H NMR (500 MHz, Chloroform-d) S67.32 (t, J=8.5 Hz, 1H), 7.26-7.21 (m, 2H), 7.18 (dd, J=12.3, 2.1 Hz, 1H), 7.11 (dd, J=8.7, 2.4 Hz, 3H), 4.23 (d, J=7.9 Hz, 1H), 4.13 (d, J=7.8 Hz, 1H), 4.06 (dd, J=9.2, 1.2 Hz, 1H), 1.60 (ddd, J=14.4, 9.2, 1.1 Hz, 1H), 1.37 (s, 9H), 1.29 (dd, J=14.3, 1.2 Hz, 1H), 0.89 (s, 9K).

### Step Three: Synthesis of tert-butyl (2R,3R,4S,5S)-4-(aminomethyl)-4-(4-chloro-2-fluorophenyl)-3-(4-chlorophenyl)-5-neopentylpyrrolidine-2-carboxylate (YI051)

YI040 (1.57 g, 3.1 mmol), Raney nickel (1.57 g) and hydrazine hydrate (10 mL) were reacted according to the procedure described in step three of intermediate 1 to obtain the target product (584 mg, yield 37%).

### Step Four. Synthesis of (2R,3R,4S,5S)-4-((((((9H-fluoren-9-yl)methoxy)carbonyl)amino)methyl)-4-(4-chloro-2-fluorophenyl)-3-(4-chlorophenyl)-5-neopentylpyrrolidine-2-carboxylic acid (YI054)

YI051 (584 mg, 1.15 mmol), FmocCl (385 mg, 1.49 mmol), diisopropylethylamine (592 mg, 4.6 mmol) and trifluoroacetic acid (4 mL) were reacted according to the procedure described in step four of intermediate 1 to obtain the target product (722 mg, yield 95%).

### Step five: methyl 4-(2R,3R,4S,5S)-4-(aminomethyl)-4-(4-chloro-2-fluorophenyl)-1-ethyl-3-(4-chlorophenyl)-5-neopentylpyrrolidine-2-carboxamido)-3-methoxybenzoate (JM155)

YI054 (300 mg, 0.44 mmol), diisopropylethylamine (284 mg, 2.2 mmol), diphenylphosphinic chloride (313 mg, 1.32 mmol), methyl 4-amino-3-methoxybenzoate (322 mg, 1.78 mmol), acetaldehyde (40 mg, 0.9 mmol), sodium borohydride acetate (191 mg, 0.9 mmol), acetic acid (0.1 mL) and piperidine (0.4 mL) were reacted according to the procedure described in step five of the final product 10 to obtain the target product (20 mg, yield 8%). ¹H NMR (400 MHz, Methanol-d₄) δ 8.26 (d, J=8.3 Hz, 1H), 7.68-7.57 (m, 2H), 7.49 (t, J=9.0 Hz, 1H), 7.39-7.30 (m, 3H), 7.26 (d, J=13.6 Hz, 1H), 7.11 (d, J=8.0 Hz, 2H), 4.42 (d, J=8.8 Hz, 1H), 4.32 (d, J=9.1 Hz, 1H), 4.03 (d, J=9.2 Hz, 1H), 3.94 (s, 3H), 3.88 (s, 3H), 3.52-3.38 (m, 2H), 3.26 (d, J=14.6 Hz, 1H), 3.05-2.93 (m, 1H), 2.00 (dd, J=15.2, 9.2 Hz, 1H), 1.45 (d, J=15.1 Hz, 1H), 1.25 (t, J=7.0 Hz, 3H), 1.03 (s, 9H).

### Step six: Synthesis of 4-((2′S,3S,4′S,5′R)-6-chloro-4′-(4-chlorophenyl)-1′-ethyl-2′-neopentylspiro[indoline-3,3′-pyrrolidine]-5′-carboxamido)-3-methoxybenzoic Acid (JN01)

JM155 (21 mg, 0.03 mmol), potassium carbonate (19 mg, 0.13 mmol) and lithium hydroxide monohydrate (13 mg, 0.3 mmol) were reacted according to the procedure described in step six of the final product 10 to obtain the target product (6.6 mg, yield 36%). ¹H NMR (400 MHz, Methanol-d₄) δ 8.22 (d, J=8.4 Hz, 1H), 7.65 (d, J=8.6 Hz, 1H), 7.60 (s, 1H), 7.41-7.29 (m, 3H), 7.23 (d, J=8.2 Hz, 2H), 6.73 (d, J=8.0 Hz, 1H), 6.46 (s, 1H), 5.09-4.89 (m, 1H), 4.40-4.15 (m, 1H), 4.13-3.98 (m, 1H), 3.84 (s, 3H), 3.76-3.61 (m, 2H), 3.60-3.45 (m, 1H), 3.29-3.12 (m, 1H), 2.14-1.91 (m, 1H), 1.89-1.62 (m, 1H), 1.43 (t, J=7.1 Hz, 3H), 0.90 (s, 9H). ESI-MS calculated for C₃H₃₈³⁵Cl₂N₃O₄ [M+H]⁺=610.2, found: 610.2.

### Final Product 13: 4-((2′S,3S,4′S,5′R)-6-chloro-4′-(2-chlorophenyl)-1′-ethyl-2′-neopentylspiro[indoline-3,3′-pyrrolidine]-5′-carboxamido)-3-methoxybenzoic Acid (JN18)

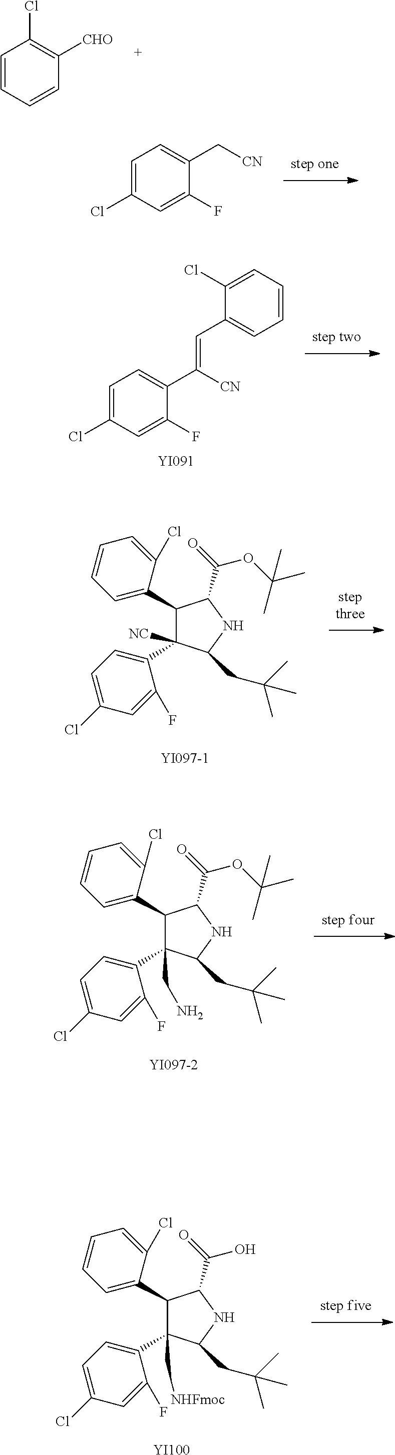

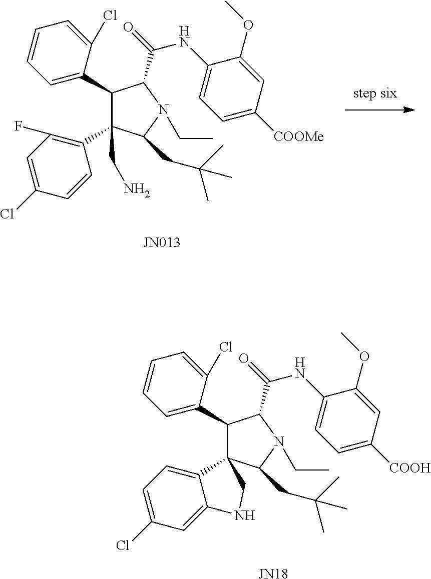 [SMILES: CCN1[C@@H](CC(C)(C)C)[C@](CN)(c2ccc(Cl)cc2F)[C@@H](c2ccccc2Cl)[C@@H]1C(=O)Nc1ccc(C(=O)OC)cc1OC ; "JN013" ; CCN1[C@@H](CC(C)(C)C)[C@@]2(CNc3cc(Cl)ccc32)[C@@H](c2ccccc2Cl)[C@@H]1C(=O)Nc1ccc(C(=O)O)cc1OC ; "JN18"]

### Step one: Synthesis of (Z)-2-(4-chloro-2-fluorophenyl)-3-(2-chlorophenyl)acrylonitrile (YI091)

2-Chlorobenzaldehyde (2.82 g, 20 mmol), 4-chloro-2-fluorophenylacetonitrile (3.4 g, 20 mmol) and 5N sodium methoxide in methanol (4.8 mL) were reacted according to the procedure described in step one of intermediate 1 to obtain the target product (5.46 g, yield 93%). ¹H NMR (500 MHz, Chloroform-d) δ 8.11 (dd, J=5.5, 3.8 Hz, 1H), 7.94 (s, 1H), 7.57 (t, J=8.3 Hz, 1H), 7.52-7.46 (m, 1H), 7.44-7.37 (m, 2H), 7.29-7.20 (m, 2H).

### Step two: Synthesis of tert-butyl (2R,3R,4R,5S)-4-(4-chloro-2-fluorophenyl)-3-(2-chlorophenyl)-4-cyano-5-neopentylpyrrolidine-2-carboxylate (YI097-1)

YI091 (5.46 g, 18.6 mmol), AgF (2.36 g, 18.6 mmol), triethylamine (4 mL) and tert-butyl (E)-2-(((3,3-dimethylbutylidene)amino)acetate (3.97 g, 18.6 mmol) were reacted according to the procedure described in step two of intermediate 1 to obtain the target product (4 g, yield 43%). ¹H NMR (500 MHz, Chloroform-d) δ 7.80 (dd, J=8.1, 1.4 Hz, 1H), 7.37 (ddd, J=8.2, 6.8, 1.8 Hz, 1H), 7.32 (t, J=8.5 Hz, 1H), 7.25-7.16 (m, 2H), 7.12 (dd, J=12.2, 2.1 Hz, 1H), 7.09-7.03 (m, 1H), 4.92 (d, J=6.7 Hz, 1H), 4.18-4.10 (m, 1H), 4.08 (d, J=6.7 Hz, 1H), 1.72-1.57 (m, 1H), 1.40 (s, 9H), 1.30 (dd, J=14.4, 1.1 Hz, 1H), 0.92 (s, 9H).

### Step Three: Synthesis of tert-butyl (2R,3R,4S,5S)-4-(aminomethyl)-4-(4-chloro-2-fluorophenyl)-3-(2-chlorophenyl)-5-neopentylpyrrolidine-2-carboxylate (YI097-2)

YI097-1 (4 g, 7.91 mmol), Raney nickel (4 g) and hydrazine hydrate (10 mL) were reacted according to the procedure described in step three of intermediate 1 to obtain the target product (1 g, yield 24%). ¹H NMR (500 MHz, Chloroform-d) δ 7.80 (dd, J=7.9, 1.6 Hz, 1H), 7.31-7.20 (m, 2H), 7.17-7.10 (m, 2H), 7.05 (dd, J=8.6, 2.3 Hz, 1H), 6.95 (dd, J=12.8, 2.2 Hz, 1H), 4.33 (d, J=8.9 Hz, 1H), 4.19 (dd, J=9.6, 1.2 Hz, 1H), 4.03 (d, J=8.9 Hz, 1H), 3.41-3.29 (m, 2H), 1.57 (dd, J=13.8, 1.3 Hz, 1H), 1.51 (d, J=9.7 Hz, 1H), 1.25 (s, 9H), 1.01 (s, 9H).

### Step Four: Synthesis of (2R,3R,4S,5S)-4-((((((9H-fluoren-9-yl)methoxy)carbonyl)amino)methyl)-4-(4-chloro-2-fluorophenyl)-3-(2-chlorophenyl)-5-neopentylpyrrolidine-2-carboxylic Acid (YI100)

YI097-2 (980 mg, 1.93 mmol), diisopropylethylamine (996 mg, 7.72 mmol), FmocCl (745 mg, 2.89 mmol) and trifluoroacetic acid (4 mL) were reacted according to the procedure described in step four of intermediate 1 to obtain the target product (1.12 g, yield 86%).

### Step five: Methyl 4-((2R,3R,4S,5S)-4-(aminomethyl)-4-(4-chloro-2-fluorophenyl)-1-ethyl-3-(2-chlorophenyl)-5-neopentylpyrrolidine-2-carboxamido)-3-methoxybenzoate (JN013)

YI100 (200 mg, 0.3 mmol), diisopropylethylamine (194 mg, 1.5 mmol), diphenylphosphinic chloride (214 mg, 0.9 mmol), methyl 4-amino-3-methoxybenzoate (215 mg, 1.2 mmol), acetaldehyde (44 mg, 1.0 mmol), sodium cyanoborohydride (212 mg, 1.0 mmol), acetic acid (0.1 mL) and piperidine (0.3 mL) were reacted according to the procedure described in step five of final product 10 to obtain the target product (15 mg, yield 17%). ¹H NMR (400 MHz, Methanol-d₄) δ 8.26 (d, J=8.3 Hz, 1H), 7.67 (d, J=8.3 Hz, 1H), 7.65-7.57 (m, 2H), 7.44 (t, J=7.5 Hz, 1H), 7.41-7.28 (m, 2H), 7.26 (d, J=8.7 Hz, 1H), 7.16 (d, J=13.4 Hz, 1H), 4.63 (d, J=9.9 Hz, 1H), 4.55 (d, J=8.1 Hz, 1H), 4.18 (d, J=9.1 Hz, 1H), 3.96 (s, 3H), 3.88 (s, 3H), 3.63 (d, J=14.6 Hz, 1H), 3.60-3.49 (m, 1H), 3.39 (d, J=14.5 Hz, 1H), 3.20-3.04 (m, 1H), 2.07 (dd, J=14.8, 10.3 Hz, 1H), 1.51 (d, J=14.9 Hz, 1H), 1.24 (t, J=7.0 Hz, 3H), 1.14 (s, 9H).

### Step six: Synthesis of 4-((2′S,3S,4′S,5′R)-6-chloro-4′-(2-chlorophenyl)-1′-ethyl-2′-neopentylspiro[indoline-3,3′-pyrrolidine]-5′-carboxamido)-3-methoxybenzoic Acid (JN18)

JN013 (15 mg, 0.03 mmol), potassium carbonate (17 mg, 0.12 mmol) and lithium hydroxide monohydrate (13 mg, 0.3 mmol) were reacted according to the procedure described in step six of the final product 10 to obtain the target product (9 mg, yield 50%). ¹H NMR (500 MHz, Methanol-d₄) δ 8.22 (d, J=8.4 Hz, 1H), 7.70-7.63 (m, 2H), 7.61 (s, 1H), 7.44 (t, J=7.6 Hz, 1H), 7.35 (d, J=7.8 Hz, 1H), 7.33-7.23 (m, 2H), 6.67 (dd, J=8.1, 1.9 Hz, 1H), 6.45 (d, J=1.9 Hz, 1H), 4.92-4.50 (m, 1H), 4.42-4.01 (m, 1H), 3.96-3.83 (m, 1H), 3.84 (s, 3H), 3.69 (d, J=10.1 Hz, 1H), 3.66-3.49 (m, 2H), 3.27-3.11 (m, 1H), 2.02-1.86 (m, 1H), 1.83-1.60 (m, 1H), 1.39 (t, J=7.0 Hz, 3H), 0.87 (s, 9H). ESI-MS calculated value C₃₃H₃₈³⁵Cl₂N₃O₄ [M+H]⁺=610.2, found: 610.6.

### Final Product 14: 4-((2′S,3S,4′S,5′R)-6-chloro-4′-(3-chlorophenyl)-1′-ethyl-2′-neopentylspiro[indoline-3,3′-pyrrolidine]-5′-carboxamido)-3-methoxybenzoic Acid (YN55)

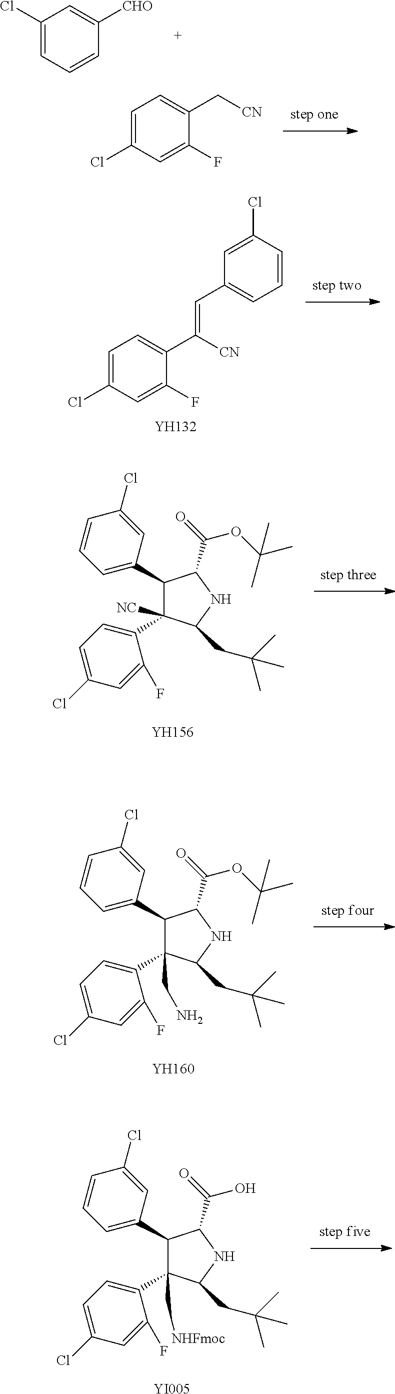

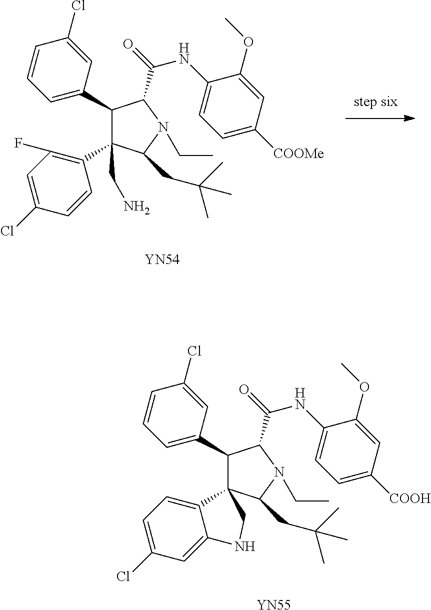 [SMILES: CCN1[C@@H](CC(C)(C)C)[C@](CN)(c2ccc(Cl)cc2F)[C@@H](c2cccc(Cl)c2)[C@@H]1C(=O)Nc1ccc(C(=O)OC)cc1OC ; "YN54" ; CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC ; "YN55"]

### Step one: Synthesis of (Z)-2-(4-chloro-2-fluorophenyl)-3-(3-chlorophenyl)acrylonitrile (YH132)

3-Chlorobenzaldehyde (1.4 g, 10 mmol), 4-chloro-2-fluorophenylacetonitrile (1.7 g, 10 mmol) and 5N sodium methoxide in methanol (3 mL) were reacted according to the procedure described in step one of intermediate 1 to obtain the target product (2.7 g, yield 92%). ¹H NMR (500 MHz, Chloroform-d) δ 7.84-7.80 (m, 1H), 7.78 (dt, J=2.6, 1.3 Hz, 1H), 7.58-7.49 (m, 2H), 7.46-7.38 (m, 2H), 7.24 (dd, J=2.0, 0.7 Hz, 1H), 7.22 (dd, J=10.9, 2.1 Hz, 1H).

### Step two: Synthesis of tert-butyl (2R,3R,4R,5S)-4-(4-chloro-2-fluorophenyl)-3-(3-chlorophenyl)-4-cyano-5-neopentylpyrrolidine-2-carboxylate (YH156)

YH132 (2.7 g, 9.3 mmol), AgF (1.17 g, 9.3 mmol), triethylamine (2.1 mL) and tert-butyl (E)-2-(((3,3-dimethylbutylidene)amino)acetate (1.97 g, 9.3 mmol) were reacted according to the procedure described in step two of intermediate 1 to obtain the target product (1.42 g, yield 30%).

### Step Three: Synthesis of tert-butyl (2R,3R,4S,5S)-4-(aminomethyl)-4-(4-chloro-2-fluorophenyl)-3-(3-chlorophenyl)-5-neopentylpyrrolidine-2-carboxylate (YH160)

YH156 (1.42 g, 2.8 mmol), Raney nickel (1.4 g) and hydrazine hydrate (10 mL) were reacted according to the procedure described in step three of intermediate 1 to obtain the target product (117 mg, yield 8%). ¹H NMR (500 MHz, Chloroform-d) δ 7.25-7.01 (m, 6H), 6.97 (dd, J=7.5, 1.7 Hz, 1H), 4.28 (d, J=8.7 Hz, 1H), 4.11 (d, J=9.1 Hz, 1H), 3.87 (dd, J=8.8, 2.1 Hz, 1H), 3.25 (d, J=13.3 Hz, 1H), 3.07 (d, J=13.3 Hz, 1H), 1.56-1.35 (m, 2H), 1.30 (s, 9H), 0.91 (s, 9H).

### Step Four: Synthesis of (2R,3R,4S,5S)-4-((((((9H-fluoren-9-yl)methoxy)carbonyl)amino)methyl)-4-(4-chloro-2-fluorophenyl)-3-(3-chlorophenyl)-5-neopentylpyrrolidine-2-carboxylic Acid (YI005)

YH160 (236 mg, 0.46 mmol), FmocCl (180 mg, 0.7 mmol), diisopropylethylamine (239 mg, 1.8 mmol) and trifluoroacetic acid (2 mL) were reacted according to the procedure described in step four of intermediate 1 to obtain the target product (219 mg, yield 70%).

### Step five: methyl 4-(2R,3R,4S,5S)-4-(aminomethyl)-4-(4-chloro-2-fluorophenyl)-1-ethyl-3-(3-chlorophenyl)-5-neopentylpyrrolidine-2-carboxamido)-3-methoxybenzoate (YN54)

YI05 (200 mg, 0.3 mmol), diisopropylethylamine (184 mg, 1.5 mmol), diphenylphosphinic chloride (214 mg, 0.9 mmol), methyl 4-amino-3-methoxybenzoate (218 mg, 1.2 mmol), acetaldehyde (20 mg, 0.46 mmol), sodium borohydride acetate (98 mg, 0.46 mmol), acetic acid (0.1 mL) and piperidine (0.2 mL) were reacted according to the procedure described in step five of the final product 10 to obtain the target product (12 mg, yield 6%). ¹H NMR (400 MHz, Methanol-d₄): δ 8.33-8.21 (m, 1H), 7.60-7.43 (m, 3H), 7.36-7.28 (m, 3H), 7.25 (d, J=13.6 Hz, 1H), 7.20-7.00 (m, 2H), 4.46 (d, J=8.9 Hz, 1H), 4.37 (d, J=9.2 Hz, 1H), 4.06 (d, J=9.3 Hz, 1H), 3.90 (s, 3H), 3.83 (s, 3H), 3.55-3.40 (m, 2H), 3.39-3.27 (m, 1H), 3.13-2.98 (m, 1H), 2.07 (dd, J=15.1, 9.1 Hz, 1H), 1.49 (d, J=14.7 Hz, 1H), 1.25 (t, J=6.9 Hz, 3H), 1.05 (s, 9H), 13C NMR (126 MHz, Methanol-d₄): δ 173.78, 167.87, 162.63 (d, J=249.7 Hz), 149.60, 138.87, 136.18 (d, J=12.1 Hz), 135.53, 132.41, 132.02 (d, J=4.9 Hz), 131.25, 130.16, 129.06, 128.40, 126.79, 126.71, 124.88 (d, J=8.9 Hz), 123.82, 119.76, 118.99 (d, J=30.2 Hz), 111.87, 69.98, 68.25, 60.93, 58.34, 56.38, 52.63, 46.75, 41.09 (d, J=5.8 Hz), 38.82, 32.01, 30.34, 15.69. ESI-MS calculated for C₃₄H₄₁Cl₂FN₃O₄ [M+H]⁺=644.2; found: 644.3.

### Step six: Synthesis of 4-((2′S,3S,4′S,5′R)-6-chloro-4′-(3-chlorophenyl)-1′-ethyl-2′-neopentylspiro[indoline-3,3′-pyrrolidine]-5′-carboxamido)-3-methoxybenzoic Acid (YN55)

YN54 (12 mg, 0.02 mmol), potassium carbonate (8 mg, 0.06 mmol) and lithium hydroxide monohydrate (4 mg, 0.1 mmol) were reacted according to the procedure described in step six of the final product 10 to obtain the target product (4 mg, yield 33%). ¹H NMR (500 MHz, Methanol-d₄) δ 8.25 (d, J=8.3 Hz, 1H), 7.64 (dd, J=8.4, 1.7 Hz, 1H), 7.60 (s, 1H), 7.42-7.20 (m, 4H), 7.10 (d, J=7.5 Hz, 1H), 6.71 (dd, J=8.1, 1.9 Hz, 1H), 6.43 (d, J=1.9 Hz, 1H), 4.89-4.65 (m, 1H), 4.40-3.92 (m, 2H), 3.86 (s, 3H), 3.72-3.55 (m, 2H), 3.55-3.40 (m, 1H), 3.21-2.82 (m, 1H), 2.16-1.84 (m, 1H), 1.76-1.44 (m, 1H), 1.37 (t, J=6.2 Hz, 3H), 0.92 (s, 9H), 13C NMR (126 MHz, CDCl₃): δ 173.15, 171.53, 152.87, 147.95, 139.46, 134.20, 134.11, 132.37, 129.53, 129.04, 128.19, 127.35, 126.93, 124.56, 124.32, 123.26, 118.80, 118.41, 111.12, 109.96, 70.59, 68.14, 62.93, 61.26, 55.71, 48.59, 44.87, 37.78, 30.75, 29.93, 14.97. ESI-MS calculated for C₃₃H₃₈³⁵Cl₂N₃O₄ [M+H]⁺=610.2, found: 610.2.

### Final Product 15: 4-((2′S,3S,4′S,5′R)-6-chloro-4′-(3-methylphenyl)-1′-ethyl-2′-neopentylspiro[indoline-3,3′-pyrrolidine]-5′-carboxamido)-3-methoxybenzoic Acid (JM157)

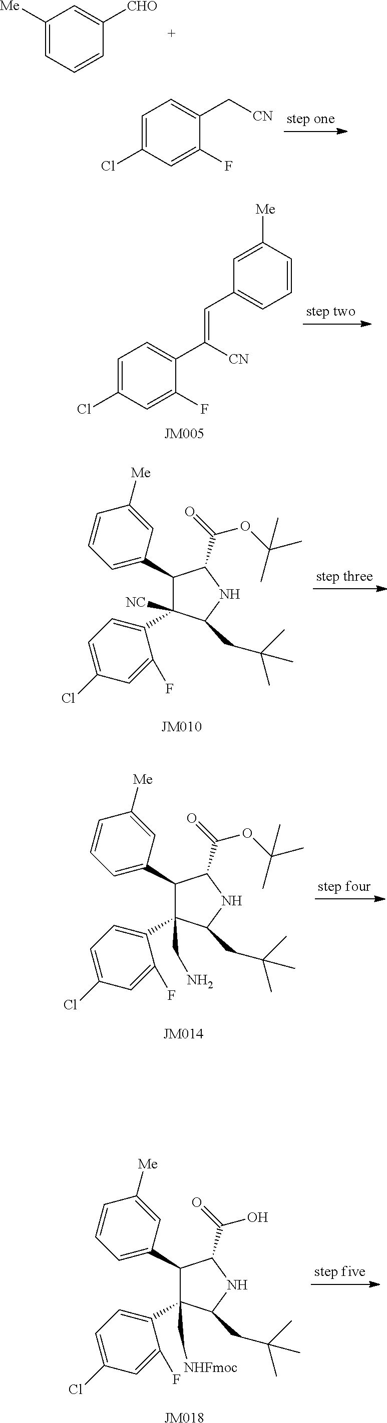

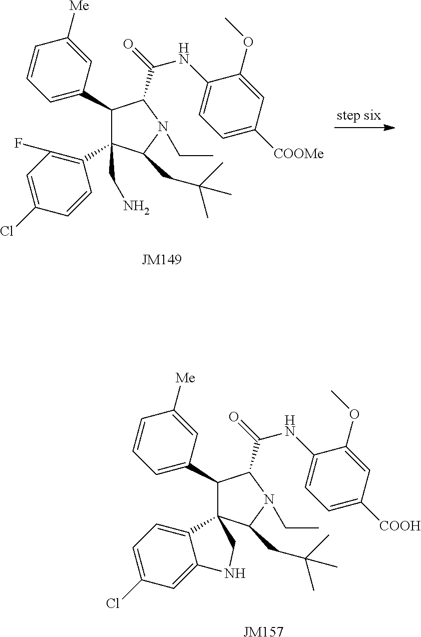 [SMILES: CCN1[C@@H](CC(C)(C)C)[C@](CN)(c2ccc(Cl)cc2F)[C@@H](c2cccc(C)c2)[C@@H]1C(=O)Nc1ccc(C(=O)OC)cc1OC ; "JM149" ; CCN1[C@@H](CC(C)(C)C)[C@@]2(CNc3cc(Cl)ccc32)[C@@H](c2cccc(C)c2)[C@@H]1C(=O)Nc1ccc(C(=O)O)cc1OC ; "JM157"]

### Step one: Synthesis of (Z)-2-(4-chloro-2-fluorophenyl)-3-(3-methylphenyl)acrylonitrile (JM005)

3-Methylbenzaldehyde (2.4 g, 20 mmol), 4-chloro-2-fluorophenylacetonitrile (3.4 g, 20 mmol) and 5N sodium methoxide in methanol (4.8 mL) were reacted according to the procedure described in step one of intermediate 1 to obtain the target product (5 g, yield 93%). ¹H NMR (500 MHz, Chloroform-d) δ 7.72 (dd, J=7.7, 1.7 Hz, 1H), 7.67 (s, 1H), 7.56-7.49 (m, 2H), 7.37 (t, J=7.7 Hz, 1H), 7.29 (dd, J=7.5, 1.1 Hz, 1H), 7.24 (ddd, J=8.3, 2.0, 0.7 Hz, 1H), 7.21 (dd, J=10.7, 2.0 Hz, 1H), 2.42 (s, 3H).

### Step two: Synthesis of tert-butyl (2R,3R,4R,5S)-4-(4-chloro-2-fluorophenyl)-3-(3-methylphenyl)-4-cyano-5-neopentylpyrrolidine-2-carboxylate (JM010)

JM005 (5 g, 20 mmol), AgF (2.54 g, 20 mmol), triethylamine 4.5 mL and tert-butyl (E)-2-(((3,3-dimethylbutylidene)amino)oxy)acetate (4.4 g, 20 mmol) were reacted according to the procedure described in step two of intermediate 1 to obtain the target product (2.8 g, yield 29%0/). ¹H NMR (500 MHz, Chloroform-d) δ 7.33 (t, J=8.5 Hz, 1H), 7.17 (dd, J=12.3, 2.1 Hz, 1H), 7.12 (t, J=7.6 Hz, 1H), 7.08 (dd, J=8.5, 2.2 Hz, 1H), 7.04 (d, J=7.6 Hz, 1H), 7.01 (s, H), 6.93 (d, J=7.9 Hz, 1H), 4.27 (d, J=7.6 Hz, 1H), 4.14 (d, J=7.6 Hz, 1H), 4.06 (d, J=9.0 Hz, 1H), 2.27 (s, 3H), 1.61 (dd, J=14.4, 9.1 Hz, 1H), 1.37 (s, 9H), 1.30 (dd, J=14.4, 1.2 Hz, 1H), 0.89 (s, 9H).

### Step Three: Synthesis of tert-butyl (2R,3R,4S,5S)-4-(aminomethyl)-4-(4-chloro-2-fluorophenyl)-3-(3-methylphenyl)-5-neopentylpyrrolidine-2-carboxylate (JM014)

JM010 (2.6 g, 5.4 mmol), Raney nickel (3.2 g) and hydrazine hydrate (10 mL) were reacted according to the reaction procedure as described in step three of intermediate 1 to obtain the target product (418 mg, yield 16%). ¹H NMR (500 MHz, Chloroform-d) δ 7.11 (t, J=8.6 Hz, 1H), 7.05-7.01 (m, 2H), 6.99 (dd, J=13.0, 2.3 Hz, 1H), 6.93 (d, J=7.6 Hz, 1H), 6.88 (s, 1H), 6.83 (d, J=7.8 Hz, 1H), 4.26 (d, J=8.6 Hz, 1H), 4.02 (dd, J=8.2, 2.3 Hz, 1H), 3.89 (dd, J=8.6, 1.7 Hz, 1H), 3.19 (d, J=13.6 Hz, 1H), 3.05 (d, J=13.6 Hz, 1H), 2.19 (s, 3H), 1.42-1.31 (m, 2H), 1.25 (s, 9H), 0.85 (s, 9H).

### Step Four. Synthesis of (2R,3R,4S,5S)-4-((((((9H-fluoren-9-yl)methoxy)carbonyl)amino)methyl)-4-(4-chloro-2-fluorophenyl)-3-(3-methylphenyl)-5-neopentylpyrrolidine-2-carboxylic Acid (JM018)

JM014 (1.0 g, 2.0 mmol), FmocCl (793 mg, 1.5 mmol), diisopropylethylamine (1.0 g, 8.2 mmol) and trifluoroacetic acid (5 mL) were reacted according to the reaction procedure as described in step four of intermediate 1 to obtain the target product (1.1 g, yield 85%).

### Step five: Methyl 4-((2R,3R,4S,5S)-4-(aminomethyl)-4-(4-chloro-2-fluorophenyl)-1-ethyl-3-(3-methylphenyl)-5-neopentylpyrrolidine-2-carboxamido)-3-methoxybenzoate (JM149)

JM018 (200 mg, 0.3 mmol), diisopropylethylamine (206 mg, 1.6 mmol), diphenylphosphinic chloride (228 mg, 0.96 mmol), methyl 4-amino-3-methoxybenzoate (226 mg, 1.25 mmol), acetaldehyde (44 mg, 1.0 mmol), sodium borohydride acetate (212 mg, 1.0 mmol), acetic acid (0.1 mL) and piperidine (0.2 mL) were reacted according to the reaction procedure as described in step five of final product 10 to obtain the target product (37 mg, yield 20%). ¹H NMR (400 MHz, Methanol-d₄): δ 8.25 (d, J=8.2 Hz, 1H), 7.66-7.55 (m, 2H), 7.49 (t, J=9.0 Hz, 1H), 7.40-7.19 (m, 3H), 7.14 (d, J=7.7 Hz, 1H), 7.01-6.89 (m, 2H), 4.44 (d, J=9.1 Hz, 1H), 4.40 (d, J=8.0 Hz, 1H), 4.12 (d, J=8.9 Hz, 1H), 3.91 (s, 3H), 3.88 (s, 3H), 3.57-3.37 (m, 2H), 3.30-3.24 (m, 1H), 3.09-3.00 (m, 1H), 2.28 (s, 3H), 1.98 (dd, J=15.9, 9.0 Hz, 1H), 1.45 (d, J=15.1 Hz, 1H), 1.26 (t, J=7.1 Hz, 3H), 0.99 (s, 9H). ESI-MS calculated for C₃₅H₄₄ClFN₃O₄ [M+H]⁺=624.3, found: 624.3.

### Step six: Synthesis of 4-((2′S,3S,4′S,5′R)-6-chloro-4′-(3-methylphenyl)-1′-ethyl-2′-neopentylspiro[indoline-3,3′-pyrrolidine]-5′-carboxamido)-3-methoxybenzoic Acid (JM157)

JM149 (26 mg, 0.04 mmol), potassium carbonate (24 mg, 0.17 mmol) and lithium hydroxide monohydrate (17 mg, 0.4 mmol) were reacted according to the reaction procedure as described in step six of final product 10 to obtain the target product (9.4 mg, yield 40%). ¹H NMR (400 MHz, Methanol-d₄) δ 8.19 (d, J=8.4 Hz, 1H), 7.63 (d, J=8.5 Hz, 1H), 7.56 (s, 1H), 7.35 (d, J=8.0 Hz, 1H), 7.21 (t, J=7.7 Hz, 1H), 7.14 (d, J=7.7 Hz, 1H), 7.10-6.98 (m, 1H), 6.72 (d, J=8.0 Hz, 1H), 6.44 (s, 1H), 5.22-5.01 (m, 1H), 4.46-3.96 (m, 2H), 3.77 (s, 3H), 3.72-3.52 (m, 3H), 3.53-3.29 (m, 1H), 2.28 (s, 3H), 2.15-1.94 (m, 1H), 1.90-1.64 (m, 1H), 1.45 (t, J=7.1 Hz, 3H), 0.85 (s, 9H). ESI-MS calculated for C₃₄H₄₀³⁵ClN₃O₄ [M+H]⁺=590.3, found: 590.3.

### Final Product 16: 4-((2′S,3S,4′S,5′R)-6-chloro-4′-(3-methoxyphenyl)-1′-ethyl-2′-neopentylspiro[indoline-3,3′-pyrrolidine]-5′-carboxamido)-3-methoxybenzoic Acid (JM160)

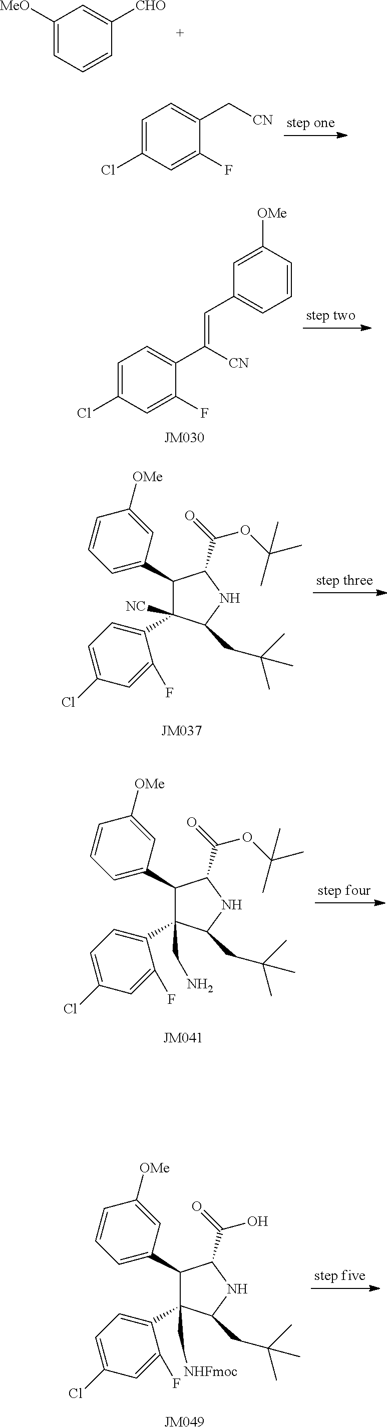

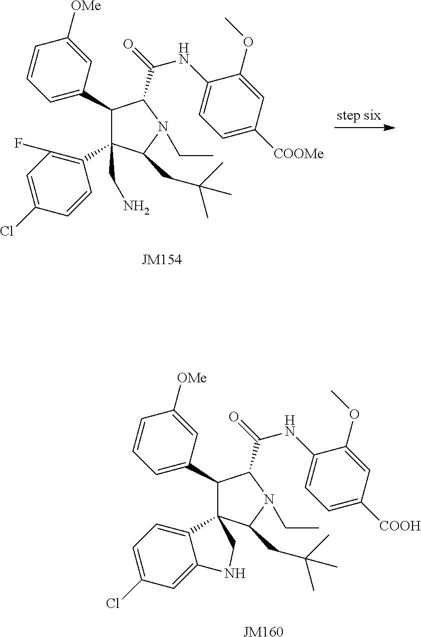 [SMILES: CCN1[C@@H](CC(C)(C)C)[C@](CN)(c2ccc(Cl)cc2F)[C@@H](c2cccc(OC)c2)[C@@H]1C(=O)Nc1ccc(C(=O)OC)cc1OC ; "JM154" ; CCN1[C@@H](CC(C)(C)C)[C@@]2(CNc3cc(Cl)ccc32)[C@@H](c2cccc(OC)c2)[C@@H]1C(=O)Nc1ccc(C(=O)O)cc1OC ; "JM160"]

### Step one: Synthesis of (Z)-2-(4-chloro-2-fluorophenyl)-3-(3-methoxyphenyl)acrylonitrile (JM030)

3-Methoxybenzaldehyde (2.7 g, 20 mmol), 4-chloro-2-fluorophenylacetonitrile (3.4 g, 20 mmol) and 5N sodium methoxide in methanol (4.8 mL) were reacted according to the reaction procedure as described in step one of intermediate 1 to obtain the target product (5.5 g, yield 96%). ¹H NMR (500 MHz, Chloroform-d) δ 7.55-7.50 (m, 2H), 7.49 (t, J=2.0 Hz, 1H), 7.39 (dt, J=8.0, 1.7 Hz, 1H), 7.37 (t, J=7.7 Hz, 1H), 7.25-7.21 (m, 1H), 7.19 (dd, J=10.7, 1.9 Hz, 1H), 7.02 (ddd, J=7.6, 2.5, 1.4 Hz, JH), 3.87 (s, 3H).

### Step two: Synthesis of tert-butyl (2R,3R,4R,5S)-4-(4-chloro-2-fluorophenyl)-3-(3-methoxyphenyl)-4-cyano-5-neopentylpyrrolidine-2-carboxylate (JM037)

JM030 (5.4 g, 20 mmol), AgF (2.54 g, 20 mmol), triethylamine 4.5 mL and tert-butyl (E)-2-(((3,3-dimethylbutylidene)amino)acetate (4.4 g, 20 mmol) were reacted according to the procedure described in step two of intermediate 1 to obtain the target product (3.9 g, yield 39%). ¹H NMR (500 MHz, Chloroform-d) δ 7.34 (t, J=8.5 Hz, 1H), 7.16 (dd, J=9.8, 2.6 Hz, 1H), 7.15-7.11 (m, 1H), 7.08 (dd, J=8.4, 2.1 Hz, JH), 6.80-6.76 (m, 1H), 6.75-6.71 (m, 2H), 4.27 (d, J=7.7 Hz, 1H), 4.14 (d, J=7.8 Hz, 1H), 4.05 (d, J=9.0 Hz, 1H), 3.71 (s, 3H), 1.67-1.56 (m, 1H), 1.37 (s, 9H), 1.31-1.27 (m, 1H), 0.88 (s, 9H).

### Step Three: Synthesis of tert-butyl (2R,3R,4S,5S)-4-(aminomethyl)-4-(4-chloro-2-fluorophenyl)-3-(3-methoxyphenyl)-5-neopentylpyrrolidine-2-carboxylate (JM041)

JM037 (5.3 g, 9.8 mmol), Raney nickel (4.6 g) and hydrazine hydrate (10 mL) were reacted according to the procedure described in step three of intermediate 1 to obtain the target product (1.3 g, yield 33%). ¹H NMR (500 MHz, Chloroform-d) δ 7.07 (t, J=8.6 Hz, 1H), 7.04-6.91 (m, 3H), 6.66-6.57 (m, 3H), 4.23 (d, J=8.6 Hz, 1H), 4.00 (dd, J=7.0, 1.8 Hz, 1H), 3.89 (dd, J=8.6, 2.0 Hz, 1H), 3.15 (d, J=13.4 Hz, 1H), 2.99 (d, J=13.4 Hz, 1H), 1.35-1.26 (m, 2H), 1.22 (s, 9H), 0.80 (s, 9H).

### Step Four: Synthesis of (2R,3R,4S,5S)-4-((((((9H-fluoren-9-yl)methoxy)carbonyl)amino)methyl)-4-(4-chloro-2-fluorophenyl)-3-(3-methoxyphenyl)-5-neopentylpyrrolidine-2-carboxylic Acid (JM049)

JM041 (1.0 g, 2.0 mmol), FmocCl (774 mg, 3.0 mmol), diisopropylethylamine (1.0 g, 8.0 mmol) and trifluoroacetic acid (4 mL) were reacted according to the procedure described in step four of intermediate 1 to obtain the target product (1.3 g, yield 94%).

### Step five: Methyl 4-((2R,3R,4S,5S)-4-(aminomethyl)-4-(4-chloro-2-fluorophenyl)-1-ethyl-3-(3-methoxyphenyl)-5-neopentylpyrrolidine-2-carboxamido)-3-methoxybenzoate (JM154)

JM049 (200 mg, 0.3 mmol), diisopropylethylamine (194 mg, 1.5 mmol), diphenylphosphinic chloride (214 mg, 0.9 mmol), methyl 4-amino-3-methoxybenzoate (221 mg, 1.2 mmol), acetaldehyde (44 mg, 1.0 mmol), sodium borohydride acetate (212 mg, 1.0 mmol), acetic acid (0.1 mL) and piperidine (0.2 mL) were reacted according to the procedure described in step five of final product 10 to obtain the target product (41 mg, yield 26%). ¹H NMR (400 MHz, Methanol-d₄) δ 8.25 (d, J=8.2 Hz, 1H), 7.66-7.56 (m, 2H), 7.50 (t, J=9.0 Hz, 1H), 7.32 (d, J=9.1 Hz, 1H), 7.31-7.20 (m, 2H), 6.88 (d, J=8.4 Hz, 1H), 6.83 (d, J=7.7 Hz, 1H), 6.56 (s, 1H), 4.53-4.34 (m, 2H), 4.15 (d, J=8.8 Hz, 1H), 3.91 (s, 3H), 3.87 (s, 3H), 3.67 (s, 3H), 3.48 (d, J=14.4 Hz, 1H), 3.45-3.36 (m, 1H), 3.26 (d, J=14.5 Hz, 1H), 3.09-2.96 (m, 1H), 1.97 (dd, J=15.3, 8.4 Hz, 1H), 1.45 (d, J=15.1 Hz, 1H), 1.25 (t, J=6.8 Hz, 3H), 0.98 (s, 9H).

### Step six: Synthesis of 4-((2′S,3S,4′S,5′R)-6-chloro-4′-(3-methoxyphenyl)-1′-ethyl-2′-neopentylspiro[indoline-3,3′-pyrrolidine]-5′-carboxamido)-3-methoxybenzoic Acid (JM160)

JM154 (39 mg, 0.06 mmol), potassium carbonate (34 mg, 0.25 mmol) and lithium hydroxide monohydrate (25 mg, 0.6 mmol) were reacted according to the procedure described in step six of final product 10 to obtain the target product (18.2 mg, yield 50%). ¹H NMR (400 MHz, Methanol-d₄) δ 8.20 (d, J=8.4 Hz, 1H), 7.63 (d, J=8.5 Hz, 1H), 7.56 (s, 1H), 7.35 (d, J=8.0 Hz, 1H), 7.25 (t, J=8.0 Hz, 1H), 6.96-6.82 (m, 2H), 6.78-6.65 (m, 2H), 6.45 (s, 1H), 5.19-5.02 (m, 1H), 4.45-3.97 (m, 2H), 3.79 (s, 3H), 3.66 (s, 3H), 3.65-3.49 (m, 3H), 3.31-3.16 (m, 1H), 2.18-1.94 (m, 1H), 1.92-1.66 (m, 1H), 1.43 (t, J=7.1 Hz, 3H), 0.85 (s, 9H). ESI-MS calculated for C₃₄H₄₁³⁵ClN₃O₅ [M+H]⁺=606.2, found: 606.2.

### Final Product 17: 4-((2′S,3S,4′S,5′R)-5-chloro-4′-(2,3-dichlorophenyl)-1′-ethyl-2′-neopentylspiro[indoline-3,3′-pyrrolidine]-5′-carboxamido)-3-methoxybenzoic Acid (JN19)

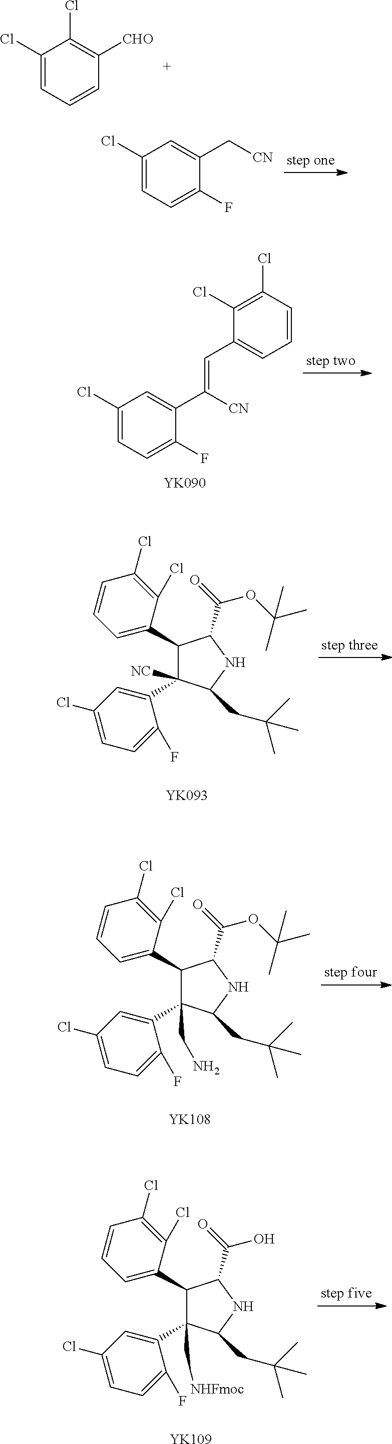

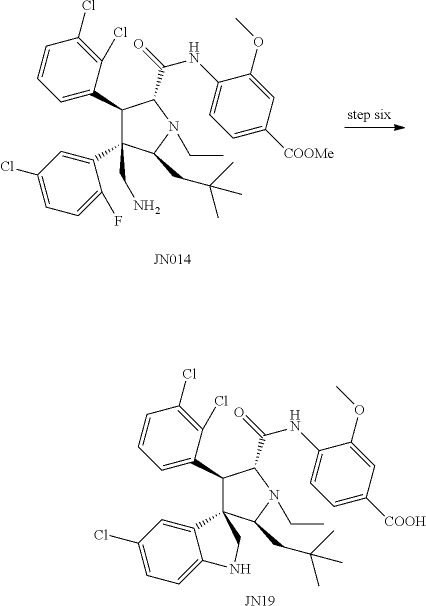

### Step one: Synthesis of (Z)-2-(5-chloro-2-fluorophenyl)-3-(2,3-dichlorophenyl)acrylonitrile (YK090)

5-chloro-2-fluorophenylacetonitrile (3.4 g, 20 mmol), 2,3-dichlorobenzaldehyde (3.5 g, 20 mmol) and 5N sodium methoxide in methanol (4.8 mL) were reacted according to the procedure described in step one of intermediate 1 to obtain the target product (6.1 g, yield 93%). ¹H NMR (500 MHz, Chloroform-d) δ 7.98-7.89 (m, 2H), 7.59 (ddd, J=14.3, 7.4, 2.0 Hz, 2H), 7.42-7.32 (m, 2H), 7.15 (dd, J=10.5, 8.8 Hz, 1H).

### Step two: Synthesis of tert-butyl (2R,3S,4R,5S)-4-(5-chloro-2-fluorophenyl)-4-cyano-3-(2,3-dichlorophenyl)-5-neopentylpyrrolidine-2-carboxylate (YK093)

YK090 (6.1 g, 18.65 mmol), AgF (2.37 g, 18.65 mmol), triethylamine (4.15 mL) and tert-butyl (E)-2-(((3,3-dimethylbutylidene)amino)acetate (3.97 g, 19 mmol) were reacted according to the procedure described in step two of intermediate 1 to obtain the target product (3.72 g, yield 37%). ¹H NMR (500 MHz, Chloroform-d) δ 7.72 (dd, J=8.0, 1.6 Hz, 1H), 7.45-7.38 (m, 2H), 7.36-7.28 (m, 2H), 7.05 (dd, J=12.0, 8.8 Hz, 1H), 5.05 (dd, J=6.3, 1.0 Hz, 1H), 4.08 (d, J=8.8 Hz, 1H), 4.02 (d, J=6.3 Hz, 1H), 1.65 (ddd, J=14.5, 9.0, 1.2 Hz, 1H), 1.42 (s, 9H), 1.31 (dd, J=14.5, 1.1 Hz, 1H), 0.90 (s, 9H).

### Step Three: Synthesis of tert-butyl (2R,3S,4S,5S)-4-(aminomethyl)-4-(5-chloro-2-fluorophenyl)-3-(2,3-dichlorophenyl)-5-neopentylpyrrolidine-2-carboxylate (YK108)

YK093 (3.72 g, 6.8 mmol), Raney nickel (3 g) and hydrazine hydrate (10 mL) were reacted according to the procedure described in step three of intermediate 1 to obtain the target product (1.23 g, yield 32%).

### Step Four: Synthesis of (2R,3S,4S,5S)-4-((((((9H-fluoren-9-yl)methoxy)carbonyl)amino)methyl)-4-(5-chloro-2-fluorophenyl)-3-(2,3-dichlorophenyl)-5-neopentylpyrrolidine-2-carboxylic Acid (YK109)

YK108 (1.2 g, 2.2 mmol), diisopropylethylamine (1.2 g, 8.8 mmol), FmocCl (854 mg, 3.3 mmol) and trifluoroacetic acid (3 mL) were reacted according to the procedure described in step four of intermediate 1 to obtain the target product (1.08 g, yield 69%).

### Step five: Methyl 4-((2R,3S,4S,5S)-4-(aminomethyl)-4-(5-chloro-2-fluorophenyl)-3-(2,3-dichlorophenyl)-1-ethyl-5-neopentylpyrrolidine-2-carboxamido)-3-methoxybenzoate (JN014)

YK109 (200 mg, 0.3 mmol), diisopropylethylamine (188 mg, 1.5 mmol), diphenylphosphinic chloride (207 mg, 0.9 mmol), methyl 4-amino-3-methoxybenzoate (208 mg, 1.2 mmol), acetaldehyde (44 mg, 1.0 mmol), sodium borohydride acetate (212 mg, 1.0 mmol), acetic acid (0.1 mL) and piperidine (0.2 mL) were reacted according to the procedure described in step five of final product 10 to obtain the target product (10 mg, yield 10%). ¹H NMR (400 MHz, Methanol-d₄) δ 8.27 (d, J=8.2 Hz, 1H), 7.72-7.57 (m, 3H), 7.56-7.47 (m, 2H), 7.46-7.37 (m, 2H), 7.07 (t, J=11.2 Hz, 1H), 4.71-4.52 (m, 2H), 4.13 (d, J=9.4 Hz, 1H), 3.98 (s, 3H), 3.89 (s, 3H), 3.71-3.54 (m, 2H), 3.41 (d, J=14.7 Hz, 1H), 3.19-3.06 (m, 1H), 2.20-2.04 (m, 1H), 1.55 (d, J=15.1 Hz, 1H), 1.24 (t, J=5.9 Hz, 3H), 1.21 (s, 9H). ESI-MS calculated for C₃₄H₄₀Cl₃FN₃O₄ [M+H]⁺=678.2, found: 678.2.

### Step six: Synthesis of 4-((2′S,3S,4′S,5′R)-5-chloro-4′-(2,3-dichlorophenyl)-1′-ethyl-2′-neopentylspiro[indoline-3,3′-pyrrolidine]-5′-carboxamido)-3-methoxybenzoic acid (JN19)

JN014 (10 mg, 0.02 mmol), potassium carbonate (12 mg, 0.08 mmol) and lithium hydroxide monohydrate (8 mg, 0.2 mmol) were reacted according to the procedure described in step six of final product 10 to obtain the target product (5 mg, yield 38%). ¹H NMR (500 MHz, Methanol-d₄) δ 8.27 (d, J=8.0 Hz, 1H), 7.68 (dd, J=8.4, 1.8 Hz, 1H), 7.65-7.56 (m, 2H), 7.50 (d, J=8.0 Hz, 1H), 7.42 (d, J=8.3 Hz, 1H), 7.40 (s, 1H), 7.06 (dd, J=8.4, 2.1 Hz, 1H), 6.54 (d, J=8.3 Hz, 1H), 4.89-4.66 (m, 1H), 4.6-4.2 (m, 1H), 4.23-4.02 (m, 1H), 3.89 (s, 3H), 3.67 (d, J=10.8 Hz, 1H), 3.62-3.47 (m, 2H), 3.15-2.92 (m, 1H), 2.01-1.83 (m, 1H), 1.74-1.52 (m, 1H), 1.36 (t, J=6.8 Hz, 3H), 0.93 (s, 9H). ESI-MS calculated for C₃H₃₇³⁵Cl₃N₃O₄ [M+H]⁺=644.2, found: 644.2.

### Final Product 18: 4-((2′S,3S,4′S,5′R)-6-chloro-4′-(3-chlorophenyl)-1′-ethyl-2′-neopentylspiro[indoline-3,3′-pyrrolidine]-5′-carboxamido)-3-methoxybenzoic acid (JN122)

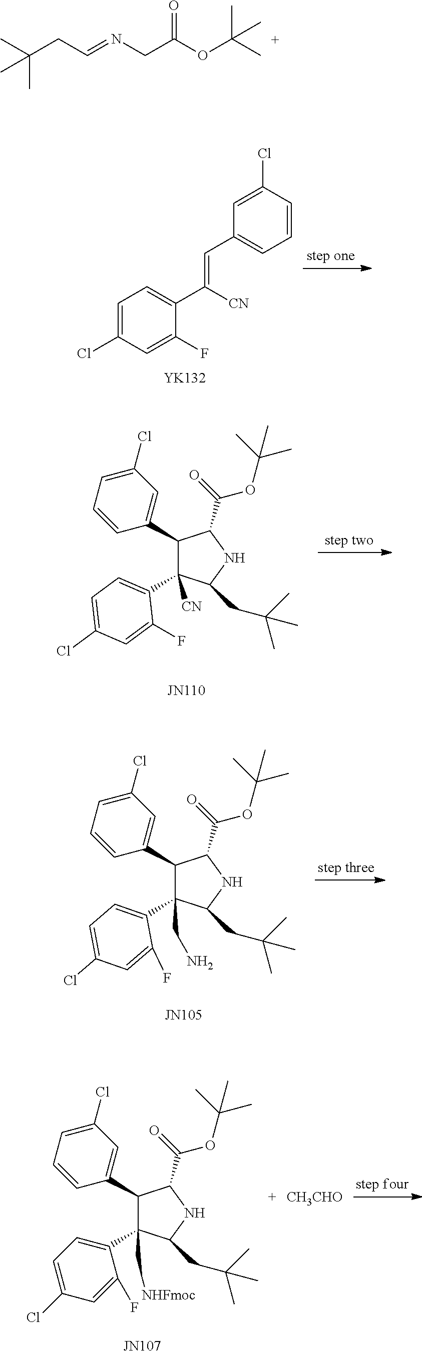

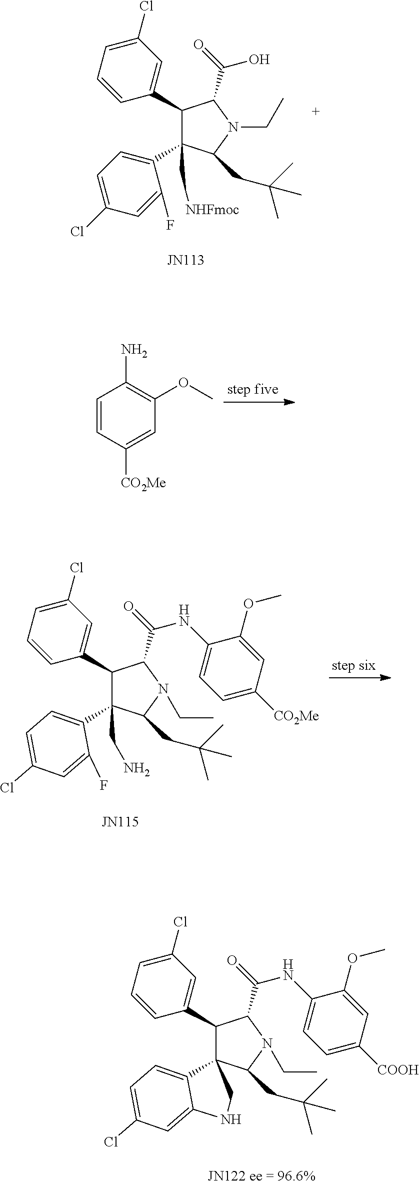

### Step one: Synthesis of tert-butyl (2R,3R,4R,5S)-4-(4-chloro-2-fluorophenyl)-3-(3-chlorophenyl)-4-cyano-5-neopentylpyrrolidine-2-carboxylate (JN110)

YH132 (2.4 g, 8.3 mmol) and tert-butyl (E)-2-(((3,3-dimethylbutylidene)amino)acetate (2.12 g, 10.0 mmol) were added into a 250 mL round-bottom flask, dissolved in 80 ml of 2-methyltetrahydrofuran, and the reaction solution was deoxygenated and purged with nitrogen. Copper(I) acetate (103 mg, 0.83 mmol) and R—(+)-1,1′-binaphthyl-2,2′-bis(diphenylphosphine) (569 mg, 0.91 mmol) were added, the reaction solution was deoxygenated and purged with nitrogen, triethylamine (839 mg, 8.3 mmol) was added dropwise, the reaction solution was deoxygenated and purged with nitrogen, and the reaction was carried out at room temperature for 24 h. After the reaction was completed, the reaction mixture was washed three times with 10% ammonium acetate, twice with saturated sodium chloride, the organic phase was dried over anhydrous sodium sulfate, concentrated, and purified by column chromatography to obtain the target product (3.2 g, yield 76%, ee value 97%). (chiral column, Daicel Corp-ChiralPak® IG, mobile phase: acetonitrile and water, 10% acetonitrile to 100% acetonitrile), specific rotation [α]²⁰=60.1° (c=1 g/100 mL in CHCl₃). ¹H NMR (500 MHz, Chloroform-d) δ 7.35 (t, J=8.5 Hz, 1H), 7.26-7.23 (m, 1H), 7.23-7.20 (m, 1H), 7.20-7.17 (m, 1H), 7.16 (t, J=1.9 Hz, 1H), 7.12 (dd, J=8.5, 2.2 Hz, 1H), 7.09 (d, J=7.6 Hz, 1H), 4.24 (d, J=7.5 Hz, 1H), 4.13 (dd, J=7.7, 1.1 Hz, 1H), 4.04 (d, J=9.0 Hz, 1H), 1.63 (dd, J=15.3, 9.7 Hz, 1H), 1.38 (s, 9H), 1.28 (d, J=14.9 Hz, 1H), 0.89 (s, 9H).

### Step two: Synthesis of tert-butyl (2R,3R,4S,5S)-4-(aminomethyl)-4-(4-chloro-2-fluorophenyl)-3-(3-chlorophenyl)-5-neopentylpyrrolidine-2-carboxylate (JN105)

JN110 (3.2 g, 2.8 mmol), Raney nickel (2.5 g) and hydrazine hydrate (8 mL) were reacted according to the reaction procedure as described in step 3 of intermediate 1 to obtain the target product (1.5 mg, yield 47%).

### Step Three: Synthesis of tert-butyl (2R,3R,4S,5S)-4-(((9H-fluoren-9-yl)methoxy)carbonyl)amino)methyl)-4-(4-chloro-2-fluorophenyl)-3-(3-chlorophenyl)-5-neopentylpyrrolidine-2-carboxylate (JN107)

JN105 (2.6 g, 5.1 mmol) was added into a 100 mL single-neck flask, dissolved in dry dichloromethane, diisopropylethylamine (2.6 g, 20.4 mmol) and FmocCl (2.0 g, 7.6 mmol) were added, and the reaction was carried out at room temperature overnight. After concentration, the mixture was purified by normal-phase column chromatography to obtain the target product (3.2 g, yield 86%).

### Step Four. Synthesis of (2R,3R,4S,5S)-4-(((9H-fluoren-9-yl)methoxy)carbonyl)amino)methyl)-4-(4-chloro-2-fluorophenyl)-3-(3-chlorophenyl)-1-ethyl-5-neopentylpyrrolidine-2-carboxylic Acid (JN113)

JN107 (3.2 g, 4.4 mmol), acetaldehyde (968 mg, 22 mmol), sodium cyanoborohydride (4.7 g, 22 mmol) and 10 mL of acetic acid were added into a 100 mL round-bottom flask, dissolved in 1,2-dichloroethane, and reacted overnight at room temperature. Saturated sodium bicarbonate solution was added, extracted with ethyl acetate, the organic phase was concentrated by rotary evaporation and purified by normal-phase column chromatography to obtain the crude product (3.2 g). The reaction material was dissolved in 10 mL of dichloromethane, 8 mL of trifluoroacetic acid was added, and the reaction was carried out overnight at room temperature. The reaction solution was concentrated by rotary evaporation, saturated sodium bicarbonate solution was added, extracted three times with dichloromethane, the organic phases were combined, concentrated, and purified by normal-phase column chromatography to obtain the target product (2.1 g, yield 70%/).

### Step five: Synthesis of methyl 4-(((2R,3R,4S,5S)-4-(aminomethyl)-4-(4-chloro-2-fluorophenyl)-3-(3-chlorophenyl)-1-ethyl-5-neopentylpyrrolidine-2-carboxamido)-3-methoxybenzoate (JN115)

JN113 (1.0 g, 1.4 mmol) was added to a 100 mL single-neck flask, dissolved in dry dichloromethane, 1-methylimidazole (345 mg, 4.2 mmol) was added at zero degrees Celsius, stirred for 10 minutes, ethylsulfonyl chloride (361 mg, 2.8 mmol) was added, stirred for half an hour, then methyl 4-amino-3-methoxybenzoate (769 mg, 4.2 mmol) was added, and the reaction was carried out at room temperature for 2 hours. Saturated sodium bicarbonate solution was added to the reaction mixture, extracted with dichloromethane, the organic phase was concentrated by rotary evaporation and purified by normal-phase column chromatography to obtain the crude product (1.47 g). The crude product (1.47 g) was dissolved in DMF (10 mL), piperidine (2 mL) was added, and the reaction was carried out at room temperature for 15 minutes. The mixture was washed three times with 1N hydrochloric acid, three times with saturated brine, the organic phase was concentrated by rotary evaporation and purified by normal-phase column chromatography to obtain the target product (800 mg, yield 89%). ¹H NMR (400 MHz, Chloroform-d) δ 8.43 (d, J=8.5 Hz, 1H), 7.62 (d, J=8.5 Hz, 1H), 7.50 (s, 1H), 7.35-7.05 (m, 6H), 7.02 (s, 1H), 4.39-4.18 (m, 3H), 3.86 (s, 6H), 3.39-3.19 (m, 2H), 3.00 (d, J=14.1 Hz, 1H), 2.90 (dd, J=13.7, 6.6 Hz, 1H), 1.82 (dd, J=15.2, 7.2 Hz, 1H), 1.38 (d, J=15.1 Hz, 1H), 1.21 (d, J=6.4 Hz, 3H), 0.83 (s, 9H).

### Step six: Synthesis of 4-((2′S,3S,4′S,5′R)-6-chloro-4′-(3-chlorophenyl)-1′-ethyl-2′-neopentylspiro[indoline-3,3′-pyrrolidine]-5′-carboxamido)-3-methoxybenzoic Acid (JN122)

JN115 (831 mg, 1.3 mmol), potassium carbonate (711 mg, 5.2 mmol) and lithium hydroxide monohydrate (242 mg, 5.8 mmol) were reacted according to the procedure described in step six of the final product 10 to obtain the trifluoroacetate salt of the target product (780 mg, yield 83%, ee value 96.6% (chiral column, Daicel Corp-ChiralPak® IG, mobile phase: acetonitrile and water, 10% acetonitrile to 100% acetonitrile), specific rotation [α]²⁰=−6.1° (c=0.85 g/100 mL in CH₃OH). ¹H NMR (400 MHz, Methanol-d₄) δ 8.17 (d, J=8.4 Hz, 1H), 7.63 (d, J=8.5 Hz, 1H), 7.57 (s, 1H), 7.45-7.21 (m, 4H), 7.14 (d, J=7.4 Hz, 1H), 6.74 (d, J=8.1 Hz, 1H), 6.46 (s, 1H), 5.22-5.05 (m, 1H), 4.37-4.05 (m, 2H), 3.82 (s, 3H), 3.71 (d, J=11.3 Hz, 2H), 3.56 (d, J=11.1 Hz, 1H), 3.42-3.34 (m, 1H), 2.18-1.94 (m, 1H), 1.96-1.75 (m, 1H), 1.44 (t, J=7.1 Hz, 3H), 0.84 (s, 9H). ESI-MS calculated for C₃₃H₃₈³⁵Cl₂N₃O₄ [M+H]⁺=610.2, found: 610.2.

### Final Product 33: 4-((2′S,3S,4′R,5′R)-6-chloro-4′-(3-chlorophenyl)-1′-ethyl-2′-((1-methylcyclobutyl)methyl)spiro[indoline-3,3′-pyrrolidine]-5′-carboxamido)-3-methoxybenzoic Acid (LCC153)

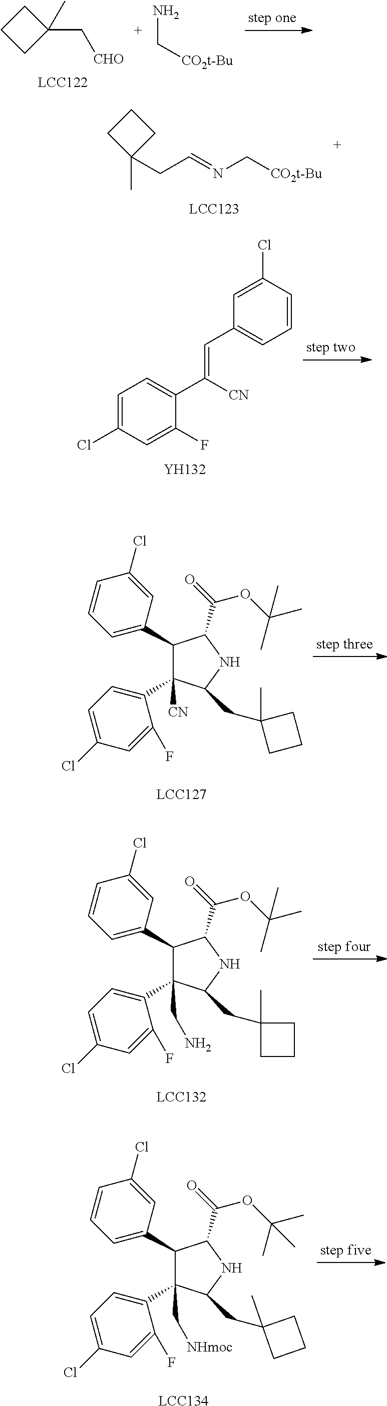

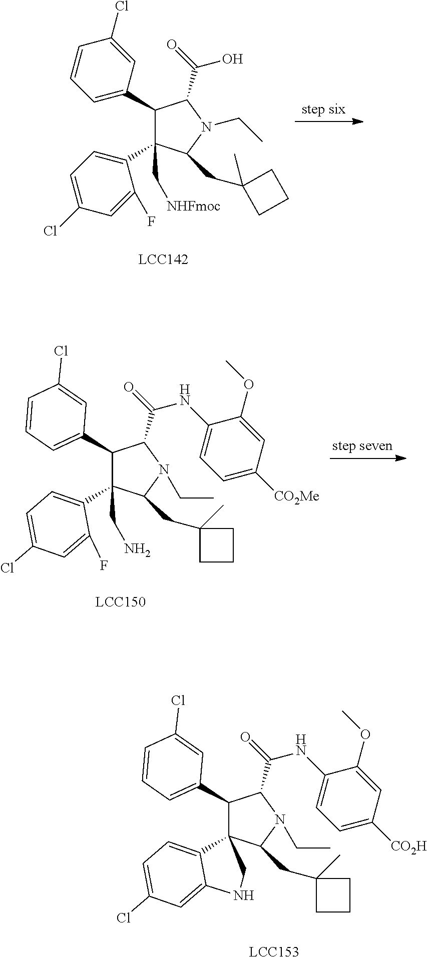

### Step one: Synthesis of tert-butyl (E)-5-(1-methylcyclobutyl)-3-pentenoate (LCC123)

LCC122 (250 mg, 2.3 mmol) was dissolved in dichloromethane, tert-butyl glycinate (293 g, 2.3 mmol) was added dropwise, and the reaction was stirred at room temperature for 18 h. The mixture was dried over sodium sulfate, filtered and the organic phase was evaporated by rotary evaporation to obtain the target product (550 mg, yield 99%).

### Step two: Synthesis of tert-butyl (2R,3R,4R,5S)-4-(4-chloro-2-fluorophenyl)-3-(3-chlorophenyl)-4-cyano-5-((1-methylcyclobutyl)methyl)pyrrolidine-2-carboxylate (LCC127)

YH123 (474 mg, 1.64 mmol), AgF (310 mg, 2.44 mmol), triethylamine (263 mg) and LCC123 (550 mg, 2.5 mmol) were reacted according to the procedure described in step two of intermediate 1 to obtain the target product (150 mg, yield 30%). ¹H NMR (500 MHz, Chloroform-d): δ 7.35 (t, J=8.5 Hz, 1H), 7.25-7.16 (m, 4H), 7.13-7.06 (m, 2H), 4.21 (d, J=7.7 Hz, 1H), 4.11 (d, J=7.7 Hz, 1H), 3.98 (dd, J=9.7, 1.9 Hz, 1H), 1.95-1.77 (m, 4H), 1.76-1.65 (m, 2H), 1.59 (t, J=7.4 Hz, 2H), 1.36 (s, 9H), 1.15 (s, 3H).

### Step Three: Synthesis of tert-butyl (2R,3R,4S,5S)-4-(aminomethyl)-4-(4-chloro-2-fluorophenyl)-3-(3-chlorophenyl)-5-((1-methylcyclobutyl)methyl)pyrrolidine-2-carboxylate (LCC132)

LCC127 (140 mg, 0.3 mmol), Raney nickel (1 g) and hydrazine hydrate 1 (mL) were reacted according to the procedure described in step three of intermediate 1 to obtain the target product (70 mg, yield 50%.

### Step Four: Synthesis of tert-butyl (2R,3R,4S,5S)-4-(((9H-fluoren-9-yl)methoxy)carbonyl)amino)methyl)-4-(4-chloro-2-fluorophenyl)-3-(3-chlorophenyl)-5-((1-methylcyclobutyl)methyl)pyrrolidine-2-carboxylate (LCC134)

LCC132 (58 mg, 0.11 mmol), diisopropylethylamine (57 mg, 0.44 mmol) and Fmoc-Cl (42 mg, 0.16 mmol) were reacted according to the procedure described in step three of final product 18 to obtain the target product (75 mg, yield 92%).

### Step five: Synthesis of (2R,3R,4S,5S)-4-(((((9H-fluoren-9-yl)methoxy)carbonyl)amino)methyl)-4-(4-chloro-2-fluorophenyl)-3-(3-chlorophenyl)-5-((1-methylcyclobutyl)methyl)pyrrolidine-2-carboxylic acid (LCC142)

LCC134 (75 mg, 0.1 mmol), acetaldehyde (44 mg, 1.0 mmol), sodium cyanoborohydride (212 mg, 1.0 mmol), acetic acid (1.0 ml) and trifluoroacetic acid (1 mL) were reacted according to the procedure described in step four of final product 18 to obtain the target product (40 mg, yield 35%).

### Step six: Synthesis of methyl 4-((2R,3R,4S,5S)-4-(aminomethyl)-4-(4-chloro-2-fluorophenyl)-3-(3-chlorophenyl)-1-ethyl-5-((1-methylcyclobutyl)methyl)pyrrolidine-2-carboxamido)-3-methoxybenzoate (LCC150)

LCC142 (40 mg, 0.05 mmol), N-methylimidazole (31 mg, 0.2 mmol), ethylsulfonyl chloride (32 mg, 0.2 mmol), methyl 4-amino-3-methoxybenzoate (32 mg, 0.2 mmol) and piperidine (1 mL) were reacted according to the procedure described in step five of final product 18 to obtain the target product (12 mg, yield 10%).

### Step seven: Synthesis of 4-((2′S,3S,4′R,5′R)-6-chloro-4′-(3-chlorophenyl)-1′-ethyl-2′-((1-methylcyclobutyl)methyl)spiro[indoline-3,3′-pyrrolidine]-5′-carboxamido)-3-methoxybenzoic Acid (LCC153)

LCC150 (12 mg, 0.02 mmol), potassium carbonate (10 mg, 0.07 mmol) and lithium hydroxide monohydrate (60 mg, 0.2 mmol) were reacted according to the procedure described in step six of final product 10 to obtain the target product (3 mg, yield 17%). ¹H NMR (400 MHz, Methanol-d₄) δ 8.22 (d, J=8.2 Hz, 1H), 7.63 (d, J=8.4 Hz, 1H), 7.57 (s, 1H), 7.47-7.18 (m, 4H), 7.09 (d, J=7.7 Hz, 1H), 6.70 (d, J=7.9 Hz, 1H), 6.38 (s, 1H), 4.53-3.69 (m, 7H), 3.62 (s, 2H), 3.12 (s, 1H), 2.20-1.96 (m, 2H), 1.94-1.79 (m, 2H), 1.76-1.52 (m, 3H), 1.38 (s, 3H), 1.22 (s, 3H). ESI-MS calculated value for C₃₄H₃₈³⁵Cl₂N₃O₄ [M+H]⁺=622.2, found: 622.3.

### Final Product 34: 4-((2′S,3S,4′R,5′R)-6-chloro-4′-(3-chlorophenyl)-1-ethyl-2′-((1-methylcyclopentyl)methyl)spiro[indoline-3,3′-pyrrolidine]-5′-carboxamido)-3-methoxybenzoic Acid (LCC63)

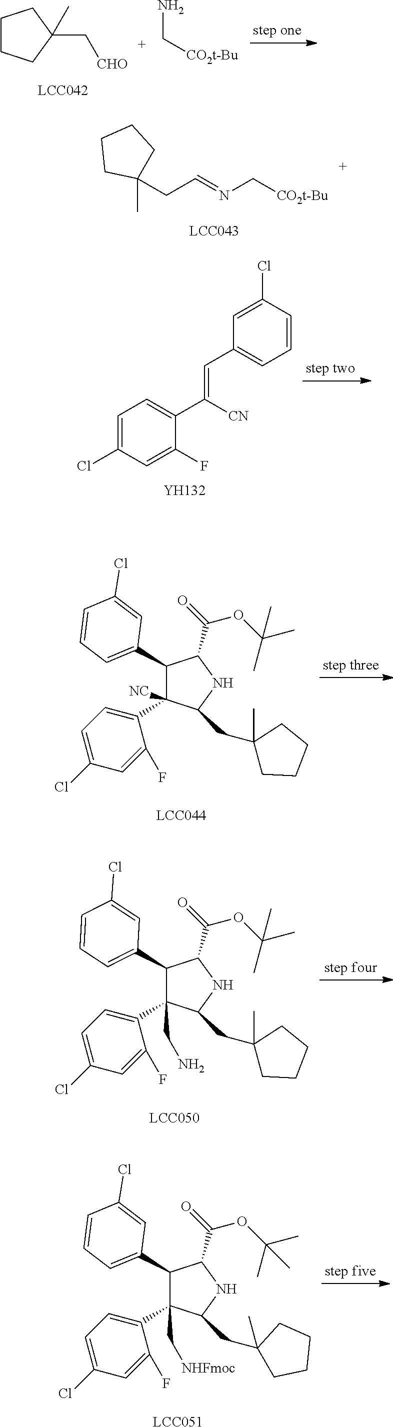

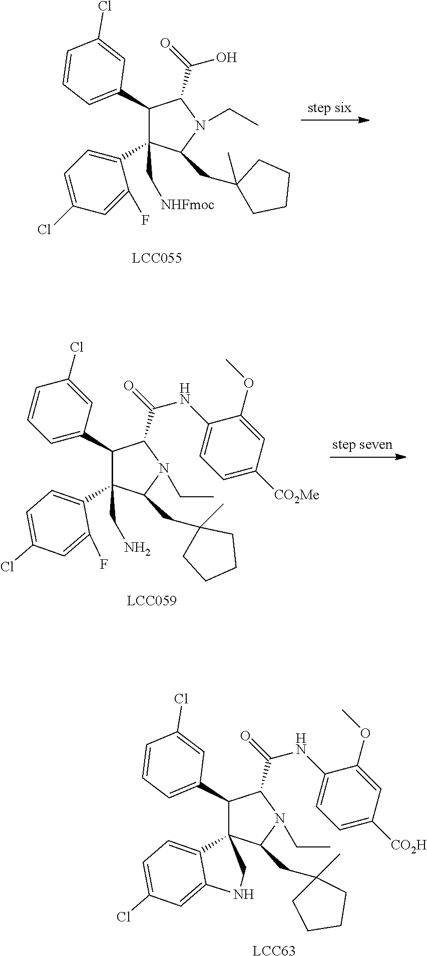

### Step one: Synthesis of tert-butyl (E)-5-(1-methylcyclopentyl)pent-3-enoate (LCC043)

LCC041 (340 mg, 2.7 mmol), tert-butyl glycinate (353 mg, 2.7 mmol) were reacted according to the procedure described in step one of final product 33 to obtain the target product (618 mg, yield 99%).

### Step two: Synthesis of tert-butyl (2R,3R,4R,5S)-4-(4-chloro-2-fluorophenyl)-3-(3-chlorophenyl)-4-cyano-5-((1-methylcyclopentyl)methyl)pyrrolidine-2-carboxylate (LCC044)

YH132 (501 mg, 1.72 mmol), AgF (328 mg, 2.58 mmol), triethylamine (218 mg) and LCC043 (618 mg, 2.58 mmol) were reacted according to the procedure described in step two of intermediate 1 to obtain the target product (234 mg, yield 30%). ¹H NMR (400 MHz, Chloroform-d) δ 7.35 (t, J=8.5 Hz, 1H), 7.25-7.20 (m, 2H), 7.20-7.15 (m, 2H), 7.11 (t, J=9.6 Hz, 2H), 4.24 (d, J=7.8 Hz, 1H), 4.13 (d, J=7.6 Hz, 1H), 4.05 (d, J=9.1 Hz, 1H), 1.77-1.55 (m, 7H), 1.40-1.36 (m, 10H), 1.21-1.08 (m, 2H), 0.94 (s, 3H).

### Step Three: Synthesis of tert-butyl (2R,3R,4S,5S)-4-(aminomethyl)-4-(4-chloro-2-fluorophenyl)-3-(3-chlorophenyl)-5-((1-methylcyclopentyl)methyl)pyrrolidine-2-carboxylate (LCC050)

LCC044 (234 mg, 0.4 mmol), Raney nickel (1 g) and hydrazine hydrate (1 mL) were reacted according to the procedure described in step three of intermediate 1 to obtain the target product (99 mg, yield 45%).

### Step Four: Synthesis of tert-butyl (2R,3R,4S,5S)-4-((((9H-fluoren-9-yl)methoxy)carbonyl)amino)methyl)-4-(4-chloro-2-fluorophenyl)-3-(3-chlorophenyl)-5-((1-methylcyclopentyl)methyl)pyrrolidine-2-carboxylate (LCC051)

LCC050 (99 mg, 0.19 mmol), diisopropylethylamine (96 mg, 0.7 mmol) and Fmoc-Cl (72 mg, 0.3 mmol) were reacted according to the procedure described in step three of final product 18 to obtain the target product (130 mg, yield 78%).

### Step five: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-5-((1-methylcyclopentyl)methyl)pyrrolidine-2-carboxylic Acid (LCC055)

LCC051 (124 mg, 0.15 mmol), acetaldehyde (76 mg, 1.7 mmol), sodium borohydride acetate (365 mg, 1.7 mmol), acetic acid (1 ml) and trifluoroacetic acid (2 mL) were reacted according to the procedure described in step four of final product 18 to obtain the target product (63 mg, yield 35%).

### Step six: Synthesis of methyl 4-((2R,3R,4S,5S)-4-(aminomethyl)-4-(4-chloro-2-fluorophenyl)-3-(3-chlorophenyl)-1-ethyl-5-((1-methylcyclopentyl)methyl)pyrrolidine-2-carboxamido)-3-methoxybenzoate (LCC059)

LCC055 (63 mg, 0.08 mmol), N-methylimidazole (22 mg, 0.3 mmol), ethylsulfonyl chloride (23 mg, 0.2 mmol), methyl 4-amino-3-methoxybenzoate (47 mg, 0.3 mmol) and piperidine (1 mL) were reacted according to the procedure described in step five of final product 18 to obtain the target product (30 mg, yield 28%). ¹H NMR (500 MHz, MeOH-d₄): δ 8.26 (d, J=8.2 Hz, 1H), 7.62 (d, J=8.3 Hz, 2H), 7.54 (d, J=8.5 Hz, 1H), 7.39-7.24 (m, 4H), 7.15 (d, J=2.0 Hz, 1H), 7.06 (d, J=7.2 Hz, 1H), 4.39 (d, J=8.9 Hz, 1H), 4.33 (d, J=9.3 Hz, 1H), 4.01 (d, J=9.3 Hz, 1H), 3.96 (s, 3H), 3.89 (s, 3H), 3.51-3.39 (m, 2H), 3.02 (dq, J=13.4, 6.8 Hz, 1H), 2.13-2.02 (m, 1H), 1.80-1.41 (m, 9H), 1.32 (dt, J=14.7, 9.2 Hz, 2H), 1.25 (t, J=7.0 Hz, 3H), 1.13 (s, 3H).

### Step seven: Synthesis of 4-((2′S,3S,4′R,5′R)-6-chloro-4′-(3-chlorophenyl)-1′-ethyl-2′-((1-methylcyclopentyl)methyl)spiro[indoline-3,3′-pyrrolidine]-5′-carboxamido)-3-methoxybenzoic Acid (LCC063)

LCC059 (20 mg, 0.03 mmol), potassium carbonate (17 mg, 0.1 mmol) and lithium hydroxide monohydrate (100 mg, 0.2 mmol) were reacted according to the procedure described in step six of the final product 10 to obtain the target product (12 mg, yield 40%). ¹H NMR (500 MHz, Methanol-d₄) δ 8.20 (d, J=8.5 Hz, 1H), 7.70-7.53 (m, 2H), 7.45-7.22 (m, 4H), 7.12 (d, J=7.6 Hz, 1H), 6.80-6.66 (m, 1H), 6.43 (d, J=1.9 Hz, 1H), 4.38-3.97 (m, 2H), 3.83 (s, 3H), 3.77-3.62 (m, 2H), 3.56 (s, 1H), 3.25 (s, 1H), 2.11 (s, 1H), 1.91 (s, 1H), 1.72-1.50 (m, 3H), 1.50-1.32 (m, 6H), 1.15 (s, 2H), 0.97 (s, 3H). ESI-MS calculated value for C₃₅H₄₀³⁵Cl₂N₃O₄ [M+H]⁺=636.2, found: 636.4.

### Final Product 35: 4-((2′S,3S,4′R,5′R)-6-chloro-4′-(3-chlorophenyl)-1′-ethyl-2′-((1-methylcyclohexyl)methyl)spiro[indoline-3,3′-pyrrolidine]-5′-carboxamido)-3-methoxybenzoic Acid (LCC131)

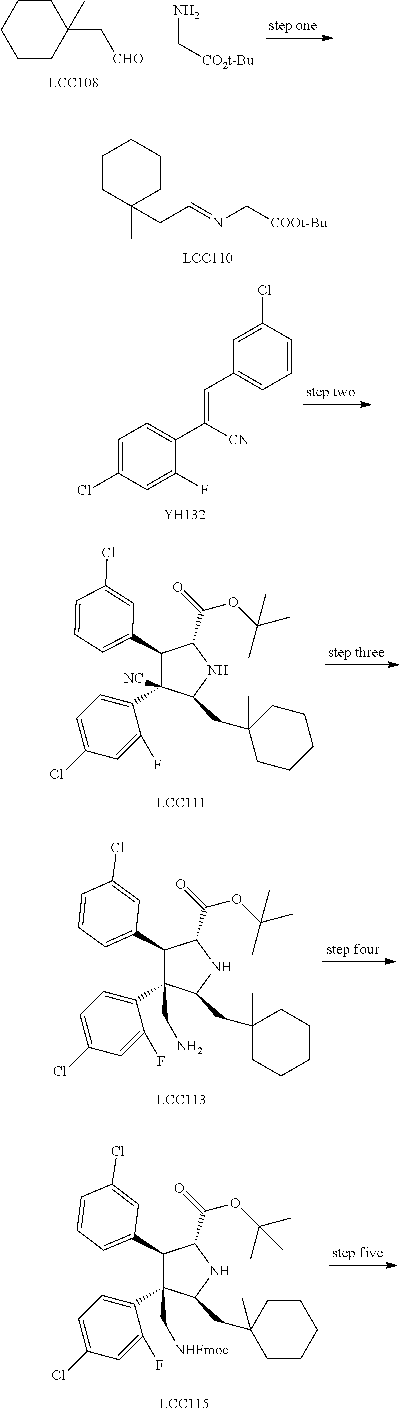

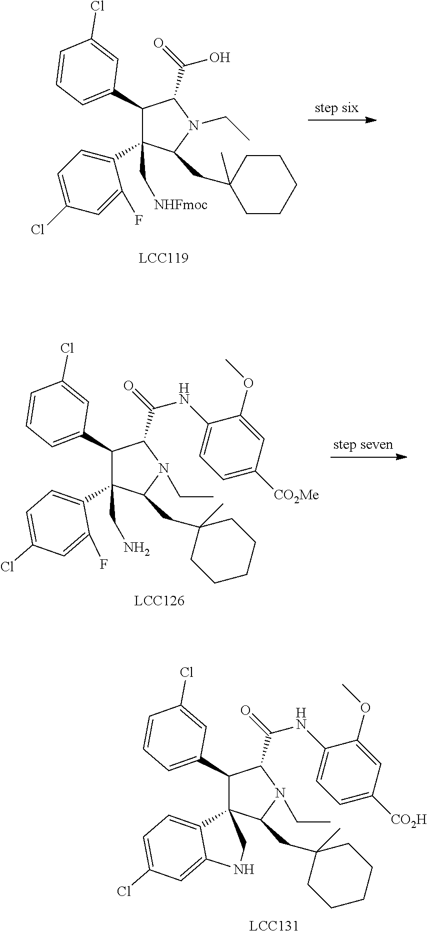

### Step one: Synthesis of tert-butyl (E)-5-(1-methylcyclohexyl)pent-3-enoate (LCC110)

Fatty aldehyde LCC108 (1.88 g, 13 mmol) and tert-butyl glycinate (1.76 g, 13 mmol) were reacted according to the procedure described in step one of final product 33 to obtain the target product (3.27 g, yield 99%).

### Step two: Synthesis of tert-butyl (2R,3R,4R,5S)-4-(4-chloro-2-fluorophenyl)-3-(3-chlorophenyl)-4-cyano-5-((1-methylcyclohexyl)methyl)pyrrolidine-2-carboxylate (LCC111)

YH132 (2.5 g, 8.6 mmol), AgF (1.64 g, 13 mmol), triethylamine (1.4 g) and LCC110 (3.27 g, 13 mmol) were reacted according to the procedure described in step two of intermediate 1 to obtain the target product (1.3 g, yield 30%). ¹H NMR (400 MHz, Chloroform-d₄) δ 7.37 (t, J=8.5 Hz, 1H), 7.25-7.22 (m, 1H), 7.22-7.15 (m, 3H), 7.15-7.08 (m, 2H), 4.23 (d, J=7.6 Hz, 1H), 4.13 (d, J=7.6 Hz, 1H), 4.04 (d, J=8.8 Hz, 1H), 1.66-1.57 (m, 2H), 1.40-1.37 (m, 10H), 1.37-1.18 (m, 8H), 0.86 (s, 3H).

### Step Three: Synthesis of tert-butyl (2R,3R,4S,5S)-4-(aminomethyl)-4-(4-chloro-2-fluorophenyl)-3-(3-chlorophenyl)-5-((1-methylcyclohexyl)methyl)pyrrolidine-2-carboxylate (LCC113)

LCC113 (1.3 g, 2.4 mmol), Raney nickel (2 g) and hydrazine hydrate (5 mL) were reacted according to the procedure described in step three of intermediate 1 to obtain the target product (655 mg, yield 50%).

### Step Four: Synthesis of tert-butyl (2R,3R,4S,5S)-4-((((9H-fluoren-9-yl)methoxy)carbonyl)amino)methyl)-4-(4-chloro-2-fluorophenyl)-3-(3-chlorophenyl)-5-((1-methylcyclohexyl)methyl)pyrrolidine-2-carboxylate (LCC115)

LCC113 (912 mg, 1.67 mmol), diisopropylethylamine (860 mg, 6.7 mmol) and Fmoc-Cl (645 mg, 2.5 mmol) were reacted according to the procedure described in step three of final product 18 to obtain the target product (1 g, yield 78%).

### Step five: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-5-((1-methylcyclopentyl)methyl)pyrrolidine-2-carboxylic Acid (LCC119)

LCC115 (200 mg, 0.3 mmol), acetaldehyde (114 mg, 2.6 mmol), sodium borohydride acetate (551 mg, 2.6 mmol), acetic acid (1.5 ml) and trifluoroacetic acid (3 mL) were reacted according to the procedure described in step four of final product 18 to obtain the target product (119 mg, yield 62%).

### Step six: Synthesis of methyl 4-((2R,3R,4S,5S)-4-(aminomethyl)-4-(4-chloro-2-fluorophenyl)-3-(3-chlorophenyl)-1-ethyl-5-((1-methylcyclohexyl)methyl)pyrrolidine-2-carboxamido)-3-methoxybenzoate (LCC126)

LCC119 (119 mg, 0.16 mmol), N-methylimidazole (39 mg, 0.5 mmol), ethylsulfonyl chloride (42 mg, 0.3 mmol), methyl 4-amino-3-methoxybenzoate (87 mg, 0.5 mmol) and piperidine (2 mL) were reacted according to the procedure described in step five of final product 18 to obtain the target product (109 mg, yield 30%). ¹H NMR (400 MHz, Methanol-d₄) δ 8.26 (d, J=8.8 Hz, 1H), 7.66-7.48 (m, 3H), 7.39-7.24 (m, 5H), 7.19-7.02 (m, 2H), 4.42 (d, J=8.2 Hz, 1H), 4.34 (d, J=9.1 Hz, 1H), 4.05 (d, J=9.2 Hz, 1H), 3.95 (s, 3H), 3.88 (s, 3H), 3.55-3.39 (m, 2H), 3.08-2.96 (m, 1H), 1.97-1.81 (m, 1H), 1.60-1.33 (m, 8H), 1.33-1.14 (m, 6H), 1.10 (s, 3H).

### Step seven: Synthesis of 4-((2′S,3S,4′R,5′R)-6-chloro-4′-(3-chlorophenyl)-1′-ethyl-2′-((1-methylcyclohexyl)methyl)spiro[indoline-3,3′-pyrrolidine]-5′-carboxamido)-3-methoxybenzoic Acid (LCC131)

LCC126 (85 mg, 0.13 mmol), potassium carbonate (69 mg, 0.5 mmol) and lithium hydroxide monohydrate (130 mg, 0.2 mmol) were reacted according to the procedure described in step six of the final product 10 to obtain the target product (24 mg, yield 39%). ¹H NMR (400 MHz, Methanol-d₄) δ 8.17 (d, J=8.4 Hz, 1H), 7.67-7.52 (m, 2H), 7.41-7.24 (m, 4H), 7.14 (d, J=7.5 Hz, 1H), 6.79-6.68 (m, 1H), 6.46 (s, 1H), 5.07 (s, 1H), 4.44-4.05 (m, 2H), 3.81 (s, 3H), 3.77-3.69 (m, 2H), 3.64-3.54 (m, 1H), 3.35 (s, 1H), 1.94 (s, 2H), 1.57-1.38 (m, 6H), 1.37-1.11 (m, 6H), 1.09-0.76 (m, 4H). ESI-MS calculated for C₃₆H₄₂³⁵Cl₂N₃O₄ [M+H]⁺=650.3, found: 650.4.

### Final Product 37: 4-((2′S,3S,4′R,5′R)-6-chloro-4′-(3-chlorophenyl)-2′-(2,2-dimethylbutyl)-1′-ethylspiro[indoline-3,3′-pyrrolidine]-5′-carboxylic acid)-3-methoxybenzoic Acid (TC90)

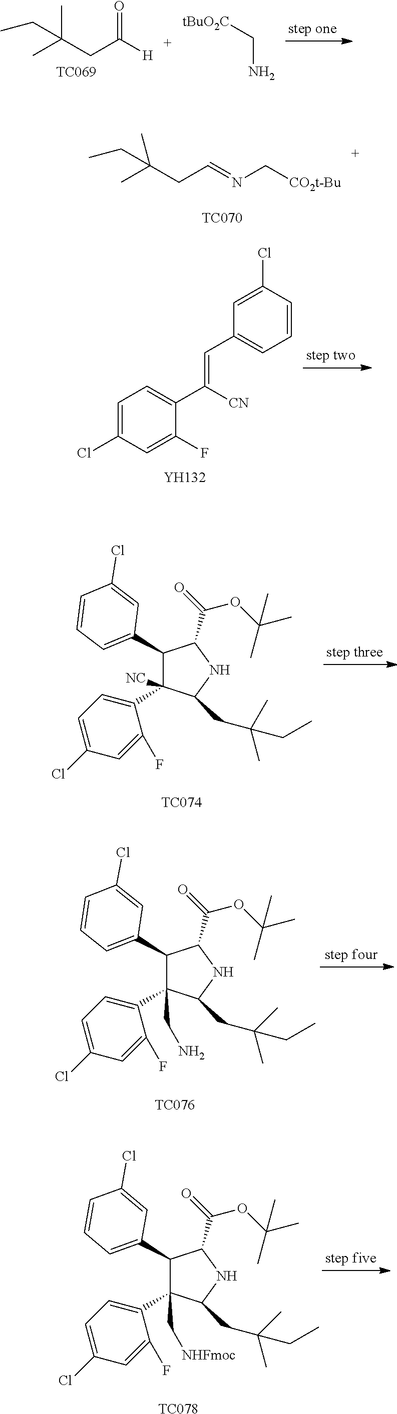

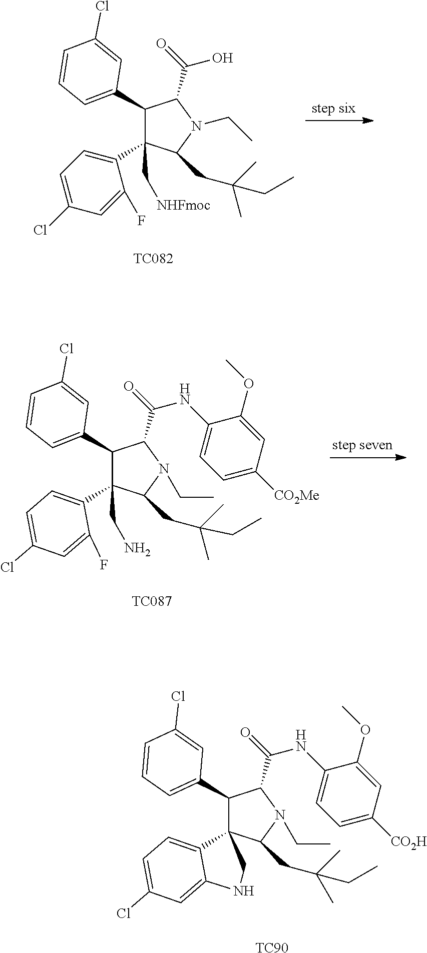

### Step one: tert-butyl (E)-2-((3,3-dimethylpentylidene)amino)acetate (TC070)

TC069 (2.5, 21.9 mmol) and tert-butyl glycinate (3.2 g, 22.9 mmol) were reacted according to the procedure described in step one of the final product 33 to obtain the target product (5.1 g, yield 99%).

### Step two: Synthesis of tert-butyl (2R,3R,4R,5S)-4-(4-chloro-2-fluorophenyl)-3-(3-chiorophenyl)-4-cyano-5-(2,2-dimethylbutyl)pyrrolidine-2-carboxylate (TC074)

TC070 (5.1 g, 22.5 mmol), YH132 (6.1 g, 20.8 mmol), AgF (2.54 g, 20 mmol) and triethylamine (4.5 mL) were reacted according to the procedure described in step two of intermediate 1 to obtain the target product (3.2 g, yield 30%). ¹H NMR (500 MHz, Chloroform-d) δ 7.36 (t, J=8.5 Hz, 1H), 7.26 (d, J=1.7 Hz, 1H), 7.25-7.22 (m, 1H), 7.22-7.19 (m, 1H), 7.19-7.16 ((m, 1H), 7.13 (dd, J=8.5, 2.2 Hz, 1H), 7.10 (dt, J=7.5, 1.6 Hz, 1H), 4.24 (d, J=7.6 Hz, 1H), 4.14 (d, J=7.6 Hz, 1H), 4.02 (d, J=8.9 Hz, 1H), 1.60 (dd, J=14.5, 9.0 Hz, 2H), 1.38 (s, 9H), 1.26-1.19 (m, 2H), 0.84 (s, 3H), 0.82 (s, 3H), 0.67 (t, J=7.5 Hz, 3H).

### Step Three: Synthesis of tert-butyl (2R,3R,4S,5S)-4-(aminomethyl)-4-(4-chloro-2-fluorophenyl)-3-(3-chlorophenyl)-5-(2,2-dimethylbutyl)pyrrolidine-2-carboxylate (TC076)

TC074 (3.2 g, 6.2 mmol), Raney nickel (3 g) and hydrazine hydrate (10 mL) were reacted according to the procedure described in step three of intermediate 1 to obtain the target product (1.7 g, yield 52%). ¹H NMR (500 MHz, Methanol-d₄) δ 7.29-7.23 (m, 5H), 7.15 (s, 1H), 7.06-6.99 (m, 1H), 4.34 (d, J=8.5 Hz, 1H), 4.13 (d, J=8.4 Hz, 1H), 4.03 (d, J=8.2 Hz, 1H), 3.25 (d, J=14.0 Hz, 1H), 3.05 (d, J=14.1 Hz, 1H), 1.48-1.41 (m, 2H), 1.34 (s, 9H), 1.30-1.26 (m, 2H), 0.84 (s, 3H), 0.82 (s, 3H), 0.71 (t, J=7.4 Hz, 3H).

### Step Four: Synthesis of tert-butyl (2R,3R,4S,5S)-4-(((((9H-fluoren-9-yl)methoxy)carbonyl)amino)methyl)-4-(4-chloro-2-fluorophenyl)-3-(3-chlorophenyl)-5-(2,2-dimethylbutyl)pyrrolidine-2-carboxylate (TC078)

TC076 (1.7 g, 3.24 mmol), diisopropylethylamine (1.68 g, 13 mmol) and Fmoc-Cl (1.27 g, 4.9 mmol) were reacted according to the procedure described in step three of final product 18 to obtain the target product (2.3 g, yield 95%).

### Step five. Synthesis of (2R,3R,4S,5S)-4-(((((9H-fluoren-9-yl)methoxy)carbonyl)amino)methyl)-4-(4-chloro-2-fluorophenyl)-3-(3-chlorophenyl)-5-(2,2-dimethylbutyl)-1-ethylpyrrolidine-2-carboxylic Acid (TC082)

TC078 (200 mg, 0.269 mmol), acetaldehyde (120 mg, 2.7 mmol), sodium borohydride acetate (570 mg, 2.7 mmol), acetic acid (3 mL) and trifluoroacetic acid (2 mL) were reacted according to the procedure described in step four of final product 18 to obtain the target product (145 mg, yield 72%).

### Step six: Synthesis of methyl 4-((2R,3R,4S,5S)-4-(aminomethyl)-4-(4-chloro-2-fluorophenyl)-3-(3-chlorophenyl)-5-(2,2-dimethylbutyl)-1-ethylpyrrolidine-2-carboxamido)-3-methoxybenzoate (TC087)

TC082 (145 mg, 0.18 mmol), methyl 4-amino-3-methoxybenzoate (100 mg, 0.53 mmol), N-methylimidazole (45 mg, 0.53 mmol), ethylsulfonyl chloride (46 mg, 0.354 mmol) and piperidine (0.2 mL) were reacted according to the procedure described in step five of the final product 18 to obtain the target product (92.5 mg, yield 78%). ¹H NMR (500 MHz, Methanol-d₄) δ 8.27 (d, J=8.8 Hz 1H), 7.62 (t, J=7.5 Hz, 2H), 7.58-7.50 (m, 1H), 7.41-7.23 (m, 4H), 7.15 (s, 1H), 7.07 (d, J=7.1 Hz, 1H), 4.40 (d, J=8.9 Hz, 1H), 4.32 (d, J=9.2 Hz, 1H), 4.03 (d, J=9.1 Hz, 1H), 3.96 (s, 3H), 3.89 (s, 3H), 3.49-3.38 (m, 2H), 3.29 (d, J=16.3 Hz, 1H), 3.05-2.95 (m, 1H), 1.95 (dd, J=15.1, 9.1 Hz, 1H), 1.45 (d, J=15.0 Hz, 1H), 1.33 (tt, J=7.3, 4.1 Hz, 2H), 1.24 (t, J=7.0 Hz, 3H), 1.06 (s, 3H), 0.93 (s, 3H), 0.80 (t, J=7.5 Hz, 3H).

### Step seven: Synthesis of 4-((2′S,3S,4′R,5′R)-6-chloro-4′-(3-chlorophenyl)-2′-(2,2-dimethylbutyl)-1′-ethylspiro[indoline-3,3′-pyrrolidine]-5′-carboxylic acid)-3-methoxybenzoic Acid (TC90)

TC087 (84 mg, 0.127 mmol), potassium carbonate (70 mg, 0.51 mmol) and lithium hydroxide monohydrate (120 mg, 2.86 mmol) were reacted according to the procedure described in step six of the final product 10 to obtain the target product (36.2 mg, yield 46%). ¹H NMR (500 MHz, Methanol-d₄) δ 8.20 (d, J=8.4 Hz, 1H), 7.63 (dd, J=8.4, 1.8 Hz, 1H), 7.58 (s, 1H), 7.49-7.21 (m, 4H), 7.13 (d, J=7.5 Hz, 1H), 6.73 (dd, J=8.0, 1.9 Hz, 1H), 6.46 (d, J=1.9 Hz, 1H), 4.31-4.07 (m, 2H), 3.83 (s, 3H), 3.69 (d, J=10.8 Hz, 2H), 3.50 (s, 1H), 3.31-3.19 (m, 2H), 2.06-1.69 (m, 2H), 1.42 (t, J=7.1 Hz, 3H), 1.34-1.06 (m, 2H), 0.89 (s, 3H), 0.72 (t, J=7.4 Hz, 6H). ESI-MS calculated for C₃₄H₃₉³⁵Cl₂N₃O₄ [M+H]⁺=624.23, found: 624.3.

### Final Product 38: 4-((2′S,3S,4′R,5′R)-6-chloro-4′-(3-chlorophenyl)-1′-ethyl-2′-(2-ethyl-2-methylbutyl)spiro[indoline-3,3′-pyrrolidine]-5′-amino)-3-methoxybenzoic Acid (TC29)

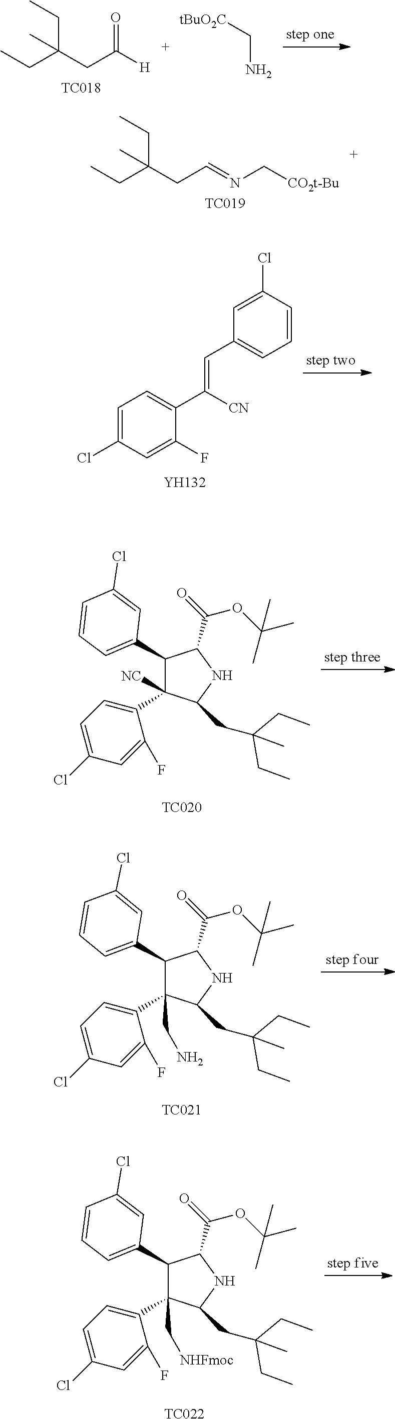

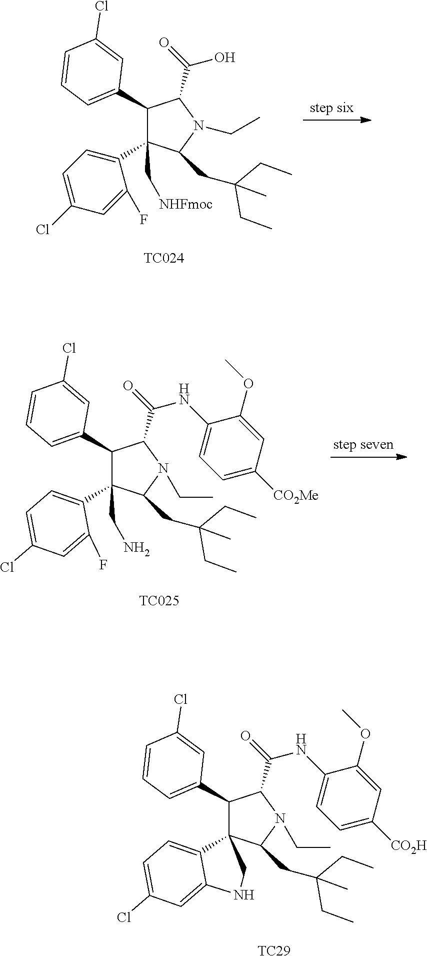

### Step one: tert-butyl (E)-2-((3-ethyl-3-methylpentylidene)amino)acetate (TC019)

TC018 (2.9, 22.7 mmol) and tert-butyl glycinate (3.5 g, 25 mmol) were reacted according to the procedure described in step one of the final product 33 to obtain the target product (5.1 g, yield 99%).

### Step two: Synthesis of tert-butyl (2R,3R,4R,5S)-4-(4-chloro-2-fluorophenyl)-3-(3-chlorophenyl)-4-cyano-5-(2-ethyl-2-methylbutyl)pyrrolidine-2-carboxylate (TC020)

YH132 (5.6 g, 19.2 mmol), TC019 (5.4 g, 22.4 mmol), AgF (2.5 g, 19.2 mmol) and triethylamine (4.3 mL) were reacted according to the procedure described in step two of intermediate 1 to obtain the target product (3.2 g, yield 31.2%). ¹H NMR (400 MHz, Chloroform-d) δ 7.38 (t, J=8.5 Hz, 1H), 7.24-7.21 (m, 1H), 7.22-7.18 (m, 1H), 7.19-7.15 (m, 2H), 7.13 (dd, J=8.4, 2.2 Hz, 1H), 7.10 (dt, J=7.1, 2.6 Hz, 1H), 4.23 (d, J=7.6 Hz, 1H), 4.15 (d, J=7.5 Hz, 1H), 4.00 (d, J=8.9 Hz, 1H), 1.59 (dd, J=14.7, 8.9 Hz, 2H), 1.38 (s, 9H), 1.32-1.12 (m, 4H), 0.79 (s, 3H), 0.66 (t, J=7.5 Hz, 3H), 0.59 (t, J=7.5 Hz, 3H).

### Step Three: Synthesis of tert-butyl (2R,3R,4S,5S)-4-(aminomethyl)-4-(4-chloro-2-fluorophenyl)-3-(3-chlorophenyl)-5-(2-ethyl-2-methylbutyl)pyrrolidine-2-carboxylate (TC021)

TC020 (3.2 g, 6.0 mmol), Raney nickel (3 g) and hydrazine hydrate (10 mL) were reacted according to the procedure described in step three of intermediate 1 to obtain the target product (1.1 g, yield 34%). ¹H NMR (400 MHz, Methanol-d₄): δ 7.38-7.27 (m, 4H), 7.20 (t, J=8.8 Hz, 1H), 7.08 (s, 1H), 6.98 (d, J=7.8 Hz, 1H), 4.34 (d, J=7.5 Hz, 1H), 4.24 (t, J=5.2 Hz, 1H), 4.15 (t, J=7.6 Hz, 1H), 3.40 (d, J=13.8 Hz, 1H), 3.05 (d, J=13.8 Hz, 1H), 1.94 (s, 2H), 1.40 (s, 9H), 1.35-1.22 (m, 4H), 0.86 (s, 3H), 0.72 (t, J=7.5 Hz, 3H), 0.63 (t, J=7.5 Hz, 3H).

### Step Four: Synthesis of tert-butyl (2R,3R,4S,5S)-4-((((9H-fluoren-9-yl)methoxy)carbonyl)amino)methyl)-4-(4-chloro-2-fluorophenyl)-3-(3-chlorophenyl)-5-(2-ethyl-2-methylbutyl)pyrrolidine-2-carboxylate (TC022)

TC021 (340 mg, 0.63 mmol), diisopropylethylamine (326 mg, 2.52 mmol) and Fmoc-Cl (243 mg, 0.94 mmol) were reacted according to the procedure described in step three of the final product 18 to obtain the target product (380 mg, yield 79%).

### Step five. Synthesis of (2R,3R,4S,5S)-4-(((((9H-fluoren-9-yl)methoxy)carbonyl)amino)methyl)-4-(4-chloro-2-fluorophenyl)-3-(3-chlorophenyl)-1-ethyl-5-(2-ethyl-2-methylbutyl)pyrrolidine-2-carboxylic Acid (TC024)

TC023 (380 g, 0.486 mmol), acetaldehyde (214 mg, 4.9 mmol), sodium borohydride acetate (1 g, 4.9 mmol), acetic acid (3 mL) and trifluoroacetic acid (3 mL) were reacted according to the procedure described in step four of the final product 18 to obtain the target product (210 mg, yield 45.6%).

### Step six: Synthesis of methyl 4-((2R,3R,4S,5S)-4-(aminomethyl)-4-(4-chloro-2-fluorophenyl)-3-(3-chlorophenyl)-1-ethyl-5-(2-ethyl-2-methylbutyl)pyrrolidine-2-carboxamido)-3-methoxybenzoate (TC025)

TC024 (100 mg, 0.132 mmol), methyl 4-amino-3-methoxybenzoate (75 mg, 0.395 mmol), N-methylimidazole (35 mg, 0.395 mmol), ethylsulfonyl chloride (35 mg, 0.264 mmol) and piperidine (0.2 mL) were reacted according to the procedure described in step five of the final product 18 to obtain the target product (85 mg, yield 99%). ¹H NMR (500 MHz, Methanol-d₄) δ 8.26 (d, J=8.8 Hz, 1H), 7.61 (td, J=4.6, 2.2 Hz, 2H), 7.55 (s, 1H), 7.37-7.24 (m, 4H), 7.17 (s, 1H), 7.09 (d, J=6.9 Hz, 1H), 4.36 (d, J=9.4 Hz, 2H), 4.11 (d, J=9.1 Hz, 1H), 3.94 (s, 3H), 3.88 (s, 3H), 3.48-3.38 (m, 2H), 3.03-2.91 (m, 1H), 1.91-1.83 (m, 1H), 1.47 (d, J=15.1 Hz, 1H), 1.41-1.27 (m, 4H), 1.25 (t, J=7.0 Hz, 3H), 1.18 (q, J=14.2, 7.3 Hz, 1H), 1.00 (s, 3H), 0.83 (t, J=7.4 Hz, 3H), 0.59 (t, J=7.4 Hz, 3H).

### Step seven: Synthesis of 4-((2′S,3S,4′R,5′R)-6-chloro-4′-(3-chlorophenyl)-1′-ethyl-2′-(2-ethyl-2-methylbutyl)spiro[indoline-3,3′-pyrrolidine]-5′-amino)-3-methoxybenzoic Acid (TC29)

TC025 (85 mg, 0.127 mmol), potassium carbonate (70 mg, 0.51 mmol) and lithium hydroxide monohydrate (120 mg, 2.86 mmol) were reacted according to the procedure described in step six of the final product 10 to obtain the target product (58 mg, yield 71.6%). ¹H NMR (400 MHz, Methanol-d₄) δ 8.23 (d, J=8.4 Hz, 1H), 7.63 (dd, J=8.4, 1.8 Hz, 1H), 7.59 (s, 1H), 7.37-7.23 (m, 4H), 7.11 (d, J=7.1 Hz, 1H), 6.71 (dd, J=8.0, 1.9 Hz, 1H), 6.44 (d, J=1.8 Hz, 1H), 4.05 (s, 2H), 3.85 (s, 3H), 3.66 (d, J=10.7 Hz, 2H), 3.52 (d, J=11.4 Hz, 1H), 3.25-3.05 (m, 1H), 2.02-1.84 (m, 1H), 1.73 (s, 1H), 1.38 (t, J=7.0 Hz, 3H), 1.34-1.24 (m, 4H), 1.22-1.01 (m, 1H), 0.86 (s, 3H), 0.77 (t, J=7.4 Hz, 3H), 0.56 (t, J=7.4 Hz, 3H). ESI-MS theoretical value C₃₅H₄₂³⁵Cl₂N₃O₄ [M+H]⁺=638.25, found: 638.0.

### Final Product 43: 4-((2′S,3S,4′R,5′R)-6-chloro-4′-(3-chlorophenyl)-1′-ethyl-2′-neopentylspiro[indoline-3,3′-pyrrolidine]-5′-carboxamido)benzoic Acid (JP16)

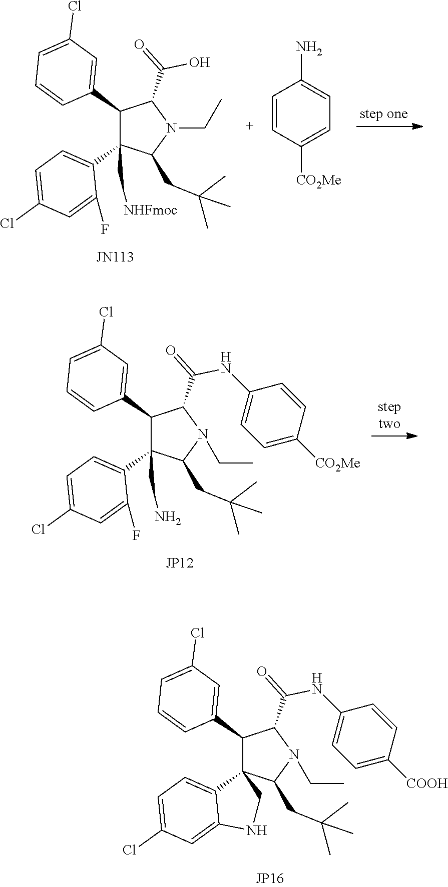

### Step one: Synthesis of methyl 4-((2R,3R,4S,5S)-4-(aminomethyl)-4-(4-chloro-2-fluorophenyl)-3-(3-chlorophenyl)-1-ethyl-5-neopentylpyrrolidine-2-carboxamido)benzoate (JP12)

JN113 (40 mg, 0.06 mmol), 1-methylimidazole (14 mg, 0.17 mmol), ethylsulfonyl chloride (15 mg, 0.11 mmol), methyl 4-aminobenzoate (26 mg, 0.17 mmol) and piperidine (1.5 mL) were reacted according to the procedure described in step five of the final product 18 to obtain the target product (50 mg, yield 99%). ¹H NMR (500 MHz, Methanol-d₄) δ 7.96 (d, J=8.4 Hz, 2H), 7.70 (d, J=8.7 Hz, 2H), 7.42-7.25 (m, 5H), 7.13-6.99 (m, 2H), 4.70-4.64 (m, 1H), 4.63 (d, J=7.3 Hz, 1H), 4.56 (d, J=7.2 Hz, 1H), 3.85 (s, 3H), 3.68 (d, J=14.2 Hz, 1H), 3.21-3.11 (m, 1H), 3.09-2.90 (m, 2H), 1.70 (dd, J=15.5, 4.7 Hz, 1H), 1.54 (dd, J=15.5, 3.1 Hz, 1H), 1.29 (t, J=7.1 Hz, 3H), 0.78 (s, 9H). ESI-MS theoretical value C₃₃H₃₉Cl₂FN₃O₃ [M+H]⁺=614.2, found: 614.2.

### Step two: Synthesis of 4-((2′S,3S,4′R,5′R)-6-chloro-4′-(3-chlorophenyl)-1′-ethyl-2′-neopentylspiro[indoline-3,3′-pyrrolidine]-5′-carboxamido)benzoic Acid (JP16)

JP12 (45 mg, 0.08 mmol), potassium carbonate (42 mg, 0.32 mmol) and lithium hydroxide monohydrate (17 mg, 0.4 mmol) were reacted according to the procedure described in step six of the final product 10 to obtain the trifluoroacetate salt of the target product (27 mg, yield 49%). ¹H NMR (400 MHz, Methanol-d₄) δ 7.99 (d, J=8.9 Hz, 2H), 7.68 (d, J=8.7 Hz, 2H), 7.41 (d, J=8.1 Hz, 1H), 7.32-7.24 (m, 3H), 7.21-7.10 (m, 1H), 6.75 (dd, J=8.1, 1.9 Hz, 1H), 6.47 (d, J=1.8 Hz, 1H), 5.05-4.93 (m, 1H), 4.34 (d, J=9.8 Hz, 1H), 4.25-4.09 (m, 1H), 3.73 (d, J=11.1 Hz, 1H), 3.70-3.60 (m, 1H), 3.58-3.40 (m, 2H), 2.03 (d, J=16.0 Hz, 1H), 1.94 (dd, J=15.6, 4.2 Hz, 1H), 1.43 (t, J=7.1 Hz, 3H), 0.77 (s, 9H). ESI-MS calculated for C₃₂H₃₆Cl₂N₃O₃ [M+H]⁺=580.2, found: 580.2.

### Final Product 49: 4-[(2′S,3S,4′R,5′R)-6-chloro-4′-(3-chlorophenyl)-1′-(2-fluoroethyl)-2′-neopentylspiro[indoline-3,3′-pyrrolidine]-5′-carboxamido]-3-methoxybenzoic Acid (JQ44)

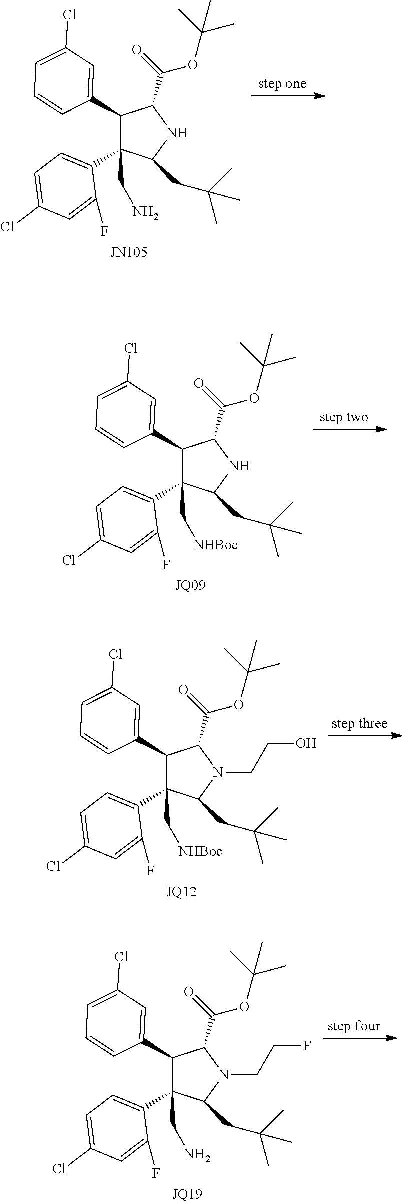

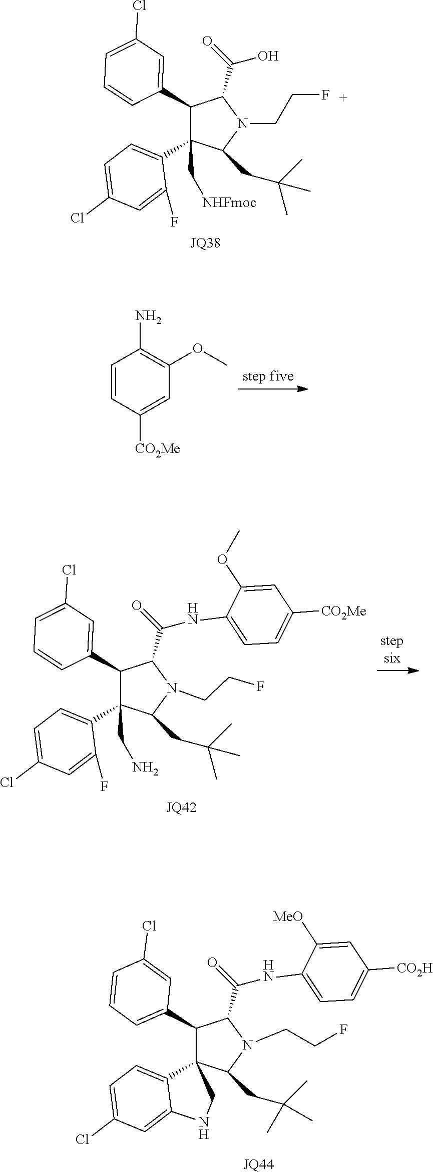

### Step one: tert-butyl (2R,3R,4S,5S)-4-((tert-butoxycarbonyl)aminomethyl)-4-(4-chloro-2-fluorophenyl)-3-(3-chlorophenyl)-5-neopentylpyrrolidine-2-carboxylate (JQ09)

JN105 (60 mg, 0.12 mmol) was placed in a 50 mL single-neck flask, dissolved in dry dichloromethane, followed by addition of diisopropylethylamine (31 mg, 0.24 mmol) and (Boc)₂O (39 mg, 0.18 mmol), and the reaction was stirred at room temperature overnight. After completion, the reaction mixture was extracted with dichloromethane, washed twice with saturated sodium chloride solution, the organic phase was dried over anhydrous sodium sulfate, concentrated by rotary evaporation and purified by column chromatography to give the target product (75 mg, yield 98%). ¹H NMR (400 MHz, Chloroform-d) δ 7.46 (d, J=7.0 Hz, 1H), 7.20-6.89 (m, 5H), 6.77 (d, J=7.7 Hz, 1H), 4.23 (d, J=7.6 Hz, 1H), 4.19 (d, J=9.0 Hz, 1H), 4.09 (d, J=7.6 Hz, 1H), 3.27 (d, J=12.7 Hz, 1H), 3.22-3.13 (m, 1H), 1.38 (s, 9H), 1.37-1.34 (m, 1H), 1.15 (d, J=14.1 Hz, 1H), 0.85 (s, 9H).

### Step two: tert-butyl (2R,3R,4S,5S)-4-((tert-butoxycarbonyl)aminomethyl)-4-(4-chloro-2-fluorophenyl)-3-(3-chlorophenyl)-1-(2-hydroxyethyl)-5-neopentylpyrrolidine-2-carboxylate (JQ12)

JQ09 (75 mg, 0.12 mmol), tert-butyl dimethylsiloxyacetaldehyde (64 mg, 0.37 mmol), sodium cyanoborohydride (77 mg, 0.36 mmol) and 1 mL acetic acid were placed in a 50 mL round-bottom flask, dissolved in 1,2-dichloroethane, and stirred at room temperature overnight. Saturated sodium bicarbonate solution was added, extracted with ethyl acetate, the organic phase was concentrated by rotary evaporation and purified by normal phase column chromatography to give the crude product (84 mg). The reaction material was dissolved in 10 mL tetrahydrofuran, 65 mg tetrabutylammonium fluoride trihydrate was added, and the reaction was stirred at room temperature for two hours. The reaction mixture was extracted three times with dichloromethane, the combined organic phases were dried over anhydrous sodium sulfate, concentrated by rotary evaporation and purified by normal phase column chromatography to give the target product (55 mg, yield 85%). ¹H NMR (400 MHz, Chloroform-d) δ 7.72 (d, J=6.2 Hz, 1H), 7.20-6.97 (m, 5H), 6.75 (d, J=7.8 Hz, 1H), 4.51-4.22 (m, 3H), 3.90 (t, J=10.6 Hz, 1H), 3.78-3.64 (m, 1H), 3.42 (d, J=13.2 Hz, 1H), 3.27-3.16 (m, 1H), 3.12-2.93 (m, 2H), 1.52 (dd, J=15.9, 4.2 Hz, 1H), 1.45 (s, 9H), 1.41-1.35 (m, 1H), 0.66 (s, 9H).

### Step Three: tert-butyl (2R,3R,4S,5S)-4-(aminomethyl)-4-(4-chloro-2-fluorophenyl)-3-(3-chlorophenyl)-1-(2-fluoroethyl)-5-neopentylpyrrolidine-2-carboxylate (JQ19)

JQ12 (56 mg, 0.09 mmol) was placed in a 50 mL single-neck flask, dissolved in dry dichloromethane, diethylaminosulfur trifluoride (28 mg, 0.18 mmol) was added at 0° C., and the reaction was stirred at room temperature overnight. After completion, the reaction mixture was extracted with dichloromethane, washed twice with saturated sodium chloride solution, the organic phase was dried over anhydrous sodium sulfate, concentrated by rotary evaporation, and purified by column chromatography to give the crude product (49 mg). The reaction material was dissolved in 4 mL dichloromethane, 0.6 mL trifluoroacetic acid was added, and the reaction was stirred at room temperature overnight. Saturated sodium bicarbonate solution was added to the reaction mixture, extracted three times with ethyl acetate, the combined organic phases were washed once with saturated sodium chloride solution, concentrated by rotary evaporation and purified by normal phase column chromatography to give the crude product (42 mg), which was directly used in the next step.

### Step Four: (2R,3R,4S,5S)-4-((((9H-fluoren-9-yl)methoxy)carbonyl)aminomethyl)-4-(4-chloro-2-fluorophenyl)-3-(3-chlorophenyl)-1-(2-fluoroethyl)-5-neopentylpyrrolidine-2-carboxylic Acid (JQ38)

JQ19 (149 mg, 0.27 mmol) was placed in a 50 mL single-neck flask, dissolved in dry dichloromethane, and then diisopropylethylamine (140 mg, 1.1 mmol) and FmocCl (105 mg, 0.31 mmol) were added. The reaction was stirred at room temperature overnight. After concentration, the crude product (130 mg) was obtained by normal phase column chromatography. The reaction material was dissolved in 3 mL dichloromethane, and 3 mL trifluoroacetic acid was added, followed by stirring at room temperature overnight. Saturated sodium bicarbonate solution was added to the reaction mixture, which was then extracted three times with ethyl acetate. The combined organic phases were washed once with saturated sodium chloride solution. After concentration, the target product (80 mg) was obtained by normal phase column chromatography with a yield of 66%). ¹H NMR (400 MHz, Chloroform-d) δ 7.78 (d, J=7.5 Hz, 2H), 7.66 (d, J=7.5 Hz, 2H), 7.46-7.38 (m, 2H), 7.38-7.31 (m, 2H), 7.17-7.09 (m, 3H), 7.06-7.00 (m, 3H), 6.74 (d, J=7.8 Hz, 1H), 4.69-4.44 (m, 4H), 4.41-4.19 (m, 4H), 3.50 (d, J=13.3 Hz, 1H), 3.46-3.23 (m, 2H), 3.14 (dd, J=13.1, 7.0 Hz, 1H), 1.56-1.36 (m, 2H), 0.67 (s, 9H).

### Step five: Methyl 4-((2R,3R,4S,5S)-4-(aminomethyl)-4-(4-chloro-2-fluorophenyl)-3-(3-chlorophenyl)-1-(2-fluoroethyl)-5-neopentylpyrrolidine-2-carboxamido)-3-methoxybenzoate (JQ42)

JQ38 (40 mg, 0.05 mmol), 1-methylimidazole (13 mg, 0.16 mmol), ethylsulfonyl chloride (13 mg, 0.1 mmol), methyl 4-amino-3-methoxybenzoate (29 mg, 0.16 mmol) and piperidine (1.5 mL) were reacted according to the procedure described in step five of the final product 18 to obtain the target product (32 mg) with a yield of 82%. ¹H NMR (400 MHz, Methanol-d₄) δ 8.23 (d, J=8.8 Hz, 1H), 7.65-7.55 (m, 2H), 7.46 (t, J=8.8 Hz, 1H), 7.36-7.24 (m, 4H), 7.11 (d, J=2.1 Hz, 1H), 7.05 (dt, J=6.6, 1.9 Hz, 1H), 4.82-4.57 (m, 2H), 4.54 (d, J=8.2 Hz, 1H), 4.45 (d, J=9.0 Hz, 1H), 4.11 (d, J=8.1 Hz, 1H), 3.92 (s, 3H), 3.87 (s, 3H), 3.72-3.55 (m, 1H), 3.49 (d, J=14.5 Hz, 1H), 3.45-3.32 (m, 1H), 3.21 (dd, J=14.8, 2.3 Hz, 1H), 1.99-1.87 (m, 1H), 1.50 (dd, J=15.3, 1.8 Hz, 1H), 1.00 (s, 9H). ESI-MS calculated for C₃₄H₄₀Cl₂F₂N₃O₄ [M+H]⁺=662.2, found: 662.2.

### Step six: 4-((2′S,3S,4′R,5′R)-6-chloro-4′-(3-chlorophenyl)-1′-(2-fluoroethyl)-2′-neopentylspiro[indole-3,3′-pyrrolidine]-5′-carboxamido)-3-methoxybenzoic Acid (JQ44)

JQ42 (32 mg, 0.05 mmol), potassium carbonate (28 mg, 0.2 mmol) and lithium hydroxide monohydrate (21 mg, 0.5 mmol) were reacted according to the procedure described in step six of the final product 10 to obtain the trifluoroacetic acid salt of the target product (20.7 mg) with a yield of 56%). ¹H NMR (400 MHz, Methanol-d₄) δ 8.34 (d, J=8.9 Hz, 1H), 7.69-7.59 (m, 2H), 7.30 (d, J=8.1 Hz, 1H), 7.26-7.19 (m, 2H), 7.18-7.15 (m, 1H), 7.04 (dt, J=6.5, 2.0 Hz, 1H), 6.85 (dd, J=8.1, 1.9 Hz, 1H), 6.60 (d, J=1.8 Hz, 1H), 4.66 (dt, J=47.5, 4.7 Hz, 2H), 4.31 (d, J=9.4 Hz, 1H), 3.93 (s, 3H), 3.84 (d, J=8.8 Hz, 2H), 3.81-3.65 (m, 1H), 3.54 (d, J=11.0 Hz, 1H), 3.40 (d, J=11.0 Hz, 1H), 3.10-2.90 (m, 1H), 1.95 (dd, J=15.4, 9.6 Hz, 1H), 1.24 (d, J=15.4 Hz, 1H), 0.99 (s, 9H). ESI-MS calculated for C₃₃H₃₇Cl₂FN₃O₄ [M+H]⁺=628.2, found: 628.2.

### Final Product 55: 4-((2′S,3S,4′R,5′R)-6-chloro-4′-(3-chlorophenyl)-2′-neopentyl-1′-(oxetan-3-ylmethyl)spiro[indoline-3,3′-pyrrolidine]-5′-carboxamido)-3-methoxybenzoic Acid (JQ97)

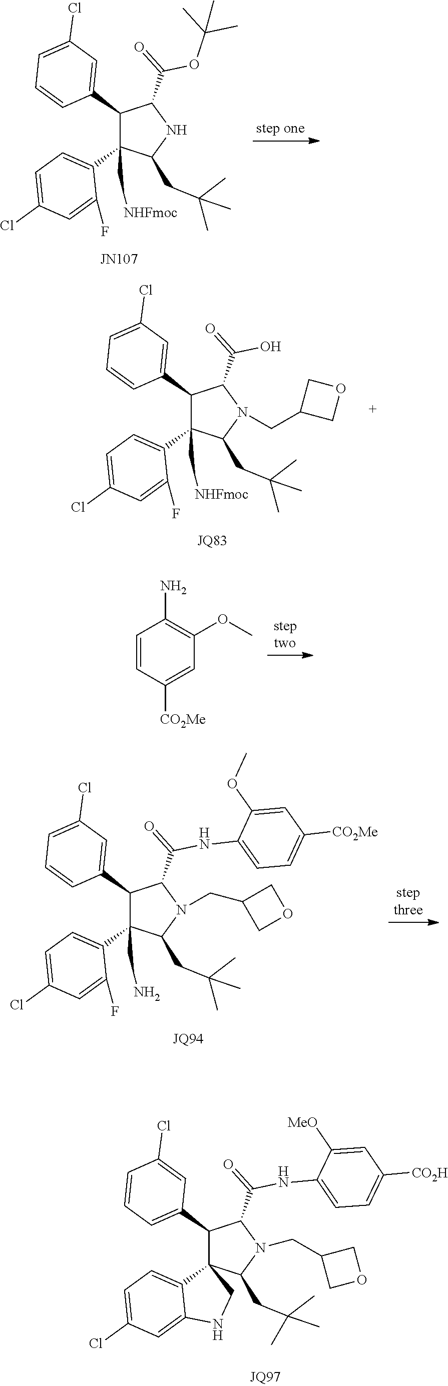

### Step one: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-5-neopentyl-1-(oxetan-3-ylmethyl)pyrrolidine-2-carboxylic Acid (JQ83)

JN107 (200 mg, 0.27 mmol), oxetan-3-carbaldehyde (118 mg, 1.37 mmol), sodium borohydride acetate (291 mg, 1.37 mmol), acetic acid (1 mL) and trifluoroacetic acid (3 mL) were reacted according to the procedure described in step four of the final product 18 to obtain the target product (46 mg) with a yield of 23%.

### Step two: Synthesis of methyl 4-((2R,3R,4S,5S)-4-(aminomethyl)-4-(4-chloro-2-fluorophenyl)-3-(3-chlorophenyl)-5-neopentyl-1-(oxetan-3-ylmethyl)pyrrolidine-2-carboxamido)-3-methoxybenzoate (JQ94)

JQ83 (46 mg, 0.06 mmol), 1-methylimidazole (15 mg, 0.18 mmol), ethylsulfonyl chloride (16 mg, 0.12 mmol), methyl 4-amino-3-methoxybenzoate (34 mg, 0.18 mmol) and piperidine (1.5 mL) were reacted according to the procedure described in step five of the final product 18 to obtain the target product (26 mg, yield 54%). ¹H NMR (400 MHz, Methanol-d₄) δ 8.26 (d, J=8.4 Hz, 1H), 7.67-7.52 (m, 3H), 7.39-7.23 (m, 4H), 7.19 (s, 1H), 7.07 (d, J=7.3 Hz, 1H), 4.79-4.73 (m, 1H), 4.63-4.56 (m, 1H), 4.56-4.51 (m, 1H), 4.47-4.37 (m, 2H), 4.31 (d, J=9.8 Hz, 1H), 4.01 (d, J=9.9 Hz, 1H), 3.95 (s, 3H), 3.88 (s, 3H), 3.49-3.42 (m, 1H), 3.37 (dd, J=14.5, 2.7 Hz, 1H), 3.30-3.24 (m, 2H), 2.09 (dd, J=15.2, 9.0 Hz, 1H), 1.49 (d, J=15.0 Hz, 1H), 1.06 (s, 9H), 0.79 (d, J=13.6 Hz, 1H). ESI-MS calculated for C₃₆H₄₃Cl₂FN₃O₅ [M+H]⁺=686.3, found: 686.3.

### Step Three: Synthesis of 4-((2′S,3S,4′R,5′R)-6-chloro-4′-(3-chlorophenyl)-2′-neopentyl-1′-(oxetan-3-ylmethyl)spiro[indole-3,3′-pyrrolidine]-5′-carboxamido)-3-methoxybenzoic Acid (JQ97)

JQ94 (26 mg, 0.04 mmol), potassium carbonate (21 mg, 0.16 mmol) and lithium hydroxide monohydrate (9 mg, 0.2 mmol) were reacted according to the procedure described in step six of the final product 10 to obtain the trifluoroacetate salt of the target product (8.5 mg, yield 28%). ¹H NMR (500 MHz, Methanol-d₄) δ 8.34 (d, J=8.8 Hz, 1H), 7.67-7.61 (m, 2H), 7.28-7.16 (m, 4H), 7.04 (dt, J=7.4, 1.6 Hz, 1H), 6.72 (dd, J=8.0, 1.9 Hz, 1H), 6.46 (d, J=1.9 Hz, 1H), 4.81 (dd, J=7.8, 6.1 Hz, 1H), 4.60 (d, J=6.6 Hz, 2H), 4.45-4.38 (m, 1H), 4.23-4.11 (m, 1H), 3.94 (s, 3H), 3.90-3.83 (m, 1H), 3.83-3.71 (m, 2H), 3.52 (d, J=10.7 Hz, 1H), 3.41 (d, J=10.7 Hz, 1H), 3.28-3.21 (m, 1H), 3.02-2.90 (m, 1H), 2.05 (dd, J=15.3, 8.7 Hz, 1H), 1.35 (d, J=15.5 Hz, 1H), 1.03 (s, 9H). ESI-MS calculated for C₃₅H₄₀Cl₂N₃O₅ [M+H]⁺=652.2, found: 652.2.

### Final Product 56: 2-(4-((2′S,3S,4′R,5′R)-6-chloro-4′-(3-chlorophenyl)-1′-ethyl-2′-neopentylspiro[indoline-3,3′-pyrrolidine]-5′-carboxamido)-3-methoxyphenyl)acetic Acid (JP27)

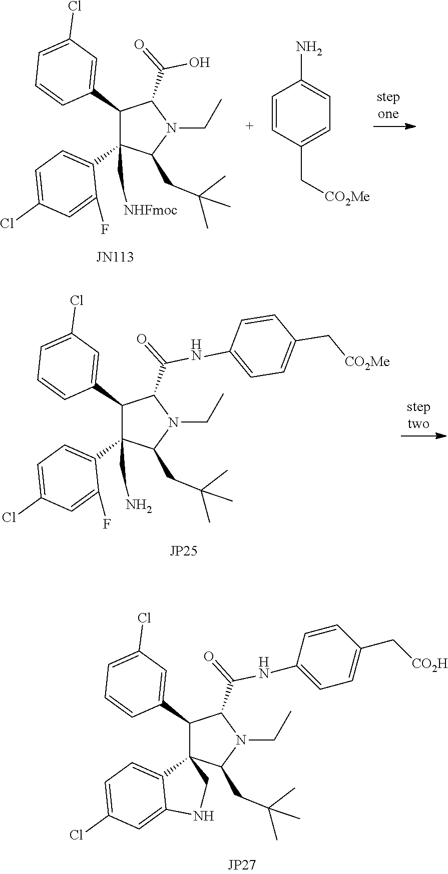

### Step one: Synthesis of methyl 2-(4-((2R,3R,4S,5S)-4-(aminomethyl)-4-(4-chloro-2-fluorophenyl)-3-(3-chlorophenyl)-1-ethyl-5-neopentylpyrrolidine-2-carboxamido)phenyl)acetate (JP25)

JN113 (40 mg, 0.06 mmol), 1-methylimidazole (14 mg, 0.17 mmol), ethylsulfonyl chloride (15 mg, 0.11 mmol), methyl 2-(4-aminophenyl)acetate (35 mg, 0.17 mmol) and piperidine (1.5 mL) were reacted according to the procedure described in step five of the final product 18 to obtain the target product (36 mg, yield 88%). ¹H NMR (500 MHz, Methanol-d₄) δ 7.50 (d, J=8.3 Hz, 2H), 7.40-7.27 (m, 5H), 7.22 (d, J=8.4 Hz, 2H), 7.07 (s, 1H), 7.06-7.00 (m, 1H), 4.68-4.61 (m, 1H), 4.61-4.51 (m, 2H), 3.68 (d, J=14.3 Hz, 1H), 3.65 (s, 3H), 3.60 (s, 2H), 3.19-3.07 (m, 1H), 3.01 (d, J=14.2 Hz, 1H), 2.99-2.90 (m, 1H), 1.68 (dd, J=15.5, 4.6 Hz, 1H), 1.54 (dd, J=15.5, 3.2 Hz, 1H), 1.30 (t, J=7.1 Hz, 3H), 0.78 (s, 9H). ESI-MS calculated for C₃₄H₄₁Cl₂FN₃O₃ [M+H]⁺=628.3, found: 628.3.

### Step two: Synthesis of 2-(4-((2′S,3S,4′R,5′R)-6-chloro-4′-(3-chlorophenyl)-1′-ethyl-2′-neopentylspiro[indole-3,3′-pyrrolidine]-5′-carboxamido)phenyl)acetic Acid (JP27)

JP25 (33 mg, 0.05 mmol), potassium carbonate (30 mg, 0.22 mmol) and lithium hydroxide monohydrate (12 mg, 0.27 mmol) were reacted according to the procedure described in step six of the final product 10 to obtain the trifluoroacetate salt of the target product (9 mg, yield 24%). ¹H NMR (400 MHz, Methanol-d₄) δ 7.49 (d, J=8.2 Hz, 2H), 7.41 (d, J=8.0 Hz, 1H), 7.33-7.22 (m, 5H), 7.17-7.09 (m, 1H), 6.75 (dd, J=8.0, 1.9 Hz, 1H), 6.47 (d, J=1.8 Hz, 1H), 4.90-4.77 (m, 1H), 4.42-4.12 (m, 2H), 3.71 (d, J=11.0 Hz, 1H), 3.68-3.63 (m, 1H), 3.58 (s, 2H), 3.55-3.42 (m, 2H), 2.10-1.85 (m, 2H), 1.45 (t, J=7.1 Hz, 3H), 0.78 (s, 9H). ESI-MS calculated for C₃₃H₃₈Cl₂N₃O₃ [M+H]⁺=594.2, found: 594.1.

### Final Product 57: 4-((((2′S,3S,4′R,5′R)-6-chloro-4′-(3-chlorophenyl)-1′-ethyl-2′-neopentylspiro[indoline-3,3′-pyrrolidine]-5′-carboxamido)methyl)-3-methoxybenzoic Acid (JP20)

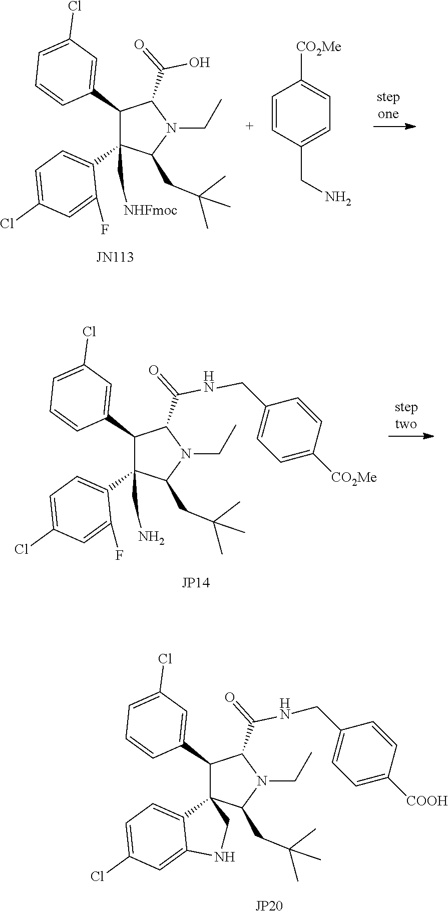

### Step one: Synthesis of methyl 2-(4-aminophenyl)acetate-4-((((2R,3R,4S,5S)-4-(aminomethyl)-4-(4-chloro-2-fluorophenyl)-3-(3-chlorophenyl)-1-ethyl-5-neopentylpyrrolidine-2-carboxamido)methyl)benzoate (JP14)

JN113 (40 mg, 0.06 mmol), 1-methylimidazole (14 mg, 0.17 mmol), ethylsulfonyl chloride (15 mg, 0.11 mmol), 4-(aminomethyl)benzoate (35 mg, 0.17 mmol) and piperidine (1.5 mL) were reacted according to the procedure described in step five of final product 18 to obtain the target product (43 mg, yield 99%). ¹H NMR (500 MHz, Methanol-d₄) δ 7.87 (d, J=8.2 Hz, 2H), 7.38-7.24 (m, 5H), 7.21 (d, J=8.1 Hz, 2H), 7.07-6.98 (m, 2H), 4.63-4.54 (m, 1H), 4.52-4.33 (m, 4H), 3.87 (s, 3H), 3.68 (d, J=14.2 Hz, 1H), 3.19-3.01 (m, 2H), 2.98-2.88 (m, 1H), 1.66 (dd, J=15.6, 4.5 Hz, 1H), 1.53 (dd, J=15.7, 3.2 Hz, 1H), 1.21 (t, J=7.2 Hz, 3H), 0.76 (s, 9H). ESI-MS calculated for C₃₄H₄₁Cl₂FN₃O₃ [M+H]⁺=628.3, found: 628.3.

### Step two: Synthesis of 4-((((2′S,3S,4′R,5′R)-6-chloro-4′-(3-chlorophenyl)-1′-ethyl-2′-neopentylspiro[indole-3,3′-pyrrolidine]-5′-carboxamido)methyl)benzoic Acid (JP20)

JP14 (43 mg, 0.07 mmol), potassium carbonate (39 mg, 0.28 mmol) and lithium hydroxide monohydrate (15 mg, 0.35 mmol) were reacted according to the procedure described in step six of final product 10 to obtain the trifluoroacetate salt of the target product (25 mg, yield 51%). ¹H NMR (400 MHz, Methanol-d₄) δ 7.82 (d, J=8.2 Hz, 2H), 7.41-7.26 (m, 3H), 7.23 (d, J=1.9 Hz, 1H), 7.13 (dt, J=7.6, 1.5 Hz, 1H), 7.03 (d, J=7.9 Hz, 2H), 6.72 (dd, J=8.0, 1.9 Hz, 1H), 6.44 (d, J=1.8 Hz, 1H), 4.79 (d, J=10.8 Hz, 1H), 4.58 (d, J=15.3 Hz, 1H), 4.28 (d, J=15.3 Hz, 1H), 4.23-4.03 (m, 2H), 3.77-3.60 (m, 2H), 3.57 (d, J=11.0 Hz, 1H), 3.51-3.35 (m, 1H), 2.14-1.82 (m, 2H), 1.35 (t, J=7.1 Hz, 3H), 0.76 (s, 9H). ESI-MS calculated for C₃₃H₃₈Cl₂N₃O₃ [M+H]⁺=594.2, found: 594.3.

### Final Product 61: (2′S,3S,4′R,5′R)—N-((3R,6S)-6-carbamoyltetrahydro-2H-pyran-3-yl)-6-chloro-4′-(3-chlorophenyl)-1′-ethyl-2′-neopentylspiro[indoline-3,3′-pyrrolidine]-5′-carboxamide (JP23)

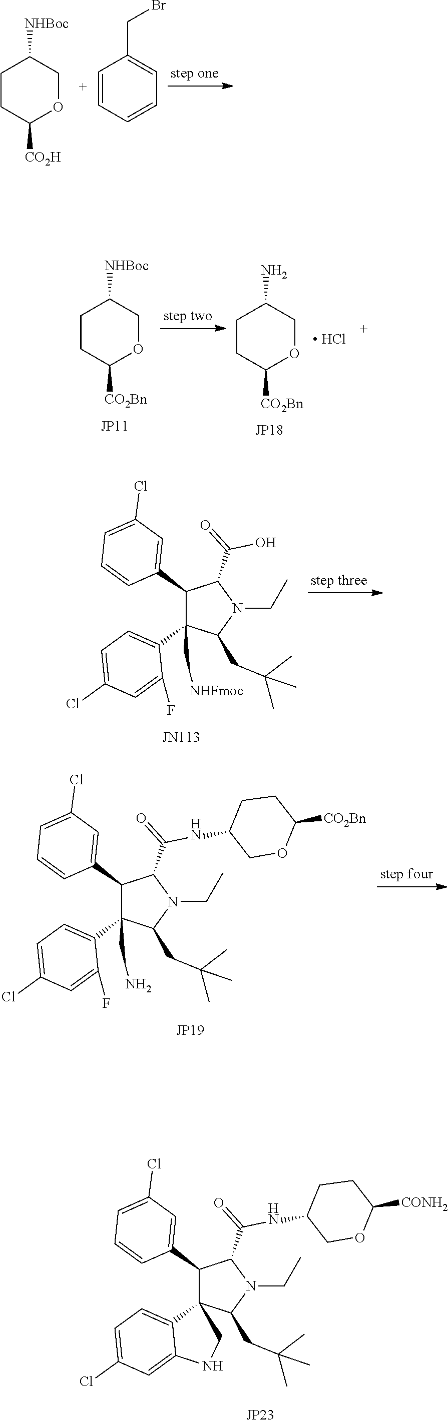

### Step one: Synthesis of benzyl (2R,5S)-5-((tert-butoxycarbonyl)amino)tetrahydro-2H-pyran-2-carboxylate (JP11)

(2R,5S)-5-((tert-butoxycarbonyl)amino)tetrahydro-2H-pyran-2-carboxylic acid (30 mg, 0.12 mmol), benzyl bromide (31 mg, 0.18 mmol) and potassium carbonate (50 mg, 0.36 mmol) were dissolved in 10 mL of anhydrous acetonitrile, and reacted at 80° C. overnight. After the reaction was complete, the reaction solution was concentrated by rotary evaporation, water was added and the mixture was extracted with ethyl acetate three times. The organic phases were combined, washed once with saturated sodium chloride solution, dried over anhydrous sodium sulfate, concentrated by rotary evaporation and purified by column chromatography to obtain the target product (44 mg, yield 99%). ¹H NMR (500 MHz, Chloroform-d) δ 7.41-7.28 (m, 5H), 5.18 (s, 2H), 4.48 (d, J=8.3 Hz, 1H), 4.16 (dd, J=11.1, 4.4 Hz, 1H), 3.96 (d, J=10.3 Hz, 1H), 3.78-3.45 (m, 1H), 3.24-2.96 (m, 1H), 2.20-1.99 (m, 2H), 1.84-1.65 (m, 1H), 1.42 (s, 9H).

### Step two: Synthesis of benzyl (2R,5S)-5-aminotetrahydro-2H-pyran-2-carboxylate Hydrochloride (JP18)

JP11 (43 mg, 0.13 mmol) was dissolved in 3 mL of dichloromethane, 3 mL of 4N hydrogen chloride in 1,4-dioxane solution was added, and the reaction was carried out at room temperature overnight. The reaction solution was concentrated to obtain the crude product (40 mg), which was directly used in the next step. ¹H NMR (500 MHz, Methanol-d₄) δ 7.42-7.26 (m, 5H), 5.31-5.12 (m, 2H), 4.27-4.07 (m, 2H), 3.51 (dd, J=11.4, 9.2 Hz, 1H), 3.31-3.21 (m, 1H), 2.26-2.09 (m, 2H), 1.85-1.67 (m, 2H).

### Step Three: Synthesis of benzyl (2S,5R)-5-((2R,3R,4S,5S)-4-(aminomethyl)-4-(4-chloro-2-fluorophenyl)-3-(3-chlorophenyl)-1-ethyl-5-neopentylpyrrolidine-2-carboxamido)tetrahydro-2H-pyran-2-carboxylate (JP19)

JN113 (35 mg, 0.05 mmol), 1-methylimidazole (13 mg, 0.15 mmol), ethylsulfonyl chloride (13 mg, 0.1 mmol), JP18 (40 mg, 0.15 mmol) and piperidine (1.5 mL) were reacted according to the procedure described in step five of the final product 18 to obtain the target product (34 mg, yield 91%). ¹H NMR (400 MHz, Methanol-d₄) δ 8.43 (d, J=7.7 Hz, 1H), 7.46-7.22 (m, 9H), 7.01 (d, J=2.2 Hz, 1H), 6.97 (dt, J=6.5, 2.0 Hz, 1H), 5.24-5.09 (m, 2H), 4.56 (d, J=3.9 Hz, 1H), 4.46 (d, J=6.3 Hz, 1H), 4.38 (d, J=7.3 Hz, 1H), 4.02 (dd, J=10.6, 2.5 Hz, 1H), 3.95-3.77 (m, 2H), 3.66 (d, J=14.2 Hz, 1H), 3.17-2.92 (m, 3H), 2.91-2.75 (m, 1H), 2.17-1.97 (m, 2H), 1.80-1.45 (m, 4H), 1.25 (t, J=7.1 Hz, 3H), 0.75 (s, 9H). ESI-MS calculated for C₃₈H₄₇Cl₂FN₃O₄ [M+H]⁺=698.3, found: 698.3.

### Step Four: Synthesis of (2′S,3S,4′R,5′R)—N-((3R,6S)-6-carbamoyltetrahydro-2H-pyran-3-yl)-6-chloro-4′-(3-chlorophenyl)-1′-ethyl-2′-neopentylspiro[indole-3,3′-pyrrolidine]-5′-carboxamide (JP23)

JP19 (33 mg, 0.05 mmol) was weighed into a flask, dissolved in 2 mL DMF, potassium carbonate (29 mg, 0.2 mmol) was added, and the mixture was stirred overnight at 110° C. After the reaction was complete, the mixture was cooled to room temperature, water was added, and the mixture was extracted with ethyl acetate. After the organic phase was evaporated by rotary evaporation, the crude product was dissolved in 5 mL of 7N hydrochloric acid in methanol and reacted at room temperature for three hours. The reaction solution was evaporated by rotary evaporation and purified by HPLC to obtain the target product (16 mg, yield 46%). ¹H NMR (400 MHz, Methanol-d₄) δ 7.37 (d, J=8.1 Hz, 1H), 7.34-7.25 (m, 2H), 7.19 (d, J=2.0 Hz, 1H), 7.08 (dt, J=6.9, 1.8 Hz, 1H), 6.73 (dd, J=8.1, 1.9 Hz, 1H), 6.45 (d, J=1.9 Hz, 1H), 4.64 (d, J=10.2 Hz, 1H), 4.36-4.00 (m, 2H), 3.93-3.82 (m, 1H), 3.78-3.55 (m, 4H), 3.54-3.37 (m, 2H), 2.88 (t, J=10.7 Hz, 1H), 2.23-1.80 (m, 4H), 1.61-1.46 (m, 2H), 1.40 (t, J=7.1 Hz, 3H), 0.76 (s, 9H). ESI-MS calculated for C₃₁H₄₁Cl₂N₄O₃ [M+H]⁺=587.3, found: 587.3.

### Final Product 65: 4-((2′S,3S,4′R,5′R)-6-chloro-4′-(3-chlorophenyl)-1′-ethyl-2′-neopentylspiro[indoline-3,3′-pyrrolidine]-5′-carboxamido)bicyclo[2.2.2]octane-1-carboxylic acid (JP04)

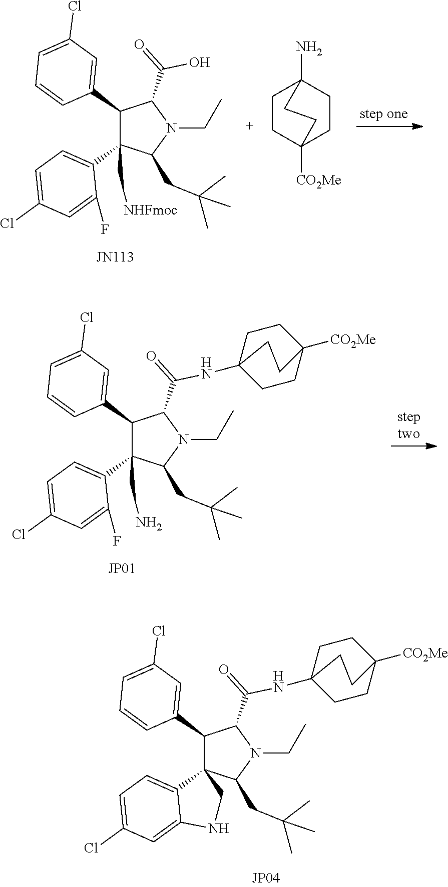

### Step one: Synthesis of methyl 4-((2R,3R,4S,5S)-4-(aminomethyl)-4-(4-chloro-2-fluorophenyl)-3-(3-chlorophenyl)-1-ethyl-5-neopentylpyrrolidine-2-carboxamido)bicyclo[2.2.2]octane-1-carboxylate (JP01)

JN113 (60 mg, 0.1 mmol), 1-methylimidazole (25 mg, 0.3 mmol), ethylsulfonyl chloride (26 mg, 0.2 mmol), methyl 4-aminobicyclo[2.2.2]octane-1-carboxylate (46 mg, 0.25 mmol) and piperidine (1.5 mL) were reacted according to the procedure described in step five of the final product 18 to obtain the target product (58 mg, yield 76%). ¹H NMR (500 MHz, Methanol-d₄) δ 7.53-7.24 (m, 5H), 7.08 (s, 1H), 7.04 (d, J=7.2 Hz, 1H), 4.60-4.37 (m, 3H), 3.65 (d, J=14.4 Hz, 1H), 3.60 (s, 3H), 3.24-3.03 (m, 3H), 1.96-1.80 (m, 12H), 1.76 (dd, J=15.9, 5.1 Hz, 1H), 1.57 (dd, J=15.8, 3.1 Hz, 1H), 1.29 (t, J=7.1 Hz, 3H), 0.79 (s, 9H).

### Step two: Synthesis of 4-((2′S,3S,4′R,5′R)-6-chloro-4′-(3-chlorophenyl)-1′-ethyl-2′-neopentylspiro[indole-3,3′-pyrrolidine]-5′-carboxamido)bicyclo[2.2.2]octane-1-carboxylic Acid (JP04)

JP01 (48 mg, 0.06 mmol), potassium carbonate (31 mg, 0.22 mmol) and lithium hydroxide monohydrate (12 mg, 0.28 mmol) were reacted according to the procedure described in step six of the final product 10 to obtain the trifluoroacetate salt of the target product (25.2 mg, yield 63%). ¹H NMR (400 MHz, Methanol-d₄) δ 7.36 (d, J=8.1 Hz, 1H), 7.33-7.24 (m, 2H), 7.20 (d, J=2.2 Hz, 1H), 7.07 (dt, J=6.9, 1.5 Hz, 1H), 6.73 (dd, J=8.0, 1.9 Hz, 1H), 6.45 (d, J=1.8 Hz, 1H), 4.76-4.52 (m, 1H), 4.39-3.84 (m, 2H), 3.67 (d, J=11.1 Hz, 1H), 3.61 (dd, J=12.9, 6.9 Hz, 1H), 3.55-3.36 (m, 2H), 2.03-1.66 (m, 14H), 1.42 (t, J=7.1 Hz, 3H), 0.83-0.60 (m, 9H). ESI-MS calculated for C₃₄H₄₄Cl₂N₃O₃ [M+H]⁺=612.3, found: 612.4.

### Final Product 66: (Pivaloyloxy)methyl 4-((2′S,3S,4′R,5′R)-6-chloro-4′-(3-chlorophenyl)-1′-ethyl-2′-neopentylspiro[indoline-3,3′-pyrrolidine]-5′-carboxamido)-3-methoxybenzoate (JP65)

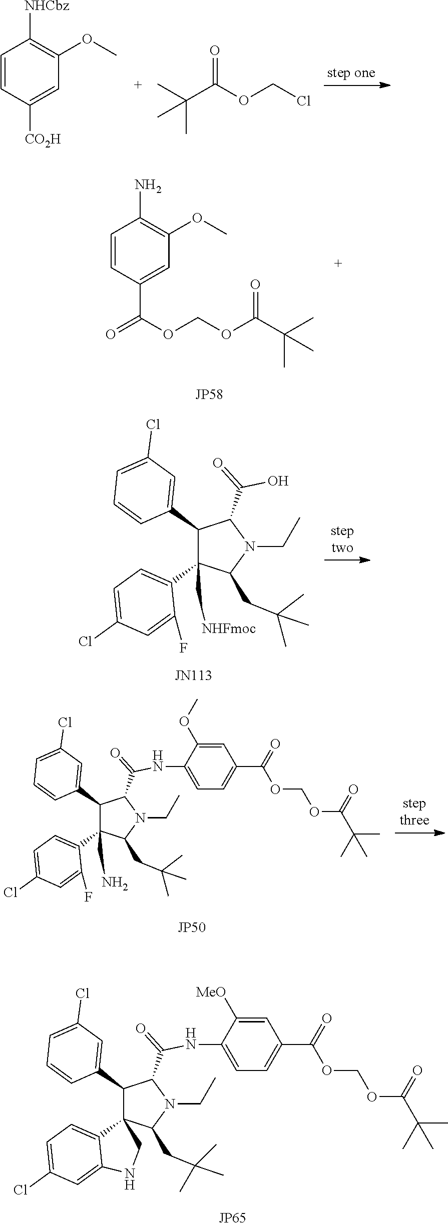

### Step one: Synthesis of (pivaloyloxy)methyl 4-amino-3-methoxybenzoate (JP58)

4-((benzyloxy)carbonyl)amino)-3-methoxybenzoic acid (300 mg, 1.0 mmol), chloromethyl pivalate (227 mg, 1.5 mmol) and potassium carbonate (414 mg, 3.0 mmol) were dissolved in 10 mL DMF and reacted at room temperature for 1 hour. After the reaction was complete, water was added, and the mixture was extracted three times with ethyl acetate. The combined organic phases were washed three times with saturated sodium chloride solution, dried over anhydrous sodium sulfate, concentrated, and purified by column chromatography to obtain 396 mg of crude product. The reaction material was dissolved in a mixed solution of tetrahydrofuran/methanol (10 mL/10 mL), deoxygenated for 5 minutes, purged with nitrogen, then 40 mg of palladium on carbon was added, deoxygenated for 5 minutes, purged with nitrogen, and reacted overnight at room temperature. The reaction solution was concentrated by rotary evaporation and purified by column chromatography to obtain 236 mg of the target product, yield 84%. ¹H NMR (400 MHz, Methanol-d₄) δ 7.49 (dd, J=8.3, 1.8 Hz, 1H), 7.41 (d, J=1.9 Hz, 1H), 6.68 (d, J=8.2 Hz, 1H), 5.92 (s, 2H), 3.88 (s, 3H), 1.20 (s, 9H).

### Step two: Synthesis of (pivaloyloxy)methyl 4-((2R,3R,4S,5S)-4-(aminomethyl)-4-(4-chloro-2-fluorophenyl)-3-(3-chlorophenyl)-1-ethyl-5-neopentylpyrrolidine-2-carboxamido)-3-methoxybenzoate (JP50)

JN113 (50 mg, 0.07 mmol), 1-methylimidazole (18 mg, 0.21 mmol), ethylsulfonyl chloride (18 mg, 0.14 mmol), JP58 (60 mg, 0.21 mmol) and piperidine (1.5 mL) were reacted according to the procedure in step five of the final product 18 to obtain 43 mg of the target product, yield 80%. ¹H NMR (400 MHz, Methanol-d₄) δ 8.31 (d, J=8.8 Hz, 1H), 7.70-7.60 (m, 2H), 7.53 (t, J=8.7 Hz, 1H), 7.39-7.30 (m, 3H), 7.29-7.23 (m, 1H), 7.14 (s, 1H), 7.05 (d, J=7.1 Hz, 1H), 5.97 (s, 2H), 4.43 (d, J=9.2 Hz, 1H), 4.31 (d, J=9.4 Hz, 1H), 3.97 (s, 3H), 3.99-3.95 (m, 1H), 3.56-3.40 (m, 2H), 3.29-3.22 (m, 1H), 3.08-2.96 (m, 1H), 2.07-1.97 (m, 1H), 1.45 (d, J=14.9 Hz, 1H), 1.24 (t, J=7.1 Hz, 3H), 1.21 (s, 9H), 1.06 (s, 9H). ESI-MS calculated for C₃₉H₄₉Cl₂FN₃O₆ [M+H]⁺=744.3, found: 744.3.

### Step Three. Synthesis of (pivaloyloxy)methyl 4-((2′S,3S,4′R,5′R)-6-chloro-4′-(3-chlorophenyl)-1′-ethyl-2′-neopentylspiro[indoline-3,3′-pyrrolidine]-5′-carboxamido)-3-methoxybenzoate (JP65)

JP62 (48 mg, 0.06 mmol) and potassium carbonate (36 mg, 0.26 mmol) were dissolved in 1.5 mL DMF and reacted at 110° C. for 8 hours. After the reaction was complete, the mixture was subjected to HPLC purification to obtain 10 mg of the trifluoroacetate salt of the target product, yield 20%. ¹H NMR (400 MHz, Methanol-d₄) δ 8.40 (d, J=8.9 Hz, 1H), 7.73-7.57 (m, 2H), 7.25-6.95 (m, 5H), 6.63 (dd, J=8.0, 1.9 Hz, 1H), 6.36 (d, J=1.8 Hz, 1H), 5.97 (s, 2H), 4.70-4.52 (m, 1H), 4.04 (d, J=9.4 Hz, 1H), 3.98 (s, 3H), 3.71-3.63 (m, 2H), 3.52-3.37 (m, 2H), 3.27 (d, J=10.4 Hz, 1H), 2.65-2.49 (m, 1H), 1.94 (dd, J=15.2, 10.3 Hz, 1H), 1.21 (s, 9H), 1.18 (t, J=7.0 Hz, 3H), 1.03 (s, 9H). ESI-MS calculated for C₃₉H₄₈Cl₂N₃O₆ [M+H]⁺=724.3, found: 724.4.

### Final Product 67; (Isobutyryloxy)methyl 4-((2′S,3S,4′R,5′R)-6-chloro-4′-(3-chlorophenyl)-1′-ethyl-2′-neopentylspiro[indoline-3,3′-pyrrolidine]-5′-carboxamido)-3-methoxybenzoate (JP90)

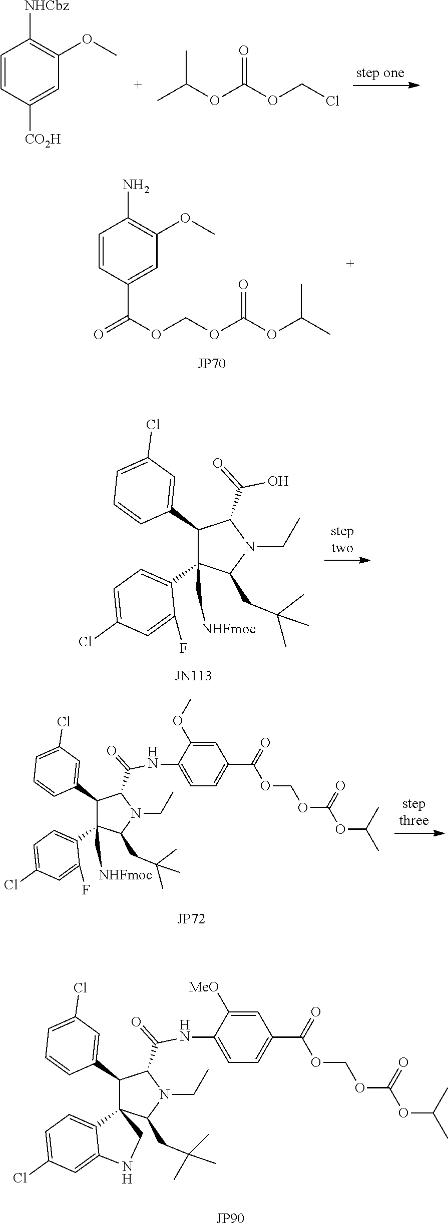

### Step one: Synthesis of methyl (isopropoxycarbonyl)oxy)4-amino-3-methoxybenzoate (JP70)

4-((benzyloxy)carbonyl)amino)-3-methoxybenzoic acid (269 mg, 0.9 mmol), chloromethyl isopropyl carbonate (203 mg, 1.34 mmol), potassium carbonate (373 mg, 2.7 mmol) and palladium on carbon (36 mg) were reacted according to the procedure in step one of final product 66 to obtain 217 mg of the target product, yield 82%. ¹H NMR (400 MHz, Chloroform-d) δ 7.59 (dd, J=8.2, 1.8 Hz, 1H), 7.46 (d, J=1.7 Hz, 1H), 6.75 (d, J=8.2 Hz, 1H), 5.95 (s, 2H), 4.91 (hept, J=6.3 Hz, 1H), 3.88 (s, 3H), 1.29 (d, J=6.3 Hz, 6H).

### Step two: Synthesis of ((isopropoxycarbonyl)oxy)methyl 4-((2R,3R,4S,5S)-4-((((9H-fluoren-9-yl)methoxy)carbonyl)amino)methyl)-4-(4-chloro-2-fluorophenyl)-3-(3-chlorophenyl)-1-ethyl-5-neopentylpyrrolidine-2-carboxamido)-3-methoxybenzoate (JP72)

JN113 (50 mg, 0.07 mmol) was added to a 50 mL single-neck flask, dissolved in dry dichloromethane, and 1-methylimidazole (18 mg, 0.21 mmol) was added at zero degrees Celsius. After stirring for 10 minutes, ethylsulfonyl chloride (18 mg, 0.14 mmol) was added. After stirring for half an hour, JP70 (88 mg, 0.21 mmol) was added and the reaction was stirred at room temperature for 2 hours. The organic phase was dried by rotary evaporation and purified by normal phase column chromatography to obtain 102 mg of crude product, which was directly used in the next step.

### Step Three: Synthesis of ((isopropoxycarbonyl)oxy)methyl 4-((2′S,3S,4′R,5′R)-6-chloro-4′-(3-chlorophenyl)-1′-ethyl-2′-neopentylspiro[indole-3,3′-pyrrolidine]-5′-carboxamido)-3-methoxybenzoate (JP90)

JP72 was dissolved in 1 mL DMF, followed by addition of 0.5 mL diethylamine, and the reaction was carried out at room temperature for 1 hour. After the reaction was completed, diethylamine was evaporated, potassium carbonate (12 mg, 0.08 mmol) was added to the reaction mixture, and the reaction was carried out at 80° C. overnight. After completion of the reaction, water was added, and the mixture was extracted three times with ethyl acetate. The combined organic phases were washed three times with saturated sodium chloride solution, dried over anhydrous sodium sulfate, and purified by HPLC to obtain 5 mg of the target product, with a yield of 13%. ¹H NMR (400 MHz, Methanol-d₄) δ 8.40 (d, J=8.4 Hz, 1H), 7.71-7.60 (m, 2H), 7.26-7.08 (m, 4H), 7.04 (d, J=5.7 Hz, 1H), 6.63 (dd, J=7.8, 1.5 Hz, 1H), 6.37 (d, J=1.9 Hz, 1H), 5.95 (s, 2H), 4.96-4.91 (m, 1H), 4.17-4.03 (m, 1H), 3.97 (s, 3H), 3.81-3.59 (m, 2H), 3.56-3.37 (m, 2H), 3.31-3.22 (m, 1H), 2.68-2.44 (m, 1H), 2.01-1.87 (m, 1H), 1.29 (d, J=6.2 Hz, 6H), 1.25-1.14 (m, 3H), 1.02 (s, 9H). ESI-MS calculated for C₃₈H₄₆Cl₂N₃O₇ [M+H]⁺=726.3, found: 726.3.

### Final Product 68: (2′S,3S,4′R,5′R)-6-chloro-4′-(3-chlorophenyl)-1′-ethyl-2′-neopentyl-N-(pyridin-4-yl)spiro[indoline-3,3′-pyrrolidine]-5′-carboxamide (JP17)

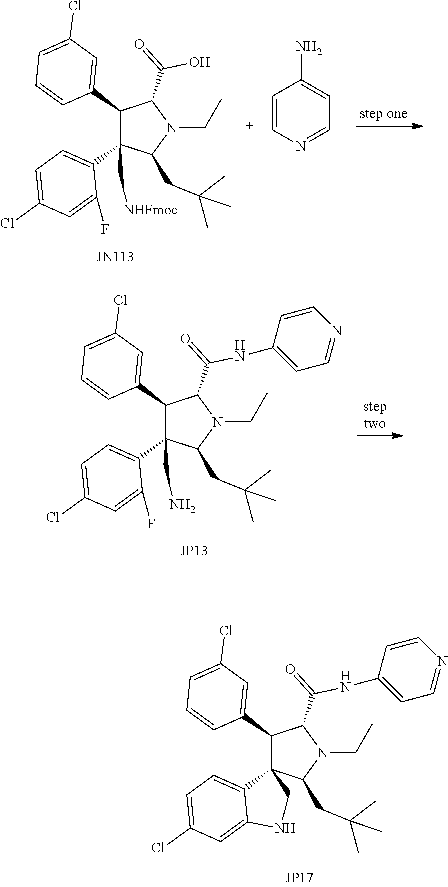

### Step one: Synthesis of (2R,3R,4S,5S)-4-(aminomethyl)-4-(4-chloro-2-fluorophenyl)-3-(3-chlorophenyl)-1-ethyl-5-neopentyl-N-(pyridin-4-yl)pyrrolidine-2-carboxamide (JP13)

JN113 (40 mg, 0.06 mmol), 1-methylimidazole (14 mg, 0.17 mmol), ethylsulfonyl chloride (15 mg, 0.11 mmol), 4-aminopyridine (16 mg, 0.17 mmol) and piperidine (1.5 mL) were reacted according to the procedure described in step five of final product 18 to obtain 40 mg of the target product with a yield of 99%. ¹H NMR (500 MHz, Methanol-d₄) δ 8.64 (d, J=7.1 Hz, 2H), 8.30-8.20 (m, 2H), 7.41-7.14 (m, 5H), 7.12-6.99 (m, 2H), 4.78 (d, J=7.0 Hz, 1H), 4.71-4.64 (m, 1H), 4.54 (dd, J=7.3, 2.5 Hz, 1H), 3.68 (d, J=14.1 Hz, 1H), 3.22-3.08 (m, 1H), 2.94-2.86 (m, 2H), 1.68 (dd, J=15.6, 4.5 Hz, 1H), 1.51 (dd, J=15.6, 3.1 Hz, 1H), 1.26 (t, J=7.2 Hz, 3H), 0.78 (s, 9H). ESI-MS calculated for C₃₀H₃₆Cl₂FN₄O [M+H]⁺=557.2, found: 557.0.

### Step two: Synthesis of (2′S,3S,4′R,5′R)-6-chloro-4′-(3-chlorophenyl)-1′-ethyl-2′-neopentyl-N-(pyridin-4-yl)spiro[indole-3,3′-pyrrolidine]-5′-carboxamide (JP17)

JP13 (34 mg, 0.06 mmol) and potassium carbonate (35 mg, 0.25 mmol) were reacted according to the procedure described in step three of final product 66 to obtain 17 mg of the trifluoroacetate salt of the target product with a yield of 44%. ¹H NMR (400 MHz, Methanol-d₄) δ 9.89-7.98 (m, 4H), 7.35 (d, J=8.0 Hz, 1H), 7.29-7.20 (m, 3H), 7.18-7.10 (m, 1H), 6.73 (dd, J=8.0, 1.9 Hz, 1H), 6.46 (d, J=1.8 Hz, 1H), 4.20 (d, J=9.5 Hz, 1H), 4.14-4.01 (m, 1H), 3.66 (d, J=11.0 Hz, 1H), 3.53-3.37 (m, 2H), 3.36-3.31 (m, 2H), 1.86 (d, J=4.1 Hz, 2H), 1.32 (t, J=7.1 Hz, 3H), 0.79 (s, 9H). ESI-MS calculated for C₃₀H³⁵ClCl₂N₄O [M+H]⁺=537.2, found: 537.2.

### Final Product 69: (1R,4r)-4-((2′S,3S,4′R,5′R)-6-chloro-4′-(3-chlorophenyl)-1′-ethyl-2′-neopentylspiro[indoline-3,3′-pyrrolidine]-5′-carboxamido)cyclohexane-1-carboxylic Acid (JP21)

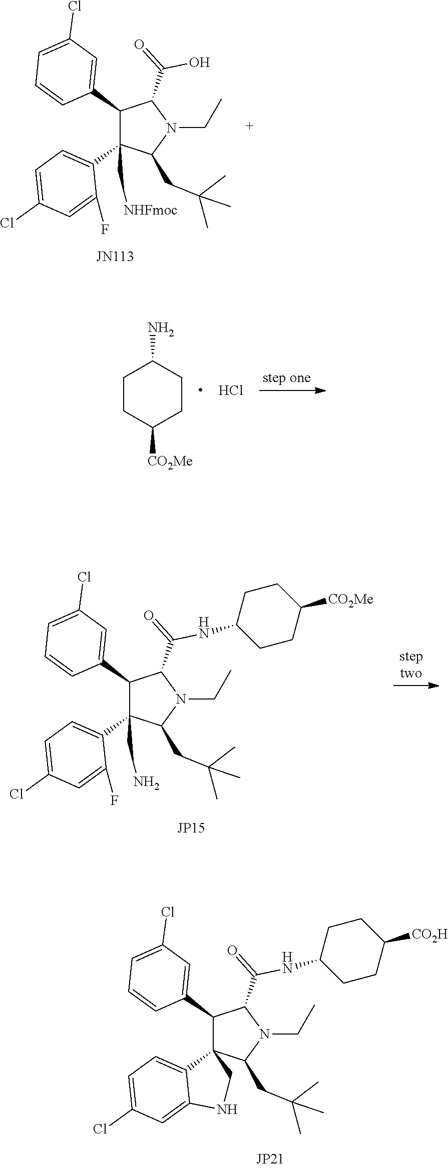

### Step one: Synthesis of methyl (1R,4r)-4-((2R,3R,4S,5S)-4-(aminomethyl)-4-(4-chloro-2-fluorophenyl)-3-(3-chlorophenyl)-1-ethyl-5-neopentylpyrrolidine-2-carboxamido)cyclohexane-1-carboxylate (JP15)

JN113 (40 mg, 0.06 mmol), 1-methylimidazole (14 mg, 0.17 mmol), ethylsulfonyl chloride (15 mg, 0.11 mmol), methyl trans-4-aminocyclohexane-1-carboxylate hydrochloride (33 mg, 0.17 mmol) and piperidine (1.5 mL) were reacted according to the procedure described in step five of the final product 18 to obtain the target product (50 mg, yield 99%). ¹H NMR (500 MHz, Methanol-d₄) δ 8.46 (d, J=7.9 Hz, 1H), 7.40-7.26 (m, 4H), 7.03 (s, 1H), 7.01-6.93 (m, 1H), 4.57 (d, J=3.8 Hz, 1H), 4.50 (d, J=7.5 Hz, 1H), 4.41 (d, J=7.5 Hz, 1H), 3.74-3.57 (m, 5H), 3.22-3.09 (m, 1H), 3.04 (d, J=14.2 Hz, 1H), 2.98-2.88 (m, 1H), 2.26 (tt, J=12.1, 3.4 Hz, 1H), 2.09-1.97 (m, 2H), 1.97-1.88 (m, 1H), 1.77-1.68 (m, 1H), 1.66 (d, J=4.6 Hz, 1H), 1.62-1.37 (m, 3H), 1.35-1.30 (m, 1H), 1.27 (t, J=7.2 Hz, 3H), 1.13 (qd, J=12.7, 3.7 Hz, 1H), 0.76 (s, 9H). ESI-MS calculated for C₃₁H₄₅Cl₂FN₃O₃ [M+H]⁺=620.3, found: 620.2.

### Step two: Synthesis of (1R,4r)-4-((2′S,3S,4′R,5′R)-6-chloro-4′-(3-chlorophenyl)-1′-ethyl-2′-neopentylspiro[indole-3,3′-pyrrolidine]-5′-carboxamido)cyclohexane-1-carboxylic Acid (JP21)

JP15 (44 mg, 0.07 mmol), potassium carbonate (41 mg, 0.29 mmol) and lithium hydroxide monohydrate (15 mg, 0.35 mmol) were reacted according to the procedure described in step six of the final product 10 to obtain the trifluoroacetate salt of the target product (24 mg, yield 49%). ¹H NMR (400 MHz, Methanol-d₄) δ 7.38 (d, J=8.1 Hz, 1H), 7.33-7.24 (m, 2H), 7.20 (d, J=2.0 Hz, 1H), 7.08 (dt, J=6.9, 1.9 Hz, 1H), 6.73 (dd, J=8.0, 1.9 Hz, 1H), 6.46 (d, J=1.8 Hz, 1H), 4.67 (d, J=10.2 Hz, 1H), 4.34-3.99 (m, 2H), 3.78-3.58 (m, 3H), 3.54-3.36 (m, 2H), 2.20 (tt, J=12.0, 3.5 Hz, 1H), 2.11-1.96 (m, 3H), 1.96-1.75 (m, 2H), 1.60 (d, J=10.5 Hz, 1H), 1.56-1.43 (m, 2H), 1.43 (t, J=7.5 Hz, 3H), 1.34-1.19 (m, 1H), 1.06 (qd, J=12.7, 3.8 Hz, 1H), 0.75 (s, 9H). ESI-MS calculated for C₃₂H₄₂Cl₂N₃O₃ [M+H]⁺=586.3, found: 586.3.

### Final Product 70: (1S,4s)-4-((2′S,3S,4′R,5′R)-6-chloro-4′-(3-chlorophenyl)-1′-ethyl-2′-neopentylspiro[indoline-3,3′-pyrrolidine]-5′-carboxamido)cyclohexane-1-carboxylic Acid (JP26)

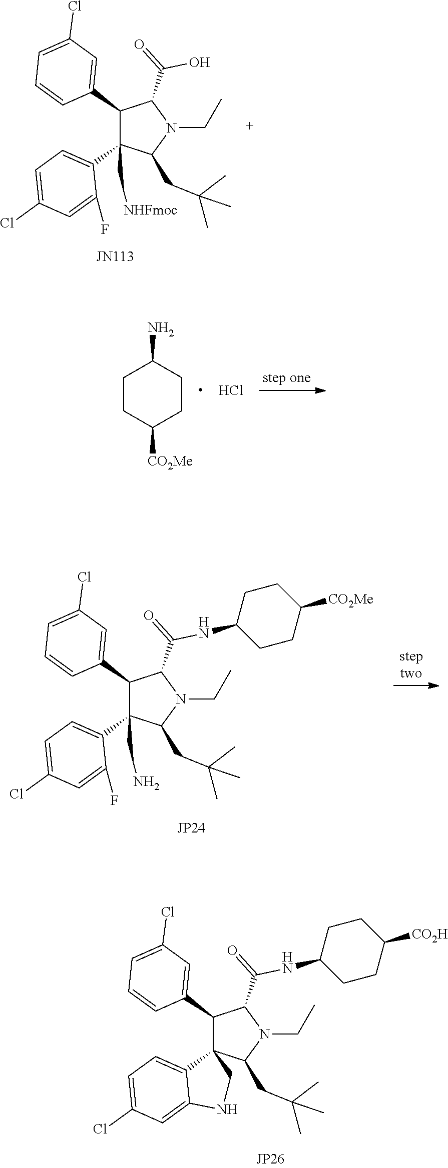

### Step 1: Synthesis of methyl (1S,4s)-4-((2R,3R,4S,5S)-4-(aminomethyl)-4-(4-chloro-2-fluorophenyl)-3-(3-chlorophenyl)-1-ethyl-5-neopentylpyrrolidine-2-carboxamido)cyclohexane-1-carboxylate (JP24)

JN113 (40 mg, 0.06 mmol), 1-methylimidazole (14 mg, 0.17 mmol), ethylsulfonyl chloride (15 mg, 0.11 mmol), methyl cis-4-aminocyclohexane-1-carboxylate hydrochloride (33 mg, 0.17 mmol) and piperidine (1.5 mL) were reacted according to the procedure described in step five of the final product 18 to obtain the target product (42 mg, yield 99%). ¹H NMR (500 MHz, Methanol-d₄) δ 7.45-7.27 (m, 5H), 7.08-6.98 (m, 2H), 4.56 (t, J=3.9 Hz, 1H), 4.51-4.28 (m, 2H), 3.85-3.76 (m, 1H), 3.70-3.66 (m, 1H), 3.65 (s, 3H), 3.21-3.05 (m, 2H), 3.03-2.90 (m, 1H), 2.56-2.45 (m, 1H), 1.97-1.87 (m, 1H), 1.80-1.65 (m, 4H), 1.63-1.48 (m, 4H), 1.46-1.36 (m, 1H), 1.28 (t, J=7.1 Hz, 3H), 0.78 (s, 9H). ESI-MS calculated for C₃₃H₄₅Cl₂FN₃O₃ [M+H]⁺=620.3, found: 620.3.

### Step two: Synthesis of (1S,4s)-4-((2′S,3S,4′R,5′R)-6-chloro-4′-(3-chlorophenyl)-1′-ethyl-2′-neopentylspiro[indole-3,3′-pyrrolidine]-5′-carboxamido)cyclohexane-1-carboxylic Acid (JP26)

JP15 (39 mg, 0.07 mmol), potassium carbonate (36 mg, 0.26 mmol) and lithium hydroxide monohydrate (14 mg, 0.33 mmol) were reacted according to the procedure described in step six of the final product 10 to obtain the trifluoroacetate of the target product (15 mg, yield 33%). ¹H NMR (400 MHz, Methanol-d₄) δ 7.37 (d, J=8.1 Hz, 1H), 7.33-7.25 (m, 2H), 7.19 (d, J=2.2 Hz, 1H), 7.13-7.05 (m, 1H), 6.73 (dd, J=8.0, 1.9 Hz, 1H), 6.45 (d, J=1.8 Hz, 1H), 4.79-4.57 (m, 1H), 4.41-3.93 (m, 2H), 3.89-3.76 (m, 1H), 3.69 (d, J=11.0 Hz, 1H), 3.63 (dd, J=12.8, 6.9 Hz, 1H), 3.56-3.37 (m, 2H), 2.57-2.30 (m, 1H), 2.09-1.80 (m, 3H), 1.78-1.48 (m, 5H), 1.41 (t, J=7.1 Hz, 3H), 1.38-1.27 (m, 2H), 0.76 (s, 9H). ESI-MS calculated for C₃₂H₄₂Cl₂N₃O₃ [M+H]⁺=586.3, found: 586.3.

### Final Product 71: (2′S,3S,4′R,5′R)-6-chloro-4′-(3-chlorophenyl)-1′-ethyl-N-((1r,4R)-4-hydroxycyclohexyl)-2′-neopentylspiro[indoline-3,3′-pyrrolidine]-5′-carboxamide (JP09)

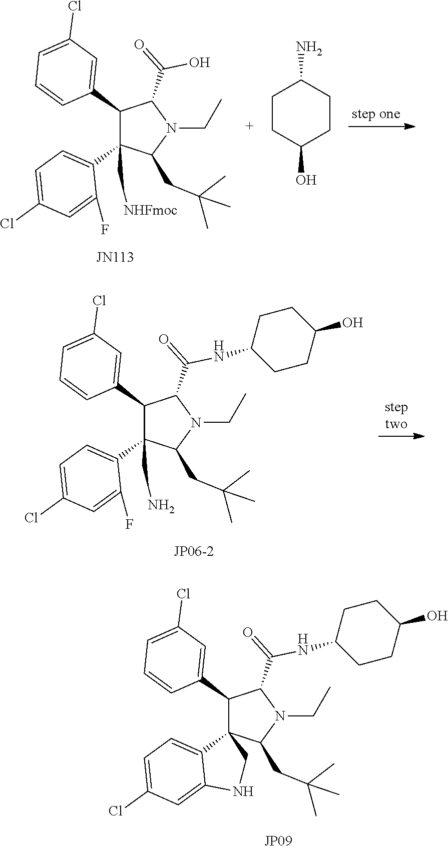

### Step one: Synthesis of (2R,3R,4S,5S)-4-(aminomethyl)-4-(4-chloro-2-fluorophenyl)-3-(3-chlorophenyl)-1-ethyl-N-((1r,4R)-4-hydroxycyclohexyl)-5-neopentylpyrrolidine-2-carboxamide (JP06-2)

JN113 (60 mg, 0.1 mmol), 1-methylimidazole (25 mg, 0.3 mmol), ethylsulfonyl chloride (26 mg, 0.2 mmol), trans-4-aminocyclohexan-1-ol (26 mg, 0.22 mmol) and piperidine (1.5 mL) were reacted according to the procedure described in step five of the final product 18 to obtain JP06-2 (29 mg, yield 42%). ¹H NMR (500 MHz, Methanol-d₄) δ 7.53-7.17 (m, 5H), 7.01 (s, 1H), 6.97 (d, J=6.6 Hz, 1H), 4.66-4.44 (m, 3H), 4.37 (d, J=7.4 Hz, 1H), 3.80-3.60 (m, 2H), 3.25-3.05 (m, 1H), 2.98 (d, J=14.7 Hz, 1H), 2.93-2.79 (m, 1H), 2.13 (d, J=12.7 Hz, 1H), 2.09-1.97 (m, 2H), 1.77 (d, J=13.3 Hz, 1H), 1.73-1.57 (m, 2H), 1.52 (dd, J=15.6, 3.4 Hz, 1H), 1.47-1.15 (m, 6H), 0.76 (s, 9H). ESI-MS calculated for C₃₁H₄₃Cl₂FN₃O₂ [M+H]⁺=578.3, found: 578.3.

### Step two: Synthesis of (2′S,3S,4′R,5′R)-6-chloro-4′-(3-chlorophenyl)-1′-ethyl-N-((1r,4R)-4-hydroxycyclohexyl)-2′-neopentylspiro[indoline-3,3′-pyrrolidine]-5′-carboxamide (JP09)

JP06-2 (27 mg, 0.05 mmol) and potassium carbonate (26 mg, 0.2 mmol) were reacted according to the procedure described in step three of the final product 66 to obtain the trifluoroacetate of the target product (6.2 mg, yield 18%). ¹H NMR (400 MHz, Methanol-d₄) δ 7.38-7.23 (m, 3H), 7.18 (d, J=1.6 Hz, 1H), 7.12-7.00 (m, 1H), 6.72 (dd, J=8.0, 1.9 Hz, 1H), 6.44 (d, J=1.8 Hz, 1H), 5.75-5.42 (m, 2H), 4.70-4.39 (m, 1H), 4.29-3.87 (m, 3H), 3.63 (d, J=10.6 Hz, 1H), 3.58-3.51 (m, 1H), 3.51-3.40 (m, 1H), 2.36 (dd, J=16.3, 4.1 Hz, 1H), 2.25-1.50 (m, 8H), 1.50-1.42 (m, 1H), 1.38 (t, J=7.1 Hz, 3H), 0.79 (s, 9H). ESI-MS calculated for C₃₁H₄₂Cl₂N₃O₂ [M+H]⁺=558.3, found: 558.3.

### Final Product 72: (2′S,3S,4′R,5′R)—N-((1r,4R)-4-aminocyclohexyl)-6-chloro-4′-(3-chlorophenyl)-1′-ethyl-2′-neopentylspiro[indoline-3,3′-pyrrolidine]-5′-carboxamide (JP33)

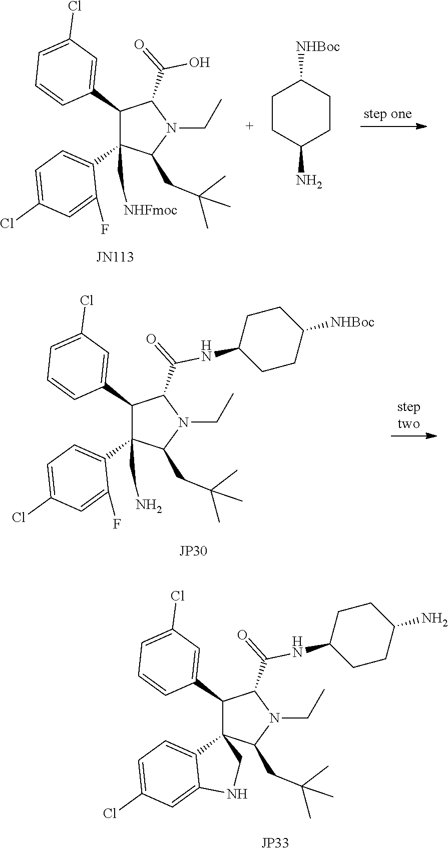

### Step one: Synthesis of tert-butyl ((1r,4R)-4-((2R,3R,4S,5S)-4-(aminomethyl)-4-(4-chloro-2-fluorophenyl)-3-(3-chlorophenyl)-1-ethyl-5-neopentylpyrrolidine-2-carboxamido)cyclohexyl)carbamate (JP30)

JN113 (40 mg, 0.06 mmol), 1-methylimidazole (14 mg, 0.17 mmol), ethylsulfonyl chloride (15 mg, 0.11 mmol), tert-butyl trans-4-aminocyclohexyl carbamate (36 mg, 0.17 mmol) and piperidine (1.5 mL) were reacted according to the procedure described in step five of the final product 18 to obtain the target product (35 mg, yield 78%). ¹H NMR (400 MHz, Methanol-d₄) δ 7.42-7.22 (m, 5H), 7.00 (s, 1H), 6.97 (dt, J=6.4, 2.0 Hz, 1H), 4.57 (d, J=3.9 Hz, 1H), 4.53-4.43 (m, 1H), 4.35 (d, J=7.3 Hz, 1H), 3.73-3.54 (m, 2H), 3.30-3.18 (m, 1H), 3.16-3.04 (m, 1H), 2.98 (d, J=14.2 Hz, 1H), 2.93-2.80 (m, 1H), 2.04-1.78 (m, 3H), 1.74-1.67 (m, 1H), 1.64 (dd, J=15.7, 4.5 Hz, 1H), 1.52 (dd, J=15.7, 3.3 Hz, 1H), 1.42 (s, 9H), 1.36-1.10 (m, 7H), 0.75 (s, 9H). ESI-MS calculated for C₃₆H₅₂Cl₂FN₄O₃ [M+H]⁺=677.3, found: 677.4.

### Step two. Synthesis of (2′S,3S,4′R,5′R)—N-((1r,4R)-4-aminocyclohexyl)-6-chloro-4′-(3-chlorophenyl)-1′-ethyl-2′-neopentylspiro[indoline-3,3′-pyrrolidine]-5′-carboxamide (JP33)

JP30 (35 mg, 0.05 mmol) was added into a flask, dissolved in 2 mL DMF, potassium carbonate (29 mg, 0.2 mmol) was added, and the mixture was stirred overnight at 110° C. After the reaction was complete, the mixture was cooled to room temperature, water was added, and the mixture was extracted with ethyl acetate. The organic phase was evaporated, and the crude product was dissolved in 3 mL dichloromethane, followed by addition of 3 mL trifluoroacetic acid, and the reaction was carried out at room temperature for two hours. The reaction solution was evaporated, and the target product (23 mg, yield 68%) was obtained by HPLC purification. ¹H NMR (500 MHz, Methanol-d₄) δ 7.37 (d, J=8.1 Hz, 1H), 7.32-7.24 (m, 2H), 7.21 (t, J=2.2 Hz, 1H), 7.10 (dt, J=6.8, 1.9 Hz, 1H), 6.72 (dd, J=8.1, 1.9 Hz, 1H), 6.45 (d, J=1.8 Hz, 1H), 4.76 (d, J=10.3 Hz, 1H), 4.27-4.03 (m, 2H), 3.79-3.56 (m, 3H), 3.47 (d, J=11.4 Hz, 2H), 3.04 (tt, J=11.9, 4.0 Hz, 1H), 2.16-1.83 (m, 5H), 1.65 (d, J=12.8 Hz, 1H), 1.56-1.43 (m, 4H), 1.39 (t, J=7.2 Hz, 3H), 1.18 (qd, J=12.9, 3.6 Hz, 1H), 0.75 (s, 9H). ESI-MS calculated for C₃₁H₄₃Cl₂N₄O [M+H]⁺=557.3, found: 557.3.

### Final Product 73: (2′S,3S,4′R,5′R)—N-((1s,4S)-4-aminocyclohexyl)-6-chloro-4′-(3-chlorophenyl)-1′-ethyl-2′-neopentylspiro[indoline-3,3′-pyrrolidine]-5′-carboxamide (JP34)

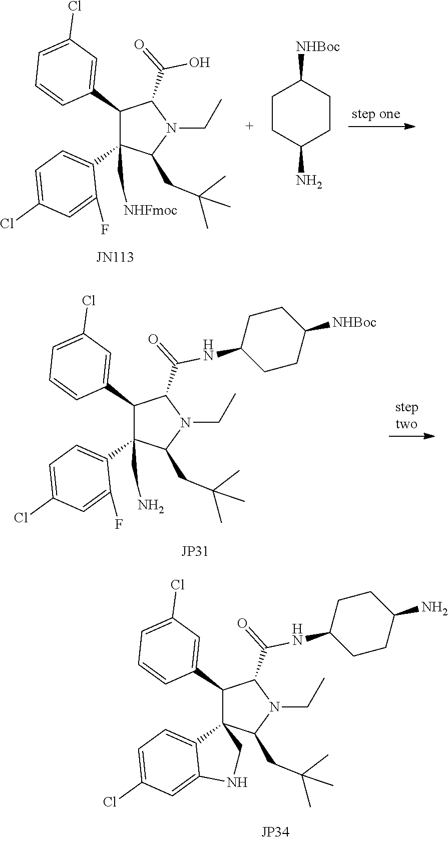

### Step 1: Synthesis of tert-butyl (1s,4S)-4-((2R,3R,4S,5S)-4-(aminomethyl)-4-(4-chloro-2-fluorophenyl)-3-(3-chlorophenyl)-1-ethyl-5-neopentylpyrrolidine-2-carboxamido)cyclohexyl)carbamate (JP31)

JN113 (40 mg, 0.06 mmol), 1-methylimidazole (14 mg, 0.17 mmol), ethylsulfonyl chloride (15 mg, 0.11 mmol), tert-butyl cis-4-aminocyclohexyl carbamate (36 mg, 0.17 mmol) and piperidine (1.5 mL) were reacted according to the procedure described in step five of the final product 18 to obtain the target product (44 mg, yield 98%). ¹H NMR (400 MHz, Methanol-d₄) δ 7.40-7.25 (m, 5H), 7.05 (s, 1H), 7.04-6.99 (m, 1H), 4.57 (t, J=3.6 Hz, 1H), 4.53-4.39 (m, 2H), 3.87-3.72 (m, 1H), 3.66 (d, J=14.2 Hz, 1H), 3.58-3.42 (m, 1H), 3.17-3.09 (m, 1H), 3.05 (d, J=14.3 Hz, 1H), 2.99-2.87 (m, 1H), 1.80-1.46 (m, 10H), 1.43 (s, 9H), 1.29 (t, J=7.1 Hz, 3H), 0.77 (s, 9H). ESI-MS calculated for C₃₆H₅₂Cl₂FN₄O₃ [M+H]⁺=677.3, found: 677.2.

### Step two: Synthesis of (2′S,3S,4′R,5′R)—N-((1s,4S)-4-aminocyclohexyl)-6-chloro-4′-(3-chlorophenyl)-1′-ethyl-2′-neopentylspiro[indoline-3,3′-pyrrolidine]-5′-carboxamide (JP34)

JP31 (44 mg, 0.07 mmol), potassium carbonate (36 mg, 0.26 mmol) and trifluoroacetic acid (3 mL) were reacted according to the procedure described in step two of the final product 72 to obtain the trifluoroacetate salt of the target product (30 mg, yield 64%). ¹H NMR (500 MHz, Methanol-d₄) δ 7.38 (d, J=8.0 Hz, 1H), 7.35-7.20 (m, 3H), 7.13 (dt, J=7.3, 1.4 Hz, 1H), 6.72 (dd, J=8.1, 1.9 Hz, 1H), 6.44 (d, J=1.9 Hz, 1H), 5.01 (d, J=11.0 Hz, 1H), 4.31-4.05 (m, 2H), 3.97 (dd, J=8.1, 4.7 Hz, 1H), 3.69 (d, J=11.0 Hz, 1H), 3.65 (dd, J=13.0, 7.0 Hz, 1H), 3.60-3.34 (m, 2H), 3.25-3.15 (m, 1H), 2.15-1.49 (m, 9H), 1.43 (t, J=7.1 Hz, 3H), 1.39-1.23 (m, 1H), 0.77 (s, 9H). ESI-MS calculated for C₃₁H₄₃Cl₂N₄O [M+H]⁺=557.3, found: 557.3.

### Final Product 74: (1r,4R)-4-aminocyclohexyl (2′S,3S,4′R,5′R)-6-chloro-4′-(3-chlorophenyl)-1′-ethyl-2′-neopentylspiro[indoline-3,3′-pyrrolidine]-5′-carboxylate (JP08)

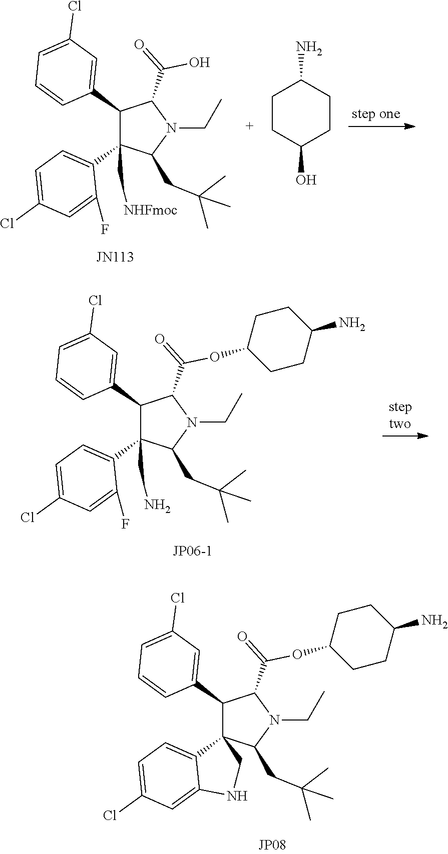

### Step one: Synthesis of (1r,4R)-4-aminocyclohexyl (2R,3R,4S,5S)-4-(aminomethyl)-4-(4-chloro-2-fluorophenyl)-3-(3-chlorophenyl)-1-ethyl-5-neopentylpyrrolidine-2-carboxylate (JP06-1)

JN113 (60 mg, 0.1 mmol), 1-methylimidazole (25 mg, 0.3 mmol), ethylsulfonyl chloride (26 mg, 0.2 mmol), trans-4-aminocyclohexan-1-ol (26 mg, 0.22 mmol) and piperidine (1.5 mL) were reacted according to the procedure described in step five of final product 18 to obtain JP06-1 (42 mg, yield 61%). ¹H NMR (500 MHz, Methanol-d₄) δ 7.46-7.19 (m, 5H), 7.09-6.89 (m, 2H), 4.57 (t, J=4.2 Hz, 1H), 4.49 (d, J=7.7 Hz, 1H), 4.43 (t, J=8.0 Hz, 1H), 3.79-3.55 (m, 2H), 3.55-3.40 (m, 1H), 3.20-3.09 (m, 1H), 3.05 (d, J=13.4 Hz, 1H), 2.93 (dt, J=12.7, 6.3 Hz, 1H), 2.02-1.81 (m, 3H), 1.81-1.59 (m, 2H), 1.55 (dd, J=15.7, 3.2 Hz, 1H), 1.39-1.30 (m, 3H), 1.27 (t, J=6.9 Hz, 3H), 1.20-1.08 (m, 1H), 0.76 (s, 9H). ESI-MS calculated for C₃₁H₄₃Cl₂FN₃O₂ [M+H]⁺=578.3, found: 578.3.

### Step two: Synthesis of (1r,4R)-4-aminocyclohexyl (2′S,3S,4′R,5′R)-6-chloro-4′-(3-chlorophenyl)-1′-ethyl-2′-neopentylspiro[indoline-3,3′-pyrrolidine]-5′-carboxylate (JP08)

JP06-1 (40 mg, 0.07 mmol) and potassium carbonate (39 mg, 0.28 mmol) were reacted according to the procedure described in step three of final product 66 to obtain the trifluoroacetate salt of the target product (16.5 mg, yield 35%). ¹H NMR (400 MHz, Methanol-d₄) δ 7.37 (d, J=8.1 Hz, 1H), 7.33-7.23 (m, 2H), 7.20 (t, J=1.7 Hz, 1H), 7.08 (dt, J=6.8, 1.9 Hz, 1H), 6.73 (dd, J=8.0, 1.9 Hz, 1H), 6.45 (d, J=1.9 Hz, 1H), 4.76-4.56 (m, 1H), 4.44-3.95 (m, 2H), 3.68 (d, J=11.1 Hz, 1H), 3.66-3.55 (m, 2H), 3.54-3.35 (m, 3H), 2.11-1.86 (m, 4H), 1.81 (d, J=12.9 Hz, 1H), 1.55 (d, J=11.3 Hz, 1H), 1.40 (t, J=7.2 Hz, 3H), 1.37-1.17 (m, 3H), 1.08 (qd, J=12.7, 3.4 Hz, 1H), 0.76 (s, 9H). ESI-MS calculated for C₃₁H₄₂Cl₂N₃O₂ [M+H]^{f}=558.3, found: 558.3.

### Final Product 75: 4-((2′S,3S,4′R,5′R)-6-chloro-4′-(3-chlorophenyl)-1′-isobutyl-2′-neopentylspiro[indoline-3,3′-pyrrolidine]-5′-carboxamido)-3-methoxybenzoic acid (JQ129)

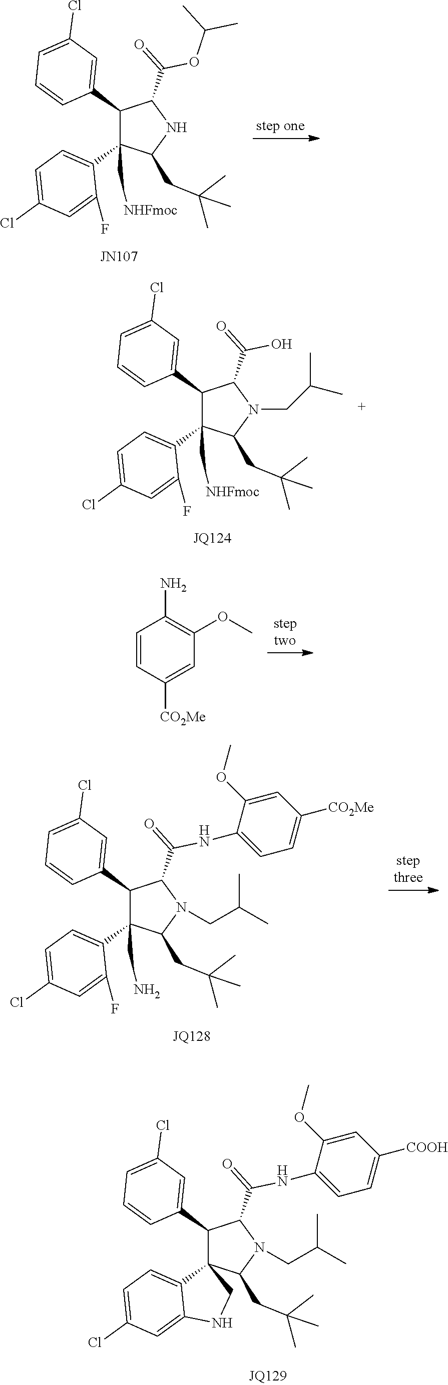

### Step one: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-1-isobutyl-5-neopentylpyrrolidine-2-carboxylic Acid (JQ124)

JN107 (100 mg, 0.14 mmol), isobutyraldehyde (98 mg, 1.4 mmol), sodium borohydride acetate (297 mg, 1.4 mmol) and 1 mL acetic acid were added to a 50 mL round-bottom flask, dissolved in 4 mL 1,2-dichloroethane, and reacted at room temperature overnight. Saturated sodium bicarbonate solution was added, extracted with ethyl acetate, the organic phase was evaporated by rotary evaporation and then dissolved in 20 mL acetic acid. Sodium cyanoborohydride (44 mg, 0.7 mmol) was added and reacted at room temperature overnight. Saturated sodium bicarbonate solution was added to the reaction mixture, extracted three times with ethyl acetate, the combined organic phases were washed once with saturated sodium chloride solution, dried over anhydrous sodium sulfate, evaporated by rotary evaporation and purified by normal phase column to give the crude product (94 mg). The reaction material was dissolved in 2 mL dichloromethane, 2 mL trifluoroacetic acid was added, and left at room temperature overnight. The reaction solution was evaporated by rotary evaporation, saturated sodium bicarbonate solution was added, extracted three times with dichloromethane, the combined organic phases were washed once with saturated sodium chloride solution, dried over anhydrous sodium sulfate, evaporated by rotary evaporation and purified by normal phase column to give the target product 60 mg, yield 59%).

### Step two: Synthesis of methyl 4-((2R,3R,4S,5S)-4-(aminomethyl)-4-(4-chloro-2-fluorophenyl)-3-(3-chlorophenyl)-1-isobutyl-5-neopentylpyrrolidine-2-carboxamido)-3-methoxybenzoate (JQ128)

JQ124 (49 mg, 0.07 mmol), 1-methylimidazole (14 mg, 0.17 mmol), ethylsulfonyl chloride (19 mg, 0.14 mmol), methyl 4-amino-3-methoxybenzoate (37 mg, 0.2 mmol) and piperidine (1.5 mL) were reacted according to the procedure described in step five of the final product 18 to obtain the target product (39 mg, yield 72%). ¹H NMR (400 MHz, Methanol-d₄) δ 8.18 (d, J=8.5 Hz, 1H), 7.64-7.54 (m, 2H), 7.43 (t, J=8.9 Hz, 1H), 7.38-7.27 (m, 4H), 7.16-7.02 (m, 2H), 4.54-4.44 (m, 2H), 4.40 (d, J=8.2 Hz, 1H), 3.87 (s, 6H), 3.59 (d, J=14.3 Hz, 1H), 3.21 (d, J=14.3 Hz, 1H), 3.02 (dd, J=12.7, 5.0 Hz, 1H), 2.65 (dd, J=12.5, 8.7 Hz, 1H), 1.97 (dd, J=14.2, 7.5 Hz, 1H), 1.78 (dd, J=15.5, 5.9 Hz, 1H), 1.53 (d, J=15.4 Hz, 1H), 1.04 (d, J=6.7 Hz, 3H), 0.95 (d, J=6.6 Hz, 3H), 0.88 (s, 9H). ESI-MS calculated for C₃₆H₄₅Cl₂FN₃O₄ [M+H]⁺=672.3, found: 672.2.

### Step Three: Synthesis of 4-((2′S,3S,4′R,5′R)-6-chloro-4′-(3-chlorophenyl)-1′-isobutyl-2′-neopentylspiro[indoline-3,3′-pyrrolidine]-5′-carboxamido)-3-methoxybenzoic Acid (JQ129)

JQ128 (39 mg, 0.06 mmol), potassium carbonate (33 mg, 0.24 mmol) and lithium hydroxide monohydrate (13 mg, 0.3 mmol) were reacted according to the procedure described in step six of the final product 10 to obtain the trifluoroacetate salt of the target product (25.2 mg, yield 56%). ¹H NMR (400 MHz, Methanol-d₄) δ 8.23 (d, J=8.4 Hz, 1H), 7.63 (dd, J=8.4, 1.7 Hz, 1H), 7.57 (d, J=1.7 Hz, 1H), 7.39-7.30 (m, 3H), 7.27 (t, J=7.8 Hz, 1H), 7.11 (dt, J=7.6, 1.5 Hz, 1H), 6.76 (dd, J=8.1, 1.9 Hz, 1H), 6.48 (d, J=1.9 Hz, 1H), 4.52-4.14 (m, 1H), 4.12-3.95 (m, 1H), 3.81 (s, 3H), 3.68 (d, J=10.9 Hz, 1H), 3.58 (d, J=11.0 Hz, 1H), 3.47-3.32 (m, 1H), 3.08-2.83 (m, 1H), 2.28-1.94 (m, 2H), 1.82-1.42 (m, 1H), 1.11 (d, J=6.7 Hz, 3H), 1.09 (d, J=7.1 Hz, 3H), 0.93 (s, 9H). ESI-MS calculated for C₃₅H₄₂Cl₂N₃O₄ [M+H]⁺=638.3, found: 638.3.

### Final Product 76: 4-((2′S,3S,4′R,5′R)-6-chloro-4′-(3-chlorophenyl)-1′-(cyclopropylmethyl)-2′-neopentylspiro[indoline-3,3′-pyrrolidine]-5′-carboxamido)-3-methoxybenzoic Acid (JQ53)

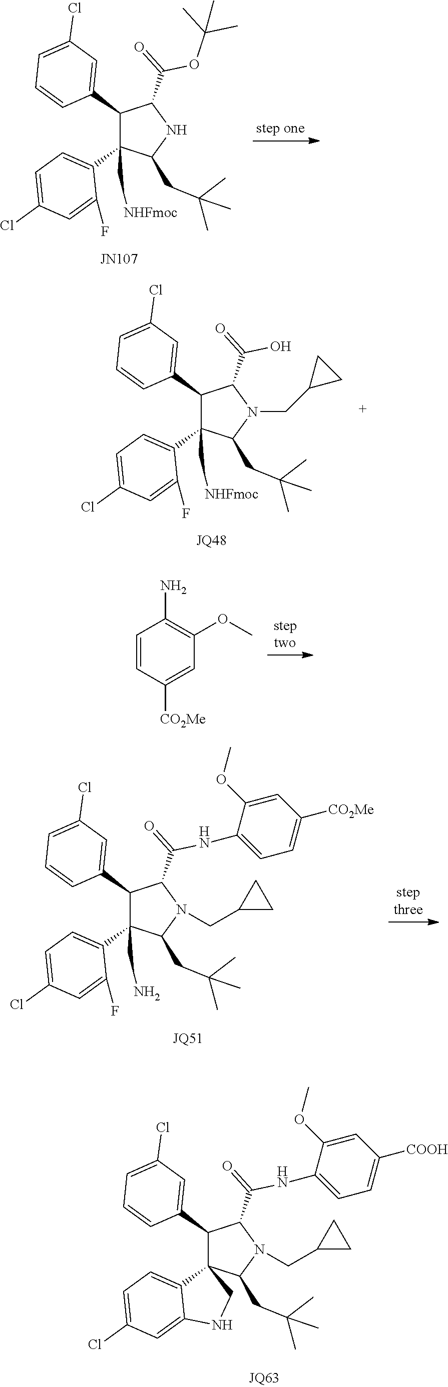

### Step one: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-1-(cyclopropylmethyl)-5-neopentylpyrrolidine-2-carboxylic Acid (JQ48)

JN107 (200 mg, 0.27 mmol), cyclopropane carboxaldehyde (96 mg, 1.37 mmol), sodium borohydride acetate (290 mg, 1.37 mmol), acetic acid (1 mL) and trifluoroacetic acid (3 mL) were reacted according to the reaction procedure described in step four of the final product 18 to obtain the target product (30 mg, yield 14%).

### Step two: Synthesis of methyl 4-((2R,3R,4S,5S)-4-(aminomethyl)-4-(4-chloro-2-fluorophenyl)-3-(3-chlorophenyl)-1-(cyclopropylmethyl)-5-neopentylpyrrolidine-2-carboxamido)-3-methoxybenzoate (JQ51)

JQ48 (25 mg, 0.03 mmol), 1-methylimidazole (8 mg, 0.09 mmol), ethylsulfonyl chloride (8 mg, 0.06 mmol), methyl 4-amino-3-methoxybenzoate (18 mg, 0.09 mmol) and piperidine (1.5 mL) were reacted according to the reaction procedure described in step five of the final product 18 to obtain the target product (23 mg, yield 98%). ¹H NMR (400 MHz, Methanol-d₄) δ 8.28 (dd, J=9.0, 1.4 Hz, 1H), 7.65-7.58 (m, 2H), 7.54 (t, J=8.8 Hz, 1H), 7.34 (dd, J=9.1, 2.0 Hz, 1H), 7.33-7.28 (m, 2H), 7.25 (dd, J=13.6, 2.2 Hz, 1H), 7.11 (t, J=2.2 Hz, 1H), 7.06 (dt, J=6.7, 1.9 Hz, 1H), 4.47 (d, J=9.2 Hz, 1H), 4.34 (d, J=9.8 Hz, 1H), 3.99 (d, J=9.8 Hz, 1H), 3.94 (s, 3H), 3.87 (s, 3H), 3.63 (dd, J=12.8, 5.4 Hz, 1H), 3.44 (d, J=14.5 Hz, 1H), 3.24 (dd, J=14.4, 2.7 Hz, 1H), 2.52 (dd, J=12.8, 8.1 Hz, 1H), 2.00 (dd, J=14.8, 9.0 Hz, 1H), 1.45 (d, J=14.9 Hz, 1H), 1.06 (s, 9H), 1.04-0.94 (m, 1H), 0.56-0.41 (m, 2H), 0.40-0.19 (m, 2H). ESI-MS calculated for C₃₆H₄₃Cl₂FN₃O₄ [M+H]⁺=670.3, found: 670.3.

### Step Three: Synthesis of 4-((2′S,3S,4′R,5′R)-6-chloro-4′-(3-chlorophenyl)-1′-(cyclopropylmethyl)-2′-neopentylspiro[indoline-3,3′-pyrrolidine]-5′-carboxamido)-3-methoxybenzoic Acid (JQ53)

JQ51 (23 mg, 0.03 mmol), potassium carbonate (18 mg, 0.14 mmol) and lithium hydroxide monohydrate (8 mg, 0.17 mmol) were reacted according to the reaction procedure described in step six of the final product 10 to obtain the trifluoroacetate salt of the target product (11 mg, yield 44%). ¹H NMR (400 MHz, Methanol-d₄) δ 8.23 (d, J=8.4 Hz, 1H), 7.63 (dd, J=8.4, 1.7 Hz, 1H), 7.58 (d, J=1.7 Hz, 1H), 7.41-7.24 (m, 4H), 7.14 (d, J=7.5 Hz, 1H), 6.73 (dd, J=8.0, 1.9 Hz, 1H), 6.45 (d, J=1.8 Hz, 1H), 4.36-4.12 (m, 1H), 4.12-3.95 (m, 1H), 3.83 (s, 3H), 3.72-3.51 (m, 2H), 3.27-3.01 (m, 1H), 2.15-1.88 (m, 1H), 1.75-1.55 (m, 1H), 1.25-1.09 (m, 1H), 0.90 (s, 9H), 0.75 (dd, J=12.4, 5.3 Hz, 1H), 0.68-0.60 (m, 1H), 0.60-0.50 (m, 1H), 0.42 (dt, J=9.8, 5.0 Hz, 1H). ESI-MS calculated for C₃₅H₄₀Cl₂N₃O₄ [M+H]⁺=636.2, found: 636.2.

### Final Product 77: 4-((2′S,3S,4′R,5′R)-6-chloro-4′-(3-chlorophenyl)-1′-(cyclobutylmethyl)-2′-neopentylspiro[indoline-3,3′-pyrrolidine]-5′-carboxamido)-3-methoxybenzoic Acid (JQ148)

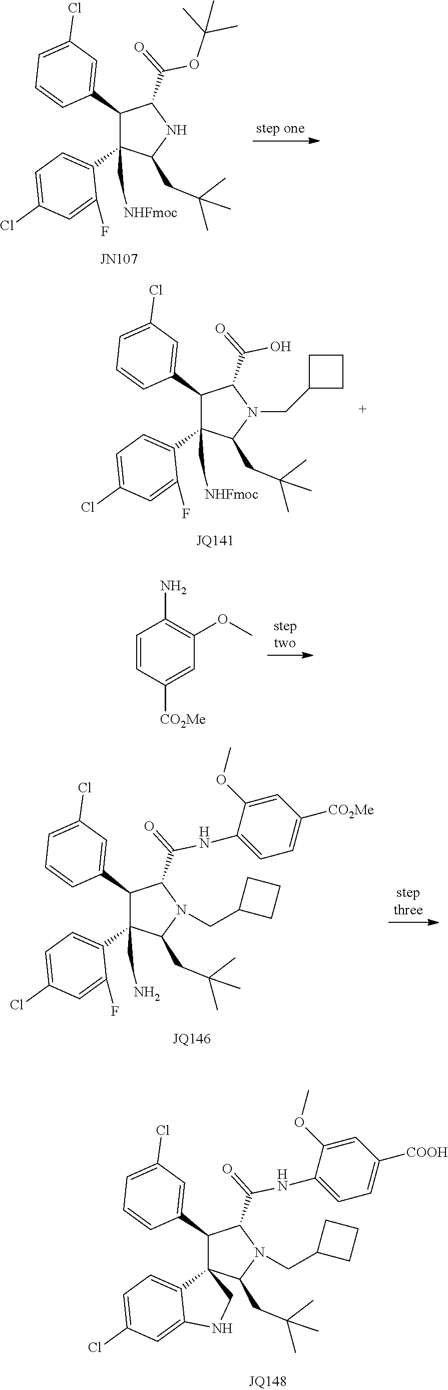

### Step one. Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-1-(cyclobutylmethyl)-5-neopentylpyrrolidine-2-carboxylic Acid (JQ141)

JN107 (146 mg, 0.2 mmol), cyclobutene carboxaldehyde (84 mg, 1.0 mmol), sodium borohydride acetate (212 mg, 1.0 mmol), acetic acid (1 mL), sodium cyanoborohydride (126 mg, 2.0 mmol) and trifluoroacetic acid (3 mL) were reacted according to the reaction procedure described in step one of the final product 75 to obtain the target product (90 mg, yield 58%).

### Step two. Synthesis of methyl 4-((2R,3R,4S,5S)-4-(aminomethyl)-4-(4-chloro-2-fluorophenyl)-3-(3-chlorophenyl)-1-(cyclobutylmethyl)-5-neopentylpyrrolidine-2-carboxamido)-3-methoxybenzoate (JQ146)

JQ141 (45 mg, 0.06 mmol), 1-methylimidazole (15 mg, 0.18 mmol), ethylsulfonyl chloride (16 mg, 0.12 mmol), methyl 4-amino-3-methoxybenzoate (31 mg, 0.18 mmol) and piperidine (1.5 mL) were reacted according to the procedure described in step five of the final product 18 to obtain the target product (39 mg, yield 81%). ¹H NMR (400 MHz, Methanol-d₄) δ 8.34-8.18 (m, 1H), 7.65-7.57 (m, 2H), 7.49 (t, J=9.0 Hz, 1H), 7.36-7.29 (m, 3H), 7.26 (dd, J=13.5, 2.3 Hz, 1H), 7.11 (s, 1H), 7.06 (dt, J=6.3, 2.0 Hz, 1H), 4.43 (d, J=8.5 Hz, 1H), 4.30 (d, J=9.4 Hz, 1H), 4.06 (d, J=9.2 Hz, 1H), 3.93 (s, 3H), 3.87 (s, 3H), 3.47 (d, J=14.5 Hz, 1H), 3.40 (dd, J=12.4, 6.7 Hz, 1H), 3.30-3.24 (m, 1H), 2.94 (dd, J=12.4, 7.7 Hz, 1H), 2.74-2.55 (m, 1H), 2.17-1.71 (m, 7H), 1.46 (d, J=15.0 Hz, 1H), 1.02 (s, 9H). ESI-MS calculated for C₃₇H₄₅Cl₂FN₃O₄ [M+H]⁺=684.3, found: 684.3.

### Step Three: Synthesis of 4-((2′S,3S,4′R,5′R)-6-chloro-4′-(3-chlorophenyl)-1′-(cyclobutylmethyl)-2′-neopentylspiro[indole-3,3′-pyrrolidine]-5′-carboxamido)-3-methoxybenzoic Acid (JQ148)

JQ146 (39 mg, 0.06 mmol), potassium carbonate (32 mg, 0.24 mmol) and lithium hydroxide monohydrate (13 mg, 0.3 mmol) were reacted according to the procedure described in step six of the final product 10 to obtain the trifluoroacetate salt of the target product (22.4 mg, yield 49%). ¹H NMR (400 MHz, Methanol-d₄) δ 8.24 (d, J=8.4 Hz, 1H), 7.64 (dd, J=8.4, 1.7 Hz, 1H), 7.58 (d, J=1.7 Hz, 1H), 7.39-7.23 (m, 4H), 7.12 (d, J=7.5 Hz, 1H), 6.74 (dd, J=8.0, 1.9 Hz, 1H), 6.47 (d, J=1.9 Hz, 1H), 4.94-4.64 (m, 1H), 4.37-4.14 (m, 1H), 3.96 (d, J=9.8 Hz, 1H), 3.82 (s, 3H), 3.74-3.51 (m, 3H), 3.21-2.98 (m, 1H), 2.83-2.65 (m, 1H), 2.36-2.02 (m, 3H), 2.01-1.76 (m, 4H), 1.73-1.43 (m, 1H), 0.96 (s, 9H). ESI-MS calculated for C₃₆H₄₂Cl₂N₃O₄ [M+H]⁺=650.3, found: 650.3.

### Final Product 78: 4-((2′S,3S,4′R,5′R)-6-chloro-4′-(3-chlorophenyl)-1′-(cyclopentylmethyl)-2′-neopentylspiro[indoline-3,3′-pyrrolidine]-5′-carboxamido)-3-methoxybenzoic Acid (JQ91)

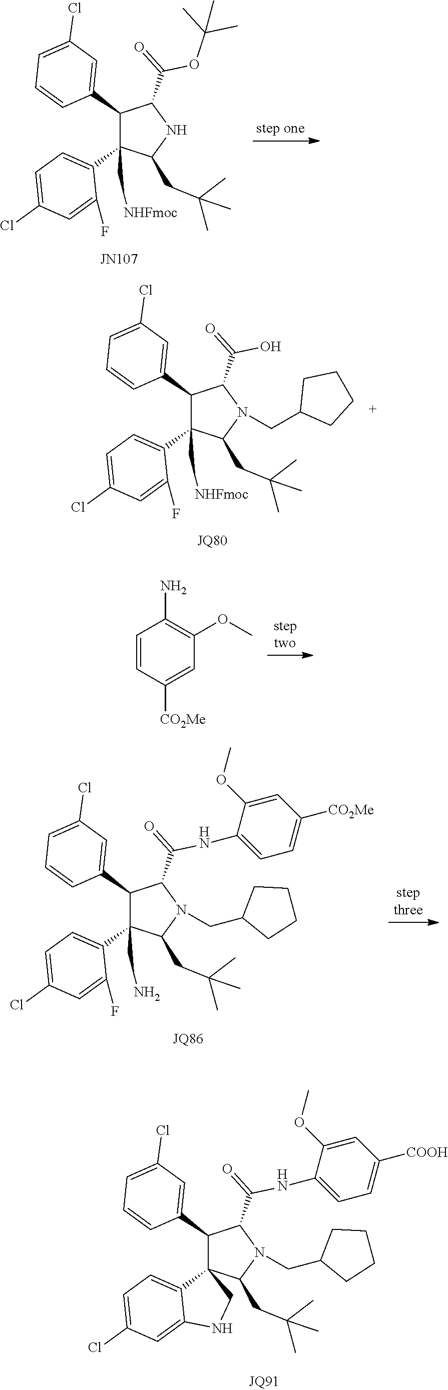

### Step one: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-1-(cyclopentylmethyl)-5-neopentylpyrrolidine-2-carboxylic Acid (JQ80)

JN107 (400 mg, 0.55 mmol), cyclopentane carboxaldehyde (268 mg, 2.74 mmol), sodium borohydride acetate (581 mg, 2.74 mmol), acetic acid (2 mL) and trifluoroacetic acid (2 mL) were reacted according to the procedure described in step four of the final product 18 to obtain the target product (52 mg, yield 12%).

### Step two: Synthesis of methyl 4-((2R,3R,4S,5S)-4-(aminomethyl)-4-(4-chloro-2-fluorophenyl)-3-(3-chlorophenyl)-1-(cyclopentylmethyl)-5-neopentylpyrrolidine-2-carboxamido)-3-methoxybenzoate (JQ86)

JQ80 (52 mg, 0.07 mmol), 1-methylimidazole (17 mg, 0.2 mmol), ethylsulfonyl chloride (17 mg, 0.13 mmol), methyl 4-amino-3-methoxybenzoate (36 mg, 0.2 mmol) and piperidine (1.5 mL) were reacted according to the procedure described in step five of the final product 18 to obtain the target product (34 mg, yield 64%). ¹H NMR (400 MHz, Methanol-d₄) δ 8.19 (d, J=8.2 Hz, 1H), 7.65-7.58 (m, 2H), 7.43 (t, J=8.9 Hz, 1H), 7.38-7.24 (m, 4H), 7.15-7.04 (m, 2H), 4.47 (d, J=8.7 Hz, 1H), 4.29 (d, J=8.4 Hz, 1H), 3.90 (s, 3H), 3.88 (s, 3H), 3.55 (d, J=14.4 Hz, 1H), 3.26-3.17 (m, 2H), 2.79 (dd, J=12.4, 8.6 Hz, 1H), 2.33-2.18 (m, 1H), 1.96-1.90 (m, 1H), 1.84 (dd, J=15.3, 7.0 Hz, 1H), 1.79-1.54 (m, 5H), 1.50 (dd, J=15.3, 2.2 Hz, 1H), 1.42-1.19 (m, 3H), 0.92 (s, 9H). ESI-MS calculated for C₃₈H₄₇Cl₂FN₃O₄ [M+H]⁺=698.3, found: 698.3.

### Step Three: Synthesis of 4-((2′S,3S,4′R,5′R)-6-chloro-4′-(3-chlorophenyl)-1′-(cyclopentylmethyl)-2′-neopentylspiro[indole-3,3′-pyrrolidine]-5′-carboxamido)-3-methoxybenzoic Acid (JQ91)

JQ86 (34 mg, 0.05 mmol), potassium carbonate (27 mg, 0.2 mmol) and lithium hydroxide monohydrate (11 mg, 0.25 mmol) were reacted according to the procedure described in step six of final product 10 to obtain the trifluoroacetate of the target product (12.7 mg, yield 33%). ¹H NMR (500 MHz, Methanol-d₄) δ 8.28 (s, 1H), 7.64 (dt, J=8.3, 1.9 Hz, 1H), 7.59 (s, 1H), 7.46-7.16 (m, 5H), 7.09 (d, J=7.6 Hz, 1H), 6.70 (dd, J=8.0, 2.0 Hz, 1H), 6.43 (d, J=2.0 Hz, 1H), 4.26-3.73 (m, 6H), 3.73-3.35 (m, 1H), 3.22-2.57 (m, 1H), 2.44-1.23 (m, 1H), 0.97 (s, 9H). ESI-MS calculated for C₃₇H₄₄Cl₂N₃O₄ [M+H]⁺=664.3, found: 664.3.

### Final Product 79: 4-((2′S,3S,4′R,5′R)-6-chloro-4′-(3-chlorophenyl)-1′-(cyclohexylmethyl)-2′-neopentylspiro[indoline-3,3′-pyrrolidine]-5′-carboxamido)-3-methoxybenzoic Acid (JQ96)

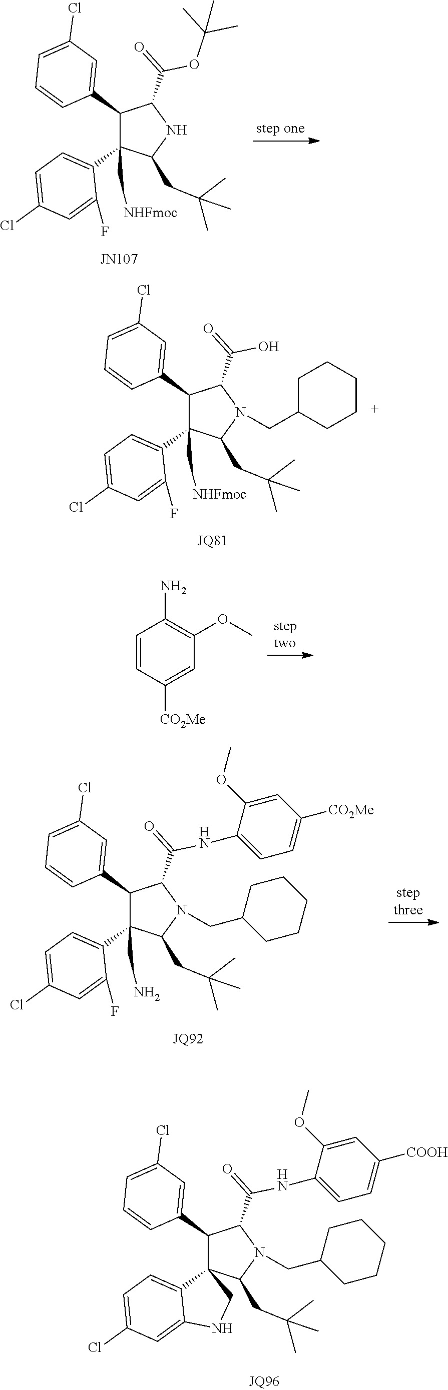

### Step one: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-1-(cyclohexylmethyl)-5-neopentylpyrrolidine-2-carboxylic Acid (JQ81)

JN107 (200 mg, 0.27 mmol), cyclohexane carboxaldehyde (153 mg, 1.37 mmol), sodium cyanoborohydride (290 mg, 1.37 mmol), acetic acid (1 mL) and trifluoroacetic acid (2 mL) were reacted according to the procedure described in step four of final product 18 to obtain the target product (21 mg, yield 10%).

### Step two: Synthesis of methyl 4-((2R,3R,4S,5S)-4-(aminomethyl)-4-(4-chloro-2-fluorophenyl)-3-(3-chlorophenyl)-1-(cyclohexylmethyl)-5-neopentylpyrrolidine-2-carboxamido)-3-methoxybenzoate (JQ92)

JQ81 (21 mg, 0.03 mmol), 1-methylimidazole (8 mg, 0.09 mmol), ethylsulfonyl chloride (8 mg, 0.06 mmol), methyl 4-amino-3-methoxybenzoate (15 mg, 0.08 mmol) and piperidine (1.5 mL) were reacted according to the procedure described in step five of final product 18 to obtain the target product (6 mg, yield 24%). ¹H NMR (400 MHz, Methanol-d₄) δ 8.19 (d, J=8.3 Hz, 1H), 7.69-7.57 (m, 2H), 7.48 (t, J=7.9 Hz, 1H), 7.39-7.28 (m, 4H), 7.15 (s, 1H), 7.09 (d, J=6.2 Hz, 1H), 4.50-4.30 (m, 3H), 3.90 (s, 3H), 3.89 (s, 3H), 3.54 (d, J=14.3 Hz, 1H), 3.21 (d, J=14.3 Hz, 1H), 3.02 (dd, J=12.6, 5.3 Hz, 1H), 2.70 (dd, J=12.5, 8.0 Hz, 1H), 1.91-1.74 (m, 3H), 1.73-1.61 (m, 3H), 1.51 (d, J=15.3 Hz, 1H), 1.45-1.13 (m, 4H), 1.09-0.94 (m, 2H), 0.90 (s, 9H). ESI-MS calculated for C₃₉H₄₉Cl₂FN₃O₄ [M+H]⁺=712.3, found: 712.3.

### Step Three: Synthesis of 4-((2′S,3S,4′R,5′R)-6-chloro-4′-(3-chlorophenyl)-1′-(cyclohexylmethyl)-2′-neopentylspiro[indoline-3,3′-pyrrolidine]-5′-carboxamido)-3-methoxybenzoic Acid (JQ96)

JQ96 (6 mg, 0.01 mmol), potassium carbonate (5 mg, 0.04 mmol) and lithium hydroxide monohydrate (2 mg, 0.05 mmol) were reacted according to the procedure described in step six of final product 10 to obtain the trifluoroacetate of the target product (2 mg, yield 25%). ¹H NMR (500 MHz, Methanol-d₄) δ 8.36 (s, 1H), 7.71-7.63 (m, 2H), 7.41-7.00 (m, 5H), 6.69 (d, J=8.1 Hz, 1H), 6.42 (d, J=2.2 Hz, 1H), 3.93 (s, 3H), 3.90-3.45 (m, 7H), 2.28-2.18 (m, 1H), 1.87-1.77 (m, 3H), 1.77-1.65 (m, 3H), 1.41-1.01 (m, 6H), 0.98 (s, 9H). ESI-MS calculated for C₃₈H₄₆Cl₂N₃O₄ [M+H]⁺=678.3, found: 678.2.

### Final Products 80 and 81: 4-((2′S,3S,4′R,5′R)-6-chloro-4′-(3-chlorophenyl)-2′-neopentyl-1′-((((R or S)-tetrahydrofuran-3-yl)methyl)spiro[indoline-3,3′-pyrrolidine]-5′-carboxamido)-3-methoxybenzoic acid (JR01 and JR02)

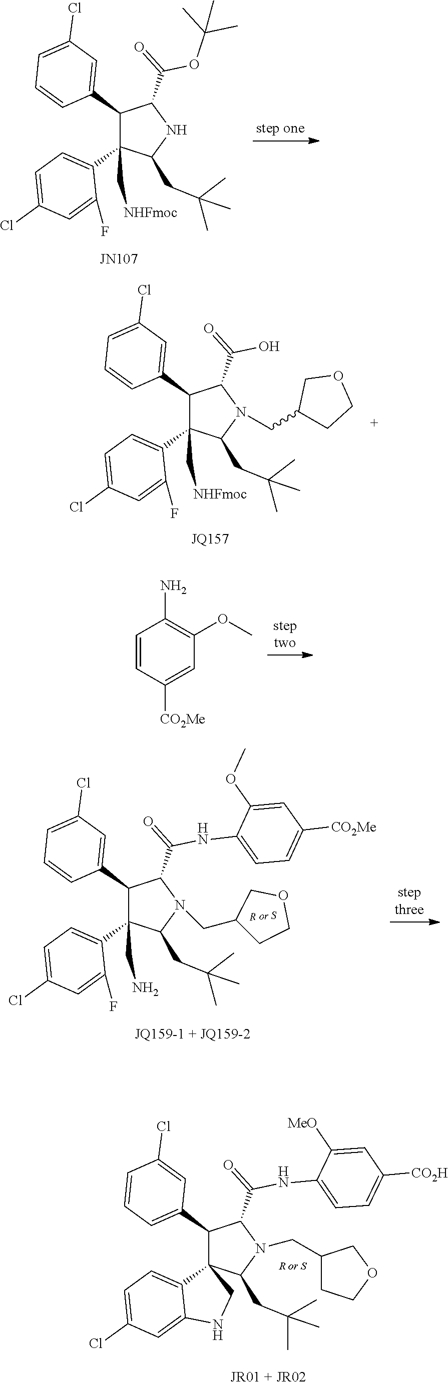

### Step one: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-5-neopentyl-1-((tetrahydrofuran-3-yl)methyl)pyrrolidine-2-carboxylic acid (JQ157)

JN107 (146 mg, 0.2 mmol), tetrahydrofuran-3-carboxaldehyde (100 mg, 1.0 mmol), sodium cyanoborohydride (212 mg, 1.0 mmol), acetic acid (1 mL), sodium cyanoborohydride (126 mg, 2.0 mmol) and trifluoroacetic acid (3 mL) were reacted according to the procedure described in step one of final product 75 to obtain the target product (64 mg, yield 40%).

### Step two: Synthesis of methyl 4-((2R,3R,4S,5S)-4-(aminomethyl)-4-(4-chloro-2-fluorophenyl)-3-(3-chlorophenyl)-5-neopentyl-1-(((R or S)-tetrahydrofuran-3-yl)methyl)pyrrolidine-2-carboxamido)-3-methoxybenzoate (JQ159-1 and JQ159-2)

JQ157 (64 mg, 0.08 mmol), 1-methylimidazole (20 mg, 0.24 mmol), ethylsulfonyl chloride (21 mg, 0.16 mmol), methyl 4-amino-3-methoxybenzoate (44 mg, 0.24 mmol) and piperidine (1.5 mL) were reacted according to the procedure described in step five of the final product 18. After the reaction was completed, HPLC purification afforded JQ159-1 (20 mg, yield 31%). ¹H NMR (400 MHz, Methanol-d₄) δ 8.23 (d, J=8.3 Hz, 1H), 7.62 (d, J=1.7 Hz, 1H), 7.60 (d, J=1.8 Hz, 1H), 7.55 (t, J=8.9 Hz, 1H), 7.41-7.25 (m, 4H), 7.20 (d, J=2.1 Hz, 1H), 7.09 (d, J=7.1 Hz, 1H), 4.50-4.37 (m, 2H), 4.15 (d, J=9.3 Hz, 1H), 3.94 (s, 3H), 3.91 (dd, J=8.3, 4.8 Hz, 1H), 3.88 (s, 3H), 3.77-3.63 (m, 3H), 3.55-3.43 (m, 1H), 3.35 (d, J=15.8 Hz, 1H), 3.28 (d, J=8.1 Hz, 1H), 2.93 (dd, J=12.3, 6.7 Hz, 1H), 2.62-2.45 (m, 1H), 2.22-2.11 (m, 1H), 1.95 (dd, J=15.2, 8.2 Hz, 1H), 1.67 (dq, J=12.1, 7.5 Hz, 1H), 1.48 (dd, J=15.2, 1.7 Hz, 1H), 0.98 (s, 9H). ESI-MS calculated for C₃₇H₄₅Cl₂FN₃₀S [M+H]⁺=700.3, found: 700.3.

JQ159-2 (24 mg, yield 37%). ¹H NMR (400 MHz, Methanol-d₄) δ 8.24 (d, J=8.9 Hz, 1H), 7.65-7.60 (m, 2H), 7.55 (t, J=9.0 Hz, 1H), 7.39-7.27 (m, 4H), 7.20 (t, J=2.2 Hz, 1H), 7.09 (dt, J=6.8, 2.0 Hz, 1H), 4.50-4.36 (m, 2H), 4.21 (d, J=8.9 Hz, 1H), 3.93 (s, 3H), 3.91-3.89 (m, 1H), 3.88 (s, 3H), 3.84 (dd, J=8.3, 5.2 Hz, 1H), 3.77-3.68 (m, 1H), 3.55 (dd, J=8.5, 5.7 Hz, 1H), 3.52-3.45 (m, 1H), 3.36-3.32 (m, 1H), 3.30-3.22 (m, 1H), 2.92 (dd, J=12.5, 7.8 Hz, 1H), 2.58 (hept, J=6.7 Hz, 1H), 2.02-1.93 (m, 1H), 1.89 (dd, J=15.3, 7.6 Hz, 1H), 1.81-1.69 (m, 1H), 1.50 (dd, J=15.0, 1.9 Hz, 1H), 0.97 (s, 9H). ESI-MS calculated for C₃₇H₄₅Cl₂FN₃O₅ [M+H]⁺=700.3, found: 700.3.

### Step Three: Synthesis of 4-[(2′S,3S,4′R,5′R)-6-chloro-4′-(3-chlorophenyl)-2′-neopentyl-1′-((R or S)-tetrahydrofuran-3-ylmethyl)spiro[indoline-3,3′-pyrrolidine]-5′-carboxamide]-3-methoxybenzoic Acid (JR01 and JR02)

JQ159-1 (20 mg, 0.03 mmol), potassium carbonate (17 mg, 0.12 mmol) and lithium hydroxide monohydrate (7 mg, 0.15 mmol) were reacted according to the procedure described in step six of the final product 10 to obtain the trifluoroacetate of JR01 (12.1 mg, yield 25%). ¹H NMR (400 MHz, Methanol-d₄) δ 8.31 (d, J=8.2 Hz, 1H), 7.69-7.59 (m, 2H), 7.33-7.20 (m, 4H), 7.08 (dt, J=7.4, 1.6 Hz, 1H), 6.80 (dt, J=8.0, 1.7 Hz, 1H), 6.56-6.51 (m, 1H), 4.64-4.39 (m, 1H), 4.07-3.80 (m, 7H), 3.76-3.65 (m, 2H), 3.58 (d, J=10.9 Hz, 1H), 3.48 (d, J=10.9 Hz, 1H), 3.36 (d, J=11.4 Hz, 1H), 2.83 (d, J=8.9 Hz, 1H), 2.62-2.43 (m, 1H), 2.28-2.11 (m, 1H), 2.02 (dd, J=15.5, 8.6 Hz, 1H), 1.75-1.62 (m, 1H), 1.36 (d, J=15.6 Hz, 1H), 0.97 (s, 9H). ESI-MS calculated for C₃₆H₄₂Cl₂N₃O₅ [M+H]⁺=666.3, found: 666.2.

JQ159-2 (24 mg, 0.03 mmol), potassium carbonate (19 mg, 0.14 mmol) and lithium hydroxide monohydrate (7 mg, 0.15 mmol) were reacted according to the procedure described in step six of the final product 10 to obtain the trifluoroacetate of JR02 (13 mg, yield 56%). ¹H NMR (400 MHz, Methanol-d₄) δ 8.30 (d, J=8.3 Hz, 1H), 7.68-7.60 (m, 2H), 7.36-7.17 (m, 4H), 7.07 (dt, J=7.4, 1.6 Hz, 1H), 6.80 (dd, J=8.1, 1.9 Hz, 1H), 6.54 (d, J=1.9 Hz, 1H), 4.69-4.36 (m, 1H), 4.06-3.97 (m, 1H), 3.96-3.82 (m, 6H), 3.73 (q, J=7.7 Hz, 1H), 3.63-3.54 (m, 2H), 3.48 (d, J=10.9 Hz, 1H), 3.39-3.32 (m, 1H), 3.01-2.81 (m, 1H), 2.66-2.48 (m, 1H), 2.12-1.93 (m, 2H), 1.93-1.78 (m, 1H), 1.41 (d, J=15.7 Hz, 1H), 0.96 (s, 9H). ESI-MS calculated for C₃₆H₄₂Cl₂N₃O₅ [M+H]⁺=666.3, found: 666.2.

### Final Product 82: 4-[(2′S,3S,4′R,5′R)-6-chloro-4′-(3-chlorophenyl)-2′-neopentyl-1′-((tetrahydro-2H-pyran-4-yl)methyl)spiro[indoline-3,3′-pyrrolidine]-5′-carboxamide]-3-methoxybenzoic Acid (JQ122)

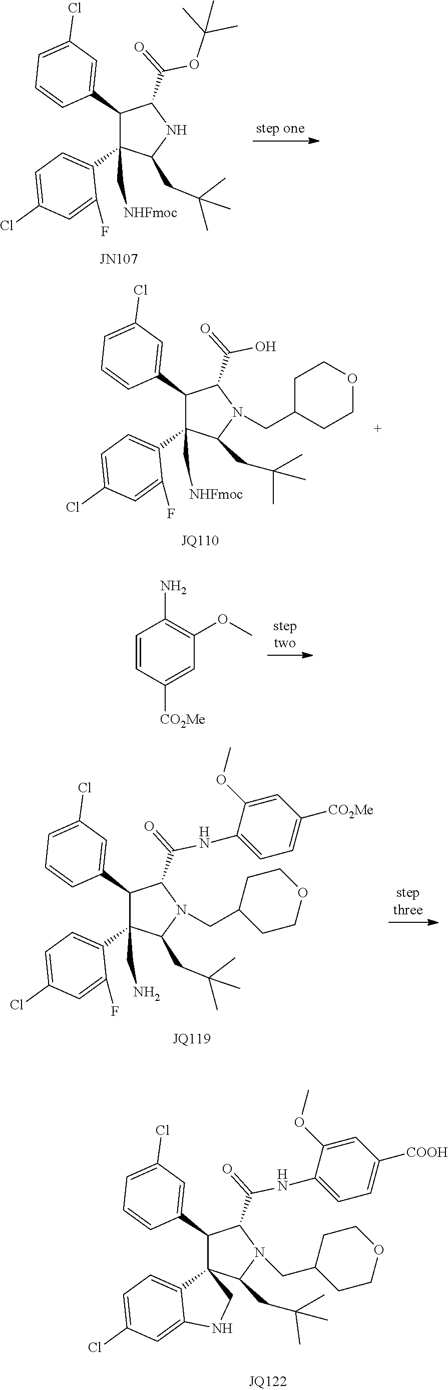

### Step one: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-5-neopentyl-1-((tetrahydro-2H-pyran-4-yl)methyl)pyrrolidine-2-carboxylic acid (JQ110)

JN107 (100 mg, 0.14 mmol), tetrahydro-2H-pyran-4-carbaldehyde (78 mg, 0.68 mmol), sodium borohydride acetate (144 mg, 0.68 mmol), acetic acid (1 mL), sodium cyanoborohydride (89 mg, 1.4 mmol) and trifluoroacetic acid (2 mL) were reacted according to the procedure described in step one of the final product 75 to obtain the target product (20 mg, yield 19%).

### Step two: Synthesis of methyl 4-((2R,3R,4S,5S)-4-(aminomethyl)-4-(4-chloro-2-fluorophenyl)-3-(3-chlorophenyl)-5-neopentyl-1-((tetrahydro-2H-pyran-4-yl)methyl)pyrrolidine-2-carboxamido)-3-methoxybenzoate (JQ119)

JQ110 (19 mg, 0.03 mmol), 1-methylimidazole (8 mg, 0.09 mmol), ethylsulfonyl chloride (8 mg, 0.06 mmol), methyl 4-amino-3-methoxybenzoate (16 mg, 0.09 mmol) and piperidine (1.5 mL) were reacted according to the procedure described in step five of the final product 18 to obtain the target product (17 mg, yield 69%). ¹H NMR (400 MHz, Methanol-d₄) δ 8.19 (d, J=8.3 Hz, 1H), 7.68-7.59 (m, 2H), 7.49 (t, J=7.9, 6.2 Hz, 1H), 7.39-7.25 (m, 4H), 7.20 (s, 1H), 7.09 (d, J=6.8 Hz, 1H), 4.51-4.39 (m, 2H), 4.34 (d, J=8.4 Hz, 1H), 3.97 (d, J=11.4 Hz, 1H), 3.90 (s, 3H), 3.89 (s, 3H), 3.87-3.84 (m, 1H), 3.54 (d, J=14.4 Hz, 1H), 3.45 (t, J=11.8 Hz, 1H), 3.27 (d, J=14.3 Hz, 1H), 3.10 (dd, J=13.1, 5.4 Hz, 1H), 2.76 (dd, J=12.7, 7.9 Hz, 1H), 1.98-1.86 (m, 1H), 1.85-1.64 (m, 3H), 1.52 (d, J=15.2 Hz, 1H), 1.43-1.12 (m, 3H), 0.91 (s, 9H). ESI-MS calculated for C₃₈H₄₇Cl₂FN₃O₅ [M+H]⁺=714.3, found: 714.3.

### Step Three: Synthesis of 4-((2′S,3S,4′R,5′R)-6-chloro-4′-(3-chlorophenyl)-2′-neopentyl-1′-((tetrahydro-2H-pyran-4-yl)methyl)spiro[indole-3,3′-pyrrolidine]-5′-carboxamido)-3-methoxybenzoic Acid (JQ122)

JQ119 (17 mg, 0.02 mmol), potassium carbonate (14 mg, 0.1 mmol) and lithium hydroxide monohydrate (5 mg, 0.1 mmol) were reacted according to the procedure described in step six of the final product 10 to obtain the trifluoroacetate salt of the target product (9.5 mg, yield 60%). ¹H NMR (400 MHz, Methanol-d₄) δ 8.28 (d, J=8.4 Hz, 1H), 7.64 (d, J=8.9 Hz, 1H), 7.61 (s, 1H), 7.37-7.20 (m, 4H), 7.07 (d, J=7.3 Hz, 1H), 6.76 (d, J=8.1 Hz, 1H), 6.49 (s, 1H), 4.71-4.34 (m, 1H), 4.25-3.89 (m, 4H), 3.87 (s, 3H), 3.60 (d, J=10.9 Hz, 1H), 3.52-3.38 (m, 2H), 3.23 (t, J=12.0 Hz, 1H), 2.92-2.55 (m, 1H), 2.16-1.84 (m, 3H), 1.76 (d, J=12.9 Hz, 1H), 1.60-1.25 (m, 3H), 0.95 (s, 9H). ESI-MS calculated for C₃₇H₄₄Cl₂N₃O₅ [M+H]⁺=680.3, found: 680.3.

### Final Product 83: 4-((2′S,3S,4′R,5′R)-6-chloro-4′-(3-chlorophenyl)-1′-(furan-2-ylmethyl)-2′-neopentylspiro[indoline-3,3′-pyrrolidine]-5′-carboxamido)-3-methoxybenzoic Acid (JQ149)

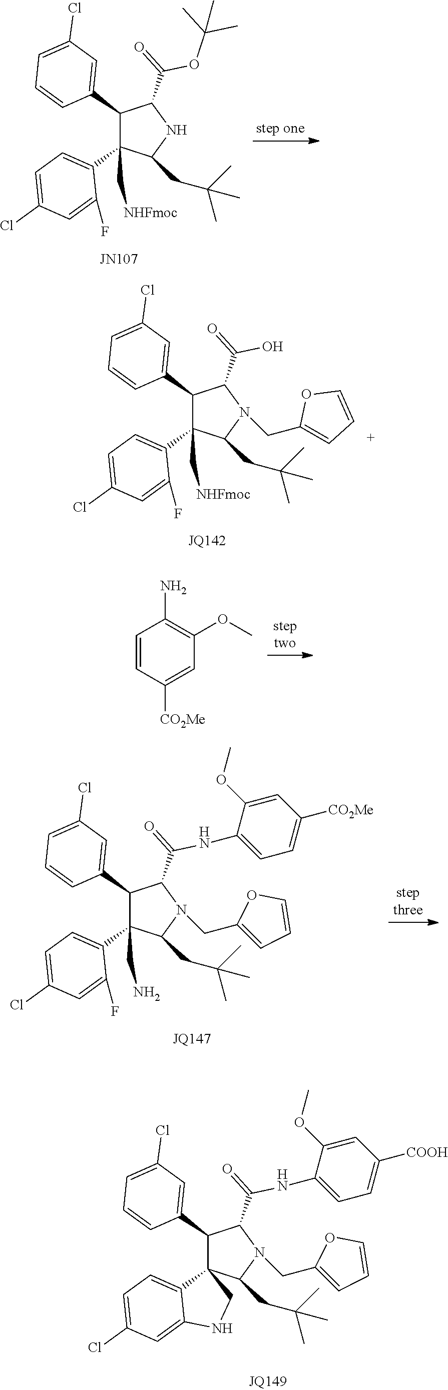

### Step one: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-1-(furan-2-ylmethyl)-5-neopentylpyrrolidine-2-carboxylic Acid (JQ142)

JN107 (146 mg, 0.2 mmol), furan-2-carbaldehyde (96 mg, 1.0 mmol), sodium borohydride acetate (212 mg, 1.0 mmol), acetic acid (1 mL), sodium cyanoborohydride (126 mg, 2.0 mmol) and trifluoroacetic acid (3 mL) were reacted according to the procedure described in step one of the final product 75 to obtain the target product (37 mg, yield 25%).

### Step two: Synthesis of methyl 4-((2R,3R,4S,5S)-4-(aminomethyl)-4-(4-chloro-2-fluorophenyl)-3-(3-chlorophenyl)-1-(furan-2-ylmethyl)-5-neopentylpyrrolidine-2-carboxamido)-3-methoxybenzoate (JQ147)

JQ142 (37 mg, 0.05 mmol), 1-methylimidazole (13 mg, 0.15 mmol), ethylsulfonyl chloride (13 mg, 0.1 mmol), methyl 4-amino-3-methoxybenzoate (27 mg, 0.15 mmol) and piperidine (1.5 mL) were reacted according to the procedure described in step five of the final product 18 to obtain the target product (40 mg, yield 99%). ¹H NMR (400 MHz, Methanol-d₄) δ 8.12 (d, J=8.4 Hz, 1H), 7.61-7.45 (m, 3H), 7.39-7.23 (m, 5H), 7.12 (s, 1H), 7.06 (dd, J=5.4, 3.2 Hz, 1H), 6.39 (d, J=3.2 Hz, 1H), 6.24 (dd, J=3.2, 1.9 Hz, 1H), 4.63-4.48 (m, 2H), 4.33 (d, J=9.4 Hz, 1H), 4.13 (d, J=14.0 Hz, 1H), 4.04 (d, J=9.3 Hz, 1H), 3.92 (s, 3H), 3.86 (s, 3H), 3.50 (d, J=14.5 Hz, 1H), 3.34 (d, J=13.3 Hz, 1H), 2.11 (dd, J=15.2, 8.8 Hz, 1H), 1.55 (d, J=15.1 Hz, 1H), 1.09 (s, 9H). ESI-MS calculated for C₃₇H₄₁Cl₂FN₃O₅ [M+H]⁺=696.2, found: 696.2.

### Step Three: Synthesis of 4-((2′S,3S,4′R,5′R)-6-chloro-4′-(3-chlorophenyl)-1′-(furan-2-ylmethyl)-2′-neopentylspiro[indole-3,3′-pyrrolidine]-5′-carboxamido)-3-methoxybenzoic Acid (JQ149)

JQ147 (40 mg, 0.06 mmol), potassium carbonate (32 mg, 0.24 mmol) and lithium hydroxide monohydrate (13 mg, 0.3 mmol) were reacted according to the procedure described in step six of the final product 10 to obtain the trifluoroacetate salt of the target product (25.4 mg, yield 55%). ¹H NMR (400 MHz, Methanol-d₄) δ 8.16 (d, J=8.3 Hz, 1H), 7.62-7.53 (m, 2H), 7.35 (d, J=1.8 Hz, 1H), 7.32 (d, J=8.1 Hz, 1H), 7.28-7.20 (m, 2H), 7.17 (t, J=2.0 Hz, 1H), 7.10-7.03 (m, 1H), 6.85 (dd, J=8.1, 1.9 Hz, 1H), 6.61 (d, J=1.9 Hz, 1H), 6.49 (d, J=3.2 Hz, 1H), 6.27 (dd, J=3.3, 1.9 Hz, 1H), 4.61 (d, J=13.8 Hz, 1H), 4.38 (d, J=9.9 Hz, 1H), 3.93 (s, 3H), 3.93-3.89 (m, 2H), 3.86 (d, J=10.0 Hz, 1H), 3.61 (d, J=11.0 Hz, 1H), 3.48 (d, J=11.0 Hz, 1H), 2.10 (dd, J=15.5, 8.9 Hz, 1H), 1.38 (d, J=15.4 Hz, 1H), 1.04 (s, 9H). ESI-MS calculated for C₃₆H₃₈Cl₂N₃O₅ [M+H]⁺=662.2, found: 662.2.

### Final Product 84: 4-((2′S,3S,4′R,5′R)-6-chloro-4′-(3-chlorophenyl)-1′-(2,2-difluoroethyl)-2′-neopentylspiro[indoline-3,3′-pyrrolidine]-5′-carboxamido)-3-methoxybenzoic Acid (JQ57)

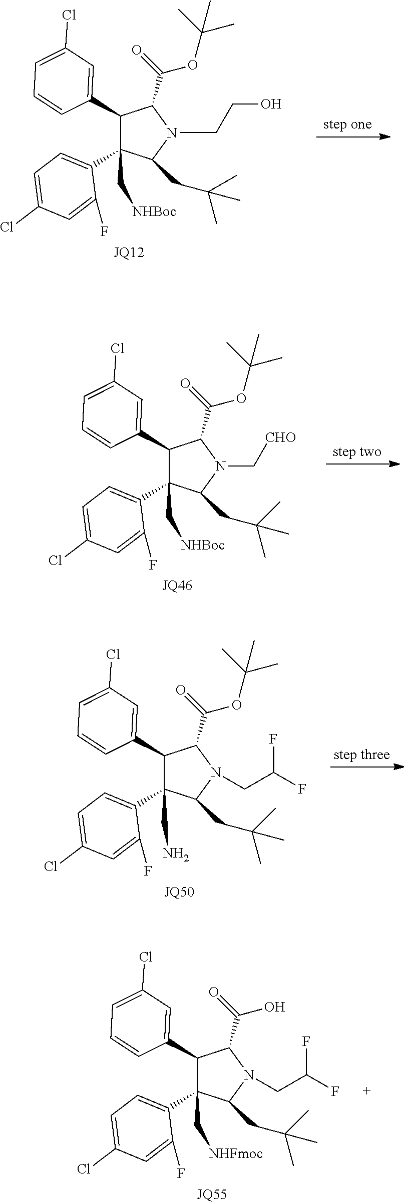

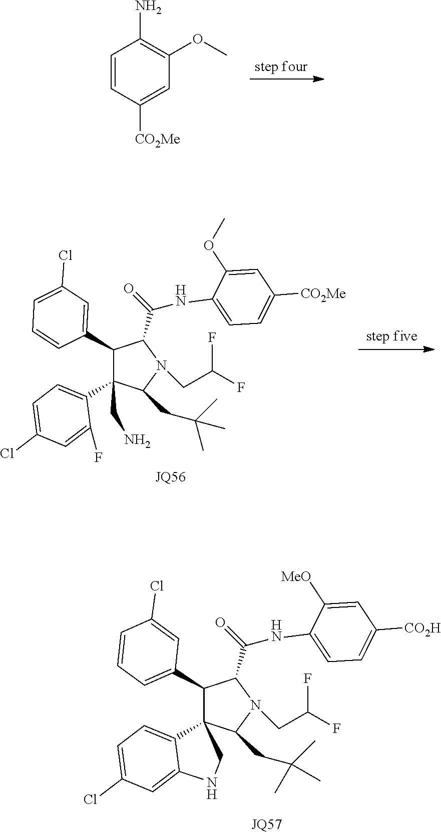

### Step one: Synthesis of tert-butyl (2R,3R,4S,5S)-4-(((tert-butoxycarbonyl)amino)methyl)-4-(4-chloro-2-fluorophenyl)-3-(3-chlorophenyl)-5-neopentyl-1-(2-oxoethyl)pyrrolidine-2-carboxylate (JQ46)

Dimethyl sulfoxide (141 mg, 1.8 mmol) was added to a 100 mL round-bottom flask, followed by addition of anhydrous dichloromethane. Oxalyl chloride (115 mg, 0.9 mmol) was added dropwise at −78° C., and the mixture was stirred for 15 minutes. A solution of JQ12 (200 mg, 0.3 mmol) in dichloromethane was then added dropwise to the reaction mixture, which was stirred at −78° C. for 1 hour. Triethylamine (273 mg, 2.7 mmol) was added dropwise, and the mixture was allowed to warm to room temperature naturally and reacted for 3 hours. After the reaction was complete, water was added, and the mixture was extracted three times with ethyl acetate. The combined organic phases were washed once with saturated sodium chloride solution, dried over anhydrous sodium sulfate, and concentrated to obtain 220 mg of crude product, which was used directly in the next step.

### Step two: Synthesis of tert-butyl (2R,3R,4S,5S)-4-(aminomethyl)-4-(4-chloro-2-fluorophenyl)-3-(3-chlorophenyl)-1-(2,2-difluoroethyl)-5-neopentylpyrrolidine-2-carboxylate (JQ50)

JQ46 (264 mg, 0.42 mmol), diethylaminosulfur trifluoride (203 mg, 1.26 mmol) and trifluoroacetic acid (1 mL) were reacted according to the procedure described in step three of the final product 49 to obtain the target product (160 mg, yield 66%).

### Step Three: Synthesis of (2R,3R,4S,5S)-4-(((9H-fluoren-9-yl)methoxy)carbonyl)amino)methyl)-4-(4-chloro-2-fluorophenyl)-3-(3-chlorophenyl)-1-(2,2-difluoroethyl)-5-neopentylpyrrolidine-2-carboxylic Acid (JQ55)

JQ50 (170 mg, 0.61 mmol), diisopropylethylamine (315 mg, 2.44 mmol), FmocCl (238 mg, 0.92 mmol) and trifluoroacetic acid (4 mL) were reacted according to the procedure described in step four of the final product 49 to obtain the target product (108 mg, yield 50%).

### Step Four: Synthesis of methyl 4-((2R,3R,4S,5S)-4-(aminomethyl)-4-(4-chloro-2-fluorophenyl)-3-(3-chlorophenyl)-1-(2,2-difluoroethyl)-5-neopentylpyrrolidine-2-carboxamido)-3-methoxybenzoate (JQ56)

JQ55 (50 mg, 0.06 mmol), 1-methylimidazole (17 mg, 0.2 mmol), ethylsulfonyl chloride (16 mg, 0.12 mmol), methyl 4-amino-3-methoxybenzoate (36 mg, 0.2 mmol) and piperidine (2 mL) were reacted according to the procedure described in step five of the final product 18 to obtain the target product (40 mg, yield 83%). ¹H NMR (400 MHz, Methanol-d₄) δ 8.22 (d, J=8.8 Hz, 1H), 7.64-7.54 (m, 2H), 7.48 (t, J=8.8 Hz, 1H), 7.37-7.30 (m, 3H), 7.28 (dd, J=13.5, 2.2 Hz, 1H), 7.13 (s, 1H), 7.06 (d, J=6.6 Hz, 1H), 6.08 (tt, J=55.5, 3.9 Hz, 1H), 4.54 (d, J=8.4 Hz, 1H), 4.42 (d, J=9.2 Hz, 1H), 4.08 (d, J=9.1 Hz, 1H), 3.91 (s, 3H), 3.86 (s, 3H), 3.84-3.76 (m, 1H), 3.55-3.35 (m, 2H), 3.24 (dd, J=14.5, 2.3 Hz, 1H), 1.95 (dd, J=15.5, 8.7 Hz, 1H), 1.55 (d, J=15.3 Hz, 1H), 1.02 (s, 9H). ESI-MS calculated for C₃₄H₃₉Cl₂F₃N₃O₄ [M+H]⁺=680.2, found: 680.2.

### Step five: Synthesis of 4-((2′S,3S,4′R,5′R)-6-chloro-4′-(3-chlorophenyl)-1′-(2,2-difluoroethyl)-2′-neopentylspiro[indole-3,3′-pyrrolidine]-5′-carboxamido)-3-methoxybenzoic Acid (JQ57)

JQ56 (40 mg, 0.06 mmol), potassium carbonate (34 mg, 0.24 mmol) and lithium hydroxide monohydrate (13 mg, 0.3 mmol) were reacted according to the procedure described in step six of the final product 10 to obtain the trifluoroacetate salt of the target product (28.2 mg, yield 62%). ¹H NMR (400 MHz, Methanol-d₄) δ 8.34 (d, J=8.8 Hz, 1H), 7.71-7.60 (m, 2H), 7.29 (d, J=8.1 Hz, 1H), 7.25-7.18 (m, 2H), 7.15 (d, J=2.1 Hz, 1H), 7.09-6.98 (m, 1H), 6.84 (dd, J=8.1, 1.9 Hz, 1H), 6.61 (d, J=1.8 Hz, 1H), 6.02 (tt, J=55.6, 3.9 Hz, 1H), 4.24 (d, J=9.1 Hz, 1H), 3.94 (s, 3H), 3.88-3.68 (m, 3H), 3.48 (d, J=10.9 Hz, 1H), 3.37 (d, J=11.0 Hz, 1H), 3.03 (qd, J=14.7, 4.1 Hz, 1H), 1.87 (dd, J=15.5, 9.3 Hz, 1H), 1.24 (d, J=15.5 Hz, 1H), 0.99 (s, 9H). ESI-MS calculated for C₃₃H₃₆Cl₂F₂N₃O₄ [M+H]⁺=646.2, found: 646.2.

### Final Product 85: 4-((2′S,3S,4′R,5′R)-6-chloro-4′-(3-chlorophenyl)-2′-neopentyl-1′-(prop-2-yn-1-yl)spiro[indoline-3,3′-pyrrolidine]-5′-carboxamido)-3-methoxybenzoic acid (JQ158)

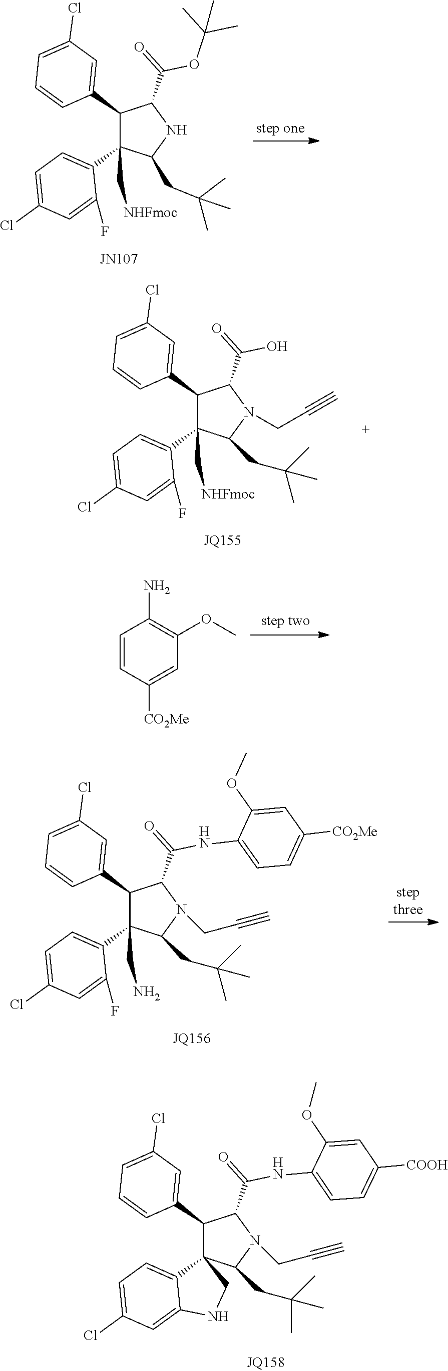

### Step one: Synthesis of (2R,3R,4S,5S)-4-((((9H-fluoren-9-yl)methoxy)carbonyl)amino)methyl)-4-(4-chloro-2-fluorophenyl)-3-(3-chlorophenyl)-5-neopentyl-1-(prop-2-yn-1-yl)pyrrolidine-2-carboxylic Acid (JQ155)

JN107 (132 mg, 0.18 mmol), propiolaldehyde (101 mg, 1.8 mmol), sodium borohydride acetate (382 mg, 1.8 mmol), acetic acid (2 mL), sodium cyanoborohydride (114 mg, 1.8 mmol) and trifluoroacetic acid (2 mL) were reacted according to the procedure described in step one of the final product 75 to obtain the target product (80 mg, yield 63%).

### Step two: Synthesis of methyl 4-((2R,3R,4S,5S)-4-(aminomethyl)-4-(4-chloro-2-fluorophenyl)-3-(3-chlorophenyl)-5-neopentyl-1-(prop-2-yn-1-yl)pyrrolidine-2-carboxamido)-3-methoxybenzoate (JQ156)

JQ155 (49 mg, 0.07 mmol), 1-methylimidazole (18 mg, 0.21 mmol), ethylsulfonyl chloride (18 mg, 0.14 mmol), methyl 4-amino-3-methoxybenzoate (38 mg, 0.21 mmol) and piperidine (2 mL) were reacted according to the procedure described in step five of the final product 18 to obtain the target product (40 mg, yield 74%). ¹H NMR (400 MHz, Methanol-d₄) δ 8.26 (d, J=8.9 Hz, 1H), 7.65-7.53 (m, 3H), 7.38-7.31 (m, 3H), 7.28 (dd, J=13.6, 2.2 Hz, 1H), 7.18 (s, 1H), 7.16-7.07 (m, 1H), 4.51 (d, J=9.3 Hz, 1H), 4.41 (d, J=8.1 Hz, 1H), 4.16-4.03 (m, 2H), 3.98-3.93 (m, 1H), 3.92 (s, 3H), 3.87 (s, 3H), 3.49 (d, J=14.6 Hz, 1H), 3.39-3.32 (m, 1H), 2.81 (t, J=2.2 Hz, 1H), 1.96 (dd, J=15.3, 8.4 Hz, 1H), 1.50 (dd, J=15.3, 1.8 Hz, 1H), 1.03 (s, 9H). ESI-MS calculated for C₃₅H₃₉Cl₂FN₃O₄ [M+H]⁺=654.2, found: 654.2.

### Step Three: Synthesis of methyl 4-((2R,3R,4S,5S)-4-(aminomethyl)-4-(4-chloro-2-fluorophenyl)-3-(3-chlorophenyl)-5-neopentyl-1-(prop-2-yn-1-yl)pyrrolidine-2-carboxamido)-3-methoxybenzoate (JQ158)

JQ156 (40 mg, 0.06 mmol), potassium carbonate (34 mg, 0.24 mmol), lithium hydroxide monohydrate (13 mg, 0.3 mmol) were reacted according to the procedure described in step six of the final product 10 to obtain the trifluoroacetate of the target product (13.7 mg, yield 31%). ¹H NMR (400 MHz, Methanol-d₄) δ 8.31 (d, J=8.8 Hz, 1H), 7.66-7.60 (m, 2H), 7.33 (d, J=8.1 Hz, 1H), 7.26-7.19 (m, 2H), 7.17 (s, 1H), 7.11-7.02 (m, 1H), 6.84 (dd, J=8.1, 1.9 Hz, 1H), 6.59 (d, J=1.9 Hz, 1H), 4.47 (d, J=10.1 Hz, 1H), 4.01 (dd, J=17.0, 2.5 Hz, 1H), 3.94 (s, 3H), 3.89-3.82 (m, 2H), 3.77 (dd, J=16.9, 2.4 Hz, 1H), 3.65 (d, J=11.2 Hz, 1H), 3.43 (d, J=11.1 Hz, 1H), 2.82 (t, J=2.3 Hz, 1H), 1.91 (dd, J=16.0, 8.0 Hz, 1H), 1.34 (dd, J=15.5, 1.5 Hz, 1H), 0.97 (s, 9H). ESI-MS calculated for C₃₄H₃₆C₂N₃O₄ [M+H]⁺=620.2, found: 620.2.

### Final Product 86: 4-[(2′S,3S,4′R,5′R)-6-chloro-4′-(3-chlorophenyl)-1′-ethyl-7-fluoro-2′-neopentylspiro[indoline-3,3′-pyrrolidine]-5′-carboxamide]-3-methoxybenzoic Acid (JQ120)

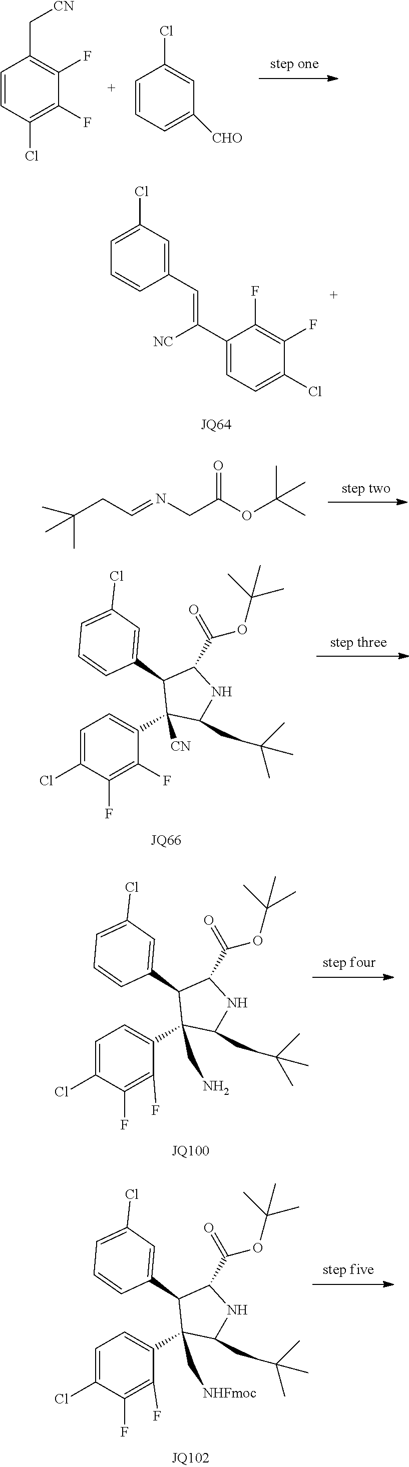

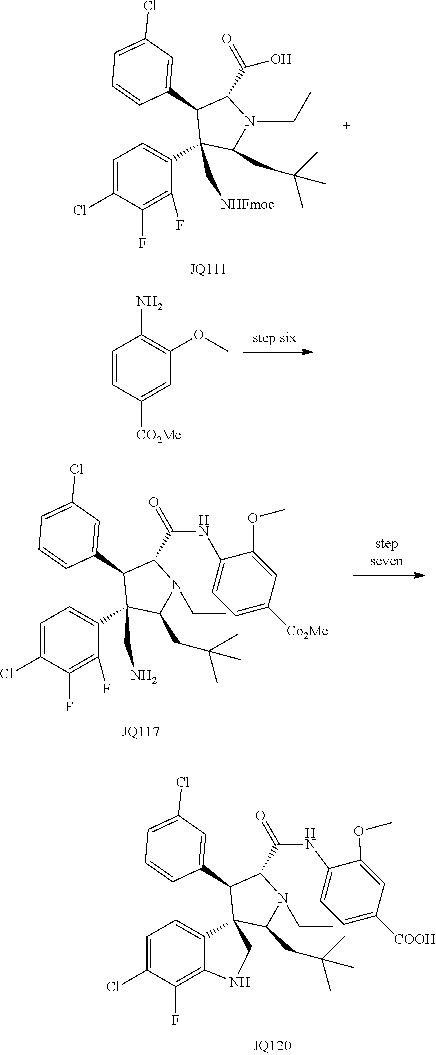

### Step one: Synthesis of (Z)-2-(4-chloro-2,3-difluorophenyl)-3-(3-chlorophenyl)acrylonitrile (JQ64)

3-Chlorobenzaldehyde (1.5 g, 10.7 mmol), 2-(4-chloro-2,3-difluorophenyl)acetonitrile (2.0 g, 10.7 mmol) and 5N sodium methoxide in methanol (2.6 mL) were reacted according to the procedure described in step one of intermediate 1 to obtain the target product (3.2 g, yield 97%). ¹H NMR (400 MHz, Chloroform-d) δ 7.83 (dt, J=7.4, 2.2 Hz, 1H), 7.81 (t, J=1.6 Hz, 1H), 7.55 (s, 1H), 7.50-7.41 (m, 2H), 7.38-7.27 (m, 2H).

### Step two: Synthesis of tert-butyl (2R,3R,4R,5S)-4-(4-chloro-2,3-difluorophenyl)-3-(3-chlorophenyl)-4-cyano-5-neopentylpyrrolidine-2-carboxylate (JQ66)

Tert-butyl (E)-2-(((3,3-dimethylbutylidene)amino)acetate (1.9 g, 9.0 mmol), JQ64 (2.3 g, 7.5 mmol), copper(I) acetate (92 mg, 0.75 mmol), R—(+)-1,1′-binaphthyl-2,2′-bis(diphenylphosphine) (514 mg, 0.83 mmol) and triethylamine (758 mg, 7.5 mmol) were reacted according to the procedure described in step one of final product 18 to obtain the target product (3.5 g, yield 90%). ¹H NMR (400 MHz, Chloroform-d) δ 7.29-7.22 (m, 2H), 7.21-7.12 (m, 3H), 7.10 (dt, J=7.3, 1.6 Hz, 1H), 4.24 (d, J=7.5 Hz, 1H), 4.11 (d, J=7.6 Hz, 1H), 4.04 (d, J=9.1 Hz, 1H), 1.64 (dd, J=14.4, 9.2 Hz, 1H), 1.39 (s, 9H), 1.29 (dd, J=14.4, 1.2 Hz, 1H), 0.91 (s, 9H).

### Step Three: Synthesis of tert-butyl (2R,3R,4S,5S)-4-(aminomethyl)-4-(4-chloro-2,3-difluorophenyl)-3-(3-chlorophenyl)-5-neopentylpyrrolidine-2-carboxylate (JQ100)

JQ66 (3.5 g, 6.7 mmol), Raney nickel (5 g) and hydrazine hydrate (10 mL) were reacted according to the procedure described in step three of intermediate 1 to obtain the target product (1.4 g, yield 40%). ¹H NMR (400 MHz, Chloroform-d) δ 7.25-7.14 (m, 3H), 7.13-7.08 (m, 1H), 7.00-6.94 (m, 1H), 6.88-6.77 (m, 1H), 4.31 (dd, J=8.0, 1.7 Hz, 1H), 4.15 (dd, J=9.8, 3.9 Hz, 1H), 4.00 (d, J=8.4 Hz, 1H), 3.33 (dd, J=13.9, 6.0 Hz, 1H), 3.04 (dd, J=13.6, 3.9 Hz, 1H), 1.54-1.45 (m, 1H), 1.35 (s, 9H), 0.96 (d, J=9.7 Hz, 1H), 0.92 (s, 9H).

### Step Four: Synthesis of tert-butyl (2R,3R,4S,5S)-4-((((9H-fluoren-9-yl)methoxy)carbonyl)amino)methyl)-4-(4-chloro-2,3-difluorophenyl)-3-(3-chlorophenyl)-5-neopentylpyrrolidine-2-carboxylate (JQ102)

JQ100 (1.2 g, 2.3 mmol), diisopropylethylamine (1.2 g, 9.0 mmol) and FmocCl (880 mg, 3.4 mmol) were reacted according to the procedure described in step three of final product 18 to obtain the target product (1.8 g, yield 99%).

### Step five: Synthesis of (2R,3R,4S,5S)-4-((((9H-fluoren-9-yl)methoxy)carbonyl)amino)methyl)-4-(4-chloro-2,3-difluorophenyl)-3-(3-chlorophenyl)-1-ethyl-5-neopentylpyrrolidine-2-carboxylic Acid (JQ111)

JQ102 (153 mg, 0.2 mmol), acetaldehyde (44 mg, 1.0 mmol), sodium cyanoborohydride (212 mg, 1.0 mmol), acetic acid (1 mL) and trifluoroacetic acid (3 mL) were reacted according to the procedure described in step four of final product 18 to obtain the target product (96 mg, yield 67%).

### Step six: Synthesis of methyl 4-((2R,3R,4S,5S)-4-(aminomethyl)-4-(4-chloro-2,3-difluorophenyl)-3-(3-chlorophenyl)-1-ethyl-5-neopentylpyrrolidine-2-carboxamido)-3-methoxybenzoate (JQ17)

JQ111 (53 mg, 0.07 mmol), 1-methylimidazole (19 mg, 0.22 mmol), ethylsulfonyl chloride (20 mg, 0.15 mmol), methyl 4-amino-3-methoxybenzoate (40 mg, 0.22 mmol) and piperidine (2 mL) were reacted according to the procedure described in step five of the final product 18 to obtain the target product (67 mg, yield 99%). ¹H NMR (400 MHz, Methanol-d₄) δ 8.26 (d, J=8.4 Hz, 1H), 7.69-7.54 (m, 2H), 7.42 (t, J=8.0 Hz, 1H), 7.38-7.28 (m, 3H), 7.16 (s, 1H), 7.05 (d, J=7.1 Hz, 1H), 4.44 (d, J=9.2 Hz, 1H), 4.34 (d, J=9.2 Hz, 1H), 4.02-3.96 (m, 1H), 3.95 (s, 3H), 3.88 (s, 3H), 3.55-3.40 (m, 2H), 3.37-3.32 (m, 1H), 3.11-2.96 (m, 1H), 2.07 (d, J=10.0 Hz, 1H), 1.46 (d, J=15.0 Hz, 1H), 1.24 (t, J=7.0 Hz, 3H), 1.06 (s, 9H). ESI-MS calculated for C₃₄H₄₀Cl₂F₂N₃O₄ [M+H]⁺=662.2, found: 662.3.

### Step seven: Synthesis of 4-[(2′S,3S,4′R,5′R)-6-chloro-4′-(3-chlorophenyl)-1′-ethyl-7-fluoro-2′-neopentylspiro[indole-3,3′-pyrrolidin]-5′-carboxamido]-3-methoxybenzoic Acid (JQ120)

JQ117 (67 mg, 0.1 mmol), potassium carbonate (56 mg, 0.4 mmol) and lithium hydroxide monohydrate (17 mg, 0.4 mmol) were reacted according to the procedure described in step six of the final product 10 to obtain the trifluoroacetic acid salt of the target product (42.6 mg, yield 57%). ¹H NMR (400 MHz, Methanol-d₄) δ 8.16 (d, J=8.3 Hz, 1H), 7.62 (d, J=8.5 Hz, 1H), 7.57 (s, 1H), 7.36-7.24 (m, 3H), 7.21 (d, J=8.2 Hz, 1H), 7.13 (d, J=7.4 Hz, 1H), 6.79 (t, J=7.2 Hz, 1H), 5.16-4.98 (m, 1H), 4.34-4.05 (m, 2H), 3.83 (s, 3H), 3.77 (d, J=11.1 Hz, 1H), 3.67 (dd, J=13.0, 7.1 Hz, 1H), 3.60 (d, J=11.1 Hz, 1H), 3.31-3.16 (m, 1H), 2.04 (dd, J=16.3, 5.9 Hz, 1H), 1.92-1.69 (m, 1H), 1.41 (t, J=7.1 Hz, 3H), 0.84 (s, 9H). ESI-MS calculated for C₃₃H₃₇Cl₂FN₃O₄ [M+H]⁺=628.2, found: 628.2.

### Final Product 87: 4-[(2′S,3S,4′R,5′R)-6-chloro-4′-(3-chlorophenyl)-1′-ethyl-5-fluoro-2′-neopentylspiro[indoline-3,3′-pyrrolidin]-5′-carboxamido]-3-methoxybenzoic Acid (JQ121)

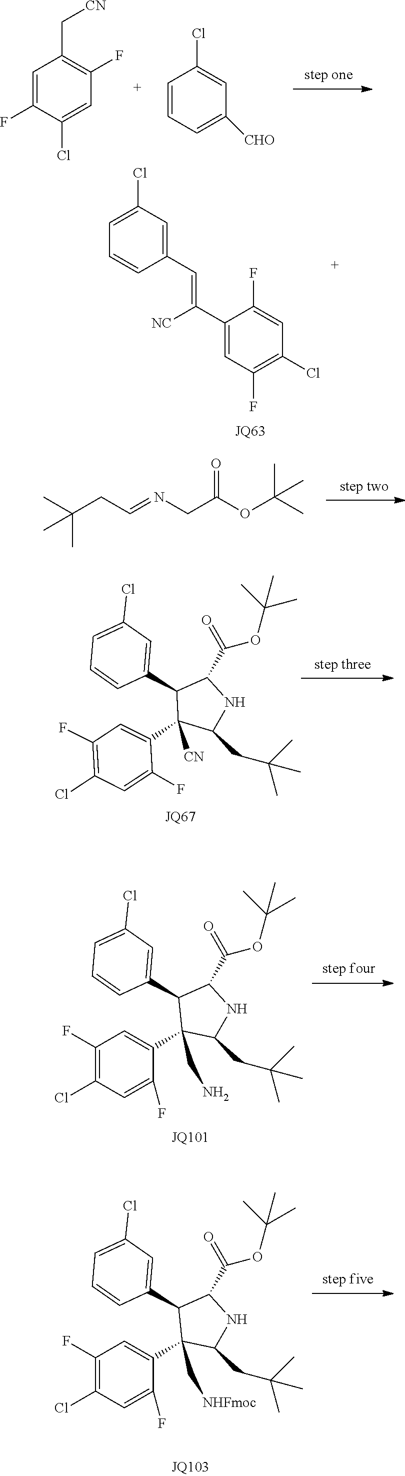

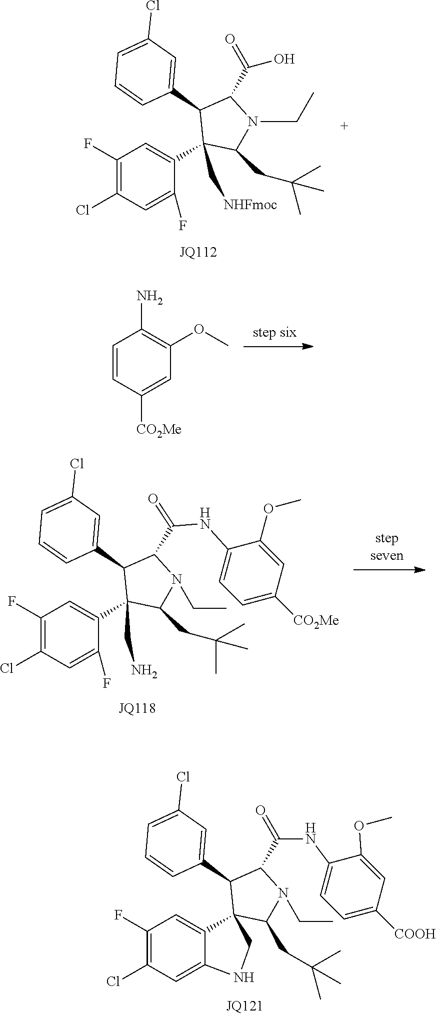

### Step one: Synthesis of (Z)-2-(4-chloro-2,5-difluorophenyl)-3-(3-chlorophenyl)acrylonitrile (JQ63)

3-chlorobenzaldehyde (2.5 g, 18.0 mmol), 2-(4-chloro-2,5-difluorophenyl)acetonitrile (3.4 g, 18.0 mmol) and 5N sodium methoxide in methanol (4.3 mL) were reacted according to the procedure described in step one of intermediate 1 to obtain the target product (4.3 g, yield 80%). ¹H NMR (400 MHz, Chloroform-d) δ 7.83 (dt, J=6.8, 1.7 Hz, 1H), 7.80 (d, J=1.7 Hz, 1H), 7.56 (s, 1H), 7.49-7.38 (m, 3H), 7.32-7.27 (m, 1H).

### Step two: Synthesis of tert-butyl (2R,3R,4R,5S)-4-(4-chloro-2,5-difluorophenyl)-3-(3-chlorophenyl)-4-cyano-5-neopentylpyrrolidine-2-carboxylate (JQ67)

Tert-butyl (E)-2-(((3,3-dimethylbutylidene)amino)acetate (2.1 g, 9.7 mmol), JQ63 (2.5 g, 8.1 mmol), copper(I) acetate (100 mg, 0.81 mmol), R—(+)-1,1′-binaphthyl-2,2′-bis(diphenylphosphine) (555 mg, 0.89 mmol) and triethylamine (818 mg, 8.1 mmol) were reacted according to the procedure described in step one of the final product 18 to obtain the target product (4.0 g, yield 95%). ¹H NMR (400 MHz, Chloroform-d) δ 7.30-7.20 (m, 5H), 7.17 (t, J=1.9 Hz, 1H), 7.10 (dt, J=7.3, 1.6 Hz, 1H), 4.22 (d, J=7.6 Hz, 1H), 4.13 (d, J=7.6 Hz, 1H), 4.02 (d, J=9.0 Hz, 1H), 1.62 (dd, J=14.3, 9.1 Hz, 1H), 1.38 (s, 9H), 1.29 (dd, J=14.3, 1.2 Hz, 1H), 0.90 (s, 9H).

### Step Three: Synthesis of tert-butyl (2R,3R,4S,5)-4-(aminomethyl)-4-(4-chloro-2,5-difluorophenyl)-3-(3-chlorophenyl)-5-neopentylpyrrolidine-2-carboxylate (JQ101)

JQ67 (4.0 g, 7.6 mmol), Raney nickel (5 g) and hydrazine hydrate (10 mL) were reacted according to the procedure described in step three of intermediate 1 to obtain the target product (1.9 g, yield 48%). ¹H NMR (400 MHz, Chloroform-d) δ 7.30-7.24 (in, 1), 7.23-7.07 (m, 3H), 7.02 (dd, J=11.1, 6.9 Hz, 1H), 6.96 (dd, J=7.6, 1.5 Hz, 1H), 4.27 (d, J=8.8 Hz, 1H), 4.07 (dd, J=8.4, 2.1 Hz, 1H), 3.83 (dd, J=8.8, 2.0 Hz, 1H), 3.22 (d, J=13.1 Hz, 1H), 3.05 (d, J=13.1 Hz, 1H), 1.50-1.38 (m, 2H), 1.30 (s, 9H), 0.93 (s, 9H).

### Step Four: Synthesis of tert-butyl (2R,3R,4S,5S)-4-((((9H-fluoren-9-yl)methoxy)carbonyl)amino)methyl)-4-(4-chloro-2,5-difluorophenyl)-3-(3-chlorophenyl)-5-neopentylpyrrolidine-2-carboxylate (JQ103)

JQ101 (1.7 g, 3.2 mmol), diisopropylethylamine (1.7 g, 12.8 mmol) and FmocCl (1.2 g, 4.8 mmol) were reacted according to the reaction procedure described in step three of the final product 18 to obtain the target product (2.5 g, yield 99%).

### Step five: Synthesis of (2R,3R,4S,5S)-4-((((9H-fluoren-9-yl)methoxy)carbonyl)amino)methyl)-4-(4-chloro-2,5-difluorophenyl)-3-(3-chlorophenyl)-1-ethyl-5-neopentylpyrrolidine-2-carboxylic Acid (JQ112)

JQ102 (160 mg, 0.21 mmol), acetaldehyde (46 mg, 1.05 mmol), sodium cyanoborohydride (223 mg, 1.05 mmol), acetic acid (I mL) and trifluoroacetic acid (3 mL) were reacted according to the reaction procedure described in step four of the final product 18 to obtain the target product (115 mg, yield 76%).

### Step six: Synthesis of methyl 4-((2R,3R,4S,5S)-4-(aminomethyl)-4-(4-chloro-2,5-difluorophenyl)-3-(3-chlorophenyl)-1-ethyl-5-neopentylpyrrolidine-2-carboxamido)-3-methoxybenzoate (JQ118)

JQ112 (67 mg, 0.09 mmol), 1-methylimidazole (23 mg, 0.27 mmol), ethylsulfonyl chloride (24 mg, 0.18 mmol), methyl 4-amino-3-methoxybenzoate (51 mg, 0.27 mmol) and piperidine (1.5 mL) were reacted according to the reaction procedure described in step five of the final product 18 to obtain the target product (57 mg, yield 81%). ¹H NMR (400 MHz, Methanol-d₄) δ 8.24 (dd, J=8.9, 1.8 Hz, 1H), 7.63-7.55 (m, 2H), 7.49 (t, J=9.3 Hz, 1H), 7.38 (dd, J=12.7, 6.5 Hz, 1H), 7.35-7.26 (m, 2H), 7.18 (s, 1H), 7.08 (d, J=6.5 Hz, 1H), 4.41 (d, J=8.9 Hz, 1H), 4.31 (d, J=9.2 Hz, 1H), 3.99 (d, J=9.3 Hz, 1H), 3.93 (s, 3H), 3.87 (s, 3H), 3.54-3.38 (m, 2H), 3.30-3.22 (m, 1H), 3.09-2.94 (m, 1H), 2.04 (dd, J=13.3, 5.6 Hz, 1H), 1.47 (d, J=15.0 Hz, 1H), 1.24 (t, J=7.0 Hz, 3H), 1.07 (s, 9H). ESI-MS calculated for C₃₄H₄₀Cl₂F₂N₃O₄ [M+H]⁺=662.2, found: 662.2.

### Step seven: Synthesis of 4-((2′S,3S,4′R,5′R)-6-chloro-4′-(3-chlorophenyl)-1′-ethyl-5-fluoro-2′-neopentylspiro[indole-3,3′-pyrrolidine]-5′-carboxamido)-3-methoxybenzoic Acid (JQ121)

JQ118 (57 mg, 0.09 mmol), potassium carbonate (50 mg, 0.36 mmol) and lithium hydroxide monohydrate (17 mg, 0.4 mmol) were reacted according to the reaction procedure described in step six of the final product 10 to obtain the trifluoroacetate of the target product (45.4 mg, yield 68%). ¹H NMR (400 MHz, Methanol-d₄) δ 8.17 (d, J=8.3 Hz, 1H), 7.62 (d, J=8.5 Hz, 1H), 7.57 (s, 1H), 7.40 (d, J=9.0 Hz, 1H), 7.35 (s, 1H), 7.33-7.24 (m, 2H), 7.15 (d, J=7.0 Hz, 1H), 6.54 (d, J=5.9 Hz, 1H), 5.06-4.97 (m, 1H), 4.27-4.04 (m, 2H), 3.83 (s, 3H), 3.74-3.62 (m, 2H), 3.55 (d, J=11.0 Hz, 1H), 3.32-3.21 (m, 1H), 2.03 (dd, J=14.7, 6.9 Hz, 1H), 1.93-1.69 (m, 1H), 1.40 (t, J=7.1 Hz, 3H), 0.87 (s, 9H). ESI-MS calculated for C₃₃H₃₇Cl₂FN₃O₄ [M+H]⁺=628.2, found: 628.2.

### Final Product 93: 4-((2′S,3S,4′R,5′R)-6-chloro-4′-(3-chlorophenyl)-1′-ethyl-2′-(2,2,3-trimethylbutyl)spiro[indoline-3,3′-pyrrolidine]-5′-carboxamido)-3-methoxybenzoic Acid (TC145)

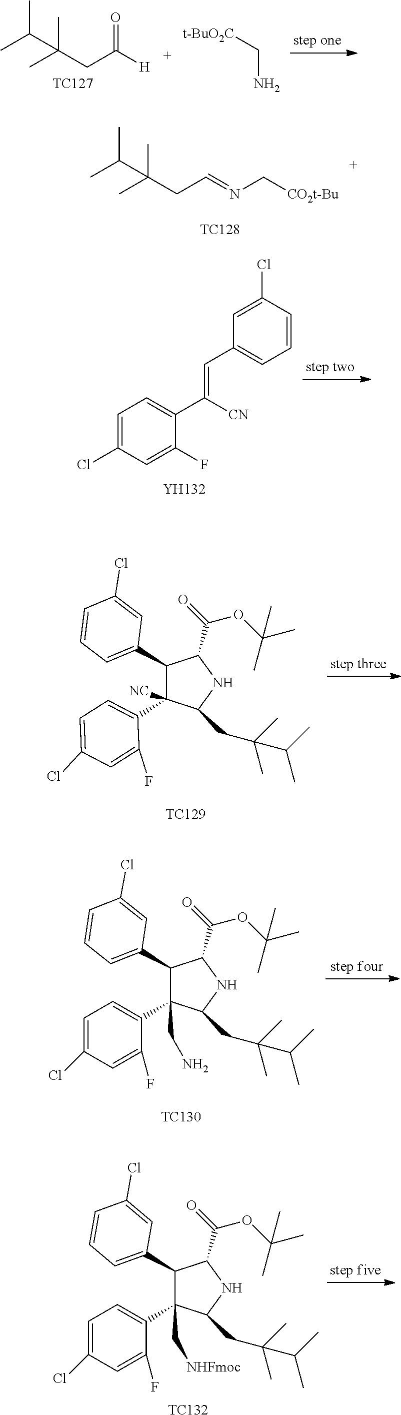

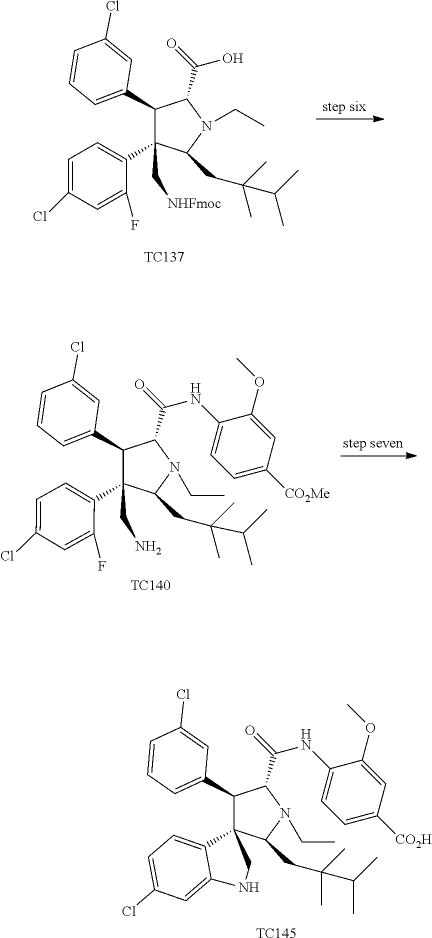

### Step one: tert-butyl (E)-2-((3,3,4-trimethylpentyl)amino)acetate (TC128)

TC127 (2.8, 22.1 mmol) and tert-butyl glycinate (3.2 g, 24.5 mmol) were reacted according to the reaction procedure refers to step one of the final product 33 to obtain the target product (5.2 g, yield 99%).

### Step two: Synthesis of tert-butyl (2R,3R,4R,5S)-4-(4-chloro-2-fluorophenyl)-3-(3-chlorophenyl)-4-cyano-5-(2,2,3-trimethylbutyl)pyrrolidine-2-carboxylate (TC129)

TC128 (5.2 g, 21.5 mmol), YH132 (5.4 g, 18.4 mmol), AgF (2.4 g, 18.4 mmol) and triethylamine (4.25 mL) were reacted according to the reaction procedure refers to step two of intermediate 1 to obtain the target product (3.1 g, yield 31%). ¹H NMR (500 MHz, Chloroform-d) δ 7.37 (t, J=8.5 Hz, 1H), 7.26-7.22 (m, 1H), 7.22-7.17 (m, 2H), 7.16 (t, J=1.9 Hz, 1H), 7.13 (dd, J=8.5, 1.7 Hz, 1H), 7.10 (dt, J=7.4, 1.6 Hz, 1H), 4.24 (d, J=7.6 Hz, 1H), 4.15 (d, J=7.6, Hz, 1H), 4.04 (d, J=8.9 Hz, 1H), 1.67-1.58 (m, 1H), 1.46 (h, J=6.7 Hz, 1H), 1.38 (s, 9H), 1.37-1.32 (m, 1H), 0.81 (d, J=6.4 Hz, 6H), 0.77 (s, 3H), 0.61 (d, J=6.8 Hz, 3H).

### Step Three: Synthesis of tert-butyl (2R,3R,4S,5S)-4-(aminomethyl)-4-(4-chloro-2-fluorophenyl)-3-(3-chlorophenyl)-5-(2,2,3-trimethylbutyl)pyrrolidine-2-carboxylate (TC130)

TC129 (3.1 g, 5.8 mmol), Raney nickel (3 g) and hydrazine hydrate (10 mL) were reacted according to the reaction procedure described in step three of intermediate 1 to obtain the target product (1.6 g, yield 43%). ¹H NMR (400 MHz, Methanol-d₄) δ 7.37-7.26 (m, 4H), 7.21 (t, J=8.7 Hz, 1H), 7.09 (s, 1H), 6.99 (d, J=5.4 Hz, 1H), 4.34 (d, J=7.7 Hz, 1H), 4.27 (t, J=5.2 Hz, 1H), 4.13 (d, J=7.8 Hz, 1H), 3.38 (d, J=13.9 Hz, 1H), 3.07 (d, J=13.8 Hz, 1H), 1.45-1.41 (m, 2H), 1.39 (s, 9H), 1.35-1.25 (m, 1H), 0.91-0.82 (m, 9H), 0.67 (d, J=6.7 Hz, 3H).

### Step Four: Synthesis of tert-butyl (2R,3R,4S,5S)-4-(((((9H-fluoren-9-yl)methoxy)carbonyl)amino)methyl)-4-(4-chloro-2-fluorophenyl)-3-(3-chlorophenyl)-5-(2,2,3-trimethylbutyl)pyrrolidine-2-carboxylate (TC132)

TC130 (1.4 g, 2.6 mmol), diisopropylethylamine (1.32 g, 10.2 mmol) and Fmoc-Cl (1.0 g, 3.8 mmol) were reacted according to the reaction procedure described in step three of final product 18 to obtain the target product (1.6 g, yield 82%).

### Step five. Synthesis of (2R,3R,4S,5S)-4-(((((9H-fluoren-9-yl)methoxy)carbonyl)amino)methyl)-4-(4-chloro-2-fluorophenyl)-3-(3-chlorophenyl)-1-ethyl-5-(2,2,3-trimethylbutyl)pyrrolidine-2-carboxylic Acid (TC137)

TC132 (200 mg, 0.26 mmol), acetaldehyde (117 mg, 2.64 mmol), sodium borohydride acetate (560 mg, 2.64 mmol), acetic acid (3 mL) and trifluoroacetic acid (4 mL) were reacted according to the reaction procedure described in step four of final product 18 to obtain the target product (170 mg, yield 85%).

### Step six: Synthesis of methyl 4-((2R,3R,4S,5S)-4-(aminomethyl)-4-(4-chloro-2-fluorophenyl)-3-(3-chlorophenyl)-1-ethyl-5-(2,2,3-trimethylbutyl)pyrrolidine-2-carboxamido)-3-methoxybenzoate (TC140)

TC137 (170 mg, 0.23 mmol), methyl 4-amino-3-methoxybenzoate (119 mg, 0.66 mmol), N-methylimidazole (54 mg, 0.66 mmol), ethylsulfonyl chloride (57 mg, 0.44 mmol) and piperidine (0.2 mL) were reacted according to the reaction procedure described in step five of final product 18 to obtain the target product (120 mg, yield 81%). ¹H NMR (500 MHz, Methanol-d₄) δ 8.27 (d, J=8.9 Hz, 1H), 7.66-7.47 (m, 3H), 7.39-7.24 (m, 4H), 7.19 (s, 1H), 7.12 (d, J=6.7 Hz, 1H), 4.33 (t, J=8.3 Hz, 2H), 4.08 (d, J=9.2 Hz, 1H), 3.95 (s, 3H), 3.88 (s, 3H), 3.51-3.35 (m, 2H), 3.35-3.32 (m, 1H), 3.09-2.91 (m, 1H), 1.95 (q, J=15.1, 8.9 Hz, 1H), 1.57-1.40 (m, 2H), 1.24 (t, J=7.0 Hz, 3H), 1.12 (s, 3H), 0.90 (d, J=6.8 Hz, 3H), 0.81 (d, J=6.8 Hz, 3H), 0.79 (s, 3H).

### Step seven: Synthesis of 4-((2′S,3S,4′R,5′R)-6-chloro-4′-(3-chlorophenyl)-2′-(2,2-dimethylbutyl)-1′-ethylspiro[indoline-3,3′-pyrrolidine]-5′-carboxyl)-3-methoxybenzoic Acid (TC145)

TC140 (103 mg, 0.15 mmol), potassium carbonate (85 mg, 0.62 mmol) and lithium hydroxide monohydrate (120 mg, 2.86 mmol) were reacted according to the reaction procedure described in step six of final product 10 to obtain the target product (23 mg, yield 41%). ¹H NMR (500 MHz, Methanol-d₄) δ 8.21 (d, J=8.4 Hz, 1H), 7.63 (dd, J=8.4, 1.8 Hz, 1H), 7.58 (s, 1H), 7.42-7.21 (m, 4H), 7.13 (d, J=7.5 Hz, 1H), 6.72 (dd, J=8.0, 1.9 Hz, 1H), 6.45 (d, J=1.9 Hz, 1H), 4.14 (s, 2H), 3.84 (s, 3H), 3.73-3.61 (m, 2H), 3.53 (s, 1H), 3.30 (s, 2H), 2.02-1.81 (m, 2H), 1.54-1.28 (m, 4H), 0.97 (s, 3H), 0.83 (d, J=6.7 Hz, 3H), 0.71 (d, J=6.8 Hz, 3H), 0.60 (s, 3H). ESI-MS calculated for C₃₅H₄₂³⁵Cl₂N₃O₄ [M+H]⁺=638.25, found: 638.2.

### Final Product 94: 4-((2′S,3S,4′R,5′R)-1′-allyl-6-chloro-4′-(3-chlorophenyl)-2′-neopentylspiro[indole-3,3′-pyrrolidine]-5′-carboxamido)-3-methoxybenzoic Acid (JR62)

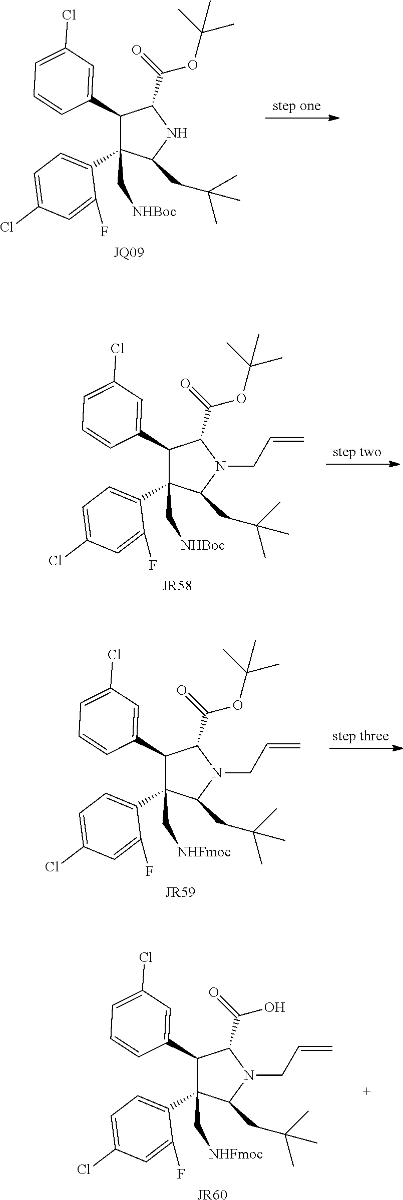

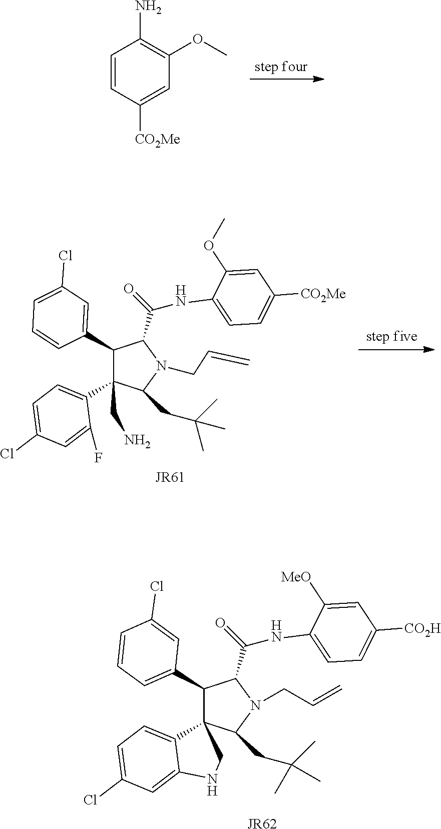

### Step one: Synthesis of tert-butyl (2R,3R,4S,5S)-1-allyl-4-(((tert-butoxycarbonyl)amino)methyl)-4-(4-chloro-2-fluorophenyl)-3-(3-chlorophenyl)-5-neopentylpyrrolidine-2-carboxylate (JR58)

JQ09 (400 mg, 0.66 mmol) was added into a 35 mL sealed tube, dissolved in 5 mL of anhydrous DMF, followed by addition of allyl bromide (799 mg, 6.6 mmol) and cesium carbonate (646 mg, 1.98 mmol), and the reaction was carried out at 90° C. overnight. After the reaction was complete, the mixture was cooled to room temperature, water was added, and the mixture was extracted with ethyl acetate, washed with saturated brine, dried over anhydrous sodium sulfate, the organic phase was concentrated by rotary evaporation and purified by normal phase column chromatography to obtain the target product (246 mg, yield 57%).

### Step two: Synthesis of tert-butyl (2R,3R,4S,5S)-4-((((9H-fluoren-9-yl)methoxy)carbonyl)amino)methyl)-1-allyl-4-(4-chloro-2-fluorophenyl)-3-(3-chlorophenyl)-5-neopentylpyrrolidine-2-carboxylate (JR59)

JR58 (280 mg, 0.43 mmol) was dissolved in 15 mL dichloromethane, and 1 mL trifluoroacetic acid was added and reacted at room temperature overnight. Saturated sodium bicarbonate solution was added to the reaction mixture, and the mixture was extracted three times with ethyl acetate. The combined organic phases were washed once with saturated sodium chloride solution and then concentrated by rotary evaporation. The reaction product was dissolved in 20 mL dichloromethane, and diisopropylethylamine (222 mg, 1.7 mmol) and FmocCl (167 mg, 0.65 mmol) were added. The reaction mixture was stirred at room temperature overnight. After concentration, the crude product was purified by normal phase column chromatography to obtain 110 mg of product, with a yield of 33%.

### Step Three: Synthesis of (2R,3R,4S,5S)-4-((((9H-fluoren-9-yl)methoxy)carbonyl)amino)methyl)-1-allyl-4-(4-chloro-2-fluorophenyl)-3-(3-chlorophenyl)-5-neopentylpyrrolidine-2-carboxylic Acid (JR60)

JR59 (110 mg, 0.14 mmol) was dissolved in 3 mL dichloromethane, and 3 mL trifluoroacetic acid was added and reacted at room temperature overnight. Saturated sodium bicarbonate solution was added to the reaction mixture, and the mixture was extracted three times with ethyl acetate. The combined organic phases were washed once with saturated sodium chloride solution, concentrated by rotary evaporation and purified by normal phase column chromatography to obtain 83 mg of the target product, with a yield of 83%. ¹H NMR (400 MHz, Chloroform-d) δ 7.78 (d, J=7.5 Hz, 2H), 7.59 (d, J=7.2 Hz, 2H), 7.41 (t, J=7.4 Hz, 2H), 7.37-7.29 (m, 2H), 7.23-7.08 (m, 4H), 7.05 (t, J=7.9 Hz, 1H), 6.97 (s, 1H), 6.76 (d, J=7.8 Hz, 1H), 6.03-5.79 (m, 1H), 5.46-5.30 (m, 2H), 4.57 (d, J=7.8 Hz, 1H), 4.50-4.29 (m, 4H), 4.27-4.17 (m, 1H), 3.84-3.68 (m, 2H), 3.52 (d, J=13.9 Hz, 1H), 3.27 (d, J=13.6 Hz, 1H), 1.60 (dd, J=15.9, 4.6 Hz, 1H), 1.46 (d, J=15.1 Hz, 1H), 0.68 (s, 9H).

### Step Four: Synthesis of methyl 4-((2R,3R,4S,5S)-1-allyl-4-(aminomethyl)-4-(4-chloro-2-fluorophenyl)-3-(3-chlorophenyl)-5-neopentylpyrrolidine-2-carboxamido)-3-methoxybenzoate (JR61)

JR60 (40 mg, 0.06 mmol), 1-methylimidazole (14 mg, 0.17 mmol), ethylsulfonyl chloride (16 mg, 0.12 mmol), methyl 4-amino-3-methoxybenzoate (31 mg, 0.17 mmol) and piperidine (1.5 mL) were reacted according to the procedure described in step five of the final product 18, to obtain 19 mg of the target product, with a yield of 41%. ¹H NMR (400 MHz, Methanol-d₄) δ 8.23 (d, J=8.9 Hz, 1H), 7.64-7.57 (m, 2H), 7.52 (t, J=8.8 Hz, 1H), 7.39-7.30 (m, 3H), 7.27 (dd, J=13.5, 2.2 Hz, 1H), 7.14-7.10 (m, 1H), 7.06 (dt, J=6.4, 2.0 Hz, 1H), 6.07-5.89 (m, 1H), 5.37 (d, J=17.1, 1H), 5.16 (d, J=10.0 Hz, 1H), 4.46 (d, J=8.6 Hz, 1H), 4.26 (d, J=9.4 Hz, 1H), 4.09 (dd, J=13.3, 5.7 Hz, 1H), 4.03 (d, J=9.4 Hz, 1H), 3.93 (s, 3H), 3.87 (s, 3H), 3.55 (dd, J=13.3, 7.7 Hz, 1H), 3.47 (d, J=14.5 Hz, 1H), 3.30-3.26 (m, 1H), 2.02 (dd, J=15.1, 8.9 Hz, 1H), 1.49 (d, J=15.0 Hz, 1H), 1.05 (s, 9H). ESI-MS calculated for C₃₅H₄₁³⁵Cl₂FN₃O₄ [M+H]⁺=656.2, found: 656.2.

### Step five: Synthesis of 4-((2′S,3S,4′R,5′R)-1′-allyl-6-chloro-4′-(3-chlorophenyl)-2′-neopentylspiro[indole-3,3′-pyrrolidine]-5′-carboxamido)-3-methoxybenzoic Acid (JR62)

JR61 (20 mg, 0.03 mmol), potassium carbonate (17 mg, 0.12 mmol) and lithium hydroxide monohydrate (7 mg, 0.15 mmol) were reacted according to the procedure described in step six of the final product 10 to obtain 7 mg of the target product as a trifluoroacetic acid salt, with a yield of 32%. ¹H NMR (400 MHz, Methanol-d₄) δ 8.28 (d, J=9.1 Hz, 1H), 7.69-7.59 (m, 2H), 7.28-7.00 (m, 5H), 6.70 (dd, J=7.9, 1.9 Hz, 1H), 6.43 (d, J=1.9 Hz, 1H), 6.04-5.89 (m, 1H), 5.55-5.43 (m, 1H), 5.34-5.19 (m, 1H), 4.38-4.23 (m, 1H), 4.21-3.67 (m, 7H), 3.55 (d, J=10.7 Hz, 1H), 3.40 (d, J=10.6 Hz, 1H), 2.03-1.90 (m, 1H), 1.54-1.30 (m, 1H), 0.98 (s, 9H). ESI-MS calculated for C₃₄H₃₈³⁵Cl₂N₃O₄ [M+H]⁺=622.2, found: 622.2.

### Final Product 95: 4-((2′S,3S,4′R,5′R)-6-chloro-4′-(3-chlorophenyl)-1-(3-fluoropropyl)-2′-neopentylspiro[indole-3,3′-pyrrolidine]-5′-carboxamido)-3-methoxybenzoic Acid (JR55)

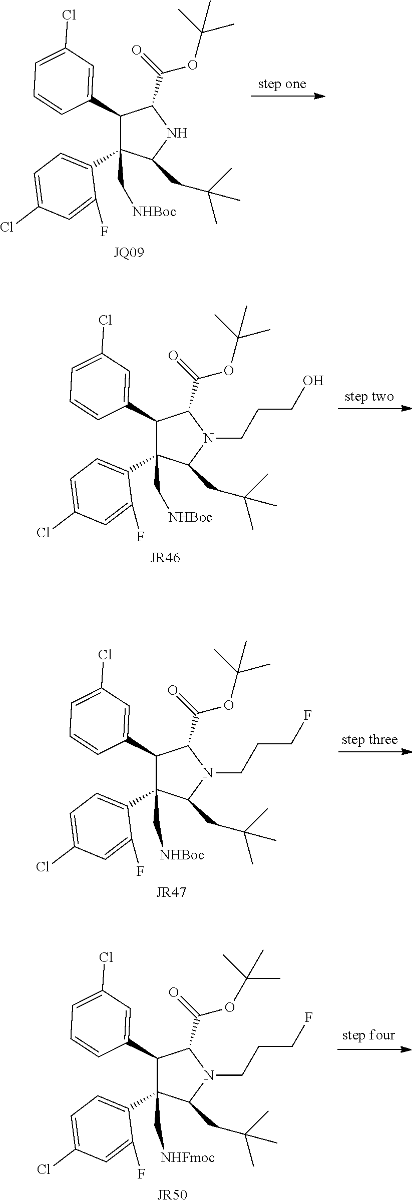

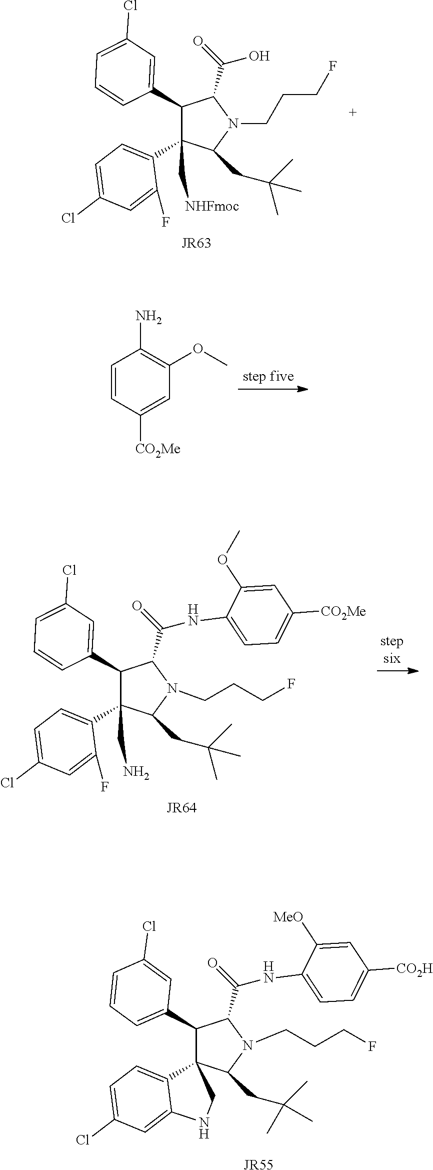

### Step one: Synthesis of tert-butyl (2R,3R,4S,5S)-4-((tert-butoxycarbonyl)amino)methyl)-4-(4-chloro-2-fluorophenyl)-3-(3-chlorophenyl)-1-(3-hydroxypropyl)-5-neopentylpyrrolidine-2-carboxylate (JR46)

JQ09 (300 mg, 0.49 mmol), 3-[(tert-butyldimethylsilyl)oxy]-1-propanal (278 mg, 1.48 mmol), sodium borohydride acetate (314 mg, 1.48 mmol), acetic acid (1 mL) and 1N tetrabutylammonium fluoride solution in tetrahydrofuran (0.9 mL, 0.88 mmol). The reaction procedure follows step two of the final product 49 to obtain the target product (207 mg, yield 63%).

### Step two: Synthesis of tert-butyl (2R,3R,4S,5S)-4-((tert-butoxycarbonyl)amino)methyl)-4-(4-chloro-2-fluorophenyl)-3-(3-chlorophenyl)-1-(3-fluoropropyl)-5-neopentylpyrrolidine-2-carboxylate (JR47)

JR46 (200 mg, 0.35 mmol) was placed in a 50 mL single-neck flask, dissolved in dry dichloromethane, and diethylaminosulfur trifluoride (114 mg, 0.71 mmol) was added at 0° C., then reacted overnight at room temperature. After the reaction was complete, the mixture was extracted with dichloromethane, washed twice with saturated sodium chloride solution, the organic phase was dried over anhydrous sodium sulfate, concentrated by rotary evaporation and purified by column chromatography to obtain the target product (158 mg, yield 67%).

### Step Three: Synthesis of tert-butyl (2R,3R,4S,5S)-4-((9H-fluoren-9-yl)methoxy)carbonyl)amino)methyl)-4-(4-chloro-2-fluorophenyl)-3-(3-chlorophenyl)-1-(3-fluoropropyl)-5-neopentylpyrrolidine-2-carboxylate (JR50)

JR46 (158 mg, 0.27 mmol), trifluoroacetic acid (1 mL), diisopropylethylamine (139 mg, 1.1 mmol) and FmocCl (105 mg, 0.4 mmol) were reacted according to the reaction procedure described in step two of the final product 94 to obtain 60 mg of the target product, with a yield of 28%.

### Step Four: Synthesis of ((2R,3R,4S,5S)-4-((9H-fluoren-9-yl)methoxy)carbonyl)amino)methyl)-4-(4-chloro-2-fluorophenyl)-3-(3-chlorophenyl)-1-(3-fluoropropyl)-5-neopentylpyrrolidine-2-carboxylic Acid (JR63)

JR50 (60 mg, 0.08 mmol) and trifluoroacetic acid (3 mL) were reacted according to the reaction procedure described in step three of the final product 94 to obtain the target product (34 mg, yield 61%).

### Step five: Synthesis of methyl 4-((2R,3R,4S,5S)-4-(aminomethyl)-4-(4-chloro-2-fluorophenyl)-3-(3-chlorophenyl)-1-(3-fluoropropyl)-5-neopentylpyrrolidine-2-carboxamido)-3-methoxybenzoate (JR64)

JR63 (47 mg, 0.06 mmol), 1-methylimidazole (15 mg, 0.18 mmol), ethylsulfonyl chloride (17 mg, 0.13 mmol), methyl 4-amino-3-methoxybenzoate (33 mg, 0.18 mmol) and piperidine (2 mL) were reacted according to the reaction procedure described in step five of the final product 18 to obtain the target product (29 mg, yield 62%). ¹H NMR (400 MHz, Methanol-d₄) δ 8.21 (d, J=8.8 Hz, 1H), 7.66-7.56 (m, 2H), 7.48 (t, J=8.8 Hz, 1H), 7.39-7.23 (m, 4H), 7.16-7.11 (m, 1H), 7.05 (dt, J=6.8, 1.8 Hz, 1H), 4.70-4.58 (m, 1H), 4.57-4.46 (m, 2H), 4.43 (d, J=8.9 Hz, 1H), 4.12 (d, J=8.9 Hz, 1H), 3.93 (s, 3H), 3.88 (s, 3H), 3.58-3.44 (m, 2H), 3.24 (d, J=14.8 Hz, 1H), 3.15-3.04 (m, 1H), 2.13-1.88 (m, 3H), 1.50 (dd, J=15.3, 2.0 Hz, 1H), 1.00 (s, 9H). ESI-MS calculated for C₃₅H₄₂³⁵Cl₂F₂N₃O₄ [M+H]⁺=676.3, found: 676.2.

### Step six: Synthesis of 4-((2′S,3S,4′R,5′R)-6-chloro-4′-(3-chlorophenyl)-1-(3-fluoropropyl)-2′-neopentylspiro[indole-3,3′-pyrrolidine]-5′-carboxamido)-3-methoxybenzoic Acid (JR55)

JR64 (29 mg, 0.04 mmol), potassium carbonate (23 mg, 0.16 mmol) and lithium hydroxide monohydrate (9 mg, 0.2 mmol) were reacted according to the reaction procedure described in step six of the final product 10 to obtain the target product as trifluoroacetic acid salt (17.6 mg, yield 59%). ¹H NMR (500 MHz, Methanol-d₄) δ 8.29 (d, J=8.4 Hz, 1H), 7.70-7.59 (m, 2H), 7.35-7.20 (m, 4H), 7.07 (d, J=7.4 Hz, 1H), 6.75 (dd, J=8.0, 1.9 Hz, 1H), 6.48 (d, J=1.9 Hz, 1H), 4.72-4.42 (m, 3H), 4.07-3.82 (m, 5H), 3.69-3.61 (m, 1H), 3.59 (d, J=10.8 Hz, 1H), 3.45 (d, J=10.6 Hz, 1H), 3.05-2.85 (m, 1H), 2.23-1.94 (m, 3H), 1.55-1.40 (m, 1H), 0.96 (s, 9H). ESI-MS calculated for C₃₄H₃₉³⁵Cl₂FN₃O₄ [M+H]⁺=642.2, found: 642.2.

By using the corresponding starting materials, the following compounds in Table 1 were synthesized:

TABLE 1

| No. | Code | Structure | Name |
|---|---|---|---|
| 1 | YM157 | 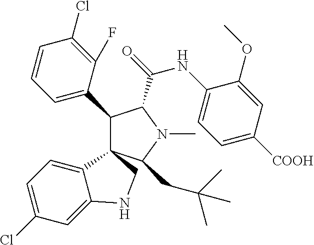 [SMILES: COc1cc(C(=O)O)ccc1NC(=O)[C@H]1[C@H](c2cccc(Cl)c2F)[C@]2(CNc3cc(Cl)ccc32)[C@H](CC(C)(C)C)N1C] | 4-((2′S,3S,4′S,5′R)-6-chloro-4′-(3-chloro- 2-fluorophenyl)-1′-methyl-2′- neopentylspiro[indoline-3,3′-pyrrolidine]- 5′-carboxamido)-3-methoxybenzoic acid |
| 2 | YN11 | 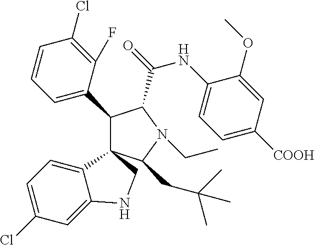 [SMILES: CCN1[C@@H](CC(C)(C)C)[C@@]2(CNc3cc(Cl)ccc32)[C@@H](c2cccc(Cl)c2F)[C@@H]1C(=O)Nc1ccc(C(=O)O)cc1OC] | 4-((2′S,3S,4′S,5′R)-6-chloro-4′-(3-chloro- 2-fluorophenyl)-1′-ethyl-2′- neopentylspiro[indoline-3,3′-pyrrolidine]- 5′-carboxamido)-3-methoxybenzoic acid |
| 3 | YN51 | 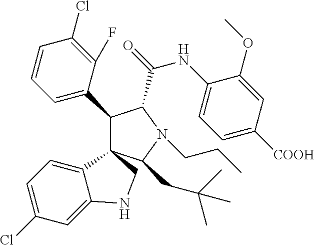 [SMILES: CCCN1[C@@H](CC(C)(C)C)[C@@]2(CNc3cc(Cl)ccc32)[C@@H](c2cccc(Cl)c2F)[C@@H]1C(=O)Nc1ccc(C(=O)O)cc1OC] | 4-((2′S,3S,4′S,5′R)-6-chloro-4′-(3-chloro- 2-fluorophenyl)-1′-propyl-2′- neopentylspiro[indoline-3,3′-pyrrolidine]- 5′-carboxamido)-3-methoxybenzoic acid |
| 4 | YN52 | 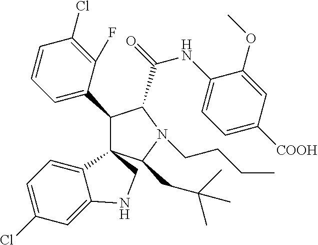 [SMILES: CCCCN1[C@@H](CC(C)(C)C)[C@@]2(CNc3cc(Cl)ccc32)[C@@H](c2cccc(Cl)c2F)[C@@H]1C(=O)Nc1ccc(C(=O)O)cc1OC] | 4-((2′S,3S,4′S,5′R)-6-chloro-4′-(3-chloro- 2-fluorophenyl)-1′-butyl-2′- neopentylspiro[indoline-3,3′-pyrrolidine]- 5′-carboxamido)-3-methoxybenzoic acid |
| 5 | JN05-2 | 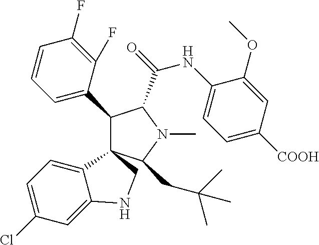 [SMILES: COc1cc(C(=O)O)ccc1NC(=O)[C@H]1[C@H](c2cccc(F)c2F)[C@]2(CNc3cc(Cl)ccc32)[C@H](CC(C)(C)C)N1C] | 4-((2′S,3S,4′S,5′R)-6-chloro-4′-(2,3- difluorophenyl)-1′-methyl-2′- neopentylspiro[indoline-3,3′-pyrrolidine]- 5′-carboxamido)-3-methoxybenzoic acid |
| 6 | JM151 | 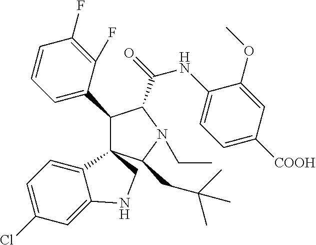 [SMILES: CCN1[C@@H](CC(C)(C)C)[C@@]2(CNc3cc(Cl)ccc32)[C@@H](c2cccc(F)c2F)[C@@H]1C(=O)Nc1ccc(C(=O)O)cc1OC] | 4-((2′S,3S,4′S,5′R)-6-chloro-4′-(2,3- difluorophenyl)-1′-ethyl-2′- neopentylspiro[indoline-3,3′-pyrrolidine]- 5′-carboxamido)-3-methoxybenzoic acid |
| 7 | JM156 | 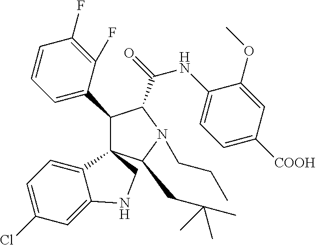 [SMILES: CCCN1[C@@H](CC(C)(C)C)[C@@]2(CNc3cc(Cl)ccc32)[C@@H](c2cccc(F)c2F)[C@@H]1C(=O)Nc1ccc(C(=O)O)cc1OC] | 4-((2′S,3S,4′S,5′R)-6-chloro-4′-(2,3- difluorophenyl)-1′-propyl-2′- neopentylspiro[indoline-3,3′-pyrrolidine]- 5′-carboxamido)-3-methoxybenzoic acid |
| 8 | JN06 | 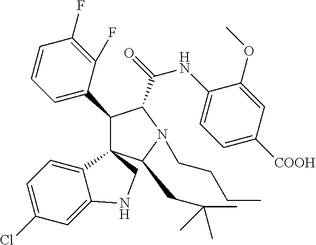 [SMILES: CCCCN1[C@@H](CC(C)(C)C)[C@@]2(CNc3cc(Cl)ccc32)[C@@H](c2cccc(F)c2F)[C@@H]1C(=O)Nc1ccc(C(=O)O)cc1OC] | 4-((2′S,3S,4′S,5′R)-6-chloro-4′-(2,3- difluorophenyl)-1′-butyl-2′- neopentylspiro[indoline-3,3′-pyrrolidine]- 5′-carboxamido)-3-methoxybenzoic acid |
| 9 | JN22 | 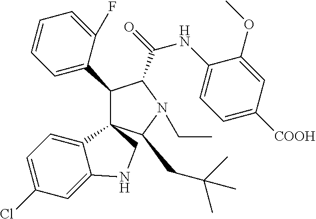 [SMILES: CCN1[C@@H](CC(C)(C)C)[C@@]2(CNc3cc(Cl)ccc32)[C@@H](c2ccccc2F)[C@@H]1C(=O)Nc1ccc(C(=O)O)cc1OC] | 4-((2′S,3S,4′S,5′R)-6-chloro-4′-(2- fluorophenyl)-1′-ethyl-2′- neopentylspiro[indoline-3,3′-pyrrolidine]- 5′-carboxamido)-3-methoxybenzoic acid |
| 10 | JM159 | 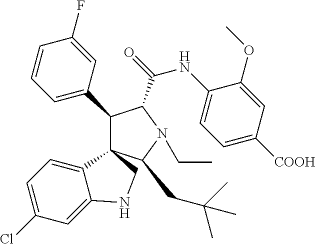 [SMILES: CCN1[C@@H](CC(C)(C)C)[C@@]2(CNc3cc(Cl)ccc32)[C@@H](c2cccc(F)c2)[C@@H]1C(=O)Nc1ccc(C(=O)O)cc1OC] | 4-((2′S,3S,4′S,5′R)-6-chloro-4′-(3- fluorophenyl)-1′-ethyl-2′- neopentylspiro[indoline-3,3′-pyrrolidine]- 5′-carboxamido)-3-methoxybenzoic acid |
| 11 | JN17 | 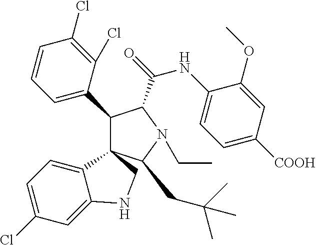 [SMILES: CCN1[C@@H](CC(C)(C)C)[C@@]2(CNc3cc(Cl)ccc32)[C@@H](c2cccc(Cl)c2Cl)[C@@H]1C(=O)Nc1ccc(C(=O)O)cc1OC] | 4-((2′S,3S,4′S,5′R)-6-chloro-4′-(2,3- dichlorophenyl)-1′-ethyl-2′- neopentylspiro[indoline-3,3′-pyrrolidine]- 5′-carboxamido)-3-methoxybenzoic acid |
| 12 | JN01 | 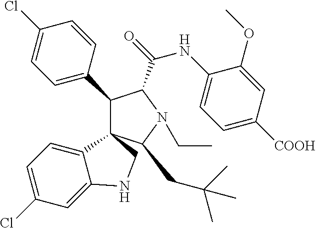 [SMILES: CCN1[C@@H](CC(C)(C)C)[C@@]2(CNc3cc(Cl)ccc32)[C@@H](c2ccc(Cl)cc2)[C@@H]1C(=O)Nc1ccc(C(=O)O)cc1OC] | 4-((2′,3S,4′S,5′R)-6-chloro-4′-(4- chlorophenyl)-1′-ethyl-2′- neopentylspiro[indoline-3,3′-pyrrolidine]- 5′-carboxamido)-3-methoxybenzoic acid |
| 13 | JN18 | 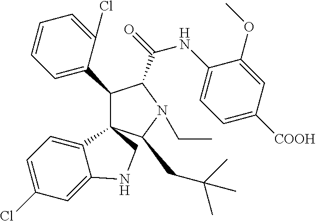 [SMILES: CCN1[C@@H](CC(C)(C)C)[C@@]2(CNc3cc(Cl)ccc32)[C@@H](c2ccccc2Cl)[C@@H]1C(=O)Nc1ccc(C(=O)O)cc1OC] | 4-((2′,3S,4′S,5′R)-6-chloro-4′-(2- chlorophenyl)-1′-ethyl-2′- neopentylspiro[indoline-3,3′-pyrrolidine]- 5′-carboxamido)-3-methoxybenzoic acid |
| 14 | YN55 | 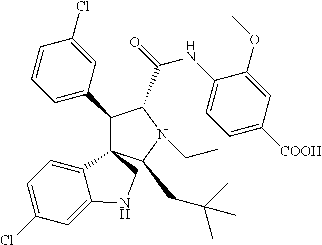 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′S,5′R)-6-chloro-4′-(3- chlorophenyl)-1′-ethyl-2′- neopentylspiro[indoline-3,3′-pyrrolidine]- 5′-carboxamido)-3-methoxybenzoic acid |
| 15 | JM157 | 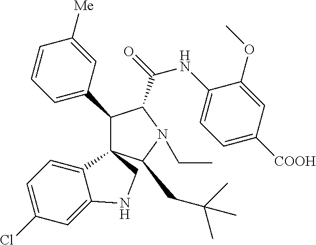 [SMILES: CCN1[C@@H](CC(C)(C)C)[C@@]2(CNc3cc(Cl)ccc32)[C@@H](c2cccc(C)c2)[C@@H]1C(=O)Nc1ccc(C(=O)O)cc1OC] | 4-((2′S,3S,4′S,5′R)-6-chloro-4′-(3- methylphenyl)-1′-ethyl-2′- neopentylspiro[indoline-3,3′-pyrrolidine]- 5′-carboxamido)-3-methoxybenzoic acid |
| 16 | JM160 | 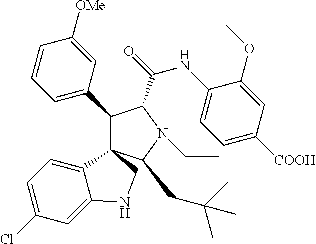 [SMILES: CCN1[C@@H](CC(C)(C)C)[C@@]2(CNc3cc(Cl)ccc32)[C@@H](c2cccc(OC)c2)[C@@H]1C(=O)Nc1ccc(C(=O)O)cc1OC] | 4-((2′,3S,4′S,5′R)-6-chloro-4′-(3- methoxyphenyl)-1′-ethyl-2′- neopentylspiro[indoline-3,3′-pyrrolidine]- 5′-carboxamido)-3-methoxybenzoic acid |
| 17 | JN19 | 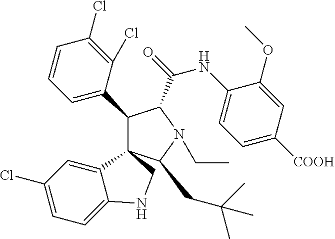 [SMILES: CCN1[C@@H](CC(C)(C)C)[C@@]2(CNc3ccc(Cl)cc32)[C@@H](c2cccc(Cl)c2Cl)[C@@H]1C(=O)Nc1ccc(C(=O)O)cc1OC] | 4-((2′S,3S,4′S,5′R)-5-chloro-4′-(2,3- dichlorophenyl)-1′-ethyl-2′- neopentylspiro[indoline-3,3′-pyrrolidine]- 5′-carboxamido)-3-methoxybenzoic acid |
| 18 | JN122 | 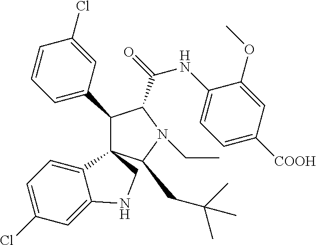 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′S,5′R)-6-chloro-4′-(3- chlorophenyl)-1′-ethyl-2′- neopentylspiro[indoline-3,3′-pyrrolidine]- 5′-carboxamido)-3-methoxybenzoic acid |
| 19 | | 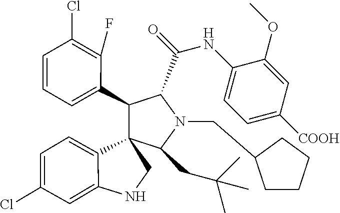 [SMILES: COc1cc(C(=O)O)ccc1NC(=O)[C@H]1[C@H](c2cccc(Cl)c2F)[C@]2(CNc3cc(Cl)ccc32)[C@H](CC(C)(C)C)N1CC1CCCC1] | 4-((2′S,3S,4′S,5′R)-6-chloro-4′-(3-chloro- 2-fluorophenyl)-1′-(cyclopentylmethyl)- 2′-neopentylspiro[indoline-3,3′- pyrrolidine]-5′-carboxamido)-3- methoxybenzoic acid |
| 20 | | 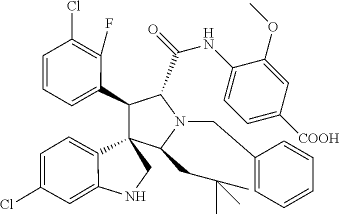 [SMILES: COc1cc(C(=O)O)ccc1NC(=O)[C@H]1[C@H](c2cccc(Cl)c2F)[C@]2(CNc3cc(Cl)ccc32)[C@H](CC(C)(C)C)N1Cc1ccccc1] | 4-((2′,3S,4′S,5′R)-1′-benzyl-6-chloro-4′- (3-chloro-2-fluorophenyl)-2′- neopentylspiro[indoline-3,3′-pyrrolidine]- 5′-carboxamido)-3-methoxybenzoic acid |
| 21 | | 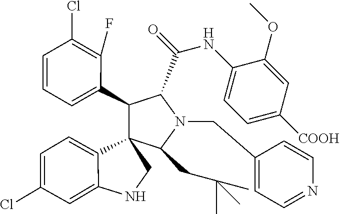 [SMILES: COc1cc(C(=O)O)ccc1NC(=O)[C@H]1[C@H](c2cccc(Cl)c2F)[C@]2(CNc3cc(Cl)ccc32)[C@H](CC(C)(C)C)N1Cc1ccncc1] | 4-((2′,3S,4′S,5′R)-6-chloro-4′-(3-chloro- 2-fluorophenyl)-2′-neopentyl-1′-(pyridin- 4-ylmethyl)spiro[indoline-3,3′- pyrrolidine]-5′-carboxamido)-3- methoxybenzoic acid |
| 22 | | 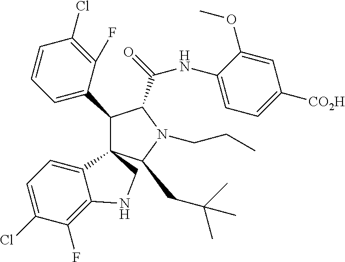 [SMILES: CCCN1[C@@H](CC(C)(C)C)[C@@]2(CNc3c2ccc(Cl)c3F)[C@@H](c2cccc(Cl)c2F)[C@@H]1C(=O)Nc1ccc(C(=O)O)cc1OC] | 4-((2′,3S,4′S,5′R)-6-chloro-4′-(3-chloro- 2-fluorophenyl)-1′-propyl-7-fluoro-2′- neopentylspiro[indoline-3,3′-pyrrolidine]- 5′-carboxamido)-3-methoxybenzoic acid |
| 23 | | 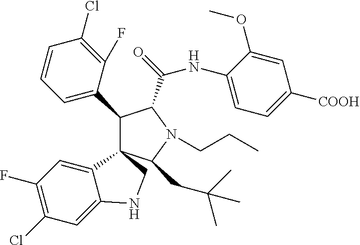 [SMILES: CCCN1[C@@H](CC(C)(C)C)[C@@]2(CNc3cc(Cl)c(F)cc32)[C@@H](c2cccc(Cl)c2F)[C@@H]1C(=O)Nc1ccc(C(=O)O)cc1OC] | 4-((2′S,3S,4′S,5′R)-6-chloro-4′-(3-chloro- 2-fluorophenyl)-1′-propyl-5-fluoro-2′- neopentylspiro[indoline-3,3′-pyrrolidine]- 5′-carboxamido)-3-methoxybenzoic acid |
| 24 | | 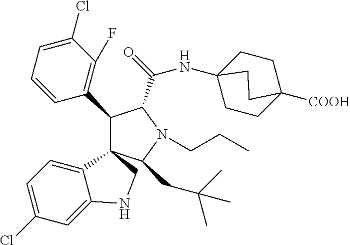 [SMILES: CCCN1[C@@H](CC(C)(C)C)[C@@]2(CNc3cc(Cl)ccc32)[C@@H](c2cccc(Cl)c2F)[C@@H]1C(=O)NC12CCC(C(=O)O)(CC1)CC2] | 4-((2′,3S,4′S,5′R)-6-chloro-4′-(3-chloro- 2-fluorophenyl)-1′-propyl-2′- neopentylspiro[indoline-3,3′-pyrrolidine]- 5′-carboxamido)bicyclo[2.2.2]octane-1- carboxylic acid |
| 25 | | 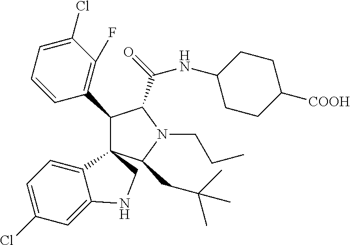 [SMILES: CCCN1[C@@H](CC(C)(C)C)[C@@]2(CNc3cc(Cl)ccc32)[C@@H](c2cccc(Cl)c2F)[C@@H]1C(=O)NC1CCC(C(=O)O)CC1] | 4-((2′S,3S,4′S,5′R)-6-chloro-4′-(3-chloro- 2-fluorophenyl)-1′-propyl-2′- neopentylspiro[indoline-3,3′-pyrrolidine]- 5′-carboxamido)cyclohexane-1- carboxylic acid |
| 26 | | 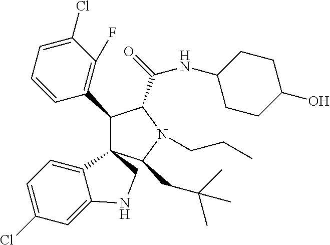 [SMILES: CCCN1[C@@H](CC(C)(C)C)[C@@]2(CNc3cc(Cl)ccc32)[C@@H](c2cccc(Cl)c2F)[C@@H]1C(=O)NC1CCC(O)CC1] | (2′S,3S,4′S,5′R)-6-chloro-4′-(3-chloro-2- fluorophenyl)-1′-propyl-N-(4- hydroxycyclohexyl)-2′- neopentylspiro[indoline-3,3′-pyrrolidine]- 5′-carboxamide |
| 27 | | 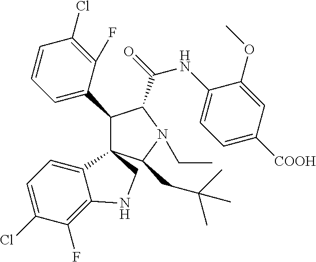 [SMILES: CCN1[C@@H](CC(C)(C)C)[C@@]2(CNc3c2ccc(Cl)c3F)[C@@H](c2cccc(Cl)c2F)[C@@H]1C(=O)Nc1ccc(C(=O)O)cc1OC] | 4-((2′,3S,4′S,5′R)-6-chloro-4′-(3-chloro- 2-fluorophenyl)-1′-ethyl-7-fluoro-2′- neopentylspiro[indoline-3,3′-pyrrolidine]- 5′-carboxamido)-3-methoxybenzoic acid |
| 28 | | 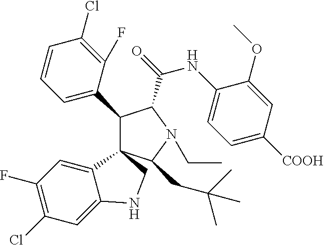 [SMILES: CCN1[C@@H](CC(C)(C)C)[C@@]2(CNc3cc(Cl)c(F)cc32)[C@@H](c2cccc(Cl)c2F)[C@@H]1C(=O)Nc1ccc(C(=O)O)cc1OC] | 4-((2′,3S,4′S,5′R)-6-chloro-4′-(3-chloro- 2-fluorophenyl)-1′-ethyl-5-fluoro-2′- neopentylspiro[indoline-3,3′-pyrrolidine]- 5′-carboxamido)-3-methoxybenzoic acid |
| 29 | | 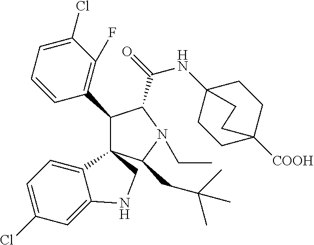 [SMILES: CCN1[C@@H](CC(C)(C)C)[C@@]2(CNc3cc(Cl)ccc32)[C@@H](c2cccc(Cl)c2F)[C@@H]1C(=O)NC12CCC(C(=O)O)(CC1)CC2] | 4-((2′S,3S,4′S,5′R)-6-chloro-4′-(3-chloro- 2-fluorophenyl)-1′-ethyl-2′- neopentylspiro[indoline-3,3′-pyrrolidine]- 5′-carboxamido)bicyclo[2.2.2]octane-1- carboxylic acid |
| 30 | | 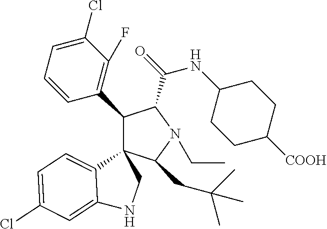 [SMILES: CCN1[C@@H](CC(C)(C)C)[C@@]2(CNc3cc(Cl)ccc32)[C@@H](c2cccc(Cl)c2F)[C@@H]1C(=O)NC1CCC(C(=O)O)CC1] | 4-((2′S,3S,4′S,5′R)-6-chloro-4′-(3-chloro- 2-fluorophenyl)-1′-ethyl-2′- neopentylspiro[indoline-3,3′-pyrrolidine]- 5′-carboxamido)cyclohexane-1- carboxylic acid |
| 31 | | 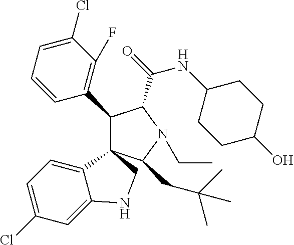 [SMILES: CCN1[C@@H](CC(C)(C)C)[C@@]2(CNc3cc(Cl)ccc32)[C@@H](c2cccc(Cl)c2F)[C@@H]1C(=O)NC1CCC(O)CC1] | (2′S,3S,4′S,5′R)-6-chloro-4′-(3-chloro-2- fluorophenyl)-1′-ethyl-N-(4- hydroxycyclohexl)-2′- neopentylspiro[indoline-3,3′-pyrrolidine]- 5′-carboxamide |
| 32 | | 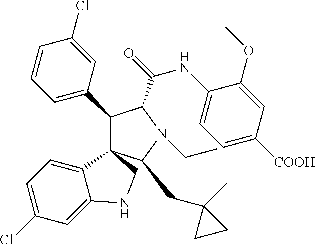 [SMILES: CCN1[C@@H](CC2(C)CC2)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-ethyl-2′-((1- methylcyclopropyl)methyl)spiro [indoline-3,3′-pyrrolidine]-5′- methoxybenzoic acid |
| 33 | LCC153 | 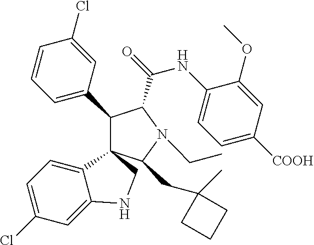 [SMILES: CCN1[C@@H](CC2(C)CCC2)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-ethyl-2′-((1- methylcyclobutyl)methyl)spiro [indoline-3,3′-pyrrolidine]-5′- methoxybenzoic acid |
| 34 | LCC63 | 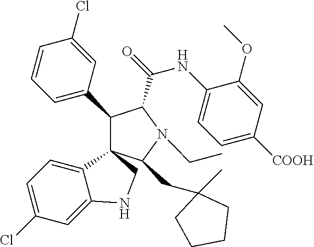 [SMILES: CCN1[C@@H](CC2(C)CCCC2)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-ethyl-2′-((1- methylcyclopentyl)methyl)spiro [indoline-3,3′-pyrrolidine]-5′- methoxybenzoic acid |
| 35 | LCC131 | 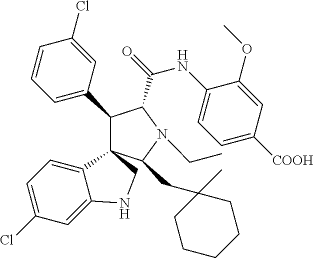 [SMILES: CCN1[C@@H](CC2(C)CCCCC2)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-ethyl-2′-((1- methylcyclohexyl)methyl)spiro [indoline-3,3′-pyrrolidine]-5′- methoxybenzoic acid |
| 36 | | 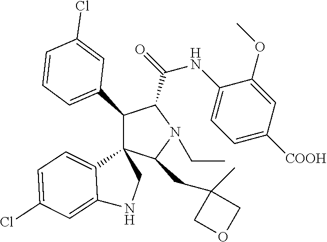 [SMILES: CCN1[C@@H](CC2(C)COC2)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-ethyl-2′-((3- methyloxetan-3-yl)methyl)spiro [indoline-3,3′-pyrrolidine]-5′- methoxybenzoic acid |
| 37 | TC90 | 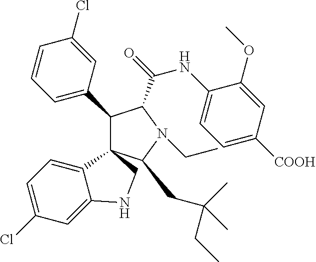 [SMILES: CCN1[C@@H](CC(C)(C)CC)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-2′-(2,2-dimethylbutyl)-1′- ethylspiro[indoline-3,3′-pyrrolidine]-5′- methoxybenzoic acid |
| 38 | TC29 | 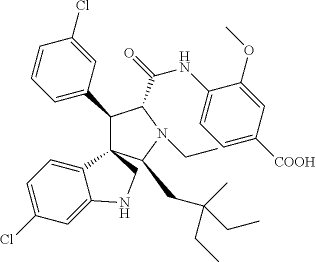 [SMILES: CCN1[C@@H](CC(C)(CC)CC)[C@@]2(CNc3cc(Cl)ccc32)[C@@H](c2cccc(Cl)c2)[C@@H]1C(=O)Nc1ccc(C(=O)O)cc1OC] | 4-((2′,3S,4′R,5′R)-6-chloro-4′-(3- chlorophenyl)-1′-ethyl-2′-(2-ethyl-3- methylbutyl)spiro[indoline-3,3′- pyrrolidine]-5′-methoxybenzoic acid |
| 39 | | 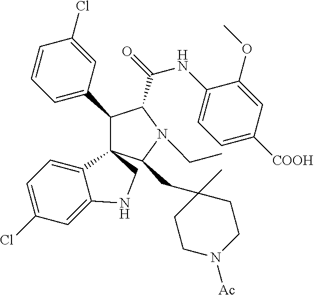 [SMILES: CCN1[C@@H](CC2(C)CCN(C(C)=O)CC2)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-2′-((1-acetyl-4- methylpiperid-4-yl)methyl)-6-chloro-4′- (3-chlorophenyl)-1′-ethylspiro[indoline- 3,3′-pyrrolidine]-5′-methoxybenzoic acid |
| 40 | | 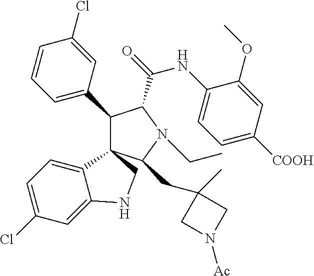 [SMILES: CCN1[C@@H](CC2(C)CN(C(C)=O)C2)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-2′((1-acetyl-3- methylazetidin-3-yl)methyl)-6-chloro-4′- (3-chlorophenyl)-1′-ethylspiro[indoline- 3,3′-pyrrolidine]-5′-methoxybenzoic acid |
| 41 | | 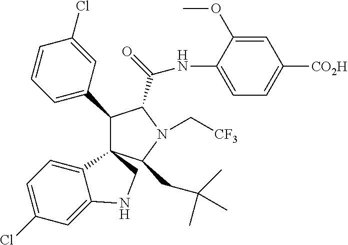 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC(F)(F)F] | 4-((2′S,3S,4′R,5′R)-6-chloro-4′-(3- chlorophenyl)-2′-neopentyl-1′-(2,2,2- trifluoroethyl)spiro[indoline-3,3′- pyrrolidine]-5′-methoxybenzoic acid |
| 42 | | 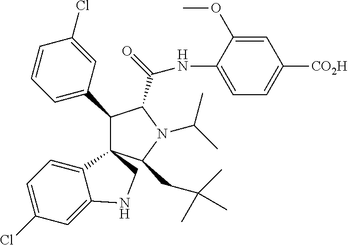 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1C(C)C] | 4-((2′S,3S,4′R,5′R)-6-chloro-4′-(3- chlorophenyl)-1′-isopropyl-2′- neopentylspiro[indoline-3,3′-pyrrolidine]- 5′-carboxamido)-3-methoxybenzoic acid |
| 43 | JP16 | 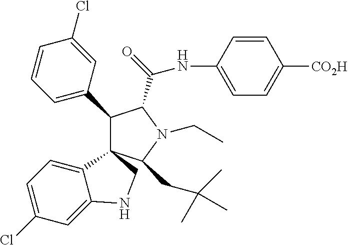 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1] | 4-((2′S,3S,4′R,5′R)-6-chloro-4′-(3- chlorophenyl)-1′-ethyl-2′- neopentylspiro[indoline-3,3′-pyrrolidine]- 5′-carboxamido)benzoic acid |
| 44 | | 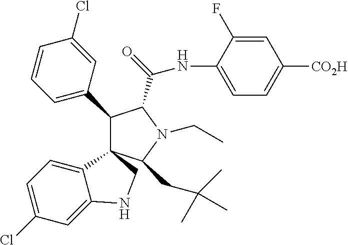 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1F] | 4-((2′S,3S,4′R,5′R)-6-chloro-4′-(3- chlorophenyl)-1′-ethyl-2′- neopentylspiro[indoline-3,3′-pyrrolidine]- 5′-carboxamido)-3-fluorobenzoic acid |
| 45 | | 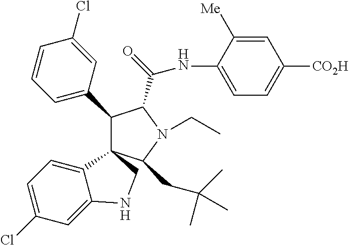 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1C] | 4-((2′S,3S,4′R,5′R)-6-chloro-4′-(3- chlorophenyl)-1′-ethyl-2′- neopentylspiro[indoline-3,3′-pyrrolidine]- 5′-carboxamido)-3-methylbenzoic acid |
| 46 | | 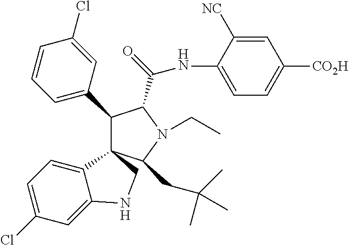 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1C#N] | 4-((2′S,3S,4′R,5′R)-6-chloro-4′-(3- chlorophenyl)-1′-ethyl-2′- neopentylspiro[indoline-3,3′-pyrrolidine]- 5′-carboxamido)-3-cyanobenzoic acid |
| 47 | | 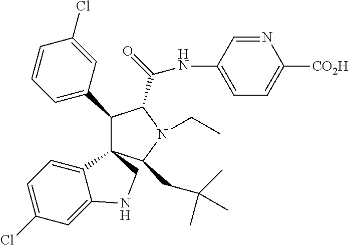 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)nc1] | 5-((2′S,3S,4′R,5′R)-6-chloro-4′-(3- chlorophenyl)-1′-ethyl-2′- neopentylspiro[indoline-3,3′-pyrrolidine]- 5′-carboxamido]pyridine acid |
| 48 | | 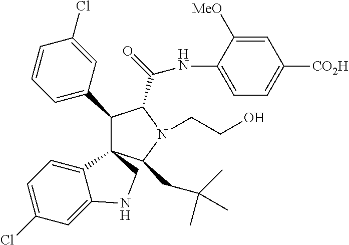 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCO] | 4-((2′S,3S,4′R,5′R)-6-chloro-4′-(3- chlorophenyl)-1′-(2-hydroxyethyl)-2′- neopentylspiro[indoline-3,3′-pyrrolidine]- 5′-methoxybenzoic acid |
| 49 | JQ44 | 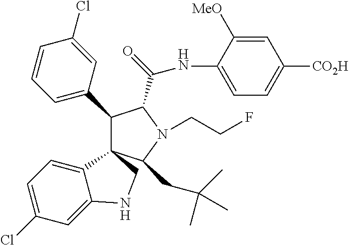 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCF] | 4-((2′S,3S,4′R,5′R)-6-chloro-4′-(3- chlorophenyl)-1′-(2-fluoroethyl)-2′- neopentylspiro[indoline-3,3′-pyrrolidine]- 5′-methoxybenzoic acid |
| 50 | | 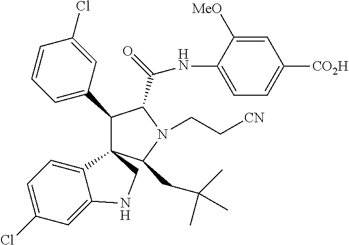 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCC#N] | 4-((2′S,3S,4′R,5′R)-6-chloro-4′-(3- chlorophenyl)-1′-(2-cyanophenyl)-2′- neopentylspiro[indoline-3,3′-pyrrolidine]- 5′-methoxybenzoic acid |
| 51 | | 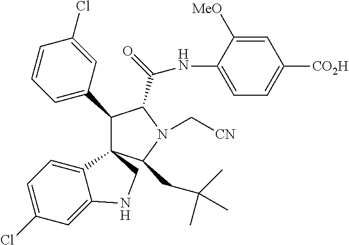 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC#N] | 4-((2′S,3S,4′R,5′R)-6-chloro-4′-(3- chlorophenyl)-1′-(cyanomethyl)-2′- neopentylspiro[indoline-3,3′-pyrrolidine]- 5′-methoxybenzoic acid |
| 52 | | 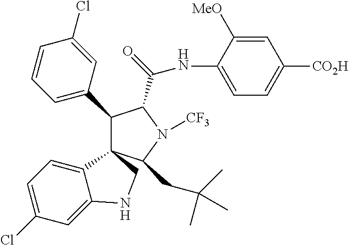 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1C(F)(F)F] | 4-((2′S,3S,4′R,5′R)-6-chloro-4′-(3- chlorophenyl)-2′-neopentyl-1′- (trifluoromethyl)spiro[indoline-3,3′- pyrrolidine]-5′-methoxybenzoic acid |
| 53 | | 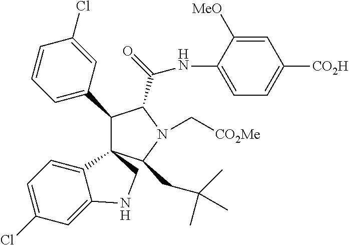 [SMILES: COC(=O)CN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-(2-methoxy-2- oxoethyl)-2′-neopentylspiro[indoline- 3,3′-pyrrolidine]-5′-methoxybenzoic acid |
| 54 | | 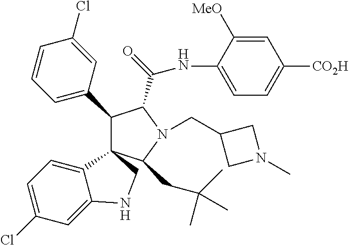 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CN(C)C1] | 4-((2′S,3S,4′R,5′R)-6-chloro-4′-(3- chlorophenyl)-1′-((1-methylazetidin-3- yl)methyl)-2′-neopentylspiro[indoline- 3,3′-pyrrolidine]-5′-methoxybenzoic acid |
| 55 | JQ97 | 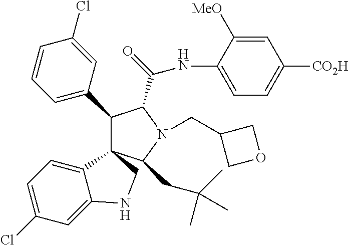 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1COC1] | 4-((2′S,3S,4′R,5′R)-6-chloro-4′-(3- chlorophenyl)-2′-neopentyl-1′-(oxetan-3- ylmethyl)spiro[indoline-3,3′-pyrrolidine]- 5′-methoxybenzoic acid |
| 56 | JP27 | 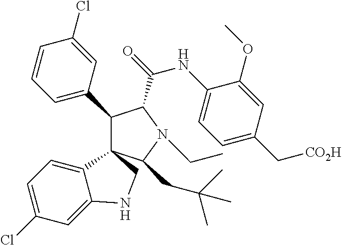 [SMILES: CCN1[C@@H](CC(C)(C)C)[C@@]2(CNc3cc(Cl)ccc32)[C@@H](c2cccc(Cl)c2)[C@@H]1C(=O)Nc1ccc(CC(=O)O)cc1OC] | 2-(4-((2′S,3S,4′R,5′R)-6-chloro-4′-(3- chlorophenyl)-1′-ethyl-2′- neopentylspiro[indoline-3,3′-pyrrolidine]- 5′-carboxamido)-3-methoxyphenyl) acetic acid |
| 57 | JP20 | 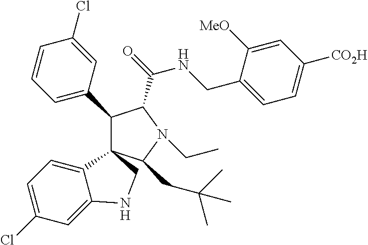 [SMILES: CCN1[C@@H](CC(C)(C)C)[C@@]2(CNc3cc(Cl)ccc32)[C@@H](c2cccc(Cl)c2)[C@@H]1C(=O)NCc1ccc(C(=O)O)cc1OC] | 4-((2′S,3S,4′R,5′R)-6-chloro-4′-(3- chlorophenyl)-1′-ethyl-2′- neopentylspiro[indoline-3,3′-pyrrolidine]- 5′-carboxamido)methyl)-3- methoxybenzoic acid |
| 58 | | 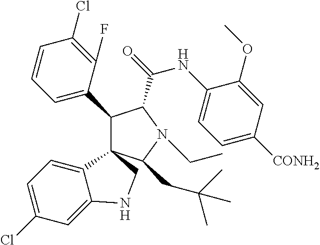 [SMILES: CCN1[C@@H](CC(C)(C)C)[C@@]2(CNc3cc(Cl)ccc32)[C@@H](c2cccc(Cl)c2F)[C@@H]1C(=O)Nc1ccc(C(N)=O)cc1OC] | (2′S,3S,4′S,5′R)-N-(4-carbamoyl-2- methoxyphenyl)-6-chloro-4′-(3-chloro-2- fluorophenyl)-1′-ethyl-2′- neopentylspiro[indoline-3,3′-pyrrolidine]- 5′-carboxamide |
| 59 | | 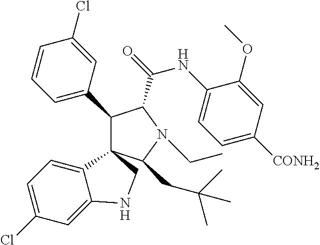 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(N)=O)cc1OC] | (2′S,3S,4′S,5′R)-N-(4-carbamoyl-2- methoxyphenyl)-6-chloro-4′-(3- chlorophenyl)-1′-ethyl-2′- neopentylspiro[indoline-3,3′-pyrrolidine]- 5′-carboxamide |
| 60 | | 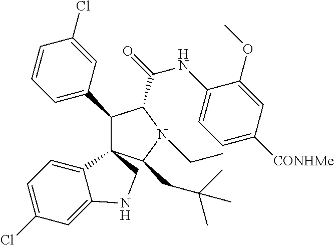 | (2′S,3S,4′R,5′R)-6-chloro-4′-(3- chlorophenyl)-1′-ethyl-N-(2-methoxy-4- (carboxamido)phenyl)-2′- neopentylspiro[indoline-3,3′-pyrrolidine]- 5′-carboxamide |
| 61 | JP23 | 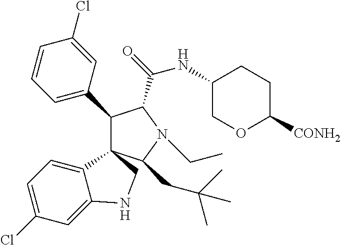 [SMILES: CCN1[C@@H](CC(C)(C)C)[C@@]2(CNc3cc(Cl)ccc32)[C@@H](c2cccc(Cl)c2)[C@@H]1C(=O)N[C@@H]1CC[C@@H](C(N)=O)OC1] | (2′S,3S,4′R,5′R)-N-((3R,6S)-6- carbamoyltetrahydro-2H-pyran-3-yl)-6- chloro-4′-(3-chlorophenyl)-1′-ethyl-2′- neopentylspiro[indoline-3,3′-pyrrolidine]- 5′-carboxamido |
| 62 | | 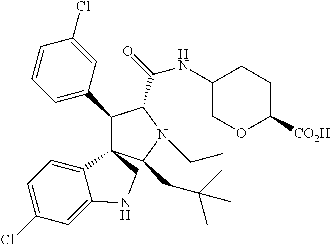 [SMILES: CCN1[C@@H](CC(C)(C)C)[C@@]2(CNc3cc(Cl)ccc32)[C@@H](c2cccc(Cl)c2)[C@@H]1C(=O)NC1CC[C@@H](C(=O)O)OC1] | (2S)-5-((2S,3S,4′R,5′R)-6-chloro-4′-(3- chlorophenyl)-1′-ethyl-2′- neopentylspiro[indoline-3,3′-pyrrolidine]- 5′-carboxamido)tetrahydro-2H-pyran-2- carboxylic acid |
| 63 | | 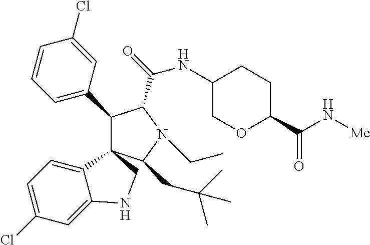 [SMILES: CCN1[C@@H](CC(C)(C)C)[C@@]2(CNc3cc(Cl)ccc32)[C@@H](c2cccc(Cl)c2)[C@@H]1C(=O)NC1CC[C@@H](C(=O)NC)OC1] | (2′S,3S,4′R,5′R)-6-chloro-4′-(3- chlorophenyl)-1′-ethyl-N-((6S)-6- (carboxamido) [symbol] -2H-pyran-3-yl)-2′- neopentylspiro[indoline-3,3′-pyrrolidine]- 5′-carboxamido |
| 64 | | 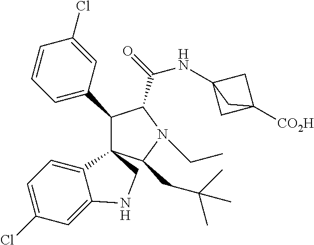 [SMILES: CCN1[C@@H](CC(C)(C)C)[C@@]2(CNc3cc(Cl)ccc32)[C@@H](c2cccc(Cl)c2)[C@@H]1C(=O)NC12CC(C(=O)O)(C1)C2] | 3-((2S′,3S,4′R,5′R)-6-chloro-4′-(3- chlorophenyl)-1′-ethyl-2′- neopentylspiro[indoline-3,3′-pyrrolidine]- 5′-carboxamido)bicyclo[1.1.1]pentane-1- carboxylic acid |
| 65 | JP04 | 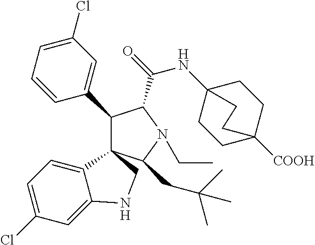 [SMILES: CCN1[C@@H](CC(C)(C)C)[C@@]2(CNc3cc(Cl)ccc32)[C@@H](c2cccc(Cl)c2)[C@@H]1C(=O)NC12CCC(C(=O)O)(CC1)CC2] | 4-((2′S,3S,4′S,5′R)-6-chloro-4′-(3-chloro- phenyl)-1′-ethyl-2′- neopentylspiro[indoline-3,3′-pyrrolidine]- 5′-carboxamido)bicyclo[2.2.2]octane-1- carboxylic acid |
| 66 | JP65 | 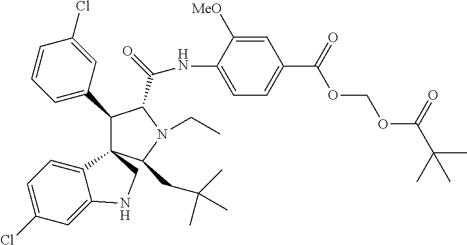 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)OCOC(=O)C(C)(C)C)cc1OC] | (pivaloyloxy)methyl-4-((2′S,3S,4′R,5′R)- 6-chloro-4′-(3-chlorophenyl)-1′-ethyl-2′- neopentylspiro[indoline-3,3′-pyrrolidine]- 5′-carboxamido)-3-methoxybenzoate |
| 67 | JP90 | 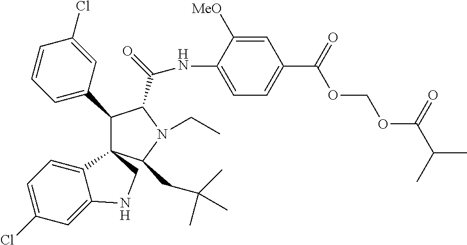 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)OCOC(=O)C(C)C)cc1OC] | (isobutyryloxy)methyl4-((2′S,3S,4′R, 5′R)-6-chloro-4′-(3-chlorophenyl)-1′- ethyl-2′-neopentylspiro[indoline-3,3′- pyrrolidine]-5′-carboxamido)-3- methoxybenzoate |
| 68 | JP17 | 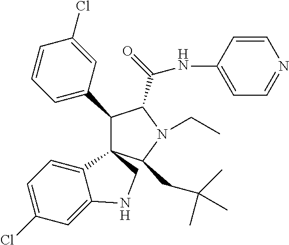 [SMILES: CCN1[C@@H](CC(C)(C)C)[C@@]2(CNc3cc(Cl)ccc32)[C@@H](c2cccc(Cl)c2)[C@@H]1C(=O)Nc1ccncc1] | (2′S,3S,4′R,5′R)-6-chloro-4′-(3- chlorophenyl)-1′-ethyl-2′-neopentyl-N- (pyridin-4-yl)spiro[indoline-3,3′- pyrrolidine]-5′-carboxamide |
| 69 | JP21 | 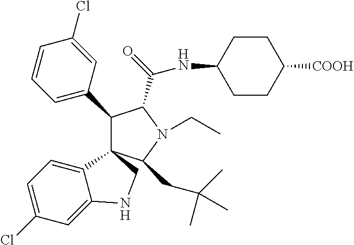 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@H](C(=O)O)CC1] | (1R,4r)-4-((2′S,3S,4′R,5′R)-6-chloro-4′- (3-chlorophenyl)-1′-ethyl-2′- neopentylspiro[indoline-3,3′-pyrrolidine]- 5′-carboxamido)cyclohexane-1- carboxylic acid |
| 70 | JP26 | 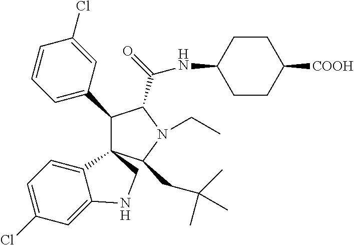 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@@H](C(=O)O)CC1] | (1S,4s)-4-((2′S,3S,4′R,5′R)-6-chloro-4′- (3-chlorophenyl)-1′-ethyl-2′- neopentylspiro[indoline-3,3′-pyrrolidine]- 5′-carboxamido)cyclohexane-1- carboxylic acid |
| 71 | JP09 | 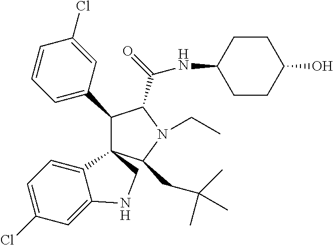 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@H](O)CC1] | (2′S,3S,4′R,5′R)-6-chloro-4′-(3- chlorophenyl)-1′-ethyl-N-((1r,4R)-4- hydroxycyclohexyl)-2′- neopentylspiro[indoline-3,3′-pyrrolidine]- 5′-carboxamide |
| 72 | JP33 | 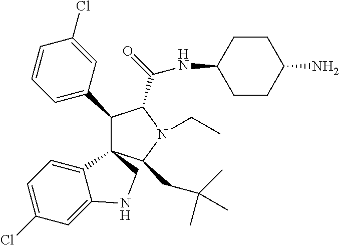 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@H](N)CC1] | (2′S,3S,4′R,5′R)-N-((1r,4R)-4- aminocyclohexyl)-6-chloro-4′-(3- chlorophenyl)-1′-ethyl-2′- neopentylspiro[indoline-3,3′-pyrrolidine]- 5′-carboxamide |
| 73 | JP34 | 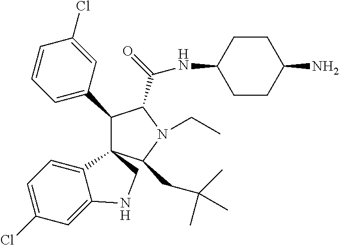 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@@H](N)CC1] | (2′S,3S,4′R,5′R)-N-((1s,4S)-4- aminocyclohexyl)-6-chloro-4′-(3- chlorophenyl)-1′-ethyl-2′- neopentylspiro[indoline-3,3′-pyrrolidine]- 5′-carboxamide |
| 74 | JP08 | 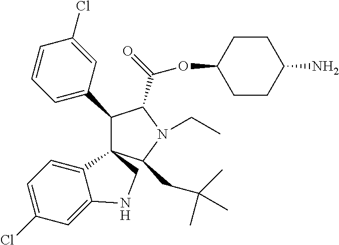 [SMILES: CCN1[C@@H](CC(C)(C)C)[C@@]2(CNc3cc(Cl)ccc32)[C@@H](c2cccc(Cl)c2)[C@@H]1C(=O)O[C@H]1CC[C@H](N)CC1] | (1r,4R)-4- aminocyclohexyl(2′S,3S,4′R,5′R)-6- chloro-4′-(3-chlorophenyl)-1′-ethyl-2′- neopentylspiro[indoline-3,3′-pyrrolidine]- 5′-carboxylate |
| 75 | JQ129 | 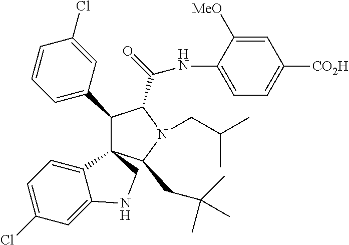 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC(C)C] | 4-((2′S,3S,4′R,5′R)-6-chloro-4′-(3- chlorophenyl)-1′-isobutyl-2′- neopentylspiro[indoline-3,3′-pyrrolidine]- 5′-carboxamido)-3-methoxybenzoic acid |
| 76 | JQ53 | 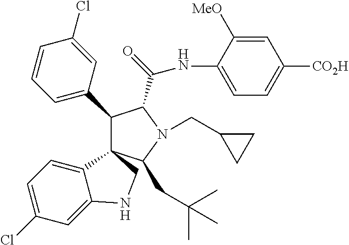 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CC1] | 4-((2′S,3S,4′R,5′R)-6-chloro-4′-(3- chlorophenyl)-1′-(cyclopropylmethyl)-2′- neopentylspiro[indoline-3,3′-pyrrolidine]- 5′-carboxamido]-3-methoxybenzoic acid |
| 77 | JQ148 | 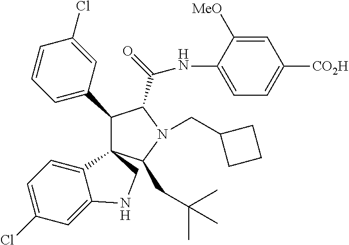 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CCC1] | 4-((2′S,3S,4′R,5′R)-6-chloro-4′-(3- chlorophenyl)-1′-(cyclobutylmethyl)-2′- neopentylspiro[indoline-3,3′-pyrrolidine]- 5′-carboxamido)-3-methoxybenzoic acid |
| 78 | JQ91 | 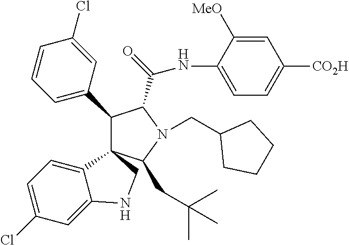 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CCCC1] | 4-((2′S,3S,4′R,5′R)-6-chloro-4′-(3- chlorophenyl)-1′-(cyclopentylmethyl)- 2′-neopentylspiro[indoline-3,3′- pyrrolidine]-5′-carboxamido)-3- methoxybenzoic acid |
| 79 | JQ96 | 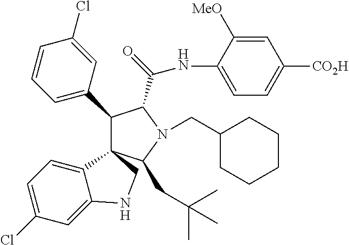 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CCCCC1] | 4-((2′S,3S,4′R,5′R)-6-chloro-4′-(3- chlorophenyl)-1′-(cyclohexylmethyl)-2′- neopentylspiro[indoline-3,3′-pyrrolidine]- 5′-carboxamido)-3-methoxybenzoic acid |
| 80 | JR01 | 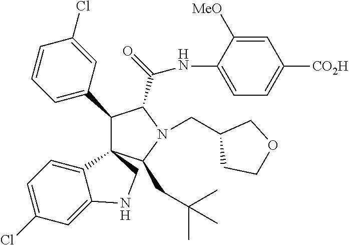 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1C[C@H]1CCOC1] | 4-((2′S,3S,4′R,5′R)-6-chloro-4′-(3- chlorophenyl)-2′-neopentyl-1′-(((R)- tetrahydrofuran-3- yl)methyl)spiro[indoline-3,3′- pyrrolidine]-5′-carboxamido]-3- methoxybenzoic acid |
| 81 | JR02 | 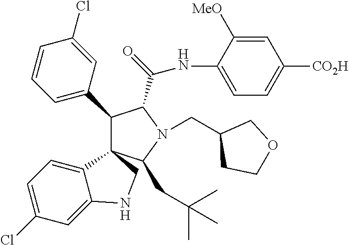 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1C[C@@H]1CCOC1] | 4-((2′S,3S,4′R,5′R)-6-chloro-4′-(3- chlorophenyl)-2′-neopentyl-1′-(((S)- tetrahydrofuran-3- yl)methyl)spiro[indoline-3,3′- pyrrolidine]-5′-carboxamido]-3- methoxybenzoic acid |
| 82 | JQ122 | 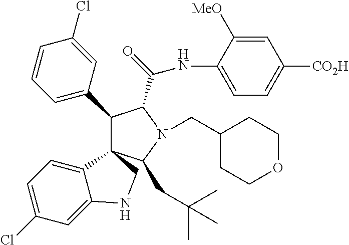 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CCOCC1] | 4-((2′S,3S,4′R,5′R)-6-chloro-4′-(3- chlorophenyl)-2′-neopentyl-1′- ((tetrahydro-2H-pyran-4- yl)methyl)spiro[indoline-3,3′- pyrrolidine]-5′-carboxamido]-3- methoxybenzoic acid |
| 83 | JQ149 | 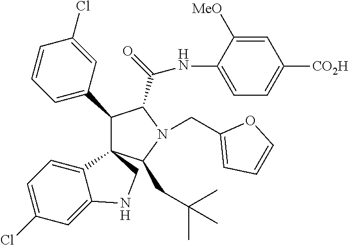 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1Cc1ccco1] | 4-((2′S,3S,4′R,5′R)-6-chloro-4′-(3- chlorophenyl)-1′-(furan-2-ylmethyl)-2′- neopentylspiro[indoline-3,3′-pyrrolidine]- 5′-carboxamido)-3-methoxybenzoic acid |
| 84 | JQ57 | 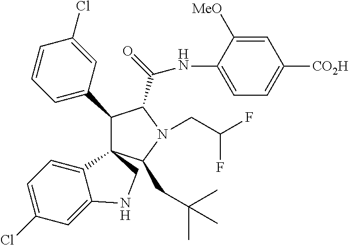 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC(F)F] | 4-((2′S,3S,4′R,5′R)-6-chloro-4′-(3- chlorophenyl)-1′-(2,2-difluoroethyl)-2′- neopentylspiro[indoline-3,3′-pyrrolidine]- 5′-carboxamido)-3-methoxybenzoic acid |
| 85 | JQ158 | 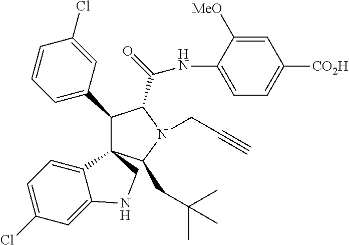 [SMILES: C#CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-2′-neopentyl-1′-(prop- 2-yn-1-yl)spiro[indoline-3,3′- pyrrolidine]-5′-carboxamido)-3- methoxybenzoic acid |
| 86 | JQ120 | 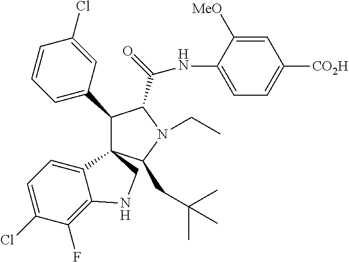 [SMILES: CCN1[C@@H](CC(C)(C)C)[C@@]2(CNc3c2ccc(Cl)c3F)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-ethyl-7-fluoro-2′- neopentylspiro[indoline-3,3′-pyrrolidine]- 5′-carboxamido]-3-methoxybenzoic acid |
| 87 | JQ121 | 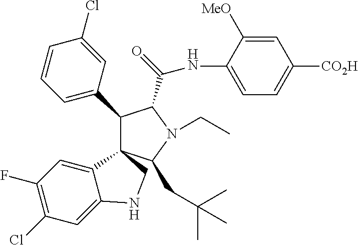 [SMILES: CCN1[C@@H](CC(C)(C)C)[C@@]2(CNc3cc(Cl)c(F)cc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-ethyl-5-fluoro-2′- neopentylspiro[indoline-3,3′-pyrrolidine]- 5′-carboxamido)-3-methoxybenzoic acid |
| 88 | | 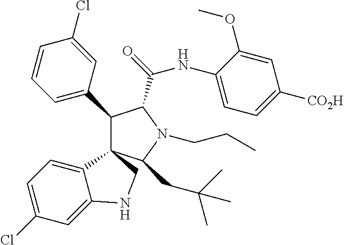 [SMILES: CCCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-2′-neopentyl-1′- propylspiro[indole-3,3′-pyrrolidine]-5′- carboxamido)-3-methoxybenzoic acid |
| 89 | | 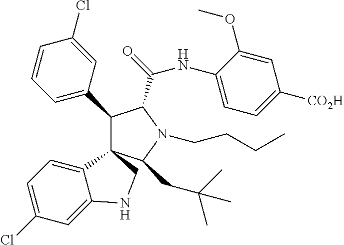 [SMILES: CCCCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-1′-butyl-6-chloro-4′- (3-chlorophenyl)-2′- neopentylspiro[indole-3,3′-pyrrolidine]- 5′-carboxamido)-3-methoxybenzoic acid |
| 90 | | 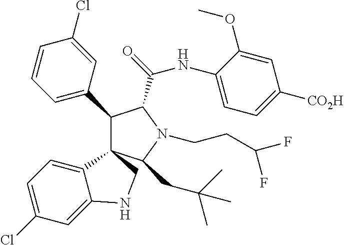 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCC(F)F] | 4-((2′S,3S,4′R,5′R)-6-chloro-4′-(3- chlorophenyl)-1′-(3,3-difluoropropyl)-2′- neopentylspiro[indole-3,3′-pyrrolidine]- 5′-carboxamido)-3-methoxybenzoic acid |
| 91 | | 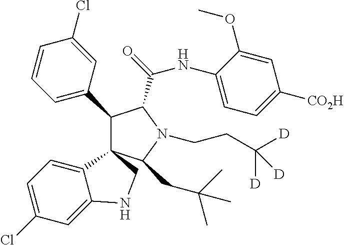 [SMILES: [2H]C([2H])([2H])CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-2′-neopentyl-1′-(propyl- 3,3,3-d3)spiro[indole-3,3′-pyrrolidine]- 5′-carboxamido)-3-methoxybenzoic acid |
| 92 | | 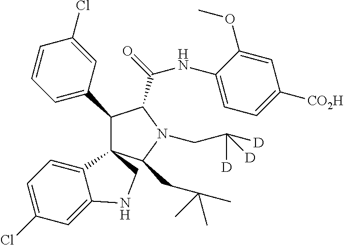 [SMILES: [2H]C([2H])([2H])CN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-(ethyl-2,2,2-d3)-2′- neopentylspiro[indole-3,3′-pyrrolidine]- 5′-carboxamido)-3-methoxybenzoic acid |
| 93 | TC145 | 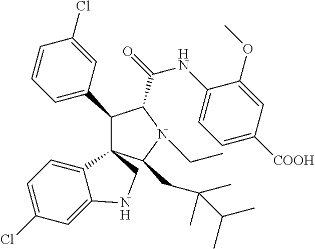 [SMILES: CCN1[C@@H](CC(C)(C)C(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-6-chloro-4′-(3- chlorophenyl)-1′-ethyl-2′-(2,2,3- trimethylbutyl)spiro[indole-3,3′- pyrrolidine]-5′-carboxamido)-3- methoxybenzoic acid |
| 94 | JR62 | 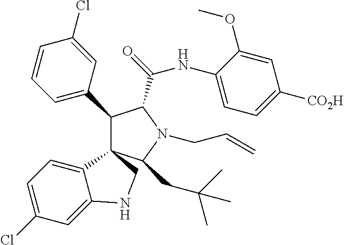 [SMILES: C=CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-1′-allyl-6-chloro-4′- (3-chlorophenyl)-2′-neopentylspiro [indole-3,3′-pyrrolidine]-5′- carboxamido)-3-methoxybenzoic acid |
| 95 | JR55 | 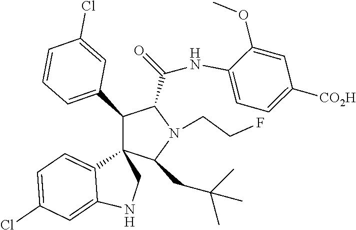 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCF] | 4-((2′S,3S,4′R,5′R)-6-chloro-4′-(3- chlorophenyl)-1-(3-fluoropropyl)-2′- neopentylspiro[indole-3,3′-pyrrolidine]- 5′-carboxamido)-3-methoxybenzoic acid |
| 96 | | 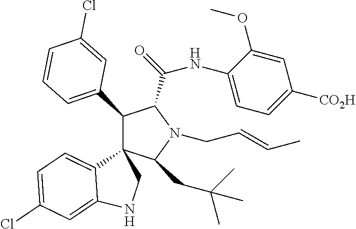 [SMILES: C/C=C/CN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] | 4-((2′S,3S,4′R,5′R)-1′-((E)-but-2-en-1- yl)-6-chloro-4′-(3-chlorophenyl)-2′- neopentylspiro[indole-3,3′-pyrrolidine]- 5′-carboxamido]-3-methoxybenzoic acid |
| 97 | | 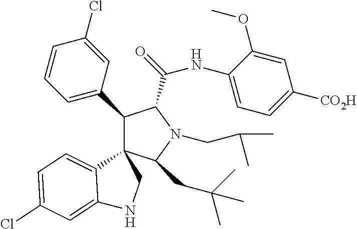 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC(C)C] | 4-((2′S,3S,4′R,5′R)-6-chloro-4′-(3- chlorophenyl)-1′-isobutyl-2′- neopentylspiro[indole-3,3′-pyrrolidine]- 5′-carboxamido)-3-methoxybenzoic acid |

### Example 2: FP Detection of Ki Value of Compounds with MDMX Protein

His-tagged MDMX (14-111, C17S) was expressed in E. coli, purified first by Ni affinity column and then by Superdex75 molecular sieves. The obtained MDMX protein had a purity greater than 95% and a concentration of 12.5 μM. FAM-labeled PDI peptide (FAM-PDI) [Cancer Res 2007, 67, 8810-8817] was used as a fluorescently labeled molecular probe, with a dissociation constant K_{d} of 2.1 nM for the MDMX/FAM-PDI interaction.

96-well plates were purchased from Corning (black, #3694). The multifunction plate reader was a TECAN product, model: SPARK 10 M. Detection buffer: 10 mM Tris (pH 7.5), 200 mM NaCl (Sigma), 0.01% Tween-20, and 0.01% Triton X-100 (Sigma). Millipore-Q purified water was used for the experiments.

First, the test compounds were dissolved in DMSO to prepare 20 mM standard stock solutions. Subsequently, in EP tubes, the standard stock solutions of the test compounds were diluted with DMSO to prepare working sample solutions. The concentration of the prepared working sample solution was 25 times the highest sample concentration required on the test plate (25× test compound solution). The compounds were diluted by a 3-fold gradient in EP tubes for later use.

The 25× test compound solution (4 μL) of compound A with gradient dilution was added to wells B1-D1 to B12-D12 of the 96-well plate, and the 25× test compound solution (4 L) of compound B with gradient dilution was added to wells E1-G1 to E12-G12. Finally, 96 μL of detection buffer containing 5.2 nM FAM-PDI and 62.5 nM MDMX protein was added to each of the above wells.

Wells A 1-A3 served as blank control group: 100 μL of detection buffer was added. Wells A4-A6 served as negative signal reference group: 100 μL of buffer containing only 5 nM fluorescently labeled molecular probe was added. Wells A7-A9 served as positive reference group: 100 μL of mixed solution containing 5 nM fluorescently labeled molecular probe and 60 nM MDMX protein was added.

The reaction plate was covered with aluminum foil, and the 96-well plate was incubated at room temperature on a 96-well plate shaker for 1 h. The fluorescence polarization mP value at Ex485 nm/Em530 nm was then read using the plate reader. The measured mP values were plotted against the compound concentration gradient, and the sample compound concentration corresponding to the midpoint between the maximum and minimum mP values was the IC₅₀ value ([1]₅₀) for the binding of the compound to the protein.

Based on this IC₅₀ value ([I]₅₀), the binding rate constant Ki of the compound to the protein was calculated using the formula:

$${K}_{i} : {K}_{i} = {[ I ]}_{50} / ( {[ L ]}_{50} / {K}_{d} + {[ P ]}_{0} / {K}_{d} + 1 ) .$$

Where [L]₅₀ represents 50% of the fluorescently labeled molecular probe concentration in the above test system; [P]₀ represents the MDMX protein concentration in the above test system, and K_{d} is the dissociation constant of the protein and the fluorescently labeled molecular probe.

Using the above method, the K, values of the Example compounds inhibiting MDMX/p53 interaction are shown in Table 2 below. The experimental data indicate that the compounds have activity in inhibiting MDMX/p53 interaction.

### Example 3: FP Detection of Ki Value of Compounds with MDM2 Protein

His-tagged MDM2 (1-118) was expressed in E. coli, purified first by Ni affinity column and then by Superdex75 molecular sieves. The obtained MDMX protein had a purity greater than 95% and a concentration of 151 μM. FAM-labeled PDI peptide (FAM-PDI) [Cancer Res 2007, 67, 8810-8817] was used as a fluorescently labeled molecular probe, with a dissociation constant K_{d} of 0.7 nM for the MDM2/FAM-PDI interaction.

96-well plates were purchased from Corning (black, #3694). The multifunction plate reader was a TECAN product, model: SPARK 10 M. Detection buffer: 100 mM potassium phosphate (pH 8.0), 100 μg/mL Bovine-r-globulin (Sigma), and 0.01% Triton X-100 (Sigma). Millipore-Q purified water was used for the experiments.

First, the test compounds were dissolved in DMSO to prepare 20 mM standard stock solutions. Subsequently, in EP tubes, the standard stock solutions of the test compounds were diluted with DMSO to prepare working sample solutions. The concentration of the prepared working sample solution was 25 times the highest sample concentration required on the test plate (25× test compound solution). The compounds were diluted by a 3-fold gradient in EP tubes for later use.

The 25× test compound solution (4 μL) of compound A with gradient dilution was added to wells B1-D1 to B12-D12 of the 96-well plate, and the 25× test compound solution (4 μL) of compound B with gradient dilution was added to wells E1-G1 to E12-G12. Finally, 96 μL of detection buffer containing 2.08 nM FAM-PDI and 20.8 nM MDM2 protein was added to each of the above wells.

Wells A 1-A3 served as blank control group: 100 μL of detection buffer was added. Wells A4-A6 served as negative signal reference group: 100 μL of buffer containing only 2 nM fluorescently labeled molecular probe was added. Wells A7-A9 served as positive reference group: 100 μL of mixed solution containing 2 nM fluorescently labeled molecular probe and 20 nM MDM2 protein was added.

The reaction plate was covered with aluminum foil, and the 96-well plate was incubated at room temperature on a 96-well plate shaker for 0.5 h. The fluorescence polarization mP value at Ex485 nm/Em530 nm was then read using the plate reader. The measured mP values were plotted against the compound concentration gradient, and the sample compound concentration corresponding to the midpoint between the maximum and minimum mP values was the IC₅₀ value ([I]₅₀) for the binding of the compound to the protein.

Based on this IC₅₀ value ([I]₅₀), the binding rate constant K, of the compound to the protein was calculated using the formula:

$${K}_{i} : {K}_{i} = {[ I ]}_{50} / ( {[ L ]}_{50} / {K}_{d} + {[ P ]}_{0} / {K}_{d} + 1 ) .$$

Where [L]₅₀ represents 50% of the fluorescently labeled molecular probe concentration in the above test system: [P]₀ represents the MDM2 protein concentration in the above test system, and K_{d} is the dissociation constant of the protein and the fluorescently labeled molecular probe.

Using the above method, the K, values of the Example compounds inhibiting MDM2/p53 interaction are shown in Table 2. The experimental data indicate that the compounds have excellent activity in inhibiting MDM2/p53 interaction.

TABLE 2
Activity of Example Compounds in Inhibiting
MDMX/p53 and MDM2/p53 Interactions

| | | MDMX | MDM2 |
|---|---|---|---|
| | | Inhibitory Activity | Inhibitory Activity |
| No. | Code | Kᵢ (nM) | Kᵢ (nM) |
| 1 | YM157 | ** | **** |
| 2 | YN11 | ** | **** |
| 3 | YN51 | ** | **** |
| 4 | YN52 | ** | **** |
| 5 | JN05-2 | ** | **** |
| 6 | JM151 | ** | **** |
| 7 | JM156 | ** | **** |
| 8 | JN06 | ** | **** |
| 9 | JN22 | ** | **** |
| 10 | JM159 | ** | **** |
| 11 | JN17 | ** | **** |
| 12 | JN01 | ** | **** |
| 13 | JN18 | ** | **** |
| 14 | YN55 | ** | **** |
| 15 | JM157 | ** | **** |
| 16 | JM160 | ** | **** |
| 17 | JN19 | ** | **** |
| 18 | JN122 | ** | **** |
| 33 | LCC153 | ** | **** |
| 34 | LCC63 | ** | **** |
| 35 | LCC131 | ** | **** |
| 37 | TC90 | ** | **** |
| 38 | TC29 | ** | **** |
| 43 | JP16 | ** | **** |
| 49 | JQ44 | ** | **** |
| 55 | JQ97 | ** | **** |
| 56 | JP27 | ** | **** |
| 57 | JP20 | ** | **** |
| 61 | JP23 | * | **** |
| 65 | JP04 | ** | **** |
| 66 | JP65 | * | ** |
| 67 | JP90 | * | *** |
| 68 | JP17 | * | **** |
| 69 | JP21 | ** | **** |
| 70 | JP26 | ** | **** |
| 71 | JP09 | * | **** |
| 72 | JP33 | * | **** |
| 73 | JP34 | * | **** |
| 74 | JP08 | * | **** |
| 75 | JQ129 | ** | **** |
| 76 | JQ53 | ** | **** |
| 77 | JQ148 | ** | **** |
| 78 | JQ91 | ** | **** |
| 79 | JQ96 | ** | **** |
| 80 | JR01 | ** | **** |
| 81 | JR02 | ** | **** |
| 82 | JQ122 | ** | **** |
| 83 | JQ149 | ** | **** |
| 84 | JQ57 | ** | **** |
| 85 | JQ158 | ** | **** |
| 86 | JQ120 | ** | **** |
| 87 | JQ121 | ** | **** |
| 93 | TC145 | ** | **** |
| 94 | JR62 | ** | **** |
| 95 | JR55 | ** | **** |

* indicates Ki is between 100,000 nM and 10,000 nM;
** indicates Ki is between 10,000 nM and 100 nM;
*** indicates Ki is between 100 nM and 10 nM;
**** indicates Ki < 10 nM.

The experimental results show that the compounds have affinity for binding to MDM2 protein and MDM4 protein. Some compounds exhibit particularly strong affinity, with binding constant Ki in the nM range.

### Example 4: Determination of the Absolute Stereochemical Configuration of the Compound

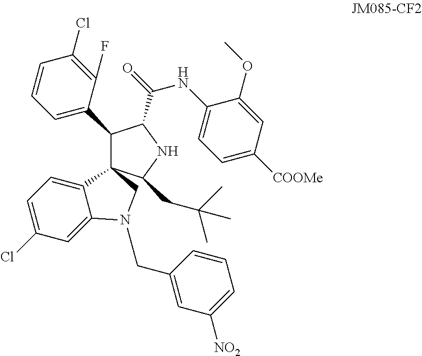 [SMILES: COC(=O)c1ccc(NC(=O)[C@@H]2N[C@@H](CC(C)(C)C)[C@@]3(CN(Cc4cccc([N+](=O)[O-])c4)c4cc(Cl)ccc43)[C@H]2c2cccc(Cl)c2F)c(OC)c1]

### (1) Synthesis of 4-(2′S,3S,4′S,5′R)-6-chloro-4′-(3-chloro-2-fluorophenyl)-2′-neopentyl-1-(3-nitrobenzyl)spiro[indoline-3,3′-pyrrolidine]-5′-carboxamido)-3-methoxybenzoic Acid (JM085-CF2)

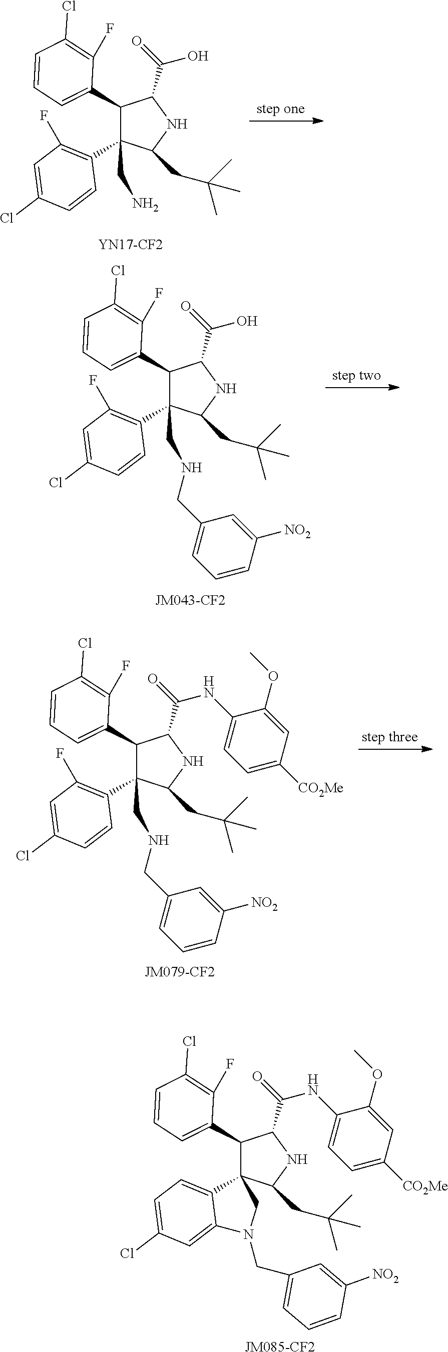

### Step one: Synthesis of (2R,3S,4S,5S)-3-(3-chloro-2-fluorophenyl)-4-(4-chloro-1-fluorophenyl)-5-neopentyl-4-((3-nitrobenzyl)amino)methyl)pyrrolidine-2-carboxylic Acid (JM043-CF2)

YN17-CF2 [239 mg, 0.5 mmol, an intermediate compound in the reaction process of synthetic intermediate 4 (YN22-CF2)] was dissolved in methanol, followed by the addition of m-nitrobenzaldehyde (154 mg, 1.0 mmol), sodium cyanoborohydride (129 mg, 2.0 mmol), and 1 mL of acetic acid. The mixture was stirred overnight at room temperature. After the reaction was complete, HPLC purification afforded 224 mg of the target compound as a trifluoroacetic acid salt, with a yield of 73%.

### Step two: Synthesis of methyl 4-((2R,3S,4S,5S)-3-(3-chloro-2-fluorophenyl)-4-(4-chloro-1-fluorophenyl)-5-neopentyl-4-(((3-nitrobenzyl)amino)methyl)pyrrolidine-2-carboxamido)-3-methoxybenzoate (JM079-CF2)

JM043-CF2 (78 mg, 0.13 mmol) was placed in a flask, dissolved in tetrahydrofuran, and then diisopropylethylamine (84 mg, 0.65 mmol) and diphenylphosphinic chloride (93 mg, 0.39 mmol) were added. After stirring at room temperature for 30 min, methyl 4-amino-3-methoxybenzoate (94 mg, 0.52 mmol) was added and the reacted overnight at room temperature. Water was added, and the mixture was extracted with dichloromethane. The organic phase was concentrated by rotary evaporation and purified by normal-phase column chromatography to obtain the crude product of JM079-CF2, which was used directly in the next step.

### Step Three: Synthesis of 4-((2′S,3S,4′S,5′R)-6-chloro-4′-(3-chloro-2-fluorophenyl)-2′-neopentyl-1-(3-nitrobenzyl)spiro[indoline-3,3′-pyrrolidine]-5′-carboxamido)-3-methoxybenzoic Acid (JM085-CF2)

The crude product of JM079-CF2 was dissolved in N,N-dimethylformamide, potassium carbonate (63 mg, 0.45 mmol) was added, and the mixture was stirred overnight at 100° C. After the reaction was complete, HPLC purification afforded 47 mg of the target compound JM085-CF2 as a trifluoroacetic acid salt, with a two-step yield of 49%. The target compound was dissolved in ethyl acetate, washed twice with saturated sodium bicarbonate solution, and once with saturated brine to obtain the free form of JM085-CF2. ¹H NMR (400 MHz, Methanol-d₄) δ 8.18 (d, J=8.4 Hz, 1H), 8.12 (dd, J=8.3, 2.2 Hz, 1H), 7.92 (d, J=2.1 Hz, 1H), 7.59 (dd, J=8.4, 1.8 Hz, 1H), 7.54-7.47 (m, 2H), 7.46-7.42 (m, 1H), 7.40 (d, J=8.1 Hz, 1H), 7.38-7.29 (m, 2H), 7.11 (t, J=7.9 Hz, 1H), 6.79 (dd, J=8.0, 1.8 Hz, 1H), 6.57 (d, J=1.9 Hz, 1H), 5.17 (d, J=10.6 Hz, 1H), 4.39 (d, J=10.6 Hz, 1H), 4.32 (d, J=15.5 Hz, 1H), 4.26-4.13 (m, 2H), 3.86 (s, 3H), 3.72 (s, 3H), 3.60 (d, J=10.9 Hz, 1H), 3.38 (d, J=10.9 Hz, 1H), 1.81 (dd, J=15.4, 8.3 Hz, 1H), 1.70 (d, J=15.1 Hz, 1H), 0.95 (s, 9H). ESI-MS theoretical calculation for C₃₉H₄₀³⁵Cl₂FN₄O₆ [M+H]⁺=749.2, found: 749.2.

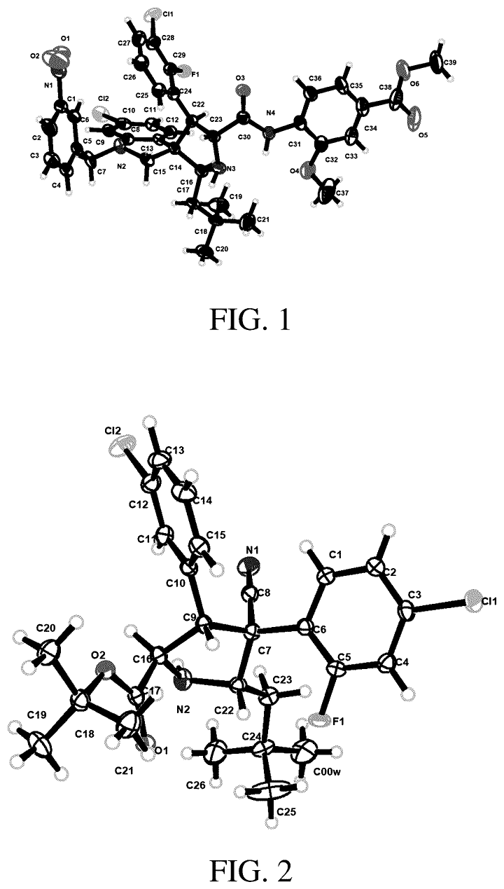

(2) Single Crystal X-ray Diffraction Test: 23 mg of JM085-CF2 was placed in a 10 mL glass sample vial, dissolved in 1.5 mL of dichloromethane, and 1.5 mL of n-hexane was added dropwise. The vial was sealed with aluminum foil and punctured with 3 small holes. After standing at room temperature for 2 days, single crystals formed. The X-ray single crystal diffraction data and results of the compound are shown in Table 3. The crystal data indicates that the absolute stereochemical configuration of JM085-CF2 is as shown in FIG. 1.

TABLE 3
X-ray single crystal diffraction data of test compound

| | | | |
|---|---|---|---|
| Bond | C—C = 0.0081 Å | Wavelength = 0.71073 | |
| precision: | | | |
| Unit cell: | a = 28.9166(19) | b = 28.9166(19) | c = 9.5366(96) |
| | alpha = 90 | beta = 90 | gamma = 90 |
| Temperature: | 150 K | | |

| | Calculated | Reported |
|---|---|---|
| Volume | 7971.7(13) | 7971.7(13) |
| Space group | I 4 | I 4 |
| Holl group | I 4 | I 4 |
| Formula unit | C39 H39 Cl2 F N4 O6 | C39 H39 Cl2 F N4 O6 |
| Total formula | C39 H39 Cl2 F N4 O6 | C39 H39 Cl2 F N4 O6 |
| Relative Molecular | 749.64 | 749.64 |
| weight | | |
| Density, g/cm³ | 1.249 | 1.249 |
| Z | 8 | 8 |
| Mu (mm − 1) | 0.216 | 0.216 |
| F000 | 3136.0 | 3136.0 |
| F000′ | 3139.78 | |
| h, k, lmax | 36, 36, 11 | 36, 36, 11 |
| Nref | 8223[4371] | 7163 |
| Tmin, Tmax | 0.979, 0.989 | 0.650, 0.745 |
| Tmin′ | 0.968 | |

Correction method = #Reported T Limits: Tmin = 0.650, Tmax = 0.745
AbsCorr = MULTI-SCAN

| | |
|---|---|
| Data completeness = 1.64/0.87 | Theta(max) = 26.456 |
| R(reflection) = 0.0664 (4017) | wR2(reflection) = 0.1879 (7163) |
| S = 1.012 | Npar = 478 |

Conclusion: Since the synthetic route of JM085-CF2 is similar to that of intermediate 2 (YM155), the stereochemistry of JM085-CF2 is consistent with the stereochemistry of intermediate 2 (YM155). Intermediate 2 (YM157) is the raw material for synthesizing the final products 1 (YM157), 2 (YN11), 3 (YN51), and 4 (YN52). Therefore, it can be inferred that the stereochemistry of the spiro core of compounds 1, 2, 3, and 4 is completely consistent with the stereochemistry of the spiro core of JM085-CF2.

The diffraction data and results of the X-ray single crystal diffraction test compound are shown in Table 4: The crystal data indicates that the absolute stereochemical configuration of JN110 is as shown in FIG. 2.

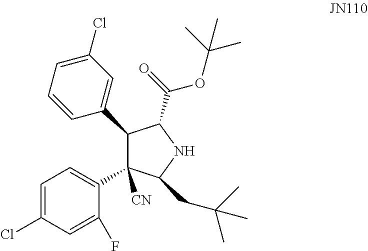 [SMILES: CC(C)(C)C[C@@H]1N[C@@H](C(=O)OC(C)(C)C)[C@H](c2cccc(Cl)c2)[C@@]1(C#N)c1ccc(Cl)cc1F]

TABLE 4
X-ray single crystal diffraction data of test compound

| | | | |
|---|---|---|---|
| Bond | C—C = | Wavelength = | |
| precision: | 0.0043 Å | 0.71073 | |
| Unit cell: | a = 9.7452(7) | b = 13.9525(12) | c = 19.3995(13) |
| | alpha = 90 | beta = 90 | gamma = 90 |
| Temperature: | 100 K | | |

| | Calculated | Reported |
|---|---|---|
| Volume | 2637.8(3) | 2637.7(3) |
| Space group | P 21 21 21 | P 21 21 21 |
| Holl group | P 2ac 2ab | P 2ac 2ab |
| Formula unit | C27 H31 Cl2 F N2 O2 | C27 H31 Cl2 F N2 O2 |
| Total formula | C27 H31 Cl2 F N2 O2 | C27 H31 Cl2 F N2 O2 |
| Relative Molecular | 505.44 | 505.44 |
| weight | | |
| Density, g/cm³ | 1.273 | 1.273 |
| Z | 4 | 4 |
| Mu (mm − 1) | 0.279 | 0.279 |
| F000 | 1064.0 | 1064.0 |
| F000′ | 1065.61 | |
| h, k, lmax | 12, 17, 24 | 36, 36, 11 |
| Nref | 5398[3052] | 7163 |
| Tmin, Tmax | 0.974, 0.986 | 0.637, 0.741 |
| Tmin′ | 0.959 | |

Correction method = #Reported T Limits: Tmin = 0.637, Tmax = 0.741
AbsCorr = MULTI-SCAN

| | |
|---|---|
| Data completeness = 1.76/1.00 | Theta(max) = 26.386 |
| R(reflection) = 0.0419 (4794) | wR2(reflection) = 0.1007 (5373) |
| S = 1.032 | Npar = 326 |

Conclusion: A single enantiomer JN110 can be obtained through the CuOAc and (R)-BINAP catalytic system. Intermediate JN110 can be used to synthesize the target product JN122 with an ee value greater than 95%. During the synthetic route from intermediate JN110 to the final product JN122, the compound does not undergo configurational changes. Therefore, the absolute stereochemical configuration of JN122 should be consistent with that of JN110.

### Example 5: Cell Growth Inhibition Activity Experiment

Cell growth inhibition assay: The test samples were dissolved in 100% dimethyl sulfoxide to prepare a 20 mM compound stock solution. The compounds were diluted with 100% dimethyl sulfoxide to the highest concentration required for the experiment (1 mM or 10 mM).

First, 145 μL of complete cell culture medium was added to wells B1-G1 of a 96-well flat-bottom transparent cell culture plate, and 100 μL of complete medium was added to wells B2-G12. Then, 5 μL of 1 mM and 10 mM compound solution were added to wells B1-D1 and E1-G1 of the 96-well flat-bottom transparent cell culture plate, respectively and serially diluted 3-fold to wells B12-D12 and E12-G12. Finally, 50 μL of the test cell solution was added to each well, with cell densities as follows: approximately 3000 cells per well for HCT116 and RKO, approximately 5000 cells per well for U2-OS, and approximately 8000 cells per well for JEG-3, with a total volume of 150 μL per well. In the experiment, in addition to the test compounds, two control groups were set up: (1) a control group with cells and culture medium but no compound; (2) a group with only complete culture medium, no cells, and no compound. The 96-well plate was incubated in a 37° C. cell incubator containing 5% carbon dioxide for 4 days, then 15 μL of CCK-8 reagent was added to each well, followed by incubation at 37° C. for 2-3 hours. The absorbance at 450 nm was measured using a TECAN microplate reader.

The effect of different concentrations of compounds on cell viability was calculated using the following formula: Cell growth inhibition rate=[absorbance for experimental group−absorbance for complete medium only (no cells, no compound group)]/[absorbance for cells without compound group−absorbance for complete medium only (no cells, no compound group)]×100%. The above data were processed using GraphPad Prism 7.0 software, and the IC₅₀ value was taken as the compound concentration corresponding to 50% cell growth inhibition.

Using the above method, the cellular activities of some compounds and literature compounds YM30, Nutlin-3a, and RG7388 were tested, and the data are shown in Table 5.

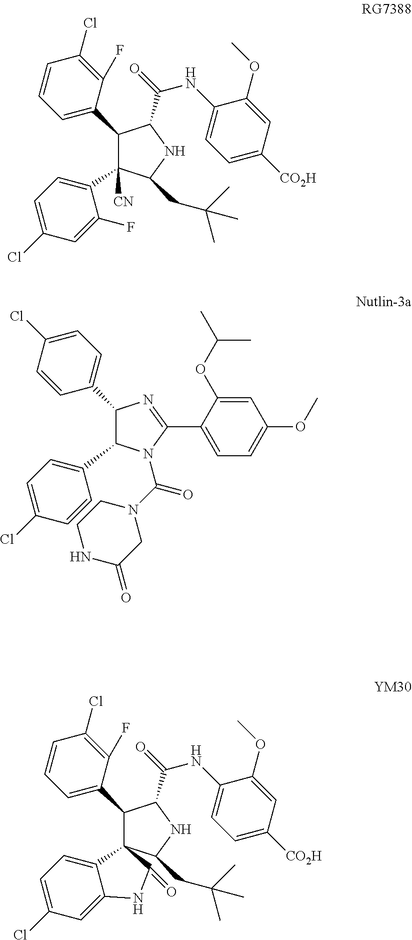 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F ; COc1ccc(C2=N[C@@H](c3ccc(Cl)cc3)[C@@H](c3ccc(Cl)cc3)N2C(=O)N2CCNC(=O)C2)c(OC(C)C)c1 ; COc1cc(C(=O)O)ccc1NC(=O)[C@@H]1N[C@@H](CC(C)(C)C)[C@@]2(C(=O)Nc3cc(Cl)ccc32)[C@H]1c1cccc(Cl)c1F]

TABLE 5
Inhibition activities of compounds on cell proliferation

| | HCT-116 | RKO | U-2 OS |
|---|---|---|---|
| Compound | IC₅₀ (nM) | IC₅₀ (nM) | IC₅₀ (nM) |
| RG7388 | *** | ** | ** |
| Nutlin-3a | * | * | * |
| YM30 | *** | ** | ** |
| 1(YM157) | **** | ** | ** |
| 2(YN11) | **** | ** | ** |
| 3(YN51) | *** | ** | ** |
| 4(YN52) | **** | ** | ** |
| 5(JN05) | ** | ** | ** |
| 6(JM151) | *** | ** | ** |
| 7(JM156) | *** | ** | ** |
| 8(JN06) | *** | ** | ** |
| 9(JN22) | ** | ** | ** |
| 10(JM159) | *** | ** | ** |
| 11(JN17) | *** | ** | ** |
| 12(JN01) | ** | ** | ** |
| 13(JN18) | ** | ** | ** |
| 14(YN55) | **** | *** | *** |
| 15(JM157) | ** | ** | ** |
| 16(JM160) | ** | ** | ** |
| 17(JN19) | ** | ** | ** |
| 18(JN122) | **** | *** | **** |
| 34(LCC63) | *** | *** | n.c. |
| 43(JP16) | *** | ** | n.c. |
| 49(JQ44) | n.c. | **** | *** |
| 55(JQ97) | n.c. | **** | *** |
| 56(JP27) | *** | ** | n.c. |
| 57(JP20) | ** | ** | n.c. |
| 61(JP23) | ** | ** | n.c. |
| 65(JP04) | *** | ** | n.c. |
| 68(JP17) | ** | ** | n.c. |
| 69(JP21) | *** | ** | n.c. |
| 70(JP26) | ** | ** | n.c. |
| 74(JP08) | ** | ** | n.c. |
| 75(JQ129) | n.c. | *** | *** |
| 76(JQ53) | n.c. | *** | ** |
| 77(JQ148) | n.c. | *** | *** |
| 78(JQ91) | n.c. | *** | ** |
| 79(JQ96) | n.c. | ** | ** |
| 80(JR01) | n.c. | **** | **** |
| 81(JR02) | n.c. | **** | **** |
| 82(JQ122) | n.c. | **** | **** |
| 83(JQ149) | n.c. | *** | **** |
| 84(JQ57) | n.c. | **** | *** |
| 85(JQ158) | n.c. | **** | **** |
| 86(JQ120) | n.c. | ** | ** |
| 87(JQ121) | n.c. | *** | *** |
| 94 (JR62) | n.c | ** | n.c |
| 95 (JR55) | n.c | ** | n.c |

* indicates IC₅₀ is between 100,000 nM and 10,000 nM;
** indicates IC₅₀ is between 10,000 nM and 100 nM;
*** indicates IC₅₀ is between 100 nM and 10 nM;
**** indicates IC₅₀ < 10 nM;
n.c. indicates not tested.

The above experimental results indicate that in tumor cell models such as HCT-116, RKO, U2-OS, some Example compounds such as YN55, JN122, JQ44, JQ97, JR01, JR02, JQ122, JQ149, JQ57, JQ158, etc., show more than 10-fold improvement in cell proliferation inhibitory activity compared to RG7388 and YM30, demonstrating significant activity advantages.

### Example 6: Comparison of Solubility of Compounds 2 (YN11), 14 (YN55) and Literature Compounds RG7388, YM30 in 5% DMSO/H₂O

### Compound Solubility Experiment

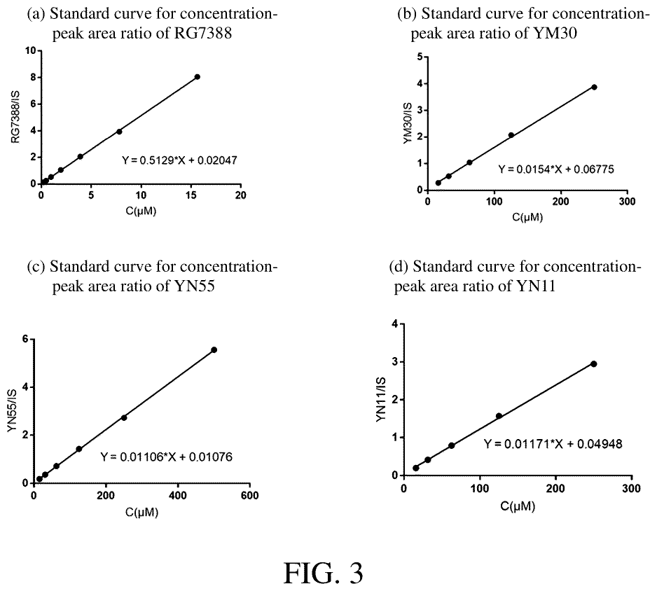

(1) Preparation of internal standard solution: methyl 3-methoxy-4-aminobenzoate was dissolved in acetonitrile:water (1:1) system to prepare internal standard solutions of appropriate concentrations (RG7388-1.4 mM, YM30-5.5 mM, YN55-5.5 mM, YN11-11.2 mM).
(2) Preparation of standard curve: 6-7 concentration gradients were set, and the test samples were diluted in two-fold gradients using acetonitrile:water (1:1) system. For each concentration, 90 μL was taken and added with 10 μL internal standard solution, then 20 μL was injected into UPLC liquid chromatograph. The ratio of the peak area of the test sample to the peak area of the internal standard (IS) was calculated and plotted against the concentration of the test sample to create a standard curve, as shown in FIG. 3.
(3) Concentration testing of compound samples: The test sample was dissolved in 50 μL DMSO, added with 950 μL water, vortexed for 5 min, sonicated for 15 min, centrifuged at 14000 rpm for 30 min. 90 μL of the supernatant was taken and added with 10 μL internal standard solution, then 20 μL was injected into UPLC liquid chromatograph. The ratio of the peak area of the test sample to the peak area of the internal standard was calculated and substituted into the standard curve equation to determine the concentration of the test sample in 5% DMSO/H₂O saturated solution.

Using the above method, the solubility data of RG7388, YM30, 14 (YN55), 2 (YN11) were determined as shown in Table 6.

TABLE 6
test results of the solubility of the compounds

| | | | Saturated |
|---|---|---|---|
| | Ratio of compound | | concen- |
| | peak area to internal | | tration |
| Code | standard peak area | Standard curve equation | (μM) |
| RG7388 | 0.70 | Y = 0.5129X + 0.02047 | 1.3 |
| YM30 | 0.60 | Y = 0.0154X + 0.06775 | 34.9 |
| 14 (YN55) | 0.87 | Y = 0.01106X + 0.01076 | 77.9 |
| 2 (YN11) | 1.0 | Y = 0.01171X + 0.04948 | 80.8 |

The experimental data show that the saturated concentrations of compounds YN55 and YN11 in 5% DMSO/H₂O are significantly higher than those of literature compounds RG7388 and YM30. In the detection system, the solubilities of YN55 and YN11 are more than 40 times that of RG7388 and more than twice that of YM30. The good solubilities of YN55 and YN11 are beneficial for improving the pharmacokinetic and pharmacodynamic properties of the compounds in vivo.

### Example 7: Comparison of Pharmacokinetic Properties of YM34, 2 (YN11) and 14 (YN55)

In vivo pharmacokinetic experiment in mice: ICR mice (species. SPF grade, source: animals transferred from the animal reserve of the experimental institution, Experimental Animal Department of Shanghai Institute of Planned Parenthood Research) were administered the test compounds by intravenous injection or oral gavage. Blood samples were collected at different time points via submandibular vein or other appropriate methods (IV group: 0.083 h, 0.25 h, 0.5 h, 1 h, 2 h, 4 h, 8 h and 24 h after administration; PO group: 0.25 h, 0.5 h, 1 h, 2 h, 4 h, 6 h, 8 h and 24 h after administration, 0.03 ml blood collected at each time point), with K2-EDTA anticoagulation. Plasma was obtained by centrifugation within 1 hour after blood collection (centrifugation conditions: 6800 g, 6 minutes, 2-8° C.). The test samples were stored at −80° C. until analysis. The concentration of the test samples was detected by LC-MS and relevant parameters were calculated. The solvent conditions used in this experiment were: 5% DMSO, 10% Cremophor and 85% PBS. The intravenous dose was 5 mg/kg and the oral dose was 15 mg/kg. Three male mice were used for each of the intravenous and oral experiments.

Using the above method, the pharmacokinetic parameters of YM34, 2 (YN11) and 14 (YN55) in mice were determined, as shown in Tables 7-9.

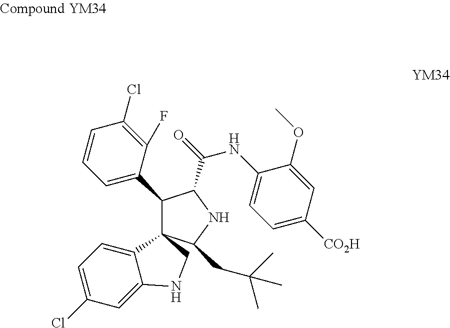 [SMILES: COc1cc(C(=O)O)ccc1NC(=O)[C@@H]1N[C@@H](CC(C)(C)C)[C@@]2(CNc3cc(Cl)ccc32)[C@H]1c1cccc(Cl)c1F]

TABLE 7
Pharmacokinetic parameters of YM34 in ICR mice

| Dose | T_{1/2} | Tₘₐₓ | Cₘₐₓ | AUC₍₀₋ₜ₎ | V_{z} | Cl | F |
|---|---|---|---|---|---|---|---|
| group | h | h | ng/ml | h*ng/mL | L/kg | mL/min/kg | % |
| IV | 3.51 | 0.08 | 18077.70 | 28283.44 | 0.89 | 2.91 | \ |
| 5 mg/kg | | | | | | | |
| PO | 3.18 | 1.00 | 2177.88 | 14423.25 | \ | \ | 17 |
| 15 mg/kg | | | | | | | |

TABLE 8
Pharmacokinetic parameters of YN11 in ICR mice

| | | | | AUC₍₀₋ₜ₎ | V_{z} | Cl | |
|---|---|---|---|---|---|---|---|
| Dose | T_{1/2} | Tₘₐₓ | Cₘₐₓ | h*ng/ | L/ | mL/ | F |
| group | h | h | ng/ml | mL | kg | min/kg | % |
| IV | 8.81 | 0.08 | 21580.93 | 66533.54 | 0.84 | 1.11 | \ |
| 5 mg/kg | | | | | | | |
| PO | 12.61 | 5.33 | 4089.43 | 68548.41 | \ | \ | 34.34 |
| 15 mg/kg | | | | | | | |

TABLE 9
Pharmacokinetic parameters of YN55 in ICR mice

| Dose | T_{1/2} | Tₘₐₓ | Cₘₐₓ | AUC₍₀₋ₜ₎ | V_{z} | Cl | F |
|---|---|---|---|---|---|---|---|
| group | h | h | ng/ml | h*ng/mL | L/kg | mL/min/kg | % |
| IV | 5.35 | 0.08 | 23760.36 | 64251.84 | 0.56 | 1.21 | \ |
| 5 mg/kg | | | | | | | |
| PO | 7.42 | 5.33 | 4151.35 | 58429.33 | \ | \ | 30.31 |
| 15 mg/kg | | | | | | | |

The experimental data show that under the same oral dose conditions, the pharmacokinetic properties of 2 (YN11) and 14 (YN55) are significantly improved compared to YM34:
(1) The in vivo exposure (AUC₍₀₋ₜ₎) of YM34 is 14423.25 h*ng/mL, while the in vivo exposures of YN11 and YN55 are 68548.41 h*ng/mL and 58429.33 h*ng/mL, respectively, showing a 4-5 fold increase.
(2) The oral bioavailability of YM34 is 17%. The oral bioavailabilities of YN11 and YN55 are 34.34% and 30.31%, respectively, which are twice that of YM34.
(3) The maximum plasma concentration (Cₘₐₓ) of YM34 is 2177.88 ng/mL, while the maximum plasma concentrations of YN11 and YN55 are 4089.43 ng/mL and 4151.35 ng/mL, respectively. The maximum plasma concentration parameters of YN11 and YN55 are improved by 1-fold compared to YM34.
(4) The half-life (T_{1/2}) of YM34 is 3.18 hours, while the half-lives of YN11 and YN55 are 12.61 hours and 7.42 hours, respectively. The half-lives of YN1l and YN55 are improved by 2-fold and 1-fold, respectively, compared to YM34.

In summary, the experimental data show that comparing the pharmacokinetic properties of 2 (YN11), 14 (YN55) with YM34, YN11 and YN55 have better oral absorption, better in vivo exposure, higher plasma drug concentration, and longer in vivo retention time, indicating that these drugs (including YN11 and YN55) can bind to drug targets for a longer time and more completely in animals, and their higher concentration in animals is more conducive to achieving better therapeutic effects for diseases.

### Example 8: Verification of the Mechanism of Action of 18 (JN122) by Western-Blot Assay

(1) Study on the Mechanism of Action of 18 (JN122) in HCT116 Cells, HCT116 (Colon Cancer) is a Cell Line with Wild-Type p53 and MDM2 Overexpression.

Western-Blot Assay: HCT-116 cells were seeded in 6-well plates and treated with inhibitors for 24 hours or 48 hours. Cells were collected by centrifugation and lysed with RIPA lysis buffer (Beyotime, product number P0013B) containing PMSF (Beyotime, product number ST506) according to the manufacturer's protocol. The lysates were centrifuged at 13000 g for 30 minutes at 4° C., and the supernatants were transferred to clear EP tubes for subsequent analysis. Proteins were normalized to 20-40 μg/lane and separated on 8% or 12% SDS-PAGE gels. Proteins were transferred to nitrocellulose (NC) membranes and blocked with 5% skim milk dissolved in TBST buffer for 1 hour. Subsequently, samples were incubated with corresponding antibodies at 4° C. with shaking overnight. The NC membranes were washed with TBST buffer for 10 minutes, repeated twice, then incubated with IgG-HRP secondary antibodies at room temperature for 2 hours, followed by washing the membranes three times again. Finally, proteins were visualized using enhanced chemiluminescence detection reagents (Thermo Scientific, product number 34577). The p53 antibody was purchased from Millipore (product number #OP43), MDM4, GAPDH, β-Actin, and BAX antibodies were purchased from Proteintech (product numbers #17914-1-AP, 10494-1-AP, 66009-1-Ig, and 50599-2-Ig, respectively), and p21, MDM2, PARP, PUMA, and CL-PARP antibodies were purchased from Cell Signaling Technology (product numbers 2947, 86934, 9542, 12450, and 5625, respectively).

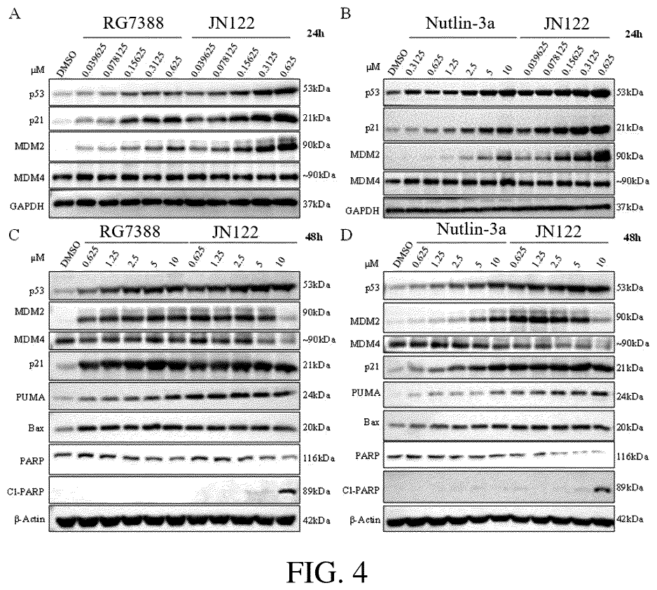

As shown in FIG. 4, consistent with RG7388 and Nutlin-3a, JN122 dose-dependently increased the expression of p53 and its target proteins p21 and MDM2, indicating that the p53/MDM2 interaction was inhibited and p53 stability was increased in HCT116 cells. After treating HCT116 cells with inhibitors for 24 hours, JN122 significantly upregulated the concentrations of p53, p21, and MDM2 at 39.6 nM, while RG7388 and Nutlin-3a achieved similar effects at 156 nM and 2.5 μM, respectively, indicating that JN122 has stronger efficacy than RG7388 and Nutlin-3a. After treating HCT116 cells with inhibitors for 48 hours, JN122, Nutlin-3a, and RG7388 significantly upregulated the expression of PUMA and BAX, indicating activation of the apoptotic pathway. At concentrations of 5-10 μM of JN122, PARP (a biomarker of cell apoptosis) cleavage was observed, and after treating HCT116 cells with inhibitors for 48 hours, the concentrations of MDM2, MDM4, and BAX decreased, possibly due to cell death.

The above experimental data indicate that in HCT-116 cells, JN122 can efficiently activate intracellular p53 function, exhibiting anticancer activity, and its activity is superior to RG7388.

### (2) Investigation of the Mechanism of Action of 18 (JN122) in Various Solid Tumor Cell Lines

RKO (colon cancer), H460 (large cell lung cancer), U2-OS (osteosarcoma), MSTO-211H (mesothelioma), HepG2 (liver cancer), A549 (non-small cell lung cancer), and Hela (cervical cancer) cells are all wild-type p53 cells, while p53 is mutated in SW480 (colon cancer) cells. Although Hela cells are wild-type p53 cells, p53 protein cannot be detected due to continuous degradation by the E6 oncoprotein. The experimental procedure was essentially the same as above, except that HCT-116 cells were replaced with the above cells.

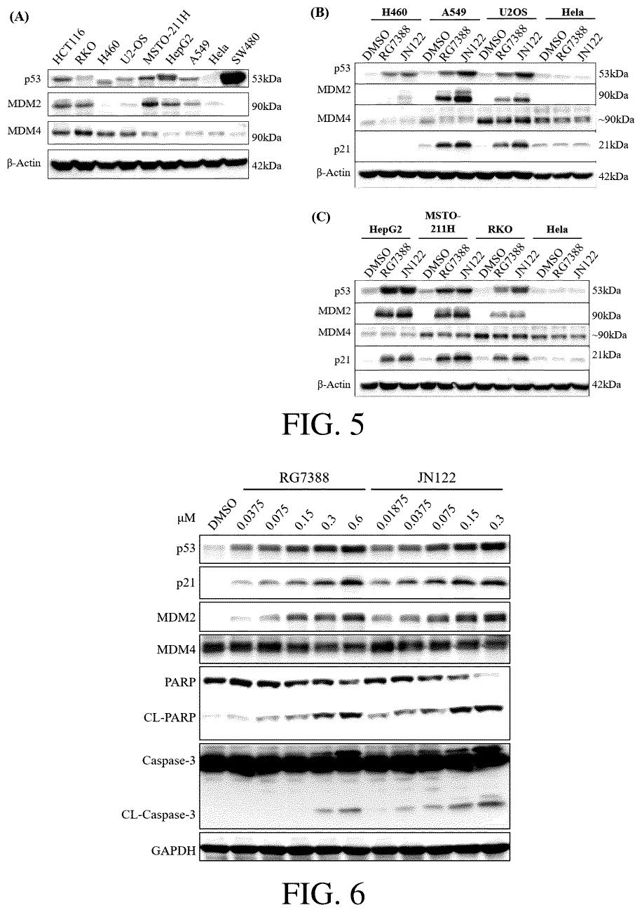

As shown in FIG. 5A, MDM4 expression levels were relatively high in RKO, H460, and U2-OS cells, which are considered MDM4-overexpressing cancer cell lines; MDM2 expression levels were high in MSTO-211H and HepG2, while MDM2 expression level was relatively low in A549 cell lines; stable p53 expression was not detected in Hela cells, and MDM2 and MDM4 expression levels were also low. For comparison purposes, all cells were treated with 0.6 μM JN122, a concentration at which JN122 exhibited strong p53 activation effects in HCT-116 cells.

As shown in FIGS. 5B and 5C, JN122 promoted significant increases in p53, p21, and MDM2 concentrations in H460, A549, U2-OS, HepG2, MSTO-211H, and RKO cells, and the promoting effect of JN122 was superior to RG7388. However, similar phenomena were not observed in Hela cells.

The above experimental data indicate that in six cell lines, H460, A549, U2-OS, HepG2, MSTO-211H, and RKO, JN122 can activate p53 and upregulate the expression levels of its target proteins, exhibiting stronger activity than RG7388. The data also indicate that activation of p53 activity by JN122 is dependent on the ability of tumor cells to express a certain concentration of wild-type p53.

### (3) Investigation of the Mechanism of Action of 18 (JN122) in Hematological Tumor MOLM-13 Cells

The experimental procedure was essentially the same as above, except that HCT-116 cells were replaced with MOLM-13 (human acute myeloid leukemia), a wild-type p53 cell line.

The WB results in FIG. 6 show that JN122 dose-dependently increased the expression of p53 and its target proteins p21 and MDM2, and dose-dependently decreased MDM4 expression, which may be due to increased E3 ubiquitin ligase activity of MDM2. JN122 at 37.5 nM significantly promoted the cleavage of apoptosis markers PARP and Caspase-3, while RG7388 achieved similar effects at 150 nM.

The above experimental data indicate that JN122 can induce p53 activation in MOLM-13 cells and promote cell cycle arrest and cell apoptosis. Additionally, the activation of p53 activity by JN122 in MOLM-13 is approximately four times stronger than that by RG7388.

### Example 9: In Vivo Efficacy of 18 (JN122) in Mouse MOLM-13 Xenograft Model

In vivo anti-tumor efficacy experiment in mice: Female NOD.CB17-PrkdcscidIl2rgtm1/Bcgen (B-NDG) mice were purchased from Biocytogen Pharmaceuticals (Haimen) Co., Ltd., China. Number of animals per group: Vehicle control group, 20 mice; treatment group, 10 mice. Mice were intravenously injected with 200 μL of MOLM-13 cells (2×10₄ cells) suspended in PBS. On day 6, the mice in JN122 25 mg/kg group were administered once daily by gavage for 21 days; the mice in JN122 50 mg/kg group were administered once daily by gavage for 21 days; the mice in JN122 100 mg/kg group were administered once daily by gavage for 21 days; the mice in RG7388 50 mg/kg group were administered once daily by gavage for 21 days; the mice in vehicle control group were administered an equal volume of 5% DMSO+10% Cremophor®EL+85% saline once daily by gavage for 21 days. Mouse survival rates were monitored daily.

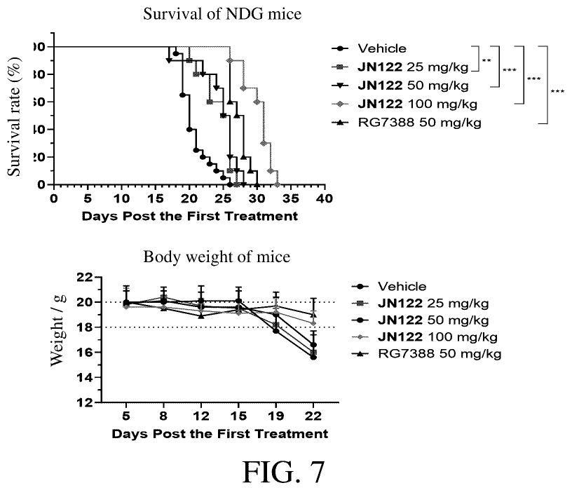

The in vivo efficacy of JN122 was evaluated in a MOLM-13 xenograft mouse model, as shown in FIG. 7. The median survival time of mice in the Vehicle control group was 20 days (survival range 18 to 26 days); once daily oral administration of JN122 at 100 mg/kg extended the median survival time of mice to 31 days (survival range 26 to 33 days, p<0.001); once daily oral administration of JN122 at 25 mg/kg resulted in a median survival time of 25.5 days (survival range 20 to 27 days, p<0.01); once daily oral administration of JN122 at 50 mg/kg resulted in a median survival time of 25.5 days (survival range 17 to 28 days, p<0.001); once daily oral administration of RG7388 at 50 mg/kg resulted in a median survival time of 27.5 days (survival range 26 to 30 days, p<0.001). Therefore, consistent with RG7388, 18(JN122) demonstrated potent anti-leukemic efficacy in the MOLM-13 mouse model, significantly extending the median survival time of tumor-bearing mice.

The above experimental data indicate that JN122 exhibits excellent anti-tumor efficacy in mouse xenograft model of human tumors.

All literatures mentioned in the present invention are incorporated herein by reference as if each literature is cited individually as a reference. Furthermore, it should be understood that after reading the above teachings of the present invention, those skilled in the art may make various modifications or alterations to the present invention, and these equivalent forms also fall within the scope defined by the appended claims.

1. A compound of formula I, an enantiomer, diastereomer, racemate or pharmaceutically acceptable salt thereof, 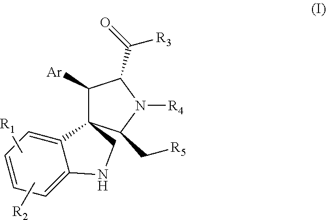
wherein, Ar is a substituted or unsubstituted phenyl, wherein the substituted refers to one or more hydrogen atoms on the phenyl being substituted by groups selected from the group consisting of halogen, deuterium, cyano, hydroxyl, amino, nitro, substituted or unsubstituted C₂-C₄ alkynyl, substituted or unsubstituted C₁-C₆ alkyl, substituted or unsubstituted C₃-C₈ cycloalkyl, substituted or unsubstituted C₁-C₄ alkoxy;
R₁ and R₂ are each independently hydrogen, deuterium, halogen, cyano, substituted or unsubstituted C₂-C₄ alkynyl, substituted or unsubstituted C₁-C₄ alkyl, substituted or unsubstituted C₃-C₈ cycloalkyl, substituted or unsubstituted C₁-C₄ alkoxy;
R₃ is 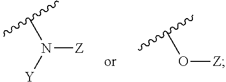
Y and Z are each independently hydrogen, —(CH₂)ₘ-substituted or unsubstituted 6-10 membered aryl, —(CH₂)ₘ-substituted or unsubstituted 5-12 membered heteroaryl, substituted or unsubstituted C₁-C₆ alkyl, —(CH₂)ₘ-substituted or unsubstituted C₃-C₈ cycloalkyl, —(CH₂)ₘ-substituted or unsubstituted 5-13 membered heterocyclyl;
R₄ is substituted or unsubstituted C₁-C₈alkyl, —(CH₂)ₘ-substituted or unsubstituted C₃-C₈ cycloalkyl, —(CH₂)ₘ-substituted or unsubstituted 4-13 membered heterocyclyl, —(CH₂)ₘ-substituted or unsubstituted 6-10 membered aryl, —(CH₂)ₘ-substituted or unsubstituted 5-12 membered heteroaryl, C₂-C₈ alkynyl, or C₂-C₈ alkenyl;
m at each occurrence is independently 0, 1, 2, 3 or 4;
R₅ is substituted or unsubstituted C₁-C₈ alkyl, substituted or unsubstituted C₃-C₈ cycloalkyl, or substituted or unsubstituted 4-12 membered heterocycloalkyl;
unless otherwise defined, each substituted as mentioned above independently refers to one or more hydrogen atoms on the group being substituted with groups selected from the group consisting of halogen, deuterium, cyano, hydroxyl, amino, nitro, C₁-C₄ alkylsulfonyl, C₁-C₄ alkylsulfonamidocarbonyl (C₁-C₄ alkyl-SO₂NHCO—), carboxyl, —CONH₂, C₁-C₄ alkyl, C₃-C₆ cycloalkyl, C₁-C₄ alkoxy, C₂-C₄ alkenyl, C₂-C₄ alkynyl, C₁-C₄ alkylethynyl, mono(C₁-C₄ alkyl)amino, di(C₁-C₄ alkyl)amino, C₁-C₄ alkylcarbonylamino, C₁-C₄ alkyl-COO—, C₁-C₄ alkoxycarbonylamino, C₁-C₄ alkoxycarbonyl, C₁-C₄ alkyl-SO₂—, C₁-C₄ alkyl-S(O₂)—C, —C₁-C₄ alkylene-, carboxyl-substituted C₁-C₄ alkyl, hydroxyl-substituted C₁-C₄ alkyl, C₁-C₄ alkyl-S—, C₂-C₁₀ acyl, C₁-C₄ alkylcarbonyl, C₁-C₄ alkylaminocarbonyl, C₁-C₄ haloalkylaminocarbonyl, 5-12 membered heteroaryl, 5-12 membered heteroarylcarbonyl, C₁-C₄ alkyl-5-12 membered heteroarylcarbonyl, C₁-C₄ alkyl-CO—O—C₁-C₄ alkylene-O—CO—.

2. (canceled)

3. The compound of claim 1, wherein the compound has a structure as shown in formula II: 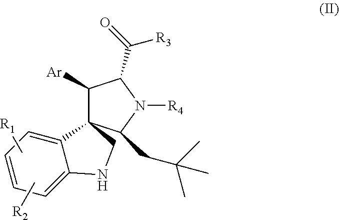
wherein Ar, R₁, R₂, R₃ and R₄ are as defined in claim 1.

4. The compound of claim 1, wherein the compound has a structure as shown in formula IV: 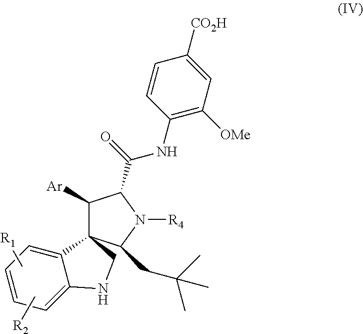
wherein Ar, R₁, R₂ and R₄ are as defined in claim 1.

5. The compound of claim 1, wherein the compound has a structure as shown in formula VI: 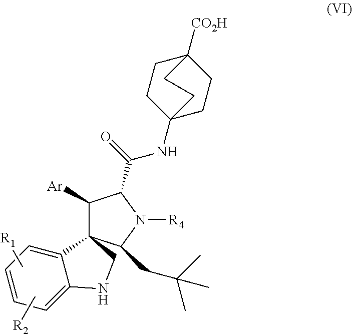
wherein Ar, R₁, R₂ and R₄ are as defined in claim 1.

6. The compound of claim 1, wherein the compound has a structure as shown in formula VII or formula VIII: 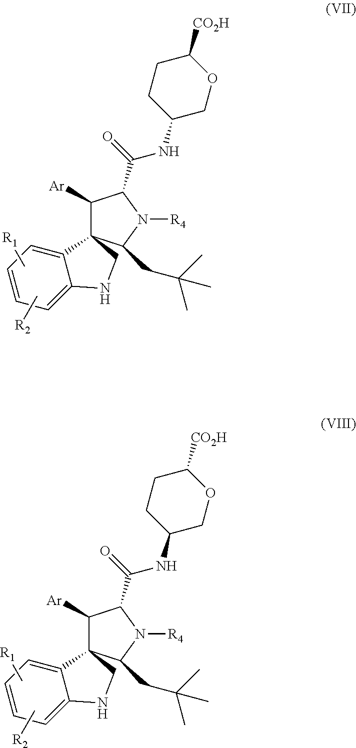
wherein Ar, R₁, R₂ and R₄ are as defined in claim 1.

7. The compound of claim 1, wherein the compound is selected from the group consisting of:

| No. | Code | Structure |
|---|---|---|
| 1 | YM157 | 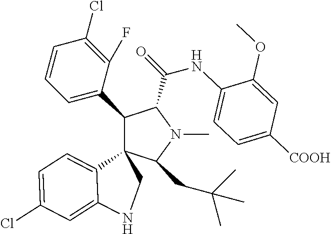 [SMILES: COc1cc(C(=O)O)ccc1NC(=O)[C@H]1[C@H](c2cccc(Cl)c2F)[C@]2(CNc3cc(Cl)ccc32)[C@H](CC(C)(C)C)N1C] |
| 2 | YN11 | 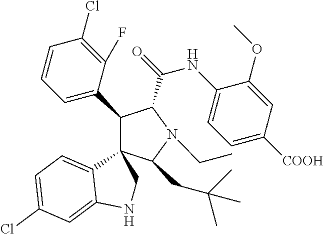 [SMILES: CCN1[C@@H](CC(C)(C)C)[C@@]2(CNc3cc(Cl)ccc32)[C@@H](c2cccc(Cl)c2F)[C@@H]1C(=O)Nc1ccc(C(=O)O)cc1OC] |
| 3 | YN51 | 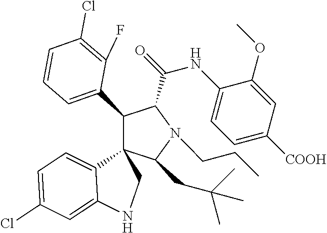 [SMILES: CCCN1[C@@H](CC(C)(C)C)[C@@]2(CNc3cc(Cl)ccc32)[C@@H](c2cccc(Cl)c2F)[C@@H]1C(=O)Nc1ccc(C(=O)O)cc1OC] |
| 4 | YN52 | 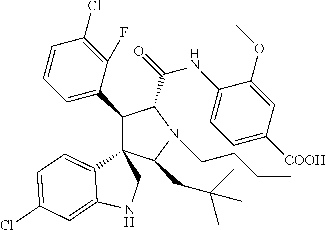 [SMILES: CCCCN1[C@@H](CC(C)(C)C)[C@@]2(CNc3cc(Cl)ccc32)[C@@H](c2cccc(Cl)c2F)[C@@H]1C(=O)Nc1ccc(C(=O)O)cc1OC] |
| 5 | JN05-2 | 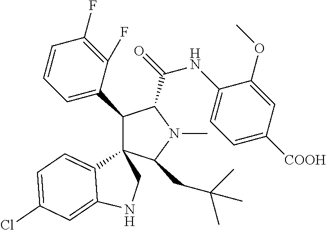 [SMILES: COc1cc(C(=O)O)ccc1NC(=O)[C@H]1[C@H](c2cccc(F)c2F)[C@]2(CNc3cc(Cl)ccc32)[C@H](CC(C)(C)C)N1C] |
| 6 | JM151 | 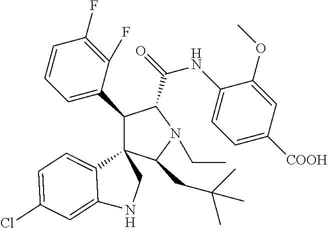 [SMILES: CCN1[C@@H](CC(C)(C)C)[C@@]2(CNc3cc(Cl)ccc32)[C@@H](c2cccc(F)c2F)[C@@H]1C(=O)Nc1ccc(C(=O)O)cc1OC] |
| 7 | JM156 | 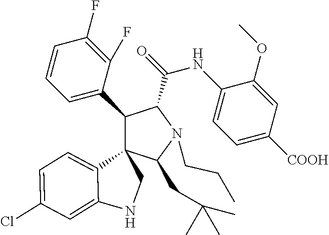 [SMILES: CCCN1[C@@H](CC(C)(C)C)[C@@]2(CNc3cc(Cl)ccc32)[C@@H](c2cccc(F)c2F)[C@@H]1C(=O)Nc1ccc(C(=O)O)cc1OC] |
| 8 | JN06 | 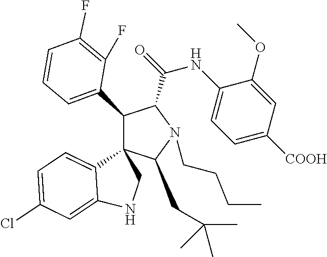 [SMILES: CCCCN1[C@@H](CC(C)(C)C)[C@@]2(CNc3cc(Cl)ccc32)[C@@H](c2cccc(F)c2F)[C@@H]1C(=O)Nc1ccc(C(=O)O)cc1OC] |
| 9 | JN22 | 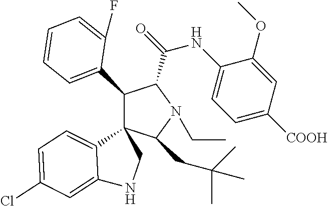 [SMILES: CCN1[C@@H](CC(C)(C)C)[C@@]2(CNc3cc(Cl)ccc32)[C@@H](c2ccccc2F)[C@@H]1C(=O)Nc1ccc(C(=O)O)cc1OC] |
| 10 | JM159 | 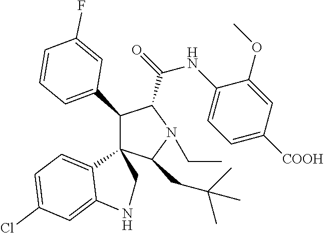 [SMILES: CCN1[C@@H](CC(C)(C)C)[C@@]2(CNc3cc(Cl)ccc32)[C@@H](c2cccc(F)c2)[C@@H]1C(=O)Nc1ccc(C(=O)O)cc1OC] |
| 11 | JN17 | 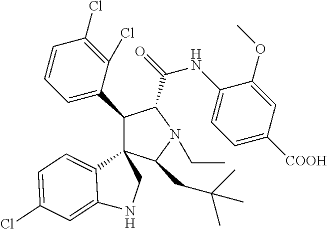 [SMILES: CCN1[C@@H](CC(C)(C)C)[C@@]2(CNc3cc(Cl)ccc32)[C@@H](c2cccc(Cl)c2Cl)[C@@H]1C(=O)Nc1ccc(C(=O)O)cc1OC] |
| 12 | JN01 | 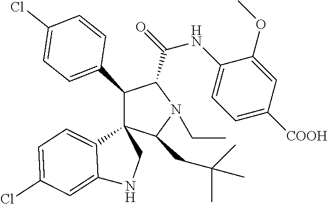 [SMILES: CCN1[C@@H](CC(C)(C)C)[C@@]2(CNc3cc(Cl)ccc32)[C@@H](c2ccc(Cl)cc2)[C@@H]1C(=O)Nc1ccc(C(=O)O)cc1OC] |
| 13 | JN18 | 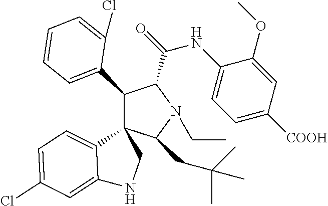 [SMILES: CCN1[C@@H](CC(C)(C)C)[C@@]2(CNc3cc(Cl)ccc32)[C@@H](c2ccccc2Cl)[C@@H]1C(=O)Nc1ccc(C(=O)O)cc1OC] |
| 14 | YN55 | 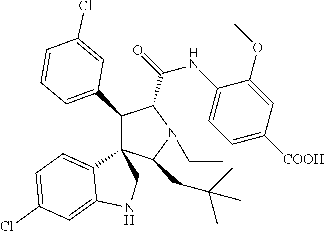 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 15 | JM157 | 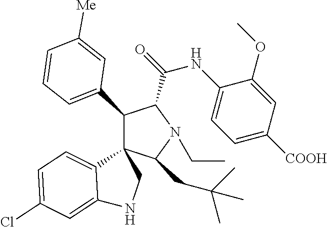 [SMILES: CCN1[C@@H](CC(C)(C)C)[C@@]2(CNc3cc(Cl)ccc32)[C@@H](c2cccc(C)c2)[C@@H]1C(=O)Nc1ccc(C(=O)O)cc1OC] |
| 16 | JM160 | 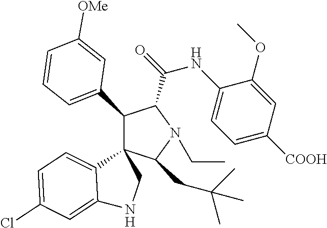 [SMILES: CCN1[C@@H](CC(C)(C)C)[C@@]2(CNc3cc(Cl)ccc32)[C@@H](c2cccc(OC)c2)[C@@H]1C(=O)Nc1ccc(C(=O)O)cc1OC] |
| 17 | JN19 | 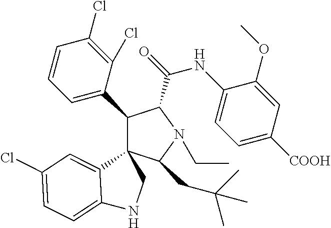 [SMILES: CCN1[C@@H](CC(C)(C)C)[C@@]2(CNc3ccc(Cl)cc32)[C@@H](c2cccc(Cl)c2Cl)[C@@H]1C(=O)Nc1ccc(C(=O)O)cc1OC] |
| 18 | JN122 | 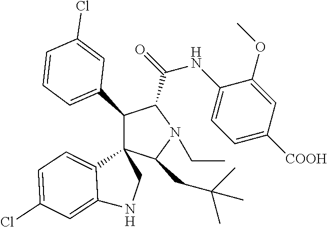 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 19 | | 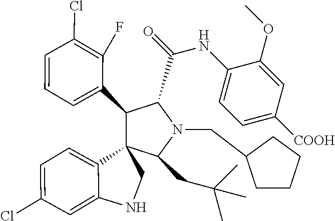 [SMILES: COc1cc(C(=O)O)ccc1NC(=O)[C@H]1[C@H](c2cccc(Cl)c2F)[C@]2(CNc3cc(Cl)ccc32)[C@H](CC(C)(C)C)N1CC1CCCC1] |
| 20 | | 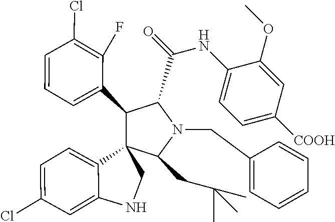 [SMILES: COc1cc(C(=O)O)ccc1NC(=O)[C@H]1[C@H](c2cccc(Cl)c2F)[C@]2(CNc3cc(Cl)ccc32)[C@H](CC(C)(C)C)N1Cc1ccccc1] |
| 21 | | 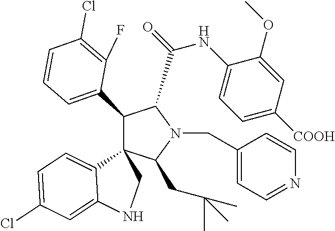 [SMILES: COc1cc(C(=O)O)ccc1NC(=O)[C@H]1[C@H](c2cccc(Cl)c2F)[C@]2(CNc3cc(Cl)ccc32)[C@H](CC(C)(C)C)N1Cc1ccncc1] |
| 22 | | 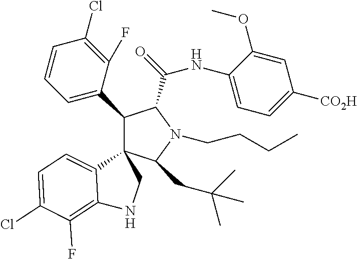 [SMILES: CCCCN1[C@@H](CC(C)(C)C)[C@@]2(CNc3c2ccc(Cl)c3F)[C@@H](c2cccc(Cl)c2F)[C@@H]1C(=O)Nc1ccc(C(=O)O)cc1OC] |
| 23 | | 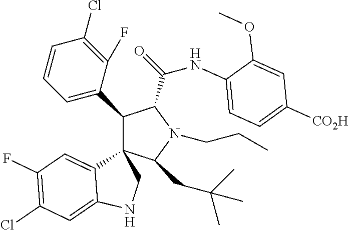 [SMILES: CCCN1[C@@H](CC(C)(C)C)[C@@]2(CNc3cc(Cl)c(F)cc32)[C@@H](c2cccc(Cl)c2F)[C@@H]1C(=O)Nc1ccc(C(=O)O)cc1OC] |
| 24 | | 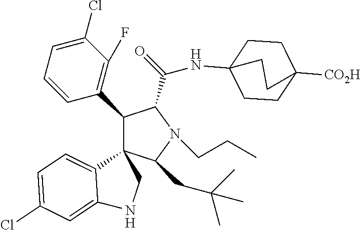 [SMILES: CCCN1[C@@H](CC(C)(C)C)[C@@]2(CNc3cc(Cl)ccc32)[C@@H](c2cccc(Cl)c2F)[C@@H]1C(=O)NC12CCC(C(=O)O)(CC1)CC2] |
| 25 | | 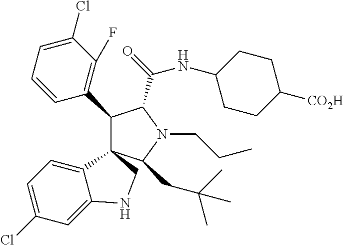 [SMILES: CCCN1[C@@H](CC(C)(C)C)[C@@]2(CNc3cc(Cl)ccc32)[C@@H](c2cccc(Cl)c2F)[C@@H]1C(=O)NC1CCC(C(=O)O)CC1] |
| 26 | | 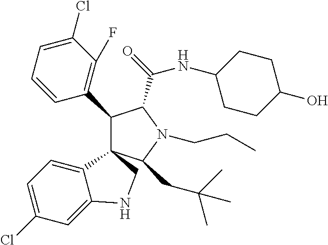 [SMILES: CCCN1[C@@H](CC(C)(C)C)[C@@]2(CNc3cc(Cl)ccc32)[C@@H](c2cccc(Cl)c2F)[C@@H]1C(=O)NC1CCC(O)CC1] |
| 27 | | 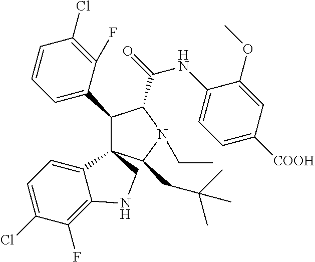 [SMILES: CCN1[C@@H](CC(C)(C)C)[C@@]2(CNc3c2ccc(Cl)c3F)[C@@H](c2cccc(Cl)c2F)[C@@H]1C(=O)Nc1ccc(C(=O)O)cc1OC] |
| 28 | | 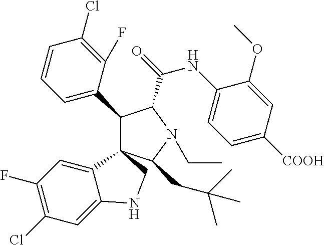 [SMILES: CCN1[C@@H](CC(C)(C)C)[C@@]2(CNc3cc(Cl)c(F)cc32)[C@@H](c2cccc(Cl)c2F)[C@@H]1C(=O)Nc1ccc(C(=O)O)cc1OC] |
| 29 | | 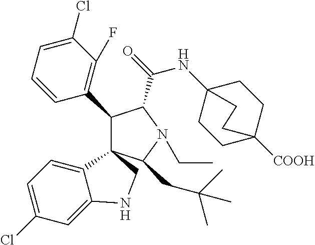 [SMILES: CCN1[C@@H](CC(C)(C)C)[C@@]2(CNc3cc(Cl)ccc32)[C@@H](c2cccc(Cl)c2F)[C@@H]1C(=O)NC12CCC(C(=O)O)(CC1)CC2] |
| 30 | | 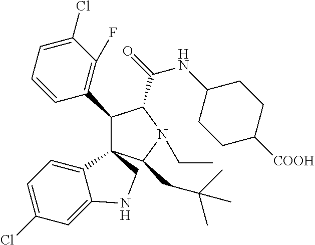 [SMILES: CCN1[C@@H](CC(C)(C)C)[C@@]2(CNc3cc(Cl)ccc32)[C@@H](c2cccc(Cl)c2F)[C@@H]1C(=O)NC1CCC(C(=O)O)CC1] |
| 31 | | 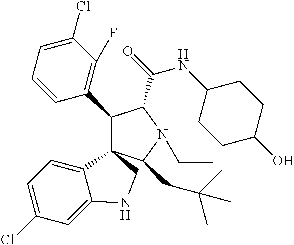 [SMILES: CCN1[C@@H](CC(C)(C)C)[C@@]2(CNc3cc(Cl)ccc32)[C@@H](c2cccc(Cl)c2F)[C@@H]1C(=O)NC1CCC(O)CC1] |
| 32 | | 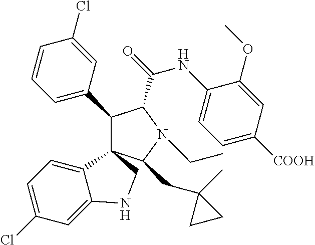 [SMILES: CCN1[C@@H](CC2(C)CC2)[C@@]2(CNc3cc(Cl)ccc32)[C@@H](c2cccc(Cl)c2)[C@@H]1C(=O)Nc1ccc(C(=O)O)cc1OC] |
| 33 | LCC153 | 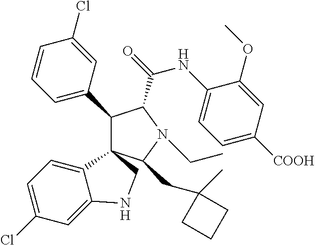 [SMILES: CCN1[C@@H](CC2(C)CCC2)[C@@]2(CNc3cc(Cl)ccc32)[C@@H](c2cccc(Cl)c2)[C@@H]1C(=O)Nc1ccc(C(=O)O)cc1OC] |
| 34 | LCC63 | 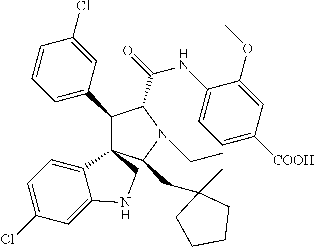 [SMILES: CCN1[C@@H](CC2(C)CCCC2)[C@@]2(CNc3cc(Cl)ccc32)[C@@H](c2cccc(Cl)c2)[C@@H]1C(=O)Nc1ccc(C(=O)O)cc1OC] |
| 35 | LCC131 | 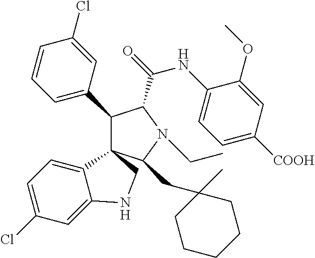 [SMILES: CCN1[C@@H](CC2(C)CCCCC2)[C@@]2(CNc3cc(Cl)ccc32)[C@@H](c2cccc(Cl)c2)[C@@H]1C(=O)Nc1ccc(C(=O)O)cc1OC] |
| 36 | | 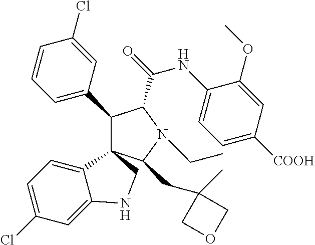 [SMILES: CCN1[C@@H](CC2(C)COC2)[C@@]2(CNc3cc(Cl)ccc32)[C@@H](c2cccc(Cl)c2)[C@@H]1C(=O)Nc1ccc(C(=O)O)cc1OC] |
| 37 | TC90 | 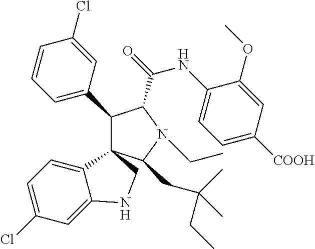 [SMILES: CCN1[C@@H](CC(C)(C)CC)[C@@]2(CNc3cc(Cl)ccc32)[C@@H](c2cccc(Cl)c2)[C@@H]1C(=O)Nc1ccc(C(=O)O)cc1OC] |
| 38 | TC29 | 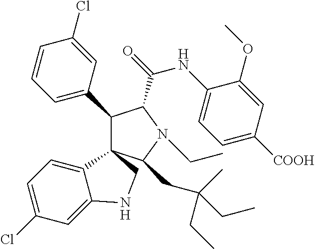 [SMILES: CCN1[C@@H](CC(C)(CC)CC)[C@@]2(CNc3cc(Cl)ccc32)[C@@H](c2cccc(Cl)c2)[C@@H]1C(=O)Nc1ccc(C(=O)O)cc1OC] |
| 39 | | 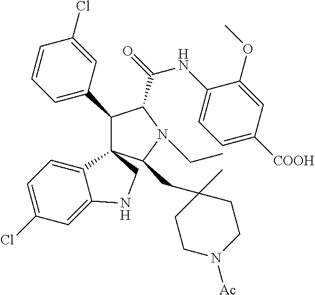 [SMILES: CCN1[C@@H](CC2(C)CCN(C(C)=O)CC2)[C@@]2(CNc3cc(Cl)ccc32)[C@@H](c2cccc(Cl)c2)[C@@H]1C(=O)Nc1ccc(C(=O)O)cc1OC] |
| 40 | | 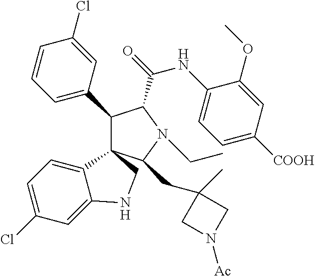 [SMILES: CCN1[C@@H](CC2(C)CN(C(C)=O)C2)[C@@]2(CNc3cc(Cl)ccc32)[C@@H](c2cccc(Cl)c2)[C@@H]1C(=O)Nc1ccc(C(=O)O)cc1OC] |
| 41 | | 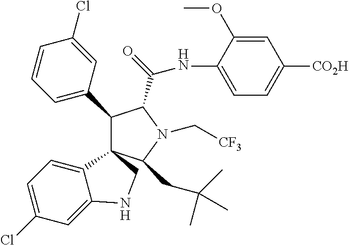 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC(F)(F)F] |
| 42 | | 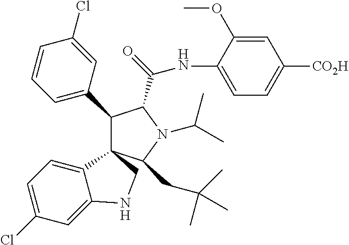 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1C(C)C] |
| 43 | JP16 | 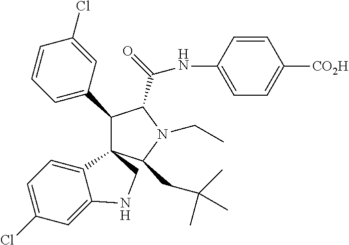 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1] |
| 44 | | 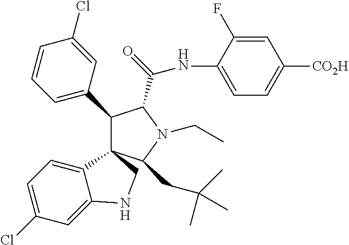 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1F] |
| 45 | | 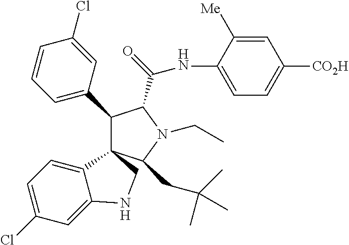 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1C] |
| 46 | | 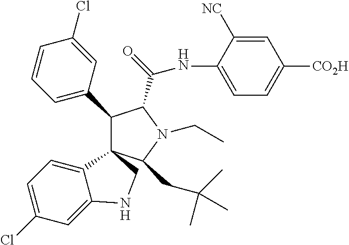 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1C#N] |
| 47 | | 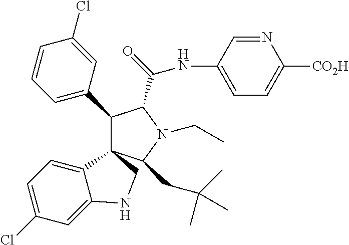 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)O)nc1] |
| 48 | | 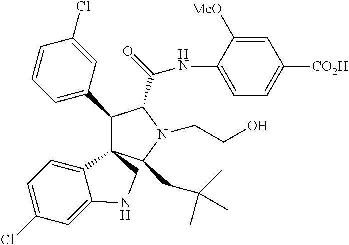 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCO] |
| 49 | JQ44 | 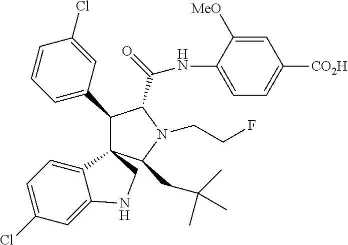 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCF] |
| 50 | | 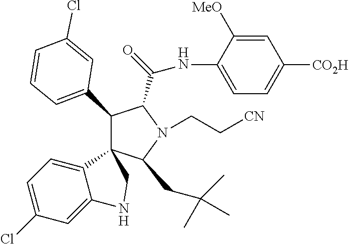 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCC#N] |
| 51 | | 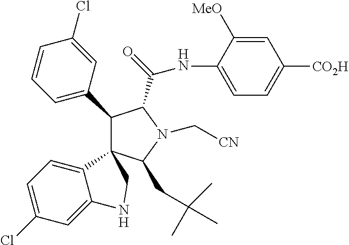 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC#N] |
| 52 | | 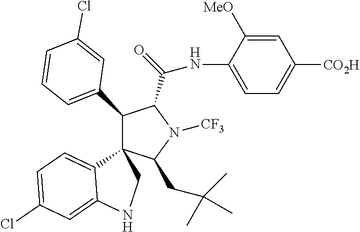 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1C(F)(F)F] |
| 53 | | 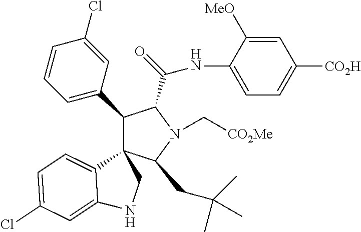 [SMILES: COC(=O)CN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 54 | | 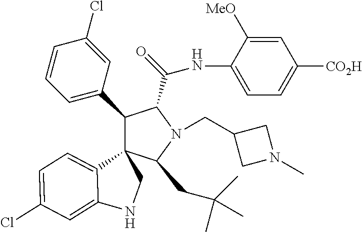 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CN(C)C1] |
| 55 | JQ97 | 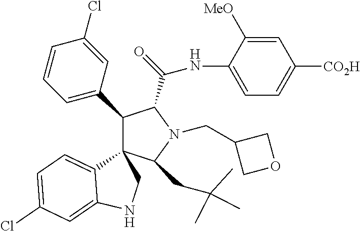 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1COC1] |
| 56 | JP27 | 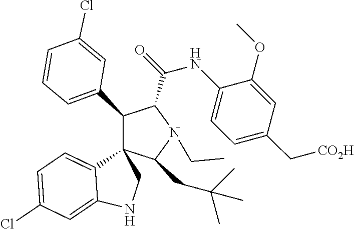 [SMILES: CCN1[C@@H](CC(C)(C)C)[C@@]2(CNc3cc(Cl)ccc32)[C@@H](c2cccc(Cl)c2)[C@@H]1C(=O)Nc1ccc(CC(=O)O)cc1OC] |
| 57 | JP20 | 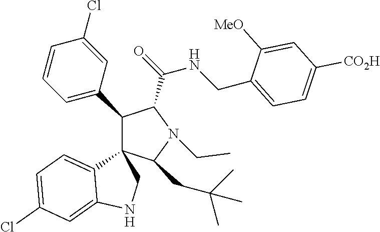 [SMILES: CCN1[C@@H](CC(C)(C)C)[C@@]2(CNc3cc(Cl)ccc32)[C@@H](c2cccc(Cl)c2)[C@@H]1C(=O)NCc1ccc(C(=O)O)cc1OC] |
| 58 | | 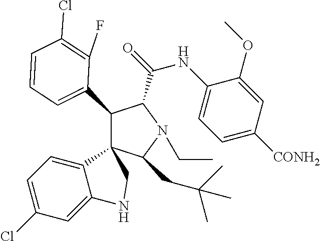 [SMILES: CCN1[C@@H](CC(C)(C)C)[C@@]2(CNc3cc(Cl)ccc32)[C@@H](c2cccc(Cl)c2F)[C@@H]1C(=O)Nc1ccc(C(N)=O)cc1OC] |
| 59 | | 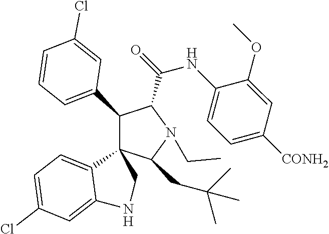 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(N)=O)cc1OC] |
| 60 | | 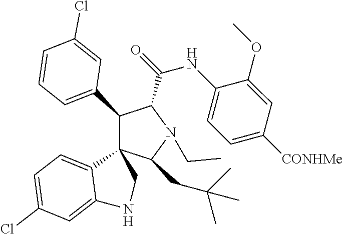 |
| 61 | JP23 | 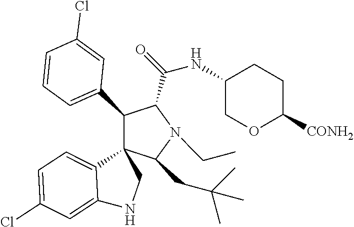 [SMILES: CCN1[C@@H](CC(C)(C)C)[C@@]2(CNc3cc(Cl)ccc32)[C@@H](c2cccc(Cl)c2)[C@@H]1C(=O)N[C@@H]1CC[C@@H](C(N)=O)OC1] |
| 62 | | 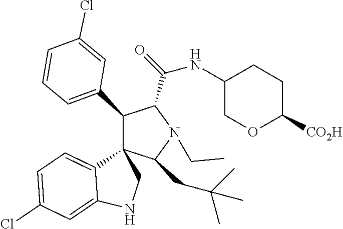 [SMILES: CCN1[C@@H](CC(C)(C)C)[C@@]2(CNc3cc(Cl)ccc32)[C@@H](c2cccc(Cl)c2)[C@@H]1C(=O)NC1CC[C@@H](C(=O)O)OC1] |
| 63 | | 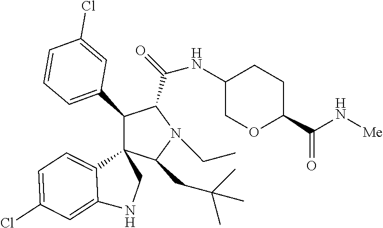 [SMILES: CCN1[C@@H](CC(C)(C)C)[C@@]2(CNc3cc(Cl)ccc32)[C@@H](c2cccc(Cl)c2)[C@@H]1C(=O)NC1CC[C@@H](C(=O)NC)OC1] |
| 64 | | 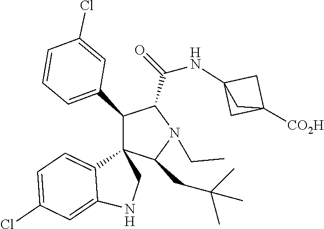 [SMILES: CCN1[C@@H](CC(C)(C)C)[C@@]2(CNc3cc(Cl)ccc32)[C@@H](c2cccc(Cl)c2)[C@@H]1C(=O)NC12CC(C(=O)O)(C1)C2] |
| 65 | JP04 | 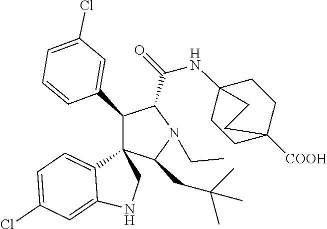 [SMILES: CCN1[C@@H](CC(C)(C)C)[C@@]2(CNc3cc(Cl)ccc32)[C@@H](c2cccc(Cl)c2)[C@@H]1C(=O)NC12CCC(C(=O)O)(CC1)CC2] |
| 66 | JP65 | 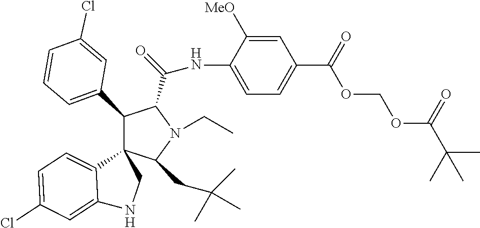 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)OCOC(=O)C(C)(C)C)cc1OC] |
| 67 | JP90 | 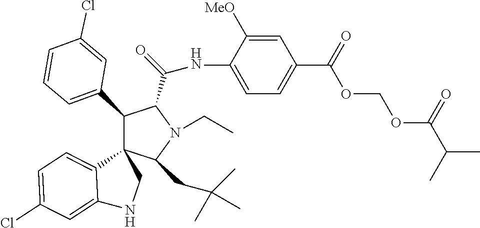 [SMILES: CCN1[C@@H](CC(C)(C)C)[C@@]2(CNc3cc(Cl)ccc32)[C@@H](c2cccc(Cl)c2)[C@@H]1C(=O)Nc1ccc(C(=O)OCOC(=O)C(C)C)cc1OC] |
| 68 | JP17 | 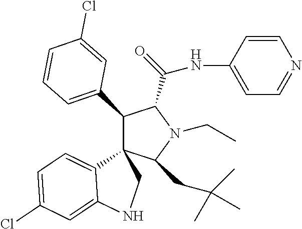 [SMILES: CCN1[C@@H](CC(C)(C)C)[C@@]2(CNc3cc(Cl)ccc32)[C@@H](c2cccc(Cl)c2)[C@@H]1C(=O)Nc1ccncc1] |
| 69 | JP21 | 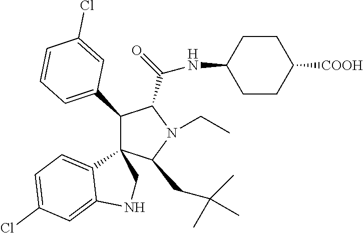 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@H](C(=O)O)CC1] |
| 70 | JP26 | 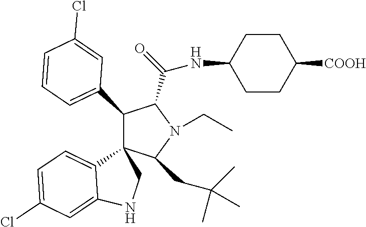 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@@H](C(=O)O)CC1] |
| 71 | JP09 | 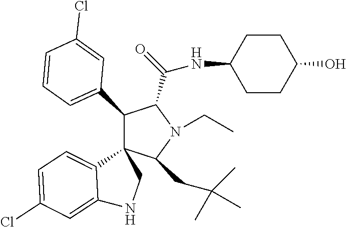 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@H](O)CC1] |
| 72 | JP33 | 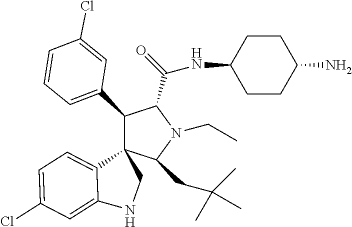 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@H](N)CC1] |
| 73 | JP34 | 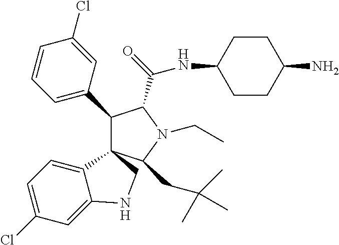 [SMILES: CCN1[C@@H](CC(C)(C)C)[C@@]2(CNc3cc(Cl)ccc32)[C@@H](c2cccc(Cl)c2)[C@@H]1C(=O)N[C@H]1CC[C@@H](N)CC1] |
| 74 | JP08 | 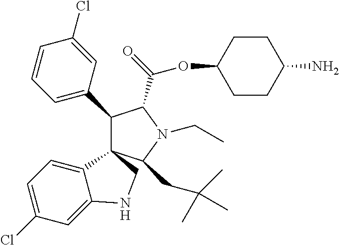 [SMILES: CCN1[C@@H](CC(C)(C)C)[C@@]2(CNc3cc(Cl)ccc32)[C@@H](c2cccc(Cl)c2)[C@@H]1C(=O)O[C@H]1CC[C@H](N)CC1] |
| 75 | JQ129 | 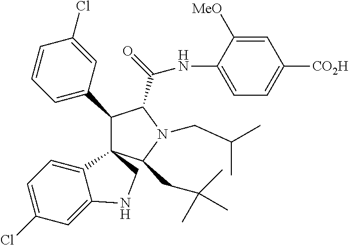 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC(C)C] |
| 76 | JQ53 | 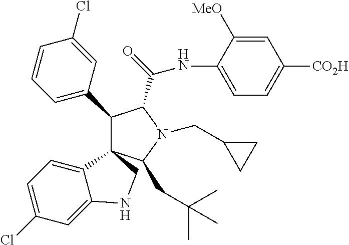 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CC1] |
| 77 | JQ148 | 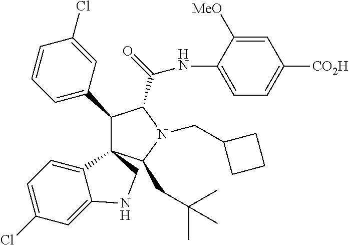 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CCC1] |
| 78 | JQ91 | 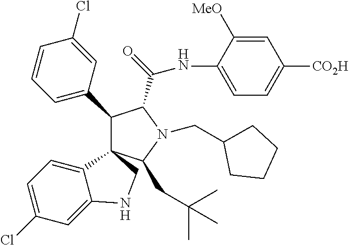 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CCCC1] |
| 79 | JQ96 | 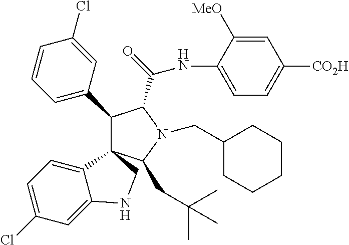 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CCCCC1] |
| 80 | JR01 | 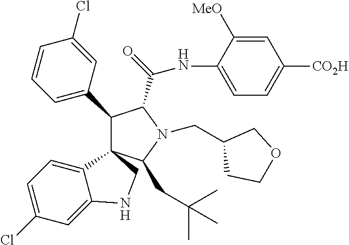 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1C[C@H]1CCOC1] |
| 81 | JR02 | 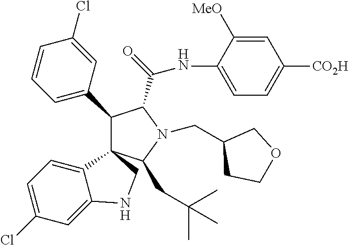 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1C[C@@H]1CCOC1] |
| 82 | JQ122 | 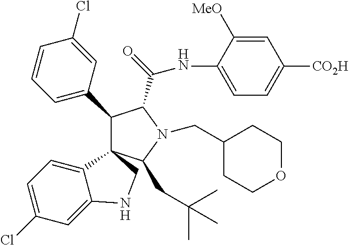 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC1CCOCC1] |
| 83 | JQ149 | 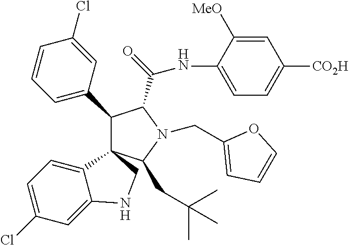 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1Cc1ccco1] |
| 84 | JQ57 | 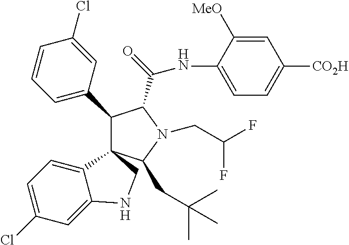 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC(F)F] |
| 85 | JQ158 | 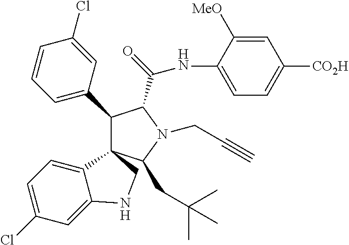 [SMILES: C#CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 86 | JQ120 | 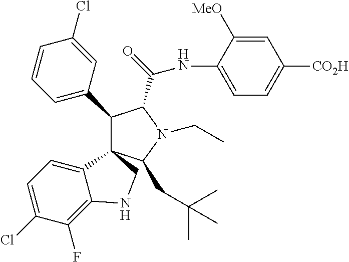 [SMILES: CCN1[C@@H](CC(C)(C)C)[C@@]2(CNc3c2ccc(Cl)c3F)[C@@H](c2cccc(Cl)c2)[C@@H]1C(=O)Nc1ccc(C(=O)O)cc1OC] |
| 87 | JQ121 | 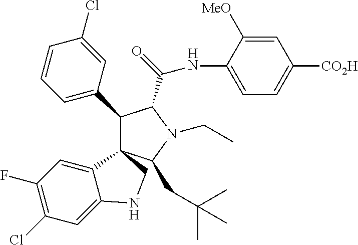 [SMILES: CCN1[C@@H](CC(C)(C)C)[C@@]2(CNc3cc(Cl)c(F)cc32)[C@@H](c2cccc(Cl)c2)[C@@H]1C(=O)Nc1ccc(C(=O)O)cc1OC] |
| 88 | | 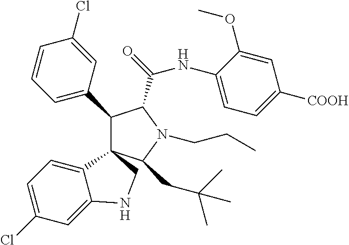 [SMILES: CCCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 89 | | 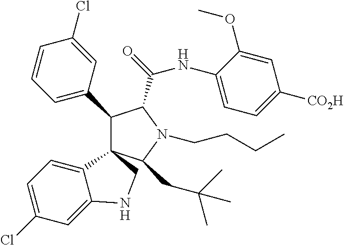 [SMILES: CCCCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 90 | | 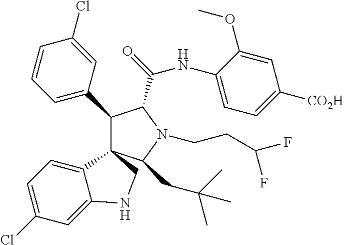 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCC(F)F] |
| 91 | | 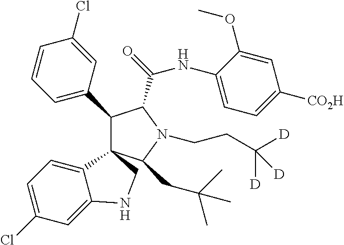 [SMILES: [2H]C([2H])([2H])CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 92 | | 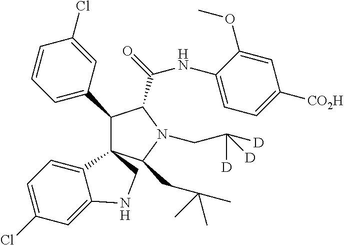 [SMILES: [2H]C([2H])([2H])CN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 93 | TC145 | 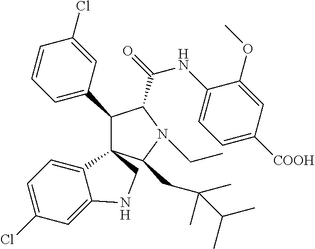 [SMILES: CCN1[C@@H](CC(C)(C)C(C)C)[C@@]2(CNc3cc(Cl)ccc32)[C@@H](c2cccc(Cl)c2)[C@@H]1C(=O)Nc1ccc(C(=O)O)cc1OC] |
| 94 | JR62 | 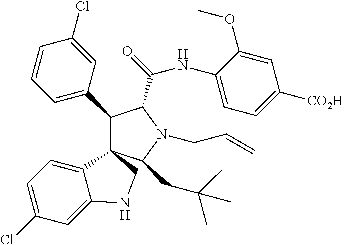 [SMILES: C=CCN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 95 | JR55 | 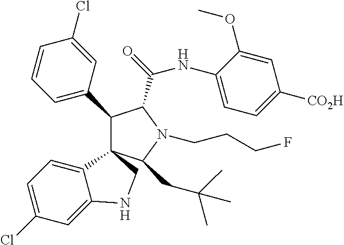 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CCCF] |
| 96 | | 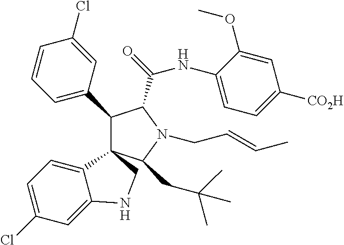 [SMILES: C/C=C/CN1[C@@H](CC(C)(C)C)[C@@]2(CNc3cc(Cl)ccc32)[C@@H](c2cccc(Cl)c2)[C@@H]1C(=O)Nc1ccc(C(=O)O)cc1OC] |
| 97 | | 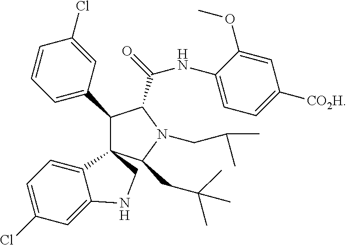 [SMILES: COc1cc(C(=O)O)ccc1NC(=O)[C@H]1[C@H](c2cccc(Cl)c2)[C@]2(CNc3cc(Cl)ccc32)[C@H](CC(C)(C)C)N1CC(C)C] |

8. A pharmaceutical composition, comprising:
one or more of the compounds of claim 1, the enantiomer, the diastereomer, the racemate or the pharmaceutically acceptable salt thereof; and
a pharmaceutically acceptable carrier.

9. A method for blocking MDM2/p53 and/or MDMX/p53 interactions or treating a disease related to the activity or expression level of MDM2 or MDMX protein comprising administering the compound of claim 1, the enantiomer, the diastereomer, the racemate or the pharmaceutically acceptable salt thereof to a subject in need thereof.

10. The method of claim 9, wherein the disease related to the activity or expression level of MDM2 or MDMX protein is selected from the group consisting of glioma, liposarcoma, skin melanoma, squamous cell carcinoma, retinoblastoma, breast cancer, esophageal cancer, lung cancer, ovarian cancer, gastric cancer, bladder cancer, liver cancer, soft tissue sarcoma, chronic lymphocytic leukemia, acute myeloid leukemia, lymphoma, osteosarcoma and colon cancer.

11. A method for preparing the compound of claim 1, wherein the method comprises the following steps: 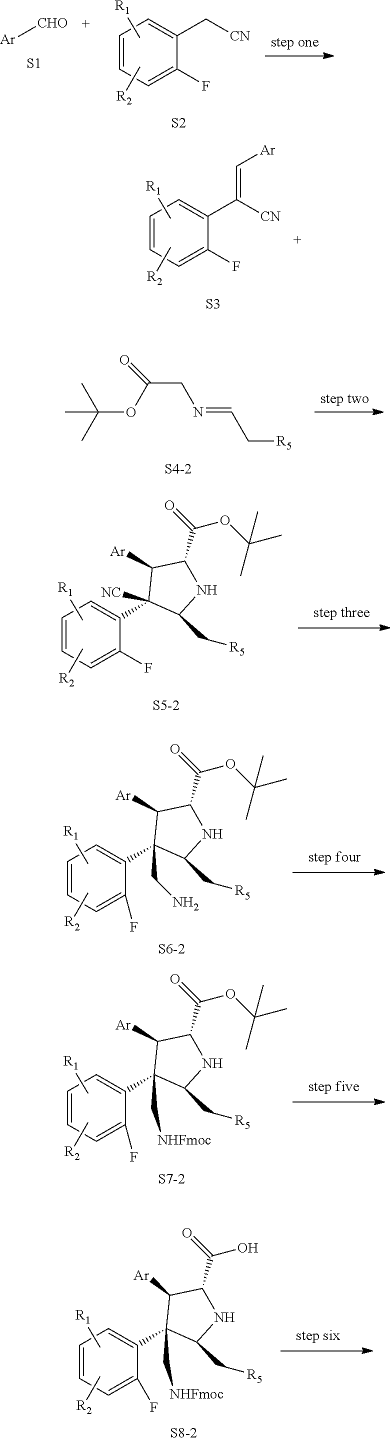 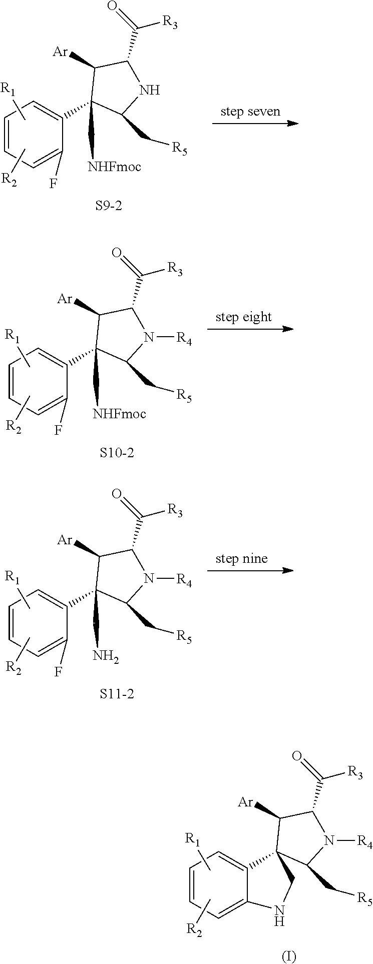
reacting aldehyde S1 with substituted 2-fluorophenylacetonitrile S2 to obtain intermediate S3;
reacting S3 with S4-2 to obtain intermediate S5-2;
subjecting S5-2 to hydrogenation reduction reaction to obtain intermediate S6-2;
reacting S6-2 with FmocCl to obtain intermediate S7-2;
removing the tert-butyl group from S7-2 to obtain intermediate S8-2;
subjecting S8-2 to condensation reaction to obtain intermediate S9-2;
reacting S9-2 with R₄CHO through reductive amination reaction to obtain intermediate S10-2;
deprotecting S10-2 to obtain intermediate S11-2;
reacting S11-2 in the presence of a base to obtain a compound of formula I, or the method comprises the following steps: 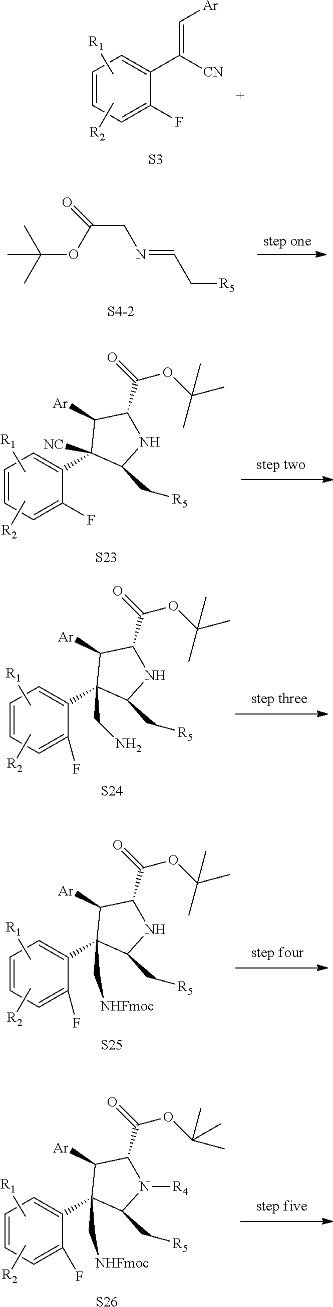 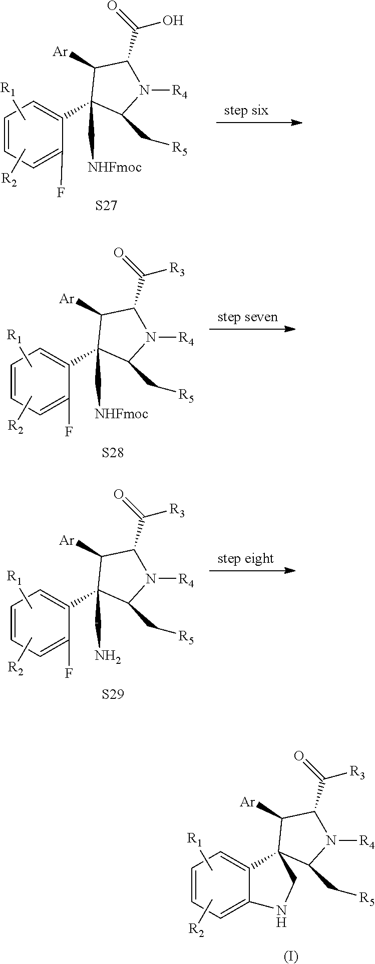
reacting S3 with S4-2 through asymmetric catalytic reaction to obtain optically active chiral intermediate S23;
subjecting S23 to hydrogenation reduction reaction to obtain intermediate S24;
reacting S24 with FmocCl to obtain intermediate S25;
reacting S25 with R₄CHO through reductive amination reaction to obtain intermediate S26;
removing the tert-butyl group from S26 to obtain intermediate S27;
subjecting S27 to condensation reaction to obtain intermediate S28;
deprotecting S28 to obtain intermediate S29;
reacting S29 in the presence of a base to obtain a compound of formula I, which is an optically active chiral compound,
wherein in each formula, Ar, R₁, R₂, R₃, R₄ and R₅ are as defined above,
or the method comprises the following steps: 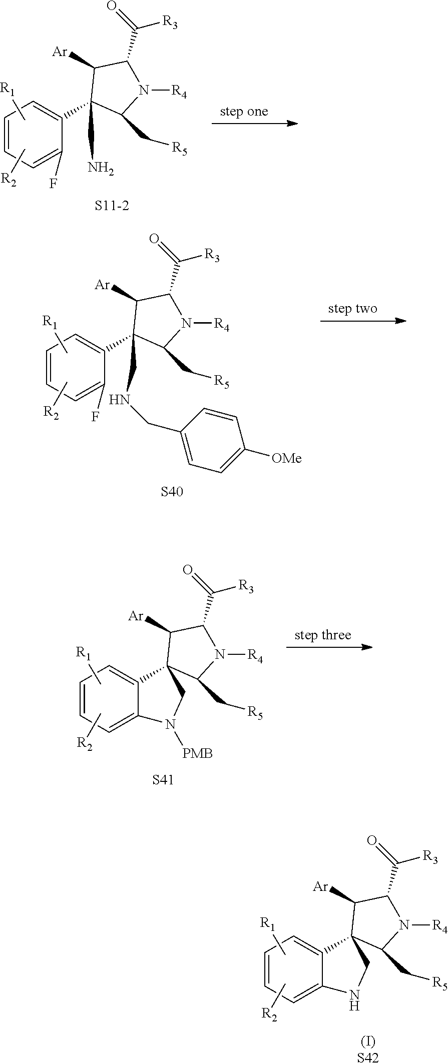
reacting S11-2 through reductive amination reaction to obtain intermediate S40;
reacting S40 in the presence of a base to intermediate S41;
deprotecting S41 to obtain a compound of formula I,
wherein in each formula, Ar, R₁, R₂, R₃, R₄ and R₅ are as defined above;
or the method comprises the following steps: 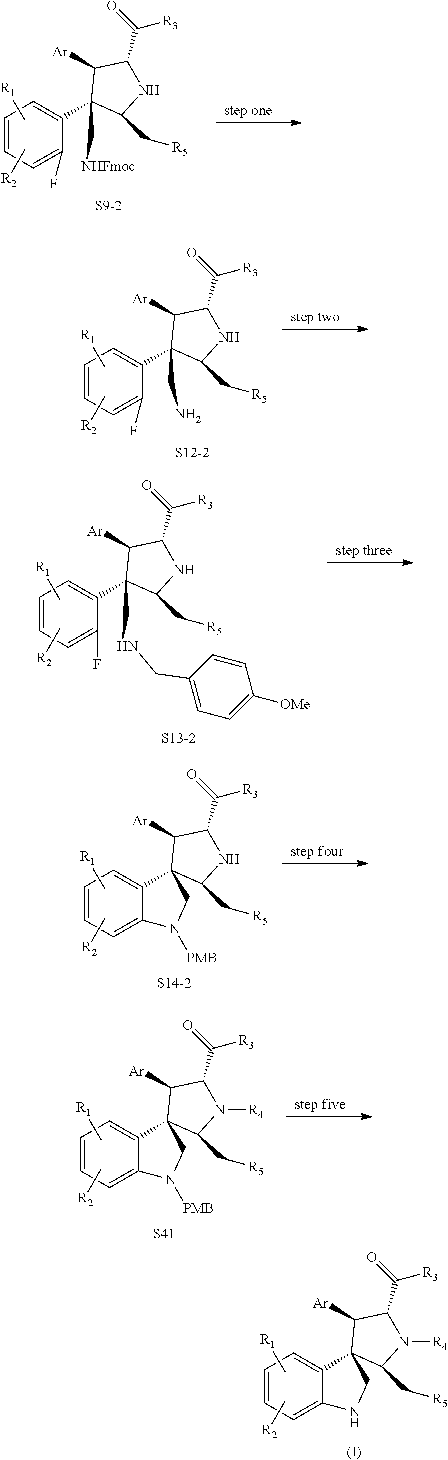
deprotecting S9-2 to obtain intermediate S12-2;
subjecting S12-2 to a reductive amination reaction to obtain intermediate S13-2;
reacting S13-2 in the presence of a base to obtain intermediate S14-2;
reacting S14-2 and R₄CHO having an aldehyde functional group through reductive amination reaction to obtain intermediate S41;
deprotecting S41 to obtain a compound of formula I,
wherein in each formula, Ar, R₁, R₂, R₃, R₄ and R₅ are as defined above.
